(19) United States
(12) Patent Application Publication
(10) Pub. No.: US 2021/0371930 A1
(43) Pub. Date: Dec. 2, 2021

(54) Solute Carrier Family 26 Member 5 (SLC26A5) Variants And Uses Thereof

(71) Applicant: Regeneron Pharmaceuticals, Inc., Tarrytown, NY (US)

(72) Inventors: Kavita Praveen, Tarrytown, NY (US); Giovanni Coppola, Tarrytown, NY (US); Manuel Allen Revez Ferreira, Tarrytown, NY (US); Lauren Gurski, Tarrytown, NY (US); Aris Baras, Tarrytown, NY (US); Meghan Drummond Samuelson, Tarrytown, NY (US); Goncalo Abecasis, Tarrytown, NY (US)

(21) Appl. No.: 17/308,563

(22) Filed: May 5, 2021

Related U.S. Application Data
(60) Provisional application No. 63/022,456, filed on May 9, 2020.

(51) Int. Cl. C12Q 1/6883; G01N 33/53

(52) U.S. Cl. CPC C12Q 1/6883; G01N 33/5308; C12Q 2600/118; G01N 2800/52; C12Q 2600/156; G01N 2800/14; G01N 2800/50; C12Q 2600/106

(57) ABSTRACT

The present disclosure provides methods of treating a subject having hearing loss, methods of identifying a subject having an increased risk of developing hearing loss, and methods of detecting Solute Carrier Family 26 Member 5 (SLC26A5) variant nucleic acid molecules and variant polypeptides.

### REFERENCE TO SEQUENCE LISTING

This application includes a Sequence Listing submitted electronically as a text file named 18923803701SEQ, created on May 1, 2021, with a size of 442 kilobytes. The Sequence Listing is incorporated herein by reference.

### FIELD

The present disclosure relates generally to the treatment of subjects having hearing loss, methods of identifying subjects having an increased risk of developing hearing loss, and methods of detecting SLC26A5 variant nucleic acid molecules and variant polypeptides.

### BACKGROUND

Hearing impairment is the most common sensory defect in humans, affecting approximately 15% of Americans. Studies have shown that approximately 1 in 1000 infants are born with a level of deafness that will affect their linguistic development and speech perception (McHugh and Friedman, Anat. Rec. A Discov. Mol. Cell Evol. Biol., 2006, 288, 370-381). Hearing loss usually has a negative impact on education, social interactions and career opportunities and is, therefore, associated with major social and financial costs. The impact of hearing loss upon an individual can be quite significant (Yoshinaga-Itano, Otolaryngol Clin. North Am., 1999, 32, 1089-1102; Mohr and Feldman, Int. J. Technol. Assess. Health Care, 2000, 16, 1120-35).

The degree of hearing loss varies from mild to profound deafness. It can be stable or the hearing loss can progress with time. The deafness can be present at birth or develop late in life. By far the largest group of people with hearing loss are amongst the elderly, many of whom develop an age-related hearing loss or presbyacusis (Petit, Trends Mol. Med., 2006, 12, 57-64). Both genetic and environmental factors can cause deafness. Genetic factors are the underlying etiology of deafness in the majority of children and young people with hearing loss. Genetic factors play a significant role in age-related hearing loss. Hearing loss can be associated with other clinical features (syndromic hearing loss) or isolated (non-syndromic hearing loss) (Petersen and Willems, Clin. Genet., 2006, 69, 371-392).

SLC26A5 (also known as Prestin) is a motor protein that converts auditory stimuli to length changes in outer hair cells and mediates sound amplification. SLC26A5 is a bidirectional voltage-to-force converter, which can operate at microsecond rates. It uses cytoplasmic anions, such as chloride and bicarbonate, as extrinsic voltage sensors. After binding to a site with millimolar affinity, these anions are translocated across the membrane in response to changes in the transmembrane voltage. They move towards the extracellular surface following hyperpolarization, and towards the cytoplasmic side in response to depolarization. As a consequence, this translocation triggers conformational changes in the protein that ultimately alter its surface area in the plane of the plasma membrane. The area decreases when the anion is near the cytoplasmic face of the membrane (short state), and increases when the ion has crossed the membrane to the outer surface (long state). In doing so, it acts as an incomplete transporter, swinging anions across the membrane, but not allowing these anions to dissociate and escape to the extracellular space.

### SUMMARY

The present disclosure provides methods of identifying a subject having an increased risk for developing hearing loss, the methods comprising: determining or having determined the presence or absence of an SLC26A5 missense variant nucleic acid molecule encoding an SLC26A5 predicted loss-of-function polypeptide in a biological sample obtained from the subject; wherein: when the subject is SLC26A5 reference, then the subject does not have an increased risk for developing hearing loss; and when the subject is heterozygous or homozygous for an SLC26A5 missense variant nucleic acid molecule encoding an SLC26A5 predicted loss-of-function polypeptide, then the subject has an increased risk for developing hearing loss.

The present disclosure also provides methods of treating a subject with a therapeutic agent that treats or inhibits hearing loss, wherein the subject has hearing loss, the methods comprising the steps of: determining whether the subject has an SLC26A5 missense variant nucleic acid molecule encoding an SLC26A5 predicted loss-of-function polypeptide by: obtaining or having obtained a biological sample from the subject; and performing or having performed a sequence analysis on the biological sample to determine if the subject has a genotype comprising the SLC26A5 missense variant nucleic acid molecule encoding an SLC26A5 predicted loss-of-function polypeptide; and administering or continuing to administer the therapeutic agent that treats or inhibits hearing loss in a standard dosage amount to a subject that is SLC26A5 reference; and administering or continuing to administer the therapeutic agent that treats or inhibits hearing loss in an amount that is the same as or greater than a standard dosage amount to a subject that is heterozygous or homozygous for the SLC26A5 missense variant nucleic acid molecule; wherein the presence of a genotype having the SLC26A5 missense variant nucleic acid molecule encoding an SLC26A5 predicted loss-of-function polypeptide indicates the subject has an increased risk of developing hearing loss.

The present disclosure also provides methods of detecting an SLC26A5 variant nucleic acid molecule in a subject comprising assaying a sample obtained from the subject to determine whether a nucleic acid molecule in the sample is: i) a genomic nucleic acid molecule comprising a nucleotide sequence comprising a cytosine at a position corresponding to position 24,774 according to SEQ ID NO:2, or the complement thereof; ii) an mRNA molecule comprising a nucleotide sequence comprising: a cytosine at a position corresponding to position 373 according to SEQ ID NO:13, or the complement thereof; a cytosine at a position corresponding to position 373 according to SEQ ID NO:14, or the complement thereof; a cytosine at a position corresponding to position 373 according to SEQ ID NO:15, or the complement thereof; a cytosine at a position corresponding to position 373 according to SEQ ID NO:16, or the complement thereof; a cytosine at a position corresponding to position 304 according to SEQ ID NO:17, or the complement thereof; a cytosine at a position corresponding to position 304 according to SEQ ID NO:18, or the complement thereof; a cytosine at a position corresponding to position 304 according to SEQ ID NO:19, or the complement thereof; a cytosine at a position corresponding to position 145 according to SEQ ID NO:20, or the complement thereof; a cytosine at a position corresponding to position 145 according to SEQ ID NO:21, or the complement thereof; or a cytosine at a position corresponding to position 205 according to SEQ ID NO:22, or the complement thereof; or iii) a cDNA molecule comprising a nucleotide sequence comprising: a cytosine at a position corresponding to position 373 according to SEQ ID NO:33, or the complement thereof; a cytosine at a position corresponding to position 373 according to SEQ ID NO:34, or the complement thereof; a cytosine at a position corresponding to position 373 according to SEQ ID NO:35, or the complement thereof; a cytosine at a position corresponding to position 373 according to SEQ ID NO:36, or the complement thereof; a cytosine at a position corresponding to position 304 according to SEQ ID NO:37, a cytosine at a position corresponding to position 304 according to SEQ ID NO:38, or the complement thereof; a cytosine at a position corresponding to position 304 according to SEQ ID NO:39, or the complement thereof; a cytosine at a position corresponding to position 145 according to SEQ ID NO:40, or the complement thereof; a cytosine at a position corresponding to position 145 according to SEQ ID NO:41, or the complement thereof; or a cytosine at a position corresponding to position 205 according to SEQ ID NO:42, or the complement thereof.

The present disclosure also provides methods of detecting the presence of an SLC26A5 variant polypeptide comprising performing an assay on a sample obtained from a subject to determine whether an SLC26A5 protein in the sample comprises: a proline at a position corresponding to position 46 according to SEQ ID NO:52, a proline at a position corresponding to position 46 according to SEQ ID NO:53, a proline at a position corresponding to position 46 according to SEQ ID NO:54, a proline at a position corresponding to position 46 according to SEQ ID NO:55, a proline at a position corresponding to position 46 according to SEQ ID NO:56, a proline at a position corresponding to position 46 according to SEQ ID NO:57, a proline at a position corresponding to position 46 according to SEQ ID NO:58, a proline at a position corresponding to position 46 according to SEQ ID NO:59, or a proline at a position corresponding to position 46 according to SEQ ID NO:60.

The present disclosure also provides therapeutic agents that treat or inhibit hearing loss for use in the treatment of hearing loss in a subject having: i) a genomic nucleic acid molecule having a nucleotide sequence encoding an SLC26A5 polypeptide, wherein the nucleotide sequence comprises a cytosine at a position corresponding to position 24,774 according to SEQ ID NO:2, or the complement thereof; ii) an mRNA molecule having a nucleotide sequence encoding an SLC26A5 polypeptide, wherein the nucleotide sequence comprises: a cytosine at a position corresponding to position 373 according to SEQ ID NO:13, or the complement thereof; a cytosine at a position corresponding to position 373 according to SEQ ID NO:14, or the complement thereof; a cytosine at a position corresponding to position 373 according to SEQ ID NO:15, or the complement thereof; a cytosine at a position corresponding to position 373 according to SEQ ID NO:16, or the complement thereof; a cytosine at a position corresponding to position 304 according to SEQ ID NO:17, or the complement thereof; a cytosine at a position corresponding to position 304 according to SEQ ID NO:18, or the complement thereof; a cytosine at a position corresponding to position 304 according to SEQ ID NO:19, or the complement thereof; a cytosine at a position corresponding to position 145 according to SEQ ID NO:20, or the complement thereof; a cytosine at a position corresponding to position 145 according to SEQ ID NO:21, or the complement thereof; or a cytosine at a position corresponding to position 205 according to SEQ ID NO:22, or the complement thereof; or iii) a cDNA molecule having a nucleotide sequence encoding an SLC26A5 polypeptide, wherein the nucleotide sequence comprises: a cytosine at a position corresponding to position 373 according to SEQ ID NO:33, or the complement thereof; a cytosine at a position corresponding to position 373 according to SEQ ID NO:34, or the complement thereof; a cytosine at a position corresponding to position 373 according to SEQ ID NO:35, or the complement thereof; a cytosine at a position corresponding to position 373 according to SEQ ID NO:36, or the complement thereof; a cytosine at a position corresponding to position 304 according to SEQ ID NO:37, a cytosine at a position corresponding to position 304 according to SEQ ID NO:38, or the complement thereof; a cytosine at a position corresponding to position 304 according to SEQ ID NO:39, or the complement thereof; a cytosine at a position corresponding to position 145 according to SEQ ID NO:40, or the complement thereof; a cytosine at a position corresponding to position 145 according to SEQ ID NO:41, or the complement thereof; or a cytosine at a position corresponding to position 205 according to SEQ ID NO:42, or the complement thereof.

### BRIEF DESCRIPTION OF THE DRAWINGS

FIG. 1 shows an association of a missense variant (Leu46Pro) in SLC26A5 with increased risk for hearing loss in a meta-analysis across 5 cohorts.

FIG. 2 shows an association with an aggregate of loss of function and deleterious missense (minor allele frequency of less than 1%) variants in SLC26A5 which suggests that several variants, in addition to Leu46Pro, in SLC26A5 may increase the risk for hearing loss.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A rare variant in the SLC26A5 gene associated with an increased risk of developing hearing loss, such as conductive hearing loss and/or sensorineural hearing loss, in subjects has been identified in accordance with the present disclosure. For example, a genetic alteration in a cDNA molecule resulting in the replacement of a leucine with a proline at position 46 in the encoded SLC26A5 polypeptide has been observed to indicate that the human having such an alteration may have an increased risk of developing hearing loss, such as conductive hearing loss and/or sensorineural hearing loss. Altogether, the genetic analyses described herein surprisingly indicate that the SLC26A5 Leu46Pro polypeptide associates with an increased risk of developing hearing loss, such as conductive hearing loss and/or neural hearing loss. Therefore, subjects that have an SLC26A5 variant nucleic acid molecule or polypeptide that associates with an increased risk of developing hearing loss, such as conductive hearing loss, sensorineural hearing loss, or neural hearing loss, may be treated such that hearing loss is prevented, the symptoms thereof are reduced, and/or development of symptoms is repressed. Accordingly, the present disclosure provides methods of leveraging the identification of such variants in subjects to identify or stratify risk in such subjects of developing hearing loss, such as conductive hearing loss, sensorineural hearing loss, or neural hearing loss, or to diagnose subjects as having an increased risk of developing hearing loss, such as conductive hearing loss, sensorineural hearing loss, or neural hearing loss, such that subjects at risk or subjects with active disease may be treated accordingly.

For purposes of the present disclosure, any particular subject can be categorized as having one of three SLC26A5 genotypes: i) SLC26A5 reference; ii) heterozygous for an SLC26A5 missense variant nucleic acid molecule encoding an SLC26A5 predicted loss-of-function polypeptide; or iii) homozygous for an SLC26A5 missense variant nucleic acid molecule encoding an SLC26A5 predicted loss-of-function polypeptide. A subject is SLC26A5 reference when the subject does not have a copy of an SLC26A5 missense variant nucleic acid molecule encoding an SLC26A5 predicted loss-of-function polypeptide. A subject is heterozygous for an SLC26A5 missense variant nucleic acid molecule encoding an SLC26A5 predicted loss-of-function polypeptide when the subject has a single copy of an SLC26A5 predicted loss-of-function variant nucleic acid molecule. An SLC26A5 missense variant nucleic acid molecule encoding an SLC26A5 predicted loss-of-function polypeptide is any SLC26A5 nucleic acid molecule (such as, a genomic nucleic acid molecule, an mRNA molecule, or a cDNA molecule) encoding an SLC26A5 polypeptide having a partial loss-of-function, a complete loss-of-function, a predicted partial loss-of-function, or a predicted complete loss-of-function. A subject who has an SLC26A5 polypeptide having a partial loss-of-function (or predicted partial loss-of-function) is hypomorphic for SLC26A5. The SLC26A5 predicted loss-of-function variant nucleic acid molecule can be any nucleic acid molecule encoding SLC26A5 Leu46Pro. A subject is homozygous for an SLC26A5 missense variant nucleic acid molecule encoding an SLC26A5 predicted loss-of-function polypeptide when the subject has two copies of an SLC26A5 predicted loss-of-function variant nucleic acid molecule.

For subjects that are genotyped or determined to be heterozygous or homozygous for an SLC26A5 missense variant nucleic acid molecule encoding an SLC26A5 predicted loss-of-function polypeptide, such subjects have an increased risk of developing hearing loss, such as conductive hearing loss, sensorineural hearing loss, or neural hearing loss. For subjects that are genotyped or determined to be heterozygous or homozygous for an SLC26A5 missense variant nucleic acid molecule encoding an SLC26A5 predicted loss-of-function polypeptide, such subjects can be treated with an agent effective to treat hearing loss, such as conductive hearing loss, sensorineural hearing loss, or neural hearing loss.

In any of the embodiments described herein, the SLC26A5 missense variant nucleic acid molecule encoding an SLC26A5 predicted loss-of-function polypeptide can be any SLC26A5 nucleic acid molecule (such as, for example, genomic nucleic acid molecule, mRNA molecule, or cDNA molecule) encoding an SLC26A5 polypeptide having a partial loss-of-function, a complete loss-of-function, a predicted partial loss-of-function, or a predicted complete loss-of-function. For example, the SLC26A5 missense variant nucleic acid molecule encoding an SLC26A5 predicted loss-of-function polypeptide can be any nucleic acid molecule encoding SLC26A5 Leu46Pro. In some embodiments, the SLC26A5 missense variant nucleic acid molecule encodes SLC26A5 Leu46Pro Isoform 1. In some embodiments, the SLC26A5 missense variant nucleic acid molecule encodes SLC26A5 Leu46Pro Isoform 2. In some embodiments, the SLC26A5 missense variant nucleic acid molecule encodes SLC26A5 Leu46Pro Isoform 3. In some embodiments, the SLC26A5 missense variant nucleic acid molecule encodes SLC26A5 Leu46Pro Isoform 4. In some embodiments, the SLC26A5 missense variant nucleic acid molecule encodes SLC26A5 Leu46Pro Isoform 5. In some embodiments, the SLC26A5 missense variant nucleic acid molecule encodes SLC26A5 Leu46Pro Isoform 6. In some embodiments, the SLC26A5 missense variant nucleic acid molecule encodes SLC26A5 Leu46Pro Isoform 7. In some embodiments, the SLC26A5 missense variant nucleic acid molecule encodes SLC26A5 Leu46Pro Isoform 8. In some embodiments, the SLC26A5 missense variant nucleic acid molecule encodes SLC26A5 Leu46Pro Isoform 9.

In any of the embodiments described herein, the SLC26A5 predicted loss-of-function polypeptide can be any SLC26A5 polypeptide having a partial loss-of-function, a complete loss-of-function, a predicted partial loss-of-function, or a predicted complete loss-of-function. In any of the embodiments described herein, the SLC26A5 predicted loss-of-function polypeptide can be any of the SLC26A5 polypeptides described herein including, for example, SLC26A5 Leu46Pro. In some embodiments, the SLC26A5 predicted loss-of-function polypeptide is SLC26A5 Leu46Pro Isoform 1. In some embodiments, the SLC26A5 predicted loss-of-function polypeptide is SLC26A5 Leu46Pro Isoform 2. In some embodiments, the SLC26A5 predicted loss-of-function polypeptide is SLC26A5 Leu46Pro Isoform 3. In some embodiments, the SLC26A5 predicted loss-of-function polypeptide is SLC26A5 Leu46Pro Isoform 4. In some embodiments, the SLC26A5 predicted loss-of-function polypeptide is SLC26A5 Leu46Pro Isoform 5. In some embodiments, the SLC26A5 predicted loss-of-function polypeptide is SLC26A5 Leu46Pro Isoform 6. In some embodiments, the SLC26A5 predicted loss-of-function polypeptide is SLC26A5 Leu46Pro Isoform 7. In some embodiments, the SLC26A5 predicted loss-of-function polypeptide is SLC26A5 Leu46Pro Isoform 8. In some embodiments, the SLC26A5 predicted loss-of-function polypeptide is SLC26A5 Leu46Pro Isoform 9.

In any of the embodiments described herein, hearing loss is conductive hearing loss, sensorineural hearing loss, or neural hearing loss. In any of the embodiments described herein, hearing loss is conductive hearing loss. In any of the embodiments described herein, hearing loss is sensorineural hearing loss. In any of the embodiments described herein, hearing loss is neural hearing loss.

Symptoms of hearing loss include, but are not limited to, hearing problem (muffling of speech and other sounds, difficulty understanding words, especially against background noise or in a crowd, or trouble hearing consonants), ringing in the ears, sensitivity to sound, or speech delay in a child.

The present disclosure also provides methods of treating a subject with a therapeutic agent that treats or inhibits hearing loss, wherein the subject has hearing loss. In some embodiments, the methods comprise determining whether the subject has an SLC26A5 missense variant nucleic acid molecule encoding an SLC26A5 predicted loss-of-function polypeptide by obtaining or having obtained a biological sample from the subject, and performing or having performed a sequence analysis on the biological sample to determine if the subject has a genotype comprising the SLC26A5 missense variant nucleic acid molecule encoding an SLC26A5 predicted loss-of-function polypeptide. The method comprises administering or continuing to administer the therapeutic agent that treats or inhibits hearing loss in a standard dosage amount to a subject that is SLC26A5 reference. The method comprises administering or continuing to administer the therapeutic agent that treats or inhibits hearing loss in an amount that is the same as or greater than a standard dosage amount to a subject that is heterozygous or homozygous for the SLC26A5 missense variant nucleic acid molecule. The presence of a genotype having the SLC26A5 missense variant nucleic acid molecule encoding an SLC26A5 predicted loss-of-function polypeptide indicates the subject has an increased risk of developing hearing loss. In some embodiments, the subject is SLC26A5 reference. In some embodiments, the subject is heterozygous for an SLC26A5 missense variant nucleic acid molecule encoding an SLC26A5 predicted loss-of-function polypeptide. In some embodiments, the subject is homozygous for an SLC26A5 missense variant nucleic acid molecule encoding an SLC26A5 predicted loss-of-function polypeptide.

In some embodiments, the methods of treatment further comprise detecting the presence or absence of an SLC26A5 missense variant nucleic acid molecule encoding an SLC26A5 predicted loss-of-function polypeptide in a biological sample from the subject. As used throughout the present disclosure, a “SLC26A5 missense variant nucleic acid molecule encoding an SLC26A5 predicted loss-of-function polypeptide” is any SLC26A5 nucleic acid molecule (such as, for example, genomic nucleic acid molecule, mRNA molecule, or cDNA molecule) encoding an SLC26A5 polypeptide having a partial loss-of-function, a complete loss-of-function, a predicted partial loss-of-function, or a predicted complete loss-of-function.

Detecting the presence or absence of an SLC26A5 missense variant nucleic acid molecule encoding an SLC26A5 predicted loss-of-function polypeptide in a biological sample from a subject and/or determining whether a subject has an SLC26A5 missense variant nucleic acid molecule encoding an SLC26A5 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of treating a subject with a therapeutic agent that treats or inhibits hearing loss, wherein the subject has hearing loss. In some embodiments, the method comprises determining whether the subject has an SLC26A5 predicted loss-of-function polypeptide by obtaining or having obtained a biological sample from the subject, and performing or having performed an assay on the biological sample to determine if the subject has an SLC26A5 predicted loss-of-function polypeptide. The method comprises administering or continuing to administer the therapeutic agent that treats or inhibits hearing loss in a standard dosage amount to a subject that does not have an SLC26A5 predicted loss-of-function polypeptide. The method comprises administering or continuing to administer the therapeutic agent that treats or inhibits hearing loss in an amount that is the same as or greater than a standard dosage amount to a subject that has an SLC26A5 predicted loss-of-function polypeptide. The presence of an SLC26A5 predicted loss-of-function polypeptide indicates the subject has an increased risk of developing hearing loss. In some embodiments, the subject has an SLC26A5 predicted loss-of-function polypeptide. In some embodiments, the subject does not have an SLC26A5 predicted loss-of-function polypeptide.

Detecting the presence or absence of an SLC26A5 predicted loss-of-function polypeptide in a biological sample from a subject and/or determining whether a subject has an SLC26A5 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of therapeutic agents that treat or inhibit hearing loss include, but are not limited to: antioxidants, calcium-channel blockers, anti-inflammatory drugs (such as steroids), apoptosis inhibitors, D-methionine, ebselen, N-acetylcysteine, lipoic acid, combination of ebselen and allopurinol, resveratrol, neurotrophic factors (such as T-817MA), caspase inhibitors (such as z-DEVD-fmk), copper transport inhibitors (such as cimetidine and copper sulphate), and micronutrients with antioxidant vitamins.

In some embodiments, the dose of the therapeutic agents that treat or inhibit hearing loss can be increased by about 10%, by about 20%, by about 30%, by about 40%, by about 50%, by about 60%, by about 70%, by about 80%, or by about 90% for subjects or subjects that are heterozygous or homozygous for an SLC26A5 missense variant nucleic acid molecule encoding an SLC26A5 predicted loss-of-function polypeptide (i.e., a greater amount than the standard dosage amount) compared to subjects or subjects that are SLC26A5 reference (who may receive a standard dosage amount). In some embodiments, the dose of the therapeutic agents that treat or inhibit hearing loss can be increased by about 10%, by about 20%, by about 30%, by about 40%, or by about 50%. In addition, the dose of therapeutic agents that treat or inhibit hearing loss in subjects or subjects that are heterozygous or homozygous for an SLC26A5 missense variant nucleic acid molecule encoding an SLC26A5 predicted loss-of-function polypeptide can be administered more frequently compared to subjects or subjects that are SLC26A5 reference.

In some embodiments, the dose of the therapeutic agents that treat or inhibit hearing loss can be increased by about 10%, by about 20%, by about 30%, by about 40%, by about 50%, by about 60%, by about 70%, by about 80%, or by about 90% for subjects or subjects that are homozygous for an SLC26A5 missense variant nucleic acid molecule encoding an SLC26A5 predicted loss-of-function polypeptide compared to subjects or subjects that are heterozygous for an SLC26A5 missense variant nucleic acid molecule encoding an SLC26A5 predicted loss-of-function polypeptide. In some embodiments, the dose of the therapeutic agents that treat or inhibit hearing loss can be increased by about 10%, by about 20%, by about 30%, by about 40%, or by about 50%. In addition, the dose of therapeutic agents that treat or inhibit hearing loss in subjects or subjects that are homozygous for an SLC26A5 missense variant nucleic acid molecule encoding an SLC26A5 predicted loss-of-function polypeptide can be administered more frequently compared to subjects or subjects that are heterozygous for an SLC26A5 missense variant nucleic acid molecule encoding an SLC26A5 predicted loss-of-function polypeptide.

Administration of the therapeutic agents that treat or inhibit hearing loss can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit hearing loss can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in hearing loss, a decrease/reduction in the severity of hearing loss (such as, for example, a reduction or inhibition of development or hearing loss), a decrease/reduction in symptoms and hearing loss-related effects, delaying the onset of symptoms and hearing loss-related effects, reducing the severity of symptoms of hearing loss-related effects, reducing the severity of an acute episode, reducing the number of symptoms and hearing loss-related effects, reducing the latency of symptoms and hearing loss-related effects, an amelioration of symptoms and hearing loss-related effects, reducing secondary symptoms, reducing secondary infections, preventing relapse to hearing loss, decreasing the number or frequency of relapse episodes, increasing latency between symptomatic episodes, increasing time to sustained progression, speeding recovery, and/or increasing efficacy of or decreasing resistance to alternative therapeutics, following administration of the agent or composition comprising the agent. A prophylactic effect may comprise a complete or partial avoidance/inhibition or a delay of hearing loss development/progression (such as, for example, a complete or partial avoidance/inhibition or a delay), following administration of a therapeutic protocol. Treatment of hearing loss encompasses the treatment of subjects already diagnosed as having any form of hearing loss at any clinical stage or manifestation, the delay of the onset or evolution or aggravation or deterioration of the symptoms or signs of hearing loss, and/or preventing and/or reducing the severity of hearing loss.

The present disclosure also provides methods of identifying a subject having an increased risk for developing hearing loss. In some embodiments, the method comprises determining or having determined in a biological sample obtained from the subject the presence or absence of an SLC26A5 missense variant nucleic acid molecule (such as a genomic nucleic acid molecule, mRNA molecule, and/or cDNA molecule) encoding an SLC26A5 predicted loss-of-function polypeptide. When the subject lacks an SLC26A5 missense variant nucleic acid molecule encoding an SLC26A5 predicted loss-of-function polypeptide (i.e., the subject is genotypically categorized as an SLC26A5 reference), then the subject does not have an increased risk for developing hearing loss. When the subject has an SLC26A5 missense variant nucleic acid molecule encoding an SLC26A5 predicted loss-of-function polypeptide (i.e., the subject is heterozygous or homozygous for an SLC26A5 missense variant nucleic acid molecule encoding an SLC26A5 predicted loss-of-function polypeptide), then the subject has an increased risk for developing hearing loss.

Determining whether a subject has an SLC26A5 missense variant nucleic acid molecule encoding an SLC26A5 predicted loss-of-function polypeptide in a biological sample from a subject and/or determining whether a subject has an SLC26A5 missense variant nucleic acid molecule encoding an SLC26A5 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing hearing loss, the subject is further treated with a therapeutic agent that treats or inhibits hearing loss, as described herein. In some embodiments, when the subject is heterozygous or homozygous for an SLC26A5 missense variant nucleic acid molecule encoding an SLC26A5 predicted loss-of-function polypeptide, the subject is administered the therapeutic agent that treats or inhibits hearing loss in a dosage amount that is the same as or greater than a standard dosage amount. In some embodiments, when the subject is homozygous for an SLC26A5 missense variant nucleic acid molecule encoding an SLC26A5 predicted loss-of-function polypeptide, the subject is administered the therapeutic agent that treats or inhibits hearing loss in a dosage amount that is the same as or greater than the dosage amount administered to a subject that is heterozygous for an SLC26A5 missense variant nucleic acid molecule encoding an SLC26A5 predicted loss-of-function polypeptide. In some embodiments, the subject is SLC26A5 reference. In some embodiments, the subject is heterozygous for an SLC26A5 missense variant nucleic acid molecule encoding an SLC26A5 predicted loss-of-function polypeptide. In some embodiments, the subject is homozygous for an SLC26A5 missense variant nucleic acid molecule encoding an SLC26A5 predicted loss-of-function polypeptide.

The present disclosure also provides methods of detecting the presence or absence of an SLC26A5 missense genomic variant nucleic acid molecule encoding an SLC26A5 predicted loss-of-function polypeptide in a biological sample from a subject, and/or an SLC26A5 missense variant mRNA molecule encoding an SLC26A5 predicted loss-of-function polypeptide in a biological sample from a subject, and/or an SLC26A5 missense variant cDNA molecule encoding an SLC26A5 predicted loss-of-function polypeptide produced from an mRNA molecule in a biological sample from a subject. It is understood that gene sequences within a population and mRNA molecules encoded by such genes can vary due to polymorphisms such as single-nucleotide polymorphisms. The sequences provided herein for the SLC26A5 variant genomic nucleic acid molecule, SLC26A5 variant mRNA molecule, and SLC26A5 variant cDNA molecule are only exemplary sequences. Other sequences for the SLC26A5 variant genomic nucleic acid molecule, variant mRNA molecule, and variant cDNA molecule are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any SLC26A5 variant nucleic acid molecule, preliminary processing designed to isolate or enrich the sample for the genomic DNA can be employed. A variety of techniques may be used for this purpose. When detecting the level of any SLC26A5 variant mRNA, different techniques can be used enrich the biological sample with mRNA. Various methods to detect the presence or level of an mRNA or the presence of a particular variant genomic DNA locus can be used.

In some embodiments, detecting an SLC26A5 missense variant nucleic acid molecule encoding an SLC26A5 predicted loss-of-function polypeptide in a subject comprises assaying or genotyping a biological sample obtained from the subject to determine whether an SLC26A5 genomic nucleic acid molecule in the biological sample, and/or an SLC26A5 mRNA molecule in the biological sample, and/or an SLC26A5 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an SLC26A5 missense variant nucleic acid molecule (such as, for example, a genomic nucleic acid molecule, an mRNA molecule, and/or a cDNA molecule produced from an mRNA molecule) encoding an SLC26A5 predicted loss-of-function polypeptide in a subject, comprise performing an assay on a biological sample obtained from the subject. The assay determines whether a nucleic acid molecule in the biological sample comprises a particular nucleotide sequence.

In some embodiments, the nucleotide sequence comprises: a cytosine at a position corresponding to position 24,774 according to SEQ ID NO:2 (for genomic nucleic acid molecules), a cytosine at a position corresponding to position 373 according to SEQ ID NO:13 (for mRNA molecules), or a cytosine at a position corresponding to position 373 according to SEQ ID NO:33 (for cDNA molecules obtained from mRNA molecules).

In some embodiments, the nucleotide sequence comprises a cytosine at a position corresponding to position 373 according to SEQ ID NO:14 (for mRNA molecules) or a cytosine at a position corresponding to position 373 according to SEQ ID NO:34 (for cDNA molecules obtained from mRNA molecules).

In some embodiments, the nucleotide sequence comprises a cytosine at a position corresponding to position 373 according to SEQ ID NO:15 (for mRNA molecules) or a cytosine at a position corresponding to position 373 according to SEQ ID NO:35 (for cDNA molecules obtained from mRNA molecules).

In some embodiments, the nucleotide sequence comprises a cytosine at a position corresponding to position 373 according to SEQ ID NO:16 (for mRNA molecules) or a cytosine at a position corresponding to position 373 according to SEQ ID NO:36 (for cDNA molecules obtained from mRNA molecules).

In some embodiments, the nucleotide sequence comprises a cytosine at a position corresponding to position 304 according to SEQ ID NO:17 (for mRNA molecules) or a cytosine at a position corresponding to position 304 according to SEQ ID NO:37 (for cDNA molecules obtained from mRNA molecules).

In some embodiments, the nucleotide sequence comprises a cytosine at a position corresponding to position 304 according to SEQ ID NO:18 (for mRNA molecules) or a cytosine at a position corresponding to position 304 according to SEQ ID NO:38 (for cDNA molecules obtained from mRNA molecules).

In some embodiments, the nucleotide sequence comprises a cytosine at a position corresponding to position 304 according to SEQ ID NO:19 (for mRNA molecules) or a cytosine at a position corresponding to position 304 according to SEQ ID NO:39 (for cDNA molecules obtained from mRNA molecules).

In some embodiments, the nucleotide sequence comprises a cytosine at a position corresponding to position 145 according to SEQ ID NO:20 (for mRNA molecules) or a cytosine at a position corresponding to position 145 according to SEQ ID NO:40 (for cDNA molecules obtained from mRNA molecules).

In some embodiments, the nucleotide sequence comprises a cytosine at a position corresponding to position 145 according to SEQ ID NO:21 (for mRNA molecules) or a cytosine at a position corresponding to position 145 according to SEQ ID NO:41 (for cDNA molecules obtained from mRNA molecules).

In some embodiments, the nucleotide sequence comprises a cytosine at a position corresponding to position 205 according to SEQ ID NO:22 (for mRNA molecules) or a cytosine at a position corresponding to position 205 according to SEQ ID NO:42 (for cDNA molecules obtained from mRNA molecules).

In some embodiments, the nucleotide sequence comprises a cytosine at a position corresponding to position 24,774 according to SEQ ID NO:2, or the complement thereof.

In some embodiments, the nucleotide sequence comprises: a cytosine at a position corresponding to position 373 according to SEQ ID NO:13, or the complement thereof; a cytosine at a position corresponding to position 373 according to SEQ ID NO:14, or the complement thereof; a cytosine at a position corresponding to position 373 according to SEQ ID NO:15, or the complement thereof; a cytosine at a position corresponding to position 373 according to SEQ ID NO:16, or the complement thereof; a cytosine at a position corresponding to position 304 according to SEQ ID NO:17, or the complement thereof; a cytosine at a position corresponding to position 304 according to SEQ ID NO:18, or the complement thereof; a cytosine at a position corresponding to position 304 according to SEQ ID NO:19, or the complement thereof; a cytosine at a position corresponding to position 145 according to SEQ ID NO:20, or the complement thereof; a cytosine at a position corresponding to position 145 according to SEQ ID NO:21, or the complement thereof; or a cytosine at a position corresponding to position 205 according to SEQ ID NO:22, or the complement thereof.

In some embodiments, the nucleotide sequence comprises: a cytosine at a position corresponding to position 373 according to SEQ ID NO:33, or the complement thereof; a cytosine at a position corresponding to position 373 according to SEQ ID NO:34, or the complement thereof; a cytosine at a position corresponding to position 373 according to SEQ ID NO:35, or the complement thereof; a cytosine at a position corresponding to position 373 according to SEQ ID NO:36, or the complement thereof; a cytosine at a position corresponding to position 304 according to SEQ ID NO:37, a cytosine at a position corresponding to position 304 according to SEQ ID NO:38, or the complement thereof; a cytosine at a position corresponding to position 304 according to SEQ ID NO:39, or the complement thereof; a cytosine at a position corresponding to position 145 according to SEQ ID NO:40, or the complement thereof; a cytosine at a position corresponding to position 145 according to SEQ ID NO:41, or the complement thereof; or a cytosine at a position corresponding to position 205 according to SEQ ID NO:42, or the complement thereof.

In some embodiments, the biological sample comprises a cell or cell lysate. Such methods can further comprise, for example, obtaining a biological sample from the subject comprising an SLC26A5 genomic nucleic acid molecule or mRNA molecule, and if mRNA, optionally reverse transcribing the mRNA into cDNA. Such assays can comprise, for example determining the identity of these positions of the particular SLC26A5 nucleic acid molecule. In some embodiments, the method is an in vitro method.

In some embodiments, the determining step, detecting step, or sequence analysis comprises sequencing at least a portion of the nucleotide sequence of the SLC26A5 genomic nucleic acid molecule, the SLC26A5 mRNA molecule, or the SLC26A5 cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of: the nucleotide sequence of the SLC26A5 genomic nucleic acid molecule in the biological sample, wherein the sequenced portion comprises a position corresponding to position 24,774 according to SEQ ID NO:2, or the complement thereof; the nucleotide sequence of the SLC26A5 mRNA molecule in the biological sample, wherein the sequenced portion comprises a position corresponding to position 373 according to SEQ ID NO:13, or the complement thereof; and/or the nucleotide sequence of the SLC26A5 cDNA molecule produced from the mRNA in the biological sample, wherein the sequenced portion comprises a position corresponding to position 373 according to SEQ ID NO:33, or the complement thereof. When the sequenced portion of the SLC26A5 nucleic acid molecule in the biological sample comprises: a cytosine at a position corresponding to position 24,774 according to SEQ ID NO:2, a cytosine at a position corresponding to position 373 according to SEQ ID NO:13, or a cytosine at a position corresponding to position 373 according to SEQ ID NO:33, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 373 according to SEQ ID NO:14, or the complement thereof; and/or the nucleotide sequence of the SLC26A5 cDNA molecule produced from the mRNA in the biological sample, wherein the sequenced portion comprises a position corresponding to position 373 according to SEQ ID NO:34, or the complement thereof. When the sequenced portion of the SLC26A5 nucleic acid molecule in the biological sample comprises: a cytosine at a position corresponding to position 373 according to SEQ ID NO:14, or a cytosine at a position corresponding to position 373 according to SEQ ID NO:34, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 373 according to SEQ ID NO:15, or the complement thereof; and/or the nucleotide sequence of the SLC26A5 cDNA molecule produced from the mRNA in the biological sample, wherein the sequenced portion comprises a position corresponding to position 373 according to SEQ ID NO:35, or the complement thereof. When the sequenced portion of the SLC26A5 nucleic acid molecule in the biological sample comprises: a cytosine at a position corresponding to position 373 according to SEQ ID NO:15, or a cytosine at a position corresponding to position 373 according to SEQ ID NO:35, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 373 according to SEQ ID NO:16, or the complement thereof; and/or the nucleotide sequence of the SLC26A5 cDNA molecule produced from the mRNA in the biological sample, wherein the sequenced portion comprises a position corresponding to position 373 according to SEQ ID NO:36, or the complement thereof. When the sequenced portion of the SLC26A5 nucleic acid molecule in the biological sample comprises: a cytosine at a position corresponding to position 373 according to SEQ ID NO:16, or a cytosine at a position corresponding to position 373 according to SEQ ID NO:36, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 304 according to SEQ ID NO:17, or the complement thereof; and/or the nucleotide sequence of the SLC26A5 cDNA molecule produced from the mRNA in the biological sample, wherein the sequenced portion comprises a position corresponding to position 304 according to SEQ ID NO:37, or the complement thereof. When the sequenced portion of the SLC26A5 nucleic acid molecule in the biological sample comprises: a cytosine at a position corresponding to position 304 according to SEQ ID NO:17, or a cytosine at a position corresponding to position 304 according to SEQ ID NO:37, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 304 according to SEQ ID NO:18, or the complement thereof; and/or the nucleotide sequence of the SLC26A5 cDNA molecule produced from the mRNA in the biological sample, wherein the sequenced portion comprises a position corresponding to position 304 according to SEQ ID NO:38, or the complement thereof. When the sequenced portion of the SLC26A5 nucleic acid molecule in the biological sample comprises: a cytosine at a position corresponding to position 304 according to SEQ ID NO:18, or a cytosine at a position corresponding to position 304 according to SEQ ID NO:38, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 304 according to SEQ ID NO:19, or the complement thereof; and/or the nucleotide sequence of the SLC26A5 cDNA molecule produced from the mRNA in the biological sample, wherein the sequenced portion comprises a position corresponding to position 304 according to SEQ ID NO:39, or the complement thereof. When the sequenced portion of the SLC26A5 nucleic acid molecule in the biological sample comprises: a cytosine at a position corresponding to position 304 according to SEQ ID NO:19, or a cytosine at a position corresponding to position 304 according to SEQ ID NO:39, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 145 according to SEQ ID NO:20, or the complement thereof; and/or the nucleotide sequence of the SLC26A5 cDNA molecule produced from the mRNA in the biological sample, wherein the sequenced portion comprises a position corresponding to position 145 according to SEQ ID NO:40, or the complement thereof. When the sequenced portion of the SLC26A5 nucleic acid molecule in the biological sample comprises: a cytosine at a position corresponding to position 145 according to SEQ ID NO:20, or a cytosine at a position corresponding to position 145 according to SEQ ID NO:40, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 145 according to SEQ ID NO:21, or the complement thereof; and/or the nucleotide sequence of the SLC26A5 cDNA molecule produced from the mRNA in the biological sample, wherein the sequenced portion comprises a position corresponding to position 145 according to SEQ ID NO:41, or the complement thereof. When the sequenced portion of the SLC26A5 nucleic acid molecule in the biological sample comprises: a cytosine at a position corresponding to position 145 according to SEQ ID NO:21, or a cytosine at a position corresponding to position 145 according to SEQ ID NO:41, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of: the nucleotide sequence of the SLC26A5 mRNA molecule in the biological sample, wherein the sequenced portion comprises a position corresponding to position 205 according to SEQ ID NO:22, or the complement thereof; and/or the nucleotide sequence of the SLC26A5 cDNA molecule produced from the mRNA in the biological sample, wherein the sequenced portion comprises a position corresponding to position 205 according to SEQ ID NO:42, or the complement thereof. When the sequenced portion of the SLC26A5 nucleic acid molecule in the biological sample comprises: a cytosine at a position corresponding to position 205 according to SEQ ID NO:22, or a cytosine at a position corresponding to position 205 according to SEQ ID NO:42, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of the nucleotide sequence of the SLC26A5 genomic nucleic acid molecule in the biological sample, wherein the sequenced portion comprises a position corresponding to position 24,774 according to SEQ ID NO:2, or the complement thereof. When the sequenced portion of the SLC26A5 nucleic acid molecule in the biological sample comprises a cytosine at a position corresponding to position 24,774 according to SEQ ID NO:2, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of the nucleotide sequence of the SLC26A5 mRNA molecule in the biological sample, wherein the sequenced portion comprises a position corresponding to: position 373 according to SEQ ID NO:13, or the complement thereof; position 373 according to SEQ ID NO:14, or the complement thereof; position 373 according to SEQ ID NO:15, or the complement thereof; position 373 according to SEQ ID NO:16, or the complement thereof; position 304 according to SEQ ID NO:17, or the complement thereof; position 304 according to SEQ ID NO:18, or the complement thereof; position 304 according to SEQ ID NO:19 the complement thereof; position 145 according to SEQ ID NO:20, the complement thereof; position 145 according to SEQ ID NO:21, or the complement thereof; or position 205 according to SEQ ID NO:22, or the complement thereof. When the sequenced portion of the SLC26A5 nucleic acid molecule in the biological sample comprises: a cytosine at a position corresponding to position 373 according to SEQ ID NO:13, a cytosine at a position corresponding to position 373 according to SEQ ID NO:14, a cytosine at a position corresponding to position 373 according to SEQ ID NO:15, a cytosine at a position corresponding to position 373 according to SEQ ID NO:16, a cytosine at a position corresponding to position 304 according to SEQ ID NO:17, a cytosine at a position corresponding to position 304 according to SEQ ID NO:18, a cytosine at a position corresponding to position 304 according to SEQ ID NO:19, a cytosine at a position corresponding to position 145 according to SEQ ID NO:20, a cytosine at a position corresponding to position 145 according to SEQ ID NO:21, or a cytosine at a position corresponding to position 205 according to SEQ ID NO:22, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises sequencing at least a portion of the nucleotide sequence of the SLC26A5 cDNA molecule in the biological sample, wherein the sequenced portion comprises a position corresponding to: position 373 according to SEQ ID NO:33, or the complement thereof; position 373 according to SEQ ID NO:34, or the complement thereof; position 373 according to SEQ ID NO:35, or the complement thereof; position 373 according to SEQ ID NO:36, or the complement thereof; position 304 according to SEQ ID NO:37, or the complement thereof; position 304 according to SEQ ID NO:38, or the complement thereof; or position 304 according to SEQ ID NO:39 the complement thereof; position 145 according to SEQ ID NO:40, the complement thereof; position 145 according to SEQ ID NO:41, or the complement thereof; or position 205 according to SEQ ID NO:42, or the complement thereof. When the sequenced portion of the SLC26A5 nucleic acid molecule in the biological sample comprises: a cytosine at a position corresponding to position 373 according to SEQ ID NO:33, a cytosine at a position corresponding to position 373 according to SEQ ID NO:34, a cytosine at a position corresponding to position 373 according to SEQ ID NO:35, a cytosine at a position corresponding to position 373 according to SEQ ID NO:36, a cytosine at a position corresponding to position 304 according to SEQ ID NO:37, a cytosine at a position corresponding to position 304 according to SEQ ID NO:38, a cytosine at a position corresponding to position 304 according to SEQ ID NO:39, a cytosine at a position corresponding to position 145 according to SEQ ID NO:40, a cytosine at a position corresponding to position 145 according to SEQ ID NO:41, or a cytosine at a position corresponding to position 205 according to SEQ ID NO:42, then the SLC26A5 nucleic acid molecule in the biological sample is an SLC26A5 missense variant nucleic acid molecule encoding an SLC26A5 predicted loss-of-function polypeptide.

In some embodiments, the determining step, detecting step, or sequence analysis comprises: a) contacting the biological sample with a primer hybridizing to a portion of the nucleotide sequence of the SLC26A5: genomic nucleic acid molecule that is proximate to a position corresponding to position 24,774 according to SEQ ID NO:2; mRNA molecule that is proximate to a position corresponding to position 373 according to SEQ ID NO:13; and/or cDNA molecule that is proximate to a position corresponding to position 373 according to SEQ ID NO:33; b) extending the primer at least through the position of the nucleotide sequence of the SLC26A5: genomic nucleic acid molecule corresponding to position 24,774 according to SEQ ID NO:2; mRNA molecule corresponding to position 373 according to SEQ ID NO:13; and/or cDNA molecule corresponding to position 373 according to SEQ ID NO:33; and c) determining whether the extension product of the primer comprises: a cytosine at a position corresponding to position 24,774 according to SEQ ID NO:2, a cytosine at a position corresponding to position 373 according to SEQ ID NO:13, and/or a cytosine at a position corresponding to position 373 according to SEQ ID NO:33.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 373 according to SEQ ID NO:14; and/or cDNA molecule that is proximate to a position corresponding to position 373 according to SEQ ID NO:34; b) extending the primer at least through the position of the nucleotide sequence of the SLC26A5: mRNA molecule corresponding to position 373 according to SEQ ID NO:14; and/or cDNA molecule corresponding to position 373 according to SEQ ID NO:34; and c) determining whether the extension product of the primer comprises: a cytosine at a position corresponding to position 373 according to SEQ ID NO:14; and/or a cytosine at a position corresponding to position 373 according to SEQ ID NO:34.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 373 according to SEQ ID NO:15; and/or cDNA molecule that is proximate to a position corresponding to position 373 according to SEQ ID NO:35; b) extending the primer at least through the position of the nucleotide sequence of the SLC26A5: mRNA molecule corresponding to position 373 according to SEQ ID NO:15; and/or cDNA molecule corresponding to position 373 according to SEQ ID NO:35; and c) determining whether the extension product of the primer comprises: a cytosine at a position corresponding to position 373 according to SEQ ID NO:15; and/or a cytosine at a position corresponding to position 373 according to SEQ ID NO:35.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 373 according to SEQ ID NO:16; and/or cDNA molecule that is proximate to a position corresponding to position 373 according to SEQ ID NO:36; b) extending the primer at least through the position of the nucleotide sequence of the SLC26A5: mRNA molecule corresponding to position 373 according to SEQ ID NO:16; and/or cDNA molecule corresponding to position 373 according to SEQ ID NO:36; and c) determining whether the extension product of the primer comprises: a cytosine at a position corresponding to position 373 according to SEQ ID NO:16; and/or a cytosine at a position corresponding to position 373 according to SEQ ID NO:36.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 304 according to SEQ ID NO:17; and/or cDNA molecule that is proximate to a position corresponding to position 304 according to SEQ ID NO:37; b) extending the primer at least through the position of the nucleotide sequence of the SLC26A5: mRNA molecule corresponding to position 304 according to SEQ ID NO:17; and/or cDNA molecule corresponding to position 304 according to SEQ ID NO:37; and c) determining whether the extension product of the primer comprises: a cytosine at a position corresponding to position 304 according to SEQ ID NO:17; and/or a cytosine at a position corresponding to position 304 according to SEQ ID NO:37.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 304 according to SEQ ID NO:18; and/or cDNA molecule that is proximate to a position corresponding to position 304 according to SEQ ID NO:38; b) extending the primer at least through the position of the nucleotide sequence of the SLC26A5: mRNA molecule corresponding to position 304 according to SEQ ID NO:18; and/or cDNA molecule corresponding to position 304 according to SEQ ID NO:38; and c) determining whether the extension product of the primer comprises: a cytosine at a position corresponding to position 304 according to SEQ ID NO:18; and/or a cytosine at a position corresponding to position 304 according to SEQ ID NO:38.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 304 according to SEQ ID NO:19; and/or cDNA molecule that is proximate to a position corresponding to position 304 according to SEQ ID NO:39; b) extending the primer at least through the position of the nucleotide sequence of the SLC26A5: mRNA molecule corresponding to position 304 according to SEQ ID NO:19; and/or cDNA molecule corresponding to position 304 according to SEQ ID NO:39; and c) determining whether the extension product of the primer comprises: a cytosine at a position corresponding to position 304 according to SEQ ID NO:19; and/or a cytosine at a position corresponding to position 304 according to SEQ ID NO:39.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 145 according to SEQ ID NO:20; and/or cDNA molecule that is proximate to a position corresponding to position 145 according to SEQ ID NO:40; b) extending the primer at least through the position of the nucleotide sequence of the SLC26A5: mRNA molecule corresponding to position 145 according to SEQ ID NO:20; and/or cDNA molecule corresponding to position 145 according to SEQ ID NO:40; and c) determining whether the extension product of the primer comprises: a cytosine at a position corresponding to position 145 according to SEQ ID NO:20; and/or a cytosine at a position corresponding to position 145 according to SEQ ID NO:40.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 145 according to SEQ ID NO:21; and/or cDNA molecule that is proximate to a position corresponding to position 145 according to SEQ ID NO:41; b) extending the primer at least through the position of the nucleotide sequence of the SLC26A5: mRNA molecule corresponding to position 145 according to SEQ ID NO:21; and/or cDNA molecule corresponding to position 145 according to SEQ ID NO:41; and c) determining whether the extension product of the primer comprises: a cytosine at a position corresponding to position 145 according to SEQ ID NO:21; and/or a cytosine at a position corresponding to position 145 according to SEQ ID NO:41.

In some embodiments, the determining step, detecting step, or sequence analysis comprises: a) contacting the biological sample with a primer hybridizing to a portion of the nucleotide sequence of the SLC26A5: mRNA molecule that is proximate to a position corresponding to position 205 according to SEQ ID NO:22; and/or cDNA molecule that is proximate to a position corresponding to position 205 according to SEQ ID NO:42; b) extending the primer at least through the position of the nucleotide sequence of the SLC26A5: mRNA molecule corresponding to position 205 according to SEQ ID NO:22; and/or cDNA molecule corresponding to position 205 according to SEQ ID NO:42; and c) determining whether the extension product of the primer comprises a cytosine at a position corresponding to position 205 according to SEQ ID NO:22; and/or a cytosine at a position corresponding to position 205 according to SEQ ID NO:42.

In some embodiments, the determining step, detecting step, or sequence analysis comprises: a) contacting the biological sample with a primer hybridizing to a portion of the nucleotide sequence of the SLC26A5 genomic nucleic acid molecule that is proximate to a position corresponding to position 24,774 according to SEQ ID NO:2; b) extending the primer at least through the position of the nucleotide sequence of the SLC26A5 genomic nucleic acid molecule corresponding to position 24,774 according to SEQ ID NO:2; and c) determining whether the extension product of the primer comprises a cytosine at a position corresponding to position 24,774 according to SEQ ID NO:2.

In some embodiments, the determining step, detecting step, or sequence analysis comprises: a) contacting the biological sample with a primer hybridizing to a portion of the nucleotide sequence of the SLC26A5 mRNA molecule that is proximate to a position corresponding to: position 373 according to SEQ ID NO:13, position 373 according to SEQ ID NO:14, position 373 according to SEQ ID NO:15, position 373 according to SEQ ID NO:16, position 304 according to SEQ ID NO:17, position 304 according to SEQ ID NO:18, position 304 according to SEQ ID NO:19, position 145 according to SEQ ID NO:20, position 145 according to SEQ ID NO:21, or position 205 according to SEQ ID NO:22; b) extending the primer at least through the position of the nucleotide sequence of the SLC26A5 mRNA molecule corresponding to: position 373 according to SEQ ID NO:13, position 373 according to SEQ ID NO:14, position 373 according to SEQ ID NO:15, position 373 according to SEQ ID NO:16, position 304 according to SEQ ID NO:17, position 304 according to SEQ ID NO:18, position 304 according to SEQ ID NO:19, position 145 according to SEQ ID NO:20, position 145 according to SEQ ID NO:21, or position 205 according to SEQ ID NO:22; and c) determining whether the extension product of the primer comprises: a cytosine at a position corresponding to position 373 according to SEQ ID NO:13, a cytosine at a position corresponding to position 373 according to SEQ ID NO:14, a cytosine at a position corresponding to position 373 according to SEQ ID NO:15, a cytosine at a position corresponding to position 373 according to SEQ ID NO:16, a cytosine at a position corresponding to position 304 according to SEQ ID NO:17, a cytosine at a position corresponding to position 304 according to SEQ ID NO:18, a cytosine at a position corresponding to position 304 according to SEQ ID NO:19, a cytosine at a position corresponding to position 145 according to SEQ ID NO:20, a cytosine at a position corresponding to position 145 according to SEQ ID NO:21, or a cytosine at a position corresponding to position 205 according to SEQ ID NO:22.

In some embodiments, the determining step, detecting step, or sequence analysis comprises: a) contacting the biological sample with a primer hybridizing to a portion of the nucleotide sequence of the SLC26A5 cDNA molecule that is proximate to a position corresponding to: position 373 according to SEQ ID NO:33, position 373 according to SEQ ID NO:34, position 373 according to SEQ ID NO:35, position 373 according to SEQ ID NO:36, position 304 according to SEQ ID NO:37, position 304 according to SEQ ID NO:38, position 304 according to SEQ ID NO:39, position 145 according to SEQ ID NO:40, position 145 according to SEQ ID NO:41, or position 205 according to SEQ ID NO:42; b) extending the primer at least through the position of the nucleotide sequence of the SLC26A5 cDNA molecule corresponding to: position 373 according to SEQ ID NO:33, position 373 according to SEQ ID NO:34, position 373 according to SEQ ID NO:35, position 373 according to SEQ ID NO:36, position 304 according to SEQ ID NO:37, position 304 according to SEQ ID NO:38, position 304 according to SEQ ID NO:39, position 145 according to SEQ ID NO:40, position 145 according to SEQ ID NO:41, or position 205 according to SEQ ID NO:42; and c) determining whether the extension product of the primer comprises: a cytosine at a position corresponding to position 373 according to SEQ ID NO:33, a cytosine at a position corresponding to position 373 according to SEQ ID NO:34, a cytosine at a position corresponding to position 373 according to SEQ ID NO:35, a cytosine at a position corresponding to position 373 according to SEQ ID NO:36, a cytosine at a position corresponding to position 304 according to SEQ ID NO:37, a cytosine at a position corresponding to position 304 according to SEQ ID NO:38, a cytosine at a position corresponding to position 304 according to SEQ ID NO:39, a cytosine at a position corresponding to position 145 according to SEQ ID NO:40, a cytosine at a position corresponding to position 145 according to SEQ ID NO:41, or a cytosine at a position corresponding to position 205 according to SEQ ID NO:42.

In some embodiments, the assay comprises sequencing the entire nucleic acid molecule. In some embodiments, only an SLC26A5 genomic nucleic acid molecule is analyzed. In some embodiments, only an SLC26A5 mRNA is analyzed. In some embodiments, only an SLC26A5 cDNA obtained from SLC26A5 mRNA is analyzed.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 24,774 according to SEQ ID NO:2, or the complement thereof; a cytosine at a position corresponding to position 373 according to SEQ ID NO:13, or the complement thereof; and/or a cytosine at a position corresponding to position 373 according to SEQ ID NO:33, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 24,774 according to SEQ ID NO:2, or the complement thereof; a cytosine at a position corresponding to position 373 according to SEQ ID NO:13, or the complement thereof; and/or a cytosine at a position corresponding to position 373 according to SEQ ID NO:33, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 373 according to SEQ ID NO:14, or the complement thereof; and/or a cytosine at a position corresponding to position 373 according to SEQ ID NO:34, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 373 according to SEQ ID NO:14, or the complement thereof; and/or a cytosine at a position corresponding to position 373 according to SEQ ID NO:34, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 373 according to SEQ ID NO:15, or the complement thereof; and/or a cytosine at a position corresponding to position 373 according to SEQ ID NO:35, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 373 according to SEQ ID NO:15, or the complement thereof; and/or a cytosine at a position corresponding to position 373 according to SEQ ID NO:35, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 373 according to SEQ ID NO:16, or the complement thereof; and/or a cytosine at a position corresponding to position 373 according to SEQ ID NO:36, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 373 according to SEQ ID NO:16, or the complement thereof; and/or a cytosine at a position corresponding to position 373 according to SEQ ID NO:36, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 304 according to SEQ ID NO:17, or the complement thereof; and/or a cytosine at a position corresponding to position 304 according to SEQ ID NO:37, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 304 according to SEQ ID NO:17, or the complement thereof; and/or a cytosine at a position corresponding to position 304 according to SEQ ID NO:37, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 304 according to SEQ ID NO:18, or the complement thereof; and/or a cytosine at a position corresponding to position 304 according to SEQ ID NO:38, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 304 according to SEQ ID NO:18, or the complement thereof; and/or a cytosine at a position corresponding to position 304 according to SEQ ID NO:38, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 304 according to SEQ ID NO:19, or the complement thereof; and/or a cytosine at a position corresponding to position 304 according to SEQ ID NO:39, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 304 according to SEQ ID NO:19, or the complement thereof; and/or a cytosine at a position corresponding to position 304 according to SEQ ID NO:39, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 145 according to SEQ ID NO:20, or the complement thereof; and/or a cytosine at a position corresponding to position 145 according to SEQ ID NO:40, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 145 according to SEQ ID NO:20, or the complement thereof; and/or a cytosine at a position corresponding to position 145 according to SEQ ID NO:40, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 145 according to SEQ ID NO:21, or the complement thereof; and/or a cytosine at a position corresponding to position 145 according to SEQ ID NO:41, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 145 according to SEQ ID NO:21, or the complement thereof; and/or a cytosine at a position corresponding to position 145 according to SEQ ID NO:41, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 205 according to SEQ ID NO:22, or the complement thereof; and/or a cytosine at a position corresponding to position 205 according to SEQ ID NO:42, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 205 according to SEQ ID NO:22, or the complement thereof; and/or a cytosine at a position corresponding to position 205 according to SEQ ID NO:42, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises a cytosine at a position corresponding to position 24,774 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a cytosine at a position corresponding to position 24,774 according to SEQ ID NO:2, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 373 according to SEQ ID NO:13, or the complement thereof; a cytosine at a position corresponding to position 373 according to SEQ ID NO:14, or the complement thereof; a cytosine at a position corresponding to position 373 according to SEQ ID NO:15, or the complement thereof; a cytosine at a position corresponding to position 373 according to SEQ ID NO:16, or the complement thereof; a cytosine at a position corresponding to position 304 according to SEQ ID NO:17, or the complement thereof; a cytosine at a position corresponding to position 304 according to SEQ ID NO:18, or the complement thereof; a cytosine at a position corresponding to position 304 according to SEQ ID NO:19, or the complement thereof; a cytosine at a position corresponding to position 145 according to SEQ ID NO:20, or the complement thereof; a cytosine at a position corresponding to position 145 according to SEQ ID NO:21, or the complement thereof; or a cytosine at a position corresponding to position 205 according to SEQ ID NO:22, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 373 according to SEQ ID NO:13, or the complement thereof; a cytosine at a position corresponding to position 373 according to SEQ ID NO:14, or the complement thereof; a cytosine at a position corresponding to position 373 according to SEQ ID NO:15, or the complement thereof; a cytosine at a position corresponding to position 373 according to SEQ ID NO:16, or the complement thereof; a cytosine at a position corresponding to position 304 according to SEQ ID NO:17, or the complement thereof; a cytosine at a position corresponding to position 304 according to SEQ ID NO:18, or the complement thereof; a cytosine at a position corresponding to position 304 according to SEQ ID NO:19, or the complement thereof; a cytosine at a position corresponding to position 145 according to SEQ ID NO:20, or the complement thereof; a cytosine at a position corresponding to position 145 according to SEQ ID NO:21, or the complement thereof; or a cytosine at a position corresponding to position 205 according to SEQ ID NO:22, or the complement thereof; and d) detecting the detectable label.

In some embodiments, the determining step, detecting step, or sequence analysis comprises: a) amplifying at least a portion of the nucleic acid molecule that encodes the SLC26A5 polypeptide, wherein the amplified portion comprises: a cytosine at a position corresponding to position 373 according to SEQ ID NO:33, or the complement thereof; a cytosine at a position corresponding to position 373 according to SEQ ID NO:34, or the complement thereof; a cytosine at a position corresponding to position 373 according to SEQ ID NO:35, or the complement thereof; a cytosine at a position corresponding to position 373 according to SEQ ID NO:36, or the complement thereof; a cytosine at a position corresponding to position 304 according to SEQ ID NO:37, a cytosine at a position corresponding to position 304 according to SEQ ID NO:38, or the complement thereof; a cytosine at a position corresponding to position 304 according to SEQ ID NO:39, or the complement thereof; a cytosine at a position corresponding to position 145 according to SEQ ID NO:40, or the complement thereof; a cytosine at a position corresponding to position 145 according to SEQ ID NO:41, or the complement thereof; or a cytosine at a position corresponding to position 205 according to SEQ ID NO:42, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 373 according to SEQ ID NO:33, or the complement thereof; a cytosine at a position corresponding to position 373 according to SEQ ID NO:34, or the complement thereof; a cytosine at a position corresponding to position 373 according to SEQ ID NO:35, or the complement thereof; a cytosine at a position corresponding to position 373 according to SEQ ID NO:36, or the complement thereof; a cytosine at a position corresponding to position 304 according to SEQ ID NO:37, a cytosine at a position corresponding to position 304 according to SEQ ID NO:38, or the complement thereof; a cytosine at a position corresponding to position 304 according to SEQ ID NO:39, or the complement thereof; a cytosine at a position corresponding to position 145 according to SEQ ID NO:40, or the complement thereof; a cytosine at a position corresponding to position 145 according to SEQ ID NO:41, or the complement thereof; or a cytosine at a position corresponding to position 205 according to SEQ ID NO:42, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 24,774 according to SEQ ID NO:2, or the complement thereof; a cytosine at a position corresponding to position 373 according to SEQ ID NO:13, or the complement thereof; and/or a cytosine at a position corresponding to position 373 according to SEQ ID NO:33, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 373 according to SEQ ID NO:14, or the complement thereof; and/or a cytosine at a position corresponding to position 373 according to SEQ ID NO:34, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 373 according to SEQ ID NO:15, or the complement thereof; and/or a cytosine at a position corresponding to position 373 according to SEQ ID NO:35, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 373 according to SEQ ID NO:16, or the complement thereof; and/or a cytosine at a position corresponding to position 373 according to SEQ ID NO:36, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 304 according to SEQ ID NO:17, or the complement thereof; and/or a cytosine at a position corresponding to position 304 according to SEQ ID NO:37, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 304 according to SEQ ID NO:18, or the complement thereof; and/or a cytosine at a position corresponding to position 304 according to SEQ ID NO:38, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 304 according to SEQ ID NO:19, or the complement thereof; and/or a cytosine at a position corresponding to position 304 according to SEQ ID NO:39, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 145 according to SEQ ID NO:20, or the complement thereof; and/or a cytosine at a position corresponding to position 145 according to SEQ ID NO:40, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 145 according to SEQ ID NO:21, or the complement thereof; and/or a cytosine at a position corresponding to position 145 according to SEQ ID NO:41, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: or a cytosine at a position corresponding to position 205 according to SEQ ID NO:22, or the complement thereof; and/or a cytosine at a position corresponding to position 205 according to SEQ ID NO:42, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a cytosine at a position corresponding to position 24,774 according to SEQ ID NO:2, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 373 according to SEQ ID NO:13, or the complement thereof; a cytosine at a position corresponding to position 373 according to SEQ ID NO:14, or the complement thereof; a cytosine at a position corresponding to position 373 according to SEQ ID NO:15, or the complement thereof; a cytosine at a position corresponding to position 373 according to SEQ ID NO:16, or the complement thereof; a cytosine at a position corresponding to position 304 according to SEQ ID NO:17, or the complement thereof; a cytosine at a position corresponding to position 304 according to SEQ ID NO:18, or the complement thereof; a cytosine at a position corresponding to position 304 according to SEQ ID NO:19, or the complement thereof; a cytosine at a position corresponding to position 145 according to SEQ ID NO:20, or the complement thereof; a cytosine at a position corresponding to position 145 according to SEQ ID NO:21, or the complement thereof; or a cytosine at a position corresponding to position 205 according to SEQ ID NO:22, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a cytosine at a position corresponding to position 373 according to SEQ ID NO:33, or the complement thereof; a cytosine at a position corresponding to position 373 according to SEQ ID NO:34, or the complement thereof; a cytosine at a position corresponding to position 373 according to SEQ ID NO:35, or the complement thereof; a cytosine at a position corresponding to position 373 according to SEQ ID NO:36, or the complement thereof; a cytosine at a position corresponding to position 304 according to SEQ ID NO:37, a cytosine at a position corresponding to position 304 according to SEQ ID NO:38, or the complement thereof; a cytosine at a position corresponding to position 304 according to SEQ ID NO:39, or the complement thereof; a cytosine at a position corresponding to position 145 according to SEQ ID NO:40, or the complement thereof; a cytosine at a position corresponding to position 145 according to SEQ ID NO:41, or the complement thereof; or a cytosine at a position corresponding to position 205 according to SEQ ID NO:42, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an SLC26A5 variant genomic sequence, variant mRNA sequence, or variant cDNA sequence and not the corresponding SLC26A5 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an SLC26A5 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an SLC26A5 nucleic acid molecule (genomic nucleic acid molecule, mRNA molecule, or cDNA molecule), or complement thereof, within a biological sample comprises a nucleotide sequence comprising a cytosine at a position corresponding to position 24,774 according to SEQ ID NO:2 (genomic nucleic acid molecule), or a cytosine at a position corresponding to position 373 according to SEQ ID NO:13 (mRNA molecule), or a cytosine at a position corresponding to position 373 according to SEQ ID NO:33 (cDNA molecule), the biological sample can be subjected to an amplification method using a primer pair that includes a first primer derived from the 5′ flanking sequence adjacent to a cytosine at a position corresponding to position 24,774 according to SEQ ID NO:2, or a cytosine at a position corresponding to position 373 according to SEQ ID NO:13, or a cytosine at a position corresponding to position 373 according to SEQ ID NO:33, and a second primer derived from the 3′ flanking sequence adjacent to a cytosine at a position corresponding to position 24,774 according to SEQ ID NO:2, or a cytosine at a position corresponding to position 373 according to SEQ ID NO:13, or a cytosine at a position corresponding to position 373 according to SEQ ID NO:33 to produce an amplicon that is indicative of the presence of the SNP at positions encoding a cytosine at a position corresponding to position 24,774 according to SEQ ID NO:2, or a cytosine at a position corresponding to position 373 according to SEQ ID NO:13, or a cytosine at a position corresponding to position 373 according to SEQ ID NO:33. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 24,774 according to SEQ ID NO:2, or a cytosine at a position corresponding to position 373 according to SEQ ID NO:13, or a cytosine at a position corresponding to position 373 according to SEQ ID NO:33, and at least 1, 2, 3, 4, 5, 6, 7, 8, 9, 10, or more nucleotides on each side of positions comprising a cytosine at a position corresponding to position 24,774 according to SEQ ID NO:2, or a cytosine at a position corresponding to position 373 according to SEQ ID NO:13, or a cytosine at a position corresponding to position 373 according to SEQ ID NO:33.

In some embodiments, to determine whether an SLC26A5 nucleic acid molecule (mRNA molecule or cDNA molecule), or complement thereof, within a biological sample comprises a cytosine at a position corresponding to position 373 according to SEQ ID NO:14 (mRNA molecule), or a cytosine at a position corresponding to position 373 according to SEQ ID NO:34 (cDNA molecule), the biological sample can be subjected to an amplification method using a primer pair that includes a first primer derived from the 5′ flanking sequence adjacent to a cytosine at a position corresponding to position 373 according to SEQ ID NO:14 or a cytosine at a position corresponding to position 373 according to SEQ ID NO:34, and a second primer derived from the 3′ flanking sequence adjacent to a cytosine at a position corresponding to position 373 according to SEQ ID NO:14 or a cytosine at a position corresponding to position 373 according to SEQ ID NO:34 to produce an amplicon that is indicative of the presence of the SNP at positions encoding a cytosine at a position corresponding to position 373 according to SEQ ID NO:14 or a cytosine at a position corresponding to position 373 according to SEQ ID NO:34. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 373 according to SEQ ID NO:14 or a cytosine at a position corresponding to position 373 according to SEQ ID NO:34, and at least 1, 2, 3, 4, 5, 6, 7, 8, 9, 10, or more nucleotides on each side of positions comprising a cytosine at a position corresponding to position 373 according to SEQ ID NO:14 or a cytosine at a position corresponding to position 373 according to SEQ ID NO:34.

In some embodiments, to determine whether an SLC26A5 nucleic acid molecule (mRNA molecule, or cDNA molecule), or complement thereof, within a biological sample comprises a nucleotide sequence comprising a cytosine at a position corresponding to position 373 according to SEQ ID NO:15 (mRNA molecule) or a cytosine at a position corresponding to position 373 according to SEQ ID NO:35 (cDNA molecule), the biological sample can be subjected to an amplification method using a primer pair that includes a first primer derived from the 5′ flanking sequence adjacent to a cytosine at a position corresponding to position 373 according to SEQ ID NO:15 or a cytosine at a position corresponding to position 373 according to SEQ ID NO:35, and a second primer derived from the 3′ flanking sequence adjacent to a cytosine at a position corresponding to position 373 according to SEQ ID NO:15 or a cytosine at a position corresponding to position 373 according to SEQ ID NO:35 to produce an amplicon that is indicative of the presence of the SNP at positions encoding a cytosine at a position corresponding to position 373 according to SEQ ID NO:15 or a cytosine at a position corresponding to position 373 according to SEQ ID NO:35. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 373 according to SEQ ID NO:15 or a cytosine at a position corresponding to position 373 according to SEQ ID NO:35, and at least 1, 2, 3, 4, 5, 6, 7, 8, 9, 10, or more nucleotides on each side of positions comprising a cytosine at a position corresponding to position 373 according to SEQ ID NO:15 or a cytosine at a position corresponding to position 373 according to SEQ ID NO:35.

In some embodiments, to determine whether an SLC26A5 nucleic acid molecule (mRNA molecule, or cDNA molecule), or complement thereof, within a biological sample comprises a nucleotide sequence comprising a cytosine at a position corresponding to position 373 according to SEQ ID NO:16 (mRNA molecule) or a cytosine at a position corresponding to position 373 according to SEQ ID NO:36 (cDNA molecule), the biological sample can be subjected to an amplification method using a primer pair that includes a first primer derived from the 5′ flanking sequence adjacent to a cytosine at a position corresponding to position 373 according to SEQ ID NO:16 or a cytosine at a position corresponding to position 373 according to SEQ ID NO:36, and a second primer derived from the 3′ flanking sequence adjacent to a cytosine at a position corresponding to position 373 according to SEQ ID NO:16 or a cytosine at a position corresponding to position 373 according to SEQ ID NO:36 to produce an amplicon that is indicative of the presence of the SNP at positions encoding a cytosine at a position corresponding to position 373 according to SEQ ID NO:16 or a cytosine at a position corresponding to position 373 according to SEQ ID NO:36. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 373 according to SEQ ID NO:16 or a cytosine at a position corresponding to position 373 according to SEQ ID NO:36, and at least 1, 2, 3, 4, 5, 6, 7, 8, 9, 10, or more nucleotides on each side of positions comprising a cytosine at a position corresponding to position 373 according to SEQ ID NO:16 or a cytosine at a position corresponding to position 373 according to SEQ ID NO:36.

In some embodiments, to determine whether an SLC26A5 nucleic acid molecule (mRNA molecule, or cDNA molecule), or complement thereof, within a biological sample comprises a nucleotide sequence comprising a cytosine at a position corresponding to position 304 according to SEQ ID NO:17 (mRNA molecule) or a cytosine at a position corresponding to position 304 according to SEQ ID NO:37 (cDNA molecule), the biological sample can be subjected to an amplification method using a primer pair that includes a first primer derived from the 5′ flanking sequence adjacent to a cytosine at a position corresponding to position 304 according to SEQ ID NO:17 or a cytosine at a position corresponding to position 304 according to SEQ ID NO:37, and a second primer derived from the 3′ flanking sequence adjacent to a cytosine at a position corresponding to position 304 according to SEQ ID NO:17 or a cytosine at a position corresponding to position 304 according to SEQ ID NO:37 to produce an amplicon that is indicative of the presence of the SNP at positions encoding a cytosine at a position corresponding to position 304 according to SEQ ID NO:17 or a cytosine at a position corresponding to position 304 according to SEQ ID NO:37. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 304 according to SEQ ID NO:17 or a cytosine at a position corresponding to position 304 according to SEQ ID NO:37, and at least 1, 2, 3, 4, 5, 6, 7, 8, 9, 10, or more nucleotides on each side of positions comprising a cytosine at a position corresponding to position 304 according to SEQ ID NO:17 or a cytosine at a position corresponding to position 304 according to SEQ ID NO:37.

In some embodiments, to determine whether an SLC26A5 nucleic acid molecule (mRNA molecule, or cDNA molecule), or complement thereof, within a biological sample comprises a nucleotide sequence comprising a cytosine at a position corresponding to position 304 according to SEQ ID NO:18 (mRNA molecule) or a cytosine at a position corresponding to position 304 according to SEQ ID NO:38 (cDNA molecule), the biological sample can be subjected to an amplification method using a primer pair that includes a first primer derived from the 5′ flanking sequence adjacent to a cytosine at a position corresponding to position 304 according to SEQ ID NO:18 or a cytosine at a position corresponding to position 304 according to SEQ ID NO:38, and a second primer derived from the 3′ flanking sequence adjacent to a cytosine at a position corresponding to position 304 according to SEQ ID NO:18 or a cytosine at a position corresponding to position 304 according to SEQ ID NO:38 to produce an amplicon that is indicative of the presence of the SNP at positions encoding a cytosine at a position corresponding to position 304 according to SEQ ID NO:18 or a cytosine at a position corresponding to position 304 according to SEQ ID NO:38. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 304 according to SEQ ID NO:18 or a cytosine at a position corresponding to position 304 according to SEQ ID NO:38, and at least 1, 2, 3, 4, 5, 6, 7, 8, 9, 10, or more nucleotides on each side of positions comprising a cytosine at a position corresponding to position 304 according to SEQ ID NO:18 or a cytosine at a position corresponding to position 304 according to SEQ ID NO:38.

In some embodiments, to determine whether an SLC26A5 nucleic acid molecule (mRNA molecule, or cDNA molecule), or complement thereof, within a biological sample comprises a nucleotide sequence comprising a cytosine at a position corresponding to position 304 according to SEQ ID NO:19 (mRNA molecule) or a cytosine at a position corresponding to position 304 according to SEQ ID NO:39 (cDNA molecule), the biological sample can be subjected to an amplification method using a primer pair that includes a first primer derived from the 5′ flanking sequence adjacent to a cytosine at a position corresponding to position 304 according to SEQ ID NO:19 or a cytosine at a position corresponding to position 304 according to SEQ ID NO:39, and a second primer derived from the 3′ flanking sequence adjacent to a cytosine at a position corresponding to position 304 according to SEQ ID NO:19 or a cytosine at a position corresponding to position 304 according to SEQ ID NO:39 to produce an amplicon that is indicative of the presence of the SNP at positions encoding a cytosine at a position corresponding to position 304 according to SEQ ID NO:19 or a cytosine at a position corresponding to position 304 according to SEQ ID NO:39. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 304 according to SEQ ID NO:19 or a cytosine at a position corresponding to position 304 according to SEQ ID NO:39, and at least 1, 2, 3, 4, 5, 7, 7, 8, 9, 10, or more nucleotides on each side of positions comprising or a cytosine at a position corresponding to position 304 according to SEQ ID NO:19 or a cytosine at a position corresponding to position 304 according to SEQ ID NO:39.

In some embodiments, to determine whether an SLC26A5 nucleic acid molecule (mRNA molecule, or cDNA molecule), or complement thereof, within a biological sample comprises a nucleotide sequence comprising a cytosine at a position corresponding to position 145 according to SEQ ID NO:20 (mRNA molecule) or a cytosine at a position corresponding to position 145 according to SEQ ID NO:40 (cDNA molecule), the biological sample can be subjected to an amplification method using a primer pair that includes a first primer derived from the 5′ flanking sequence adjacent to a cytosine at a position corresponding to position 145 according to SEQ ID NO:20 or a cytosine at a position corresponding to position 145 according to SEQ ID NO:40, and a second primer derived from the 3′ flanking sequence adjacent to a cytosine at a position corresponding to position 145 according to SEQ ID NO:20 or a cytosine at a position corresponding to position 145 according to SEQ ID NO:40 to produce an amplicon that is indicative of the presence of the SNP at positions encoding a cytosine at a position corresponding to position 145 according to SEQ ID NO:20 or a cytosine at a position corresponding to position 145 according to SEQ ID NO:40. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 145 according to SEQ ID NO:20 or a cytosine at a position corresponding to position 145 according to SEQ ID NO:40, and at least 1, 2, 3, 4, 5, 8, 7, 8, 9, 10, or more nucleotides on each side of positions comprising a cytosine at a position corresponding to position 145 according to SEQ ID NO:20 or a cytosine at a position corresponding to position 145 according to SEQ ID NO:40.

In some embodiments, to determine whether an SLC26A5 nucleic acid molecule (mRNA molecule, or cDNA molecule), or complement thereof, within a biological sample comprises a nucleotide sequence comprising a cytosine at a position corresponding to position 145 according to SEQ ID NO:21 (mRNA molecule) or a cytosine at a position corresponding to position 145 according to SEQ ID NO:41 (cDNA molecule), the biological sample can be subjected to an amplification method using a primer pair that includes a first primer derived from the 5′ flanking sequence adjacent to a cytosine at a position corresponding to position 145 according to SEQ ID NO:21 or a cytosine at a position corresponding to position 145 according to SEQ ID NO:41, and a second primer derived from the 3′ flanking sequence adjacent to a cytosine at a position corresponding to position 145 according to SEQ ID NO:21 or a cytosine at a position corresponding to position 145 according to SEQ ID NO:41 to produce an amplicon that is indicative of the presence of the SNP at positions encoding a cytosine at a position corresponding to position 145 according to SEQ ID NO:21 or a cytosine at a position corresponding to position 145 according to SEQ ID NO:41. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 145 according to SEQ ID NO:21 or a cytosine at a position corresponding to position 145 according to SEQ ID NO:41, and at least 1, 2, 3, 4, 5, 9, 7, 8, 9, 10, or more nucleotides on each side of positions comprising a cytosine at a position corresponding to position 145 according to SEQ ID NO:21 or a cytosine at a position corresponding to position 145 according to SEQ ID NO:41.

In some embodiments, to determine whether an SLC26A5 nucleic acid molecule (mRNA molecule, or cDNA molecule), or complement thereof, within a biological sample comprises a nucleotide sequence comprising a cytosine at a position corresponding to position 205 according to SEQ ID NO:22 (mRNA molecule) or a cytosine at a position corresponding to position 205 according to SEQ ID NO:42 (cDNA molecule), the biological sample can be subjected to an amplification method using a primer pair that includes a first primer derived from the 5′ flanking sequence adjacent cytosine at a position corresponding to position 205 according to SEQ ID NO:22 or a cytosine at a position corresponding to position 205 according to SEQ ID NO:42, and a second primer derived from the 3′ flanking sequence adjacent to a cytosine at a position corresponding to position 205 according to SEQ ID NO:22 or a cytosine at a position corresponding to position 205 according to SEQ ID NO:42 to produce an amplicon that is indicative of the presence of the SNP at positions encoding a cytosine at a position corresponding to position 205 according to SEQ ID NO:22 or a cytosine at a position corresponding to position 205 according to SEQ ID NO:42. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 205 according to SEQ ID NO:22 or a cytosine at a position corresponding to position 205 according to SEQ ID NO:42, and at least 1, 2, 3, 4, 5, 10, 7, 8, 9, 10, or more nucleotides on each side of positions comprising a cytosine at a position corresponding to position 205 according to SEQ ID NO:22 or a cytosine at a position corresponding to position 205 according to SEQ ID NO:42.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of an SLC26A5 predicted loss-of-function polypeptide comprising performing an assay on a sample obtained from a subject to determine whether an SLC26A5 polypeptide in the subject contains one or more variations that causes the polypeptide to have a loss-of-function (partial or complete) or predicted loss-of-function (partial or complete). The SLC26A5 predicted loss-of-function polypeptide can be any of the SLC26A5 truncated variant polypeptides described herein. In some embodiments, the methods detect the presence of SLC26A5 Leu46Pro. In some embodiments, the methods detect the presence of SLC26A5 Leu46Pro Isoform 1. In some embodiments, the methods detect the presence of SLC26A5 Leu46Pro Isoform 2. In some embodiments, the methods detect the presence of SLC26A5 Leu46Pro Isoform 3. In some embodiments, the methods detect the presence of SLC26A5 Leu46Pro Isoform 4. In some embodiments, the methods detect the presence of SLC26A5 Leu46Pro Isoform 5. In some embodiments, the methods detect the presence of SLC26A5 Leu46Pro Isoform 6. In some embodiments, the methods detect the presence of SLC26A5 Leu46Pro Isoform 7. In some embodiments, the methods detect the presence of SLC26A5 Leu46Pro Isoform 8. In some embodiments, the methods detect the presence of SLC26A5 Leu46Pro Isoform 9.

In some embodiments, the methods comprise performing an assay on a sample obtained from a subject to determine whether an SLC26A5 polypeptide in the sample comprises a proline at a position corresponding to position 46 according to SEQ ID NO:52. In some embodiments, the methods comprise performing an assay on a sample obtained from a subject to determine whether an SLC26A5 polypeptide in the sample comprises a proline at a position corresponding to position 46 according to SEQ ID NO:53. In some embodiments, the methods comprise performing an assay on a sample obtained from a subject to determine whether an SLC26A5 polypeptide in the sample comprises a proline at a position corresponding to position 46 according to SEQ ID NO:54. In some embodiments, the methods comprise performing an assay on a sample obtained from a subject to determine whether an SLC26A5 polypeptide in the sample comprises a proline at a position corresponding to position 46 according to SEQ ID NO:55. In some embodiments, the methods comprise performing an assay on a sample obtained from a subject to determine whether an SLC26A5 polypeptide in the sample comprises a proline at a position corresponding to position 46 according to SEQ ID NO:56. In some embodiments, the methods comprise performing an assay on a sample obtained from a subject to determine whether an SLC26A5 polypeptide in the sample comprises a proline at a position corresponding to position 46 according to SEQ ID NO:57. In some embodiments, the methods comprise performing an assay on a sample obtained from a subject to determine whether an SLC26A5 polypeptide in the sample comprises a proline at a position corresponding to position 46 according to SEQ ID NO:58. In some embodiments, the methods comprise performing an assay on a sample obtained from a subject to determine whether an SLC26A5 polypeptide in the sample comprises a proline at a position corresponding to position 46 according to SEQ ID NO:59. In some embodiments, the methods comprise performing an assay on a sample obtained from a subject to determine whether an SLC26A5 polypeptide in the sample comprises a proline at a position corresponding to position 46 according to SEQ ID NO:60.

In some embodiments, the detecting step comprises sequencing at least a portion of the polypeptide that comprises a position corresponding to position 46 according to SEQ ID NO:52 or SEQ ID NO:43. In some embodiments, the detecting step comprises sequencing at least a portion of the polypeptide that comprises a position corresponding to position 46 according to SEQ ID NO:53 or SEQ ID NO:44. In some embodiments, the detecting step comprises sequencing at least a portion of the polypeptide that comprises a position corresponding to position 46 according to SEQ ID NO:54 or SEQ ID NO:45. In some embodiments, the detecting step comprises sequencing at least a portion of the polypeptide that comprises a position corresponding to position 46 according to SEQ ID NO:55 or SEQ ID NO:46. In some embodiments, the detecting step comprises sequencing at least a portion of the polypeptide that comprises a position corresponding to position 46 according to SEQ ID NO:56 or SEQ ID NO:47. In some embodiments, the detecting step comprises sequencing at least a portion of the polypeptide that comprises a position corresponding to position 46 according to SEQ ID NO:57 or SEQ ID NO:48. In some embodiments, the detecting step comprises sequencing at least a portion of the polypeptide that comprises a position corresponding to position 46 according to SEQ ID NO:58 or SEQ ID NO:49. In some embodiments, the detecting step comprises sequencing at least a portion of the polypeptide that comprises a position corresponding to position 46 according to SEQ ID NO:59 or SEQ ID NO:50. In some embodiments, the detecting step comprises sequencing at least a portion of the polypeptide that comprises a position corresponding to position 46 according to SEQ ID NO:60 or SEQ ID NO:51.

In some embodiments, the detecting step comprises an immunoassay for detecting the presence of a polypeptide that comprises a position corresponding to position 46 according to SEQ ID NO:52 or SEQ ID NO:43. In some embodiments, the detecting step comprises an immunoassay for detecting the presence of a polypeptide that comprises a position corresponding to position 46 according to SEQ ID NO:53 or SEQ ID NO:44. In some embodiments, the detecting step comprises an immunoassay for detecting the presence of a polypeptide that comprises a position corresponding to position 46 according to SEQ ID NO:54 or SEQ ID NO:45. In some embodiments, the detecting step comprises an immunoassay for detecting the presence of a polypeptide that comprises a position corresponding to position 46 according to SEQ ID NO:55 or SEQ ID NO:46. In some embodiments, the detecting step comprises an immunoassay for detecting the presence of a polypeptide that comprises a position corresponding to position 46 according to SEQ ID NO:56 or SEQ ID NO:47. In some embodiments, the detecting step comprises an immunoassay for detecting the presence of a polypeptide that comprises a position corresponding to position 46 according to SEQ ID NO:57 or SEQ ID NO:48. In some embodiments, the detecting step comprises an immunoassay for detecting the presence of a polypeptide that comprises a position corresponding to position 46 according to SEQ ID NO:58 or SEQ ID NO:49. In some embodiments, the detecting step comprises an immunoassay for detecting the presence of a polypeptide that comprises a position corresponding to position 46 according to SEQ ID NO:59 or SEQ ID NO:50. In some embodiments, the detecting step comprises an immunoassay for detecting the presence of a polypeptide that comprises a position corresponding to position 46 according to SEQ ID NO:60 or SEQ ID NO:51.

In some embodiments, when the subject does not have an SLC26A5 predicted loss-of-function polypeptide, then the subject does not have an increased risk for developing hearing loss or any of conductive hearing loss, sensorineural hearing loss, or neural hearing loss.

The present disclosure also provides isolated nucleic acid molecules that hybridize to SLC26A5 variant genomic nucleic acid molecules, SLC26A5 variant mRNA molecules, and/or SLC26A5 variant cDNA molecules (such as any of the genomic variant nucleic acid molecules, mRNA variant molecules, and cDNA variant molecules disclosed herein). In some embodiments, the isolated nucleic acid molecules hybridize to a portion of the SLC26A5 nucleic acid molecule that includes a position corresponding to: position 24,774 according to SEQ ID NO:2, position 373 according to SEQ ID NO:13, or position 373 according to SEQ ID NO:33. In some embodiments, the isolated nucleic acid molecules hybridize to a portion of the SLC26A5 nucleic acid molecule that includes a position corresponding to position 373 according to SEQ ID NO:14, or position 373 according to SEQ ID NO:34. In some embodiments, the isolated nucleic acid molecules hybridize to a portion of the SLC26A5 nucleic acid molecule that includes a position corresponding to position 373 according to SEQ ID NO:15, or position 373 according to SEQ ID NO:35. In some embodiments, the isolated nucleic acid molecules hybridize to a portion of the SLC26A5 nucleic acid molecule that includes a position corresponding to position 373 according to SEQ ID NO:16, or position 373 according to SEQ ID NO:36. In some embodiments, the isolated nucleic acid molecules hybridize to a portion of the SLC26A5 nucleic acid molecule that includes a position corresponding to position 304 according to SEQ ID NO:17, or position 304 according to SEQ ID NO:37. In some embodiments, the isolated nucleic acid molecules hybridize to a portion of the SLC26A5 nucleic acid molecule that includes a position corresponding to position 304 according to SEQ ID NO:18, or position 304 according to SEQ ID NO:38. In some embodiments, the isolated nucleic acid molecules hybridize to a portion of the SLC26A5 nucleic acid molecule that includes a position corresponding to position 304 according to SEQ ID NO:19, or position 304 according to SEQ ID NO:39. In some embodiments, the isolated nucleic acid molecules hybridize to a portion of the SLC26A5 nucleic acid molecule that includes a position corresponding to position 145 according to SEQ ID NO:20, or position 145 according to SEQ ID NO:40. In some embodiments, the isolated nucleic acid molecules hybridize to a portion of the SLC26A5 nucleic acid molecule that includes a position corresponding to position 145 according to SEQ ID NO:21, or position 145 according to SEQ ID NO:41. In some embodiments, the isolated nucleic acid molecules hybridize to a portion of the SLC26A5 nucleic acid molecule that includes a position corresponding to position 205 according to SEQ ID NO:22, or position 205 according to SEQ ID NO:42.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to SLC26A5 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to SLC26A5 variant genomic nucleic acid molecules, SLC26A5 variant mRNA molecules, and/or SLC26A5 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 24,774 according to SEQ ID NO:2, or the complement thereof; position 373 according to SEQ ID NO:13, or the complement thereof; or position 373 according to SEQ ID NO:33, or the complement thereof. In some embodiments, the alteration-specific probe or alteration-specific primer comprises a nucleotide sequence which is complementary to a portion of a nucleotide sequence comprising positions corresponding to: positions 24,773-24,775 according to SEQ ID NO:2, or the complement thereof; positions 372-374 according to SEQ ID NO:13, or the complement thereof; and/or positions 372-374 according to SEQ ID NO:33, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 373 according to SEQ ID NO:14, or the complement thereof; or position 373 according to SEQ ID NO:34, or the complement thereof. In some embodiments, the alteration-specific probe or alteration-specific primer comprises a nucleotide sequence which is complementary to a portion of a nucleotide sequence comprising positions corresponding to: positions 372-374 according to SEQ ID NO:14, or the complement thereof; and/or positions 372-374 according to SEQ ID NO:34, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 373 according to SEQ ID NO:15, or the complement thereof; or position 373 according to SEQ ID NO:35, or the complement thereof. In some embodiments, the alteration-specific probe or alteration-specific primer comprises a nucleotide sequence which is complementary to a portion of a nucleotide sequence comprising positions corresponding to: positions 372-374 according to SEQ ID NO:15, or the complement thereof; and/or positions 372-374 according to SEQ ID NO:35, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 373 according to SEQ ID NO:16, or the complement thereof; or position 373 according to SEQ ID NO:36, or the complement thereof. In some embodiments, the alteration-specific probe or alteration-specific primer comprises a nucleotide sequence which is complementary to a portion of a nucleotide sequence comprising positions corresponding to: positions 372-374 according to SEQ ID NO:16, or the complement thereof; and/or positions 372-374 according to SEQ ID NO:36, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 304 according to SEQ ID NO:17, or the complement thereof; or position 304 according to SEQ ID NO:37, or the complement thereof. In some embodiments, the alteration-specific probe or alteration-specific primer comprises a nucleotide sequence which is complementary to a portion of a nucleotide sequence comprising positions corresponding to: positions 303-305 according to SEQ ID NO:17, or the complement thereof; and/or positions 303-305 according to SEQ ID NO:37, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 304 according to SEQ ID NO:18, or the complement thereof; or position 304 according to SEQ ID NO:38, or the complement thereof. In some embodiments, the alteration-specific probe or alteration-specific primer comprises a nucleotide sequence which is complementary to a portion of a nucleotide sequence comprising positions corresponding to: positions 303-305 according to SEQ ID NO:18, or the complement thereof; and/or positions 303-305 according to SEQ ID NO:38, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 304 according to SEQ ID NO:19, or the complement thereof; or position 304 according to SEQ ID NO:39, or the complement thereof. In some embodiments, the alteration-specific probe or alteration-specific primer comprises a nucleotide sequence which is complementary to a portion of a nucleotide sequence comprising positions corresponding to: positions 303-305 according to SEQ ID NO:19, or the complement thereof; and/or positions 303-305 according to SEQ ID NO:39, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 145 according to SEQ ID NO:20, or the complement thereof; or position 145 according to SEQ ID NO:40, or the complement thereof. In some embodiments, the alteration-specific probe or alteration-specific primer comprises a nucleotide sequence which is complementary to a portion of a nucleotide sequence comprising positions corresponding to: positions 144-146 according to SEQ ID NO:20, or the complement thereof; and/or positions 144-146 according to SEQ ID NO:40, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 145 according to SEQ ID NO:21, or the complement thereof; or position 145 according to SEQ ID NO:41, or the complement thereof. In some embodiments, the alteration-specific probe or alteration-specific primer comprises a nucleotide sequence which is complementary to a portion of a nucleotide sequence comprising positions corresponding to: positions 144-146 according to SEQ ID NO:21, or the complement thereof; and/or positions 144-146 according to SEQ ID NO:41, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding an SLC26A5 polypeptide, wherein the portion comprises a position corresponding to: position 205 according to SEQ ID NO:22, or the complement thereof; or position 205 according to SEQ ID NO:42, or the complement thereof. In some embodiments, the alteration-specific probe or alteration-specific primer comprises a nucleotide sequence which is complementary to a portion of a nucleotide sequence comprising positions corresponding to: positions 204-206 according to SEQ ID NO:22, or the complement thereof; and/or positions 204-206 according to SEQ ID NO:42, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the SLC26A5 variant genomic nucleic acid molecules, SLC26A5 variant mRNA molecules, and/or SLC26A5 variant cDNA molecules disclosed herein. The primers described herein can be used to amplify SLC26A5 variant genomic nucleic acid molecules, SLC26A5 variant mRNA molecules, or SLC26A5 variant cDNA molecules, or a fragment thereof.

The present disclosure also provides pairs of primers comprising any of the primers described above. For example, if one of the primers' 3′-ends hybridizes to a thymine at a position corresponding to position 24,773 according to SEQ ID NO:1 (rather than cytosine) in a particular SLC26A5 nucleic acid molecule, then the presence of the amplified fragment would indicate the presence of an SLC26A5 reference genomic nucleic acid molecule. Conversely, if one of the primers' 3′-ends hybridizes to a cytosine at a position corresponding to position 24,774 according to SEQ ID NO:2 (rather than thymine) in a particular SLC26A5 nucleic acid molecule, then the presence of the amplified fragment would indicate the presence of the SLC26A5 variant genomic nucleic acid molecule. In some embodiments, the nucleotide of the primer complementary to the cytosine at a position corresponding to position 24,774 according to SEQ ID NO:2 can be at the 3′ end of the primer. In addition, if one of the primers' 3′-ends hybridizes to a uracil at a position corresponding to position 373 according to SEQ ID NO:3 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 373 according to SEQ ID NO:13 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 373 according to SEQ ID NO:13 can be at the 3′ end of the primer. In addition, if one of the primers' 3′-ends hybridizes to a thymine at a position corresponding to position 373 according to SEQ ID NO:23 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 373 according to SEQ ID NO:33 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 373 according to SEQ ID NO:33 can be at the 3′ end of the primer.

If, for example, if one of the primers' 3′-ends hybridizes to a uracil at a position corresponding to position 373 according to SEQ ID NO:4 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 373 according to SEQ ID NO:14 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 373 according to SEQ ID NO:14 can be at the 3′ end of the primer. In addition, if one of the primers' 3′-ends hybridizes to a thymine at a position corresponding to position 373 according to SEQ ID NO:24 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 373 according to SEQ ID NO:34 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 373 according to SEQ ID NO:34 can be at the 3′ end of the primer.

If, for example, one of the primers' 3′-ends hybridizes to a uracil at a position corresponding to position 373 according to SEQ ID NO:5 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 373 according to SEQ ID NO:15 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 373 according to SEQ ID NO:15 can be at the 3′ end of the primer. In addition, if one of the primers' 3′-ends hybridizes to a thymine at a position corresponding to position 373 according to SEQ ID NO:25 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 373 according to SEQ ID NO:35 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 373 according to SEQ ID NO:35 can be at the 3′ end of the primer.

If, for example, one of the primers' 3′-ends hybridizes to a uracil at a position corresponding to position 373 according to SEQ ID NO:6 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 373 according to SEQ ID NO:16 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 373 according to SEQ ID NO:16 can be at the 3′ end of the primer. In addition, if one of the primers' 3′-ends hybridizes to a thymine at a position corresponding to position 373 according to SEQ ID NO:26 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 373 according to SEQ ID NO:36 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 373 according to SEQ ID NO:36 can be at the 3′ end of the primer.

If, for example, if one of the primers' 3′-ends hybridizes to a uracil at a position corresponding to position 304 according to SEQ ID NO:7 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 304 according to SEQ ID NO:17 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 304 according to SEQ ID NO:17 can be at the 3′ end of the primer. In addition, if one of the primers' 3′-ends hybridizes to a thymine at a position corresponding to position 304 according to SEQ ID NO:27 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 304 according to SEQ ID NO:37 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 304 according to SEQ ID NO:37 can be at the 3′ end of the primer.

If, for example, one of the primers' 3′-ends hybridizes to a uracil at a position corresponding to position 304 according to SEQ ID NO:8 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 304 according to SEQ ID NO:18 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 304 according to SEQ ID NO:18 can be at the 3′ end of the primer. In addition, if one of the primers' 3′-ends hybridizes to a thymine at a position corresponding to position 304 according to SEQ ID NO:28 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 304 according to SEQ ID NO:38 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 304 according to SEQ ID NO:38 can be at the 3′ end of the primer.

If, for example, one of the primers' 3′-ends hybridizes to a uracil at a position corresponding to position 304 according to SEQ ID NO:9 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 304 according to SEQ ID NO:19 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 304 according to SEQ ID NO:19 can be at the 3′ end of the primer. In addition, if one of the primers' 3′-ends hybridizes to a thymine at a position corresponding to position 304 according to SEQ ID NO:29 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 304 according to SEQ ID NO:39 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 304 according to SEQ ID NO:39 can be at the 3′ end of the primer.

If, for example, one of the primers' 3′-ends hybridizes to a uracil at a position corresponding to position 145 according to SEQ ID NO:10 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 145 according to SEQ ID NO:20 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 145 according to SEQ ID NO:20 can be at the 3′ end of the primer. In addition, if one of the primers' 3′-ends hybridizes to a thymine at a position corresponding to position 145 according to SEQ ID NO:30 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 145 according to SEQ ID NO:40 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 145 according to SEQ ID NO:40 can be at the 3′ end of the primer.

If, for example, one of the primers' 3′-ends hybridizes to a uracil at a position corresponding to position 145 according to SEQ ID NO:11 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 145 according to SEQ ID NO:21 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 145 according to SEQ ID NO:21 can be at the 3′ end of the primer. In addition, if one of the primers' 3′-ends hybridizes to a thymine at a position corresponding to position 145 according to SEQ ID NO:31 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 145 according to SEQ ID NO:41 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 145 according to SEQ ID NO:41 can be at the 3′ end of the primer.

If, for example, one of the primers' 3′-ends hybridizes to a uracil at a position corresponding to position 205 according to SEQ ID NO:12 (rather than cytosine) in a particular SLC26A5 nucleic acid molecule, then the presence of the amplified fragment would indicate the presence of an SLC26A5 reference mRNA molecule. Conversely, if one of the primers' 3′-ends hybridizes to a cytosine at a position corresponding to position 205 according to SEQ ID NO:22 (rather than uracil) in a particular SLC26A5 mRNA molecule, then the presence of the amplified fragment would indicate the presence of the SLC26A5 variant mRNA molecule. In some embodiments, the nucleotide of the primer complementary to the cytosine at a position corresponding to position 205 according to SEQ ID NO:22 can be at the 3′ end of the primer. In addition, if one of the primers' 3′-ends hybridizes to a thymine at a position corresponding to position 205 according to SEQ ID NO:32 (rather than cytosine) in a particular SLC26A5 nucleic acid molecule, then the presence of the amplified fragment would indicate the presence of an SLC26A5 reference cDNA molecule. Conversely, if one of the primers' 3′-ends hybridizes to a cytosine at a position corresponding to position 205 according to SEQ ID NO:42 (rather than thymine) in a particular SLC26A5 cDNA molecule, then the presence of the amplified fragment would indicate the presence of the SLC26A5 variant cDNA molecule. In some embodiments, the nucleotide of the primer complementary to the cytosine at a position corresponding to position 205 according to SEQ ID NO:42 can be at the 3′ end of the primer.

In the context of the disclosure “specifically hybridizes” means that the probe or primer (such as, for example, the alteration-specific probe or alteration-specific primer) does not hybridize to a nucleic acid sequence encoding an SLC26A5 reference genomic nucleic acid molecule, an SLC26A5 reference mRNA molecule, and/or an SLC26A5 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The present disclosure also provides molecular complexes comprising or consisting of any of the SLC26A5 nucleic acid molecules (genomic nucleic acid molecules, mRNA molecules, or cDNA molecules), or complement thereof, described herein and any of the alteration-specific primers or alteration-specific probes described herein. In some embodiments, the SLC26A5 nucleic acid molecules (genomic nucleic acid molecules, mRNA molecules, or cDNA molecules), or complement thereof, in the molecular complexes are single-stranded. In some embodiments, the SLC26A5 nucleic acid molecule is any of the genomic nucleic acid molecules described herein. In some embodiments, the SLC26A5 nucleic acid molecule is any of the mRNA molecules described herein. In some embodiments, the SLC26A5 nucleic acid molecule is any of the cDNA molecules described herein. In some embodiments, the molecular complex comprises or consists of any of the SLC26A5 nucleic acid molecules (genomic nucleic acid molecules, mRNA molecules, or cDNA molecules), or complement thereof, described herein and any of the alteration-specific primers described herein. In some embodiments, the molecular complex comprises or consists of any of the SLC26A5 nucleic acid molecules (genomic nucleic acid molecules, mRNA molecules, or cDNA molecules), or complement thereof, described herein and any of the alteration-specific probes described herein.

In some embodiments, the molecular complex comprises or consists of an alteration-specific primer or an alteration-specific probe hybridized to a genomic nucleic acid molecule comprising a nucleotide sequence encoding an SLC26A5 polypeptide, wherein the alteration-specific primer or the alteration-specific probe is hybridized to a cytosine at a position corresponding to position 24,774 according to SEQ ID NO:2, or the complement thereof.

In some embodiments, the molecular complex comprises or consists of an alteration-specific primer or an alteration-specific probe that is hybridized to a CCG codon at positions corresponding to positions 24,773-24,775 according to SEQ ID NO:2.

In some embodiments, the molecular complex comprises or consists of a genomic nucleic acid molecule that comprises SEQ ID NO:2.

In some embodiments, the molecular complex comprises or consists of an alteration-specific primer or an alteration-specific probe hybridized to an mRNA molecule comprising a nucleotide sequence encoding an SLC26A5 polypeptide, wherein the alteration-specific primer or the alteration-specific probe is hybridized to: a cytosine at a position corresponding to position 373 according to SEQ ID NO:13, or the complement thereof; a cytosine at a position corresponding to position 373 according to SEQ ID NO:14, or the complement thereof; a cytosine at a position corresponding to position 373 according to SEQ ID NO:15, or the complement thereof; a cytosine at a position corresponding to position 373 according to SEQ ID NO:16, or the complement thereof; a cytosine at a position corresponding to position 304 according to SEQ ID NO:17, or the complement thereof; a cytosine at a position corresponding to position 304 according to SEQ ID NO:18, or the complement thereof; a cytosine at a position corresponding to position 304 according to SEQ ID NO:19, or the complement thereof; a cytosine at a position corresponding to position 145 according to SEQ ID NO:20, or the complement thereof; a cytosine at a position corresponding to position 145 according to SEQ ID NO:21, or the complement thereof; or a cytosine at a position corresponding to position 205 according to SEQ ID NO:22, or the complement thereof.

In some embodiments, the molecular complex comprises or consists of an alteration-specific primer or an alteration-specific probe that is hybridized to: a CCG codon at positions corresponding to positions 372-374 according to SEQ ID NO:13, a CCG codon at positions corresponding to positions 372-374 according to SEQ ID NO:14, a CCG codon at positions corresponding to positions 372-374 according to SEQ ID NO:15, a CCG codon at positions corresponding to positions 372-374 according to SEQ ID NO:16, a CCG codon at positions corresponding to positions 303-305 according to SEQ ID NO:17, a CCG codon at positions corresponding to positions 303-305 according to SEQ ID NO:18, a CCG codon at positions corresponding to positions 303-305 according to SEQ ID NO:19, a CCG codon at positions corresponding to positions 144-146 according to SEQ ID NO:20, a CCG codon at positions corresponding to positions 144-146 according to SEQ ID NO:21, or a CCG codon at positions corresponding to positions 204-206 according to SEQ ID NO:22.

In some embodiments, the molecular complex comprises or consists of an mRNA molecule that comprises SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

In some embodiments, the molecular complex comprises or consists of an alteration-specific primer or an alteration-specific probe hybridized to a cDNA molecule comprising a nucleotide sequence encoding an SLC26A5 polypeptide, wherein the alteration-specific primer or the alteration-specific probe is hybridized to: a cytosine at a position corresponding to position 373 according to SEQ ID NO:33, or the complement thereof; a cytosine at a position corresponding to position 373 according to SEQ ID NO:34, or the complement thereof; a cytosine at a position corresponding to position 373 according to SEQ ID NO:35, or the complement thereof; a cytosine at a position corresponding to position 373 according to SEQ ID NO:36, or the complement thereof; a cytosine at a position corresponding to position 304 according to SEQ ID NO:37, a cytosine at a position corresponding to position 304 according to SEQ ID NO:38, or the complement thereof; a cytosine at a position corresponding to position 304 according to SEQ ID NO:39, or the complement thereof; a cytosine at a position corresponding to position 145 according to SEQ ID NO:40, or the complement thereof; a cytosine at a position corresponding to position 145 according to SEQ ID NO:41, or the complement thereof; or a cytosine at a position corresponding to position 205 according to SEQ ID NO:42, or the complement thereof.

In some embodiments, the molecular complex comprises or consists of an alteration-specific primer or an alteration-specific probe that is hybridized to: a CCG codon at positions corresponding to positions 372-374 according to SEQ ID NO:33, a CCG codon at positions corresponding to positions 372-374 according to SEQ ID NO:34, a CCG codon at positions corresponding to positions 372-374 according to SEQ ID NO:35, a CCG codon at positions corresponding to positions 372-374 according to SEQ ID NO:36, a CCG codon at positions corresponding to positions 303-305 according to SEQ ID NO:37, a CCG codon at positions corresponding to positions 303-305 according to SEQ ID NO:38, a CCG codon at positions corresponding to positions 303-305 according to SEQ ID NO:39, a CCG codon at positions corresponding to positions 144-146 according to SEQ ID NO:40, a CCG codon at positions corresponding to positions 144-146 according to SEQ ID NO:41, or a CCG codon at positions corresponding to positions 204-206 according to SEQ ID NO:42.

In some embodiments, the molecular complex comprises or consists of a cDNA molecule that comprises SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, or SEQ ID NO:42.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of an SLC26A5 reference genomic nucleic acid molecule is set forth in SEQ ID NO:1. Referring to SEQ ID NO:1, position 24,773 is a thymine.

A variant genomic nucleic acid molecule of SLC26A5 exists, wherein the thymine at position 24,773, is replaced with cytosine. The nucleotide sequence of this SLC26A5 variant genomic nucleic acid molecule is set forth in SEQ ID NO:2.

The nucleotide sequence of an SLC26A5 reference mRNA molecule is set forth in SEQ ID NO:3. Referring to SEQ ID NO:3, position 373 is a uracil.

The nucleotide sequence of another SLC26A5 reference mRNA molecule is set forth in SEQ ID NO:4. Referring to SEQ ID NO:4, position 373 is a uracil.

The nucleotide sequence of another SLC26A5 reference mRNA molecule is set forth in SEQ ID NO:5. Referring to SEQ ID NO:5, position 373 is a uracil.

The nucleotide sequence of another SLC26A5 reference mRNA molecule is set forth in SEQ ID NO:6. Referring to SEQ ID NO:6, position 373 is a uracil.

The nucleotide sequence of another SLC26A5 reference mRNA molecule is set forth in SEQ ID NO:7. Referring to SEQ ID NO:7, position 304 is a uracil.

The nucleotide sequence of another SLC26A5 reference mRNA molecule is set forth in SEQ ID NO:8. Referring to SEQ ID NO:8, position 304 is a uracil.

The nucleotide sequence of another SLC26A5 reference mRNA molecule is set forth in SEQ ID NO:9. Referring to SEQ ID NO:9, position 304 is a uracil.

The nucleotide sequence of another SLC26A5 reference mRNA molecule is set forth in SEQ ID NO:10. Referring to SEQ ID NO:10, position 145 is a uracil.

The nucleotide sequence of another SLC26A5 reference mRNA molecule is set forth in SEQ ID NO:11. Referring to SEQ ID NO:11, position 145 is a uracil.

The nucleotide sequence of another SLC26A5 reference mRNA molecule is set forth in SEQ ID NO:12. Referring to SEQ ID NO:12, position 205 is a uracil.

NCBI RefSeq numbers for the reference mRNA molecules include: NM_001167962.1, NM_001321787.2, NM_198999.3, NM_206883.3, NM_206884.3, NM_206885.3, NM_001321787.2, NR_135802.2, NR_120441.1, NR_120443.1, NR_120442.1, and NR_135801.1.

A variant mRNA molecule of SLC26A5 exists, wherein the uracil at position 373, is replaced with cytosine. The nucleotide sequence of this SLC26A5 variant mRNA molecule is set forth in SEQ ID NO:13.

Another variant mRNA molecule of SLC26A5 exists, wherein the uracil at position 373 is replaced with cytosine. The nucleotide sequence of this SLC26A5 variant mRNA molecule is set forth in SEQ ID NO:14.

Another variant mRNA molecule of SLC26A5 exists, wherein the uracil at position 373 is replaced with cytosine. The nucleotide sequence of this SLC26A5 variant mRNA molecule is set forth in SEQ ID NO:15.

Another variant mRNA molecule of SLC26A5 exists, wherein the uracil at position 373 is replaced with cytosine. The nucleotide sequence of this SLC26A5 variant mRNA molecule is set forth in SEQ ID NO:16.

Another variant mRNA molecule of SLC26A5 exists, wherein the uracil at position 304 is replaced with cytosine. The nucleotide sequence of this SLC26A5 variant mRNA molecule is set forth in SEQ ID NO:17.

Another variant mRNA molecule of SLC26A5 exists, wherein the uracil at position 304 is replaced with cytosine. The nucleotide sequence of this SLC26A5 variant mRNA molecule is set forth in SEQ ID NO:18.

Another variant mRNA molecule of SLC26A5 exists, wherein the uracil at position 304 is replaced with cytosine. The nucleotide sequence of this SLC26A5 variant mRNA molecule is set forth in SEQ ID NO:19.

Another variant mRNA molecule of SLC26A5 exists, wherein the uracil at position 145 is replaced with cytosine. The nucleotide sequence of this SLC26A5 variant mRNA molecule is set forth in SEQ ID NO:20.

Another variant mRNA molecule of SLC26A5 exists, wherein the uracil at position 145 is replaced with cytosine. The nucleotide sequence of this SLC26A5 variant mRNA molecule is set forth in SEQ ID NO:21.

Another variant mRNA molecule of SLC26A5 exists, wherein the uracil at position 205 is replaced with cytosine. The nucleotide sequence of this SLC26A5 variant mRNA molecule is set forth in SEQ ID NO:22.

The nucleotide sequence of an SLC26A5 reference cDNA molecule is set forth in SEQ ID NO:23. Referring to SEQ ID NO:23, position 373 is a thymine.

The nucleotide sequence of another SLC26A5 reference cDNA molecule is set forth in SEQ ID NO:24. Referring to SEQ ID NO:24, position 373 is a thymine.

The nucleotide sequence of another SLC26A5 reference cDNA molecule is set forth in SEQ ID NO:25. Referring to SEQ ID NO:25, position 373 is a thymine.

The nucleotide sequence of another SLC26A5 reference cDNA molecule is set forth in SEQ ID NO:26. Referring to SEQ ID NO:26, position 373 is a thymine.

The nucleotide sequence of another SLC26A5 reference cDNA molecule is set forth in SEQ ID NO:27. Referring to SEQ ID NO:27, position 304 is a thymine.

The nucleotide sequence of another SLC26A5 reference cDNA molecule is set forth in SEQ ID NO:28. Referring to SEQ ID NO:28, position 304 is a thymine.

The nucleotide sequence of another SLC26A5 reference cDNA molecule is set forth in SEQ ID NO:29. Referring to SEQ ID NO:29, position 304 is a thymine.

The nucleotide sequence of another SLC26A5 reference cDNA molecule is set forth in SEQ ID NO:30. Referring to SEQ ID NO:30, position 145 is a thymine.

The nucleotide sequence of another SLC26A5 reference cDNA molecule is set forth in SEQ ID NO:31. Referring to SEQ ID NO:31, position 145 is a thymine.

The nucleotide sequence of another SLC26A5 reference cDNA molecule is set forth in SEQ ID NO:32. Referring to SEQ ID NO:32, position 205 is a thymine.

A variant cDNA molecule of SLC26A5 exists, wherein the thymine at position 373 is replaced with cytosine. The nucleotide sequence of this SLC26A5 variant cDNA molecule is set forth in SEQ ID NO:33.

Another variant cDNA molecule of SLC26A5 exists, wherein the thymine at position 373 is replaced with cytosine. The nucleotide sequence of this SLC26A5 variant cDNA molecule is set forth in SEQ ID NO:34.

Another variant cDNA molecule of SLC26A5 exists, wherein the thymine at position 373 is replaced with cytosine. The nucleotide sequence of this SLC26A5 variant cDNA molecule is set forth in SEQ ID NO:35.

Another variant cDNA molecule of SLC26A5 exists, wherein the thymine at position 373 is replaced with cytosine. The nucleotide sequence of this SLC26A5 variant cDNA molecule is set forth in SEQ ID NO:36.

Another variant cDNA molecule of SLC26A5 exists, wherein the thymine at position 304 is replaced with cytosine. The nucleotide sequence of this SLC26A5 variant cDNA molecule is set forth in SEQ ID NO:37.

Another variant cDNA molecule of SLC26A5 exists, wherein the thymine at position 304 is replaced with cytosine. The nucleotide sequence of this SLC26A5 variant cDNA molecule is set forth in SEQ ID NO:38.

Another variant mRNA molecule of SLC26A5 exists, wherein the uracil at position 304 is replaced with cytosine. The nucleotide sequence of this SLC26A5 variant mRNA molecule is set forth in SEQ ID NO:19.

Another variant cDNA molecule of SLC26A5 exists, wherein the thymine at position 145 is replaced with cytosine. The nucleotide sequence of this SLC26A5 variant cDNA molecule is set forth in SEQ ID NO:40.

Another variant cDNA molecule of SLC26A5 exists, wherein the thymine at position 145 is replaced with cytosine. The nucleotide sequence of this SLC26A5 variant cDNA molecule is set forth in SEQ ID NO:41.

Another variant cDNA molecule of SLC26A5 exists, wherein the thymine at position 205 is replaced with cytosine. The nucleotide sequence of this SLC26A5 variant cDNA molecule is set forth in SEQ ID NO:42.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (1), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety.

Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:3, or SEQ ID NO:23). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence encoding an SLC26A5 polypeptide, wherein the nucleotide sequence comprises a cytosine at a position corresponding to position 24,774 according to SEQ ID NO:2 means that if the nucleotide sequence of the SLC26A5 genomic nucleic acid molecule is aligned to the sequence of SEQ ID NO:2, the SLC26A5 sequence has a cytosine residue at the position that corresponds to position 24,774 of SEQ ID NO:2. The same applies for mRNA molecules comprising a nucleotide sequence encoding an SLC26A5 polypeptide, wherein the nucleotide sequence comprises a cytosine at a position corresponding to position 373 according to SEQ ID NO:13, and cDNA molecules comprising a nucleotide sequence encoding an SLC26A5 polypeptide, wherein the nucleotide sequence comprises a cytosine at a position corresponding to position 373 according to SEQ ID NO:33. In other words, these phrases refer to a nucleic acid molecule encoding an SLC26A5 polypeptide, wherein the genomic nucleic acid molecule has a nucleotide sequence that comprises a cytosine residue that is homologous to the cytosine residue at position 24,774 of SEQ ID NO:2 (or wherein the mRNA molecule has a nucleotide sequence that comprises a cytosine residue that is homologous to the cytosine residue at position 373 of SEQ ID NO:13, or wherein the cDNA molecule has a nucleotide sequence that comprises a cytosine residue that is homologous to the cytosine residue at position 373 of SEQ ID NO:33).

As described herein, a position within an SLC26A5 genomic nucleic acid molecule that corresponds to position 24,774 according to SEQ ID NO:2, for example, can be identified by performing a sequence alignment between the nucleotide sequence of a particular SLC26A5 nucleic acid molecule and the nucleotide sequence of SEQ ID NO:2. A variety of computational algorithms exist that can be used for performing a sequence alignment to identify a nucleotide position that corresponds to, for example, position 24,774 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of an SLC26A5 reference polypeptide is set forth in SEQ ID NO:43. Referring to SEQ ID NO:43, the SLC26A5 reference polypeptide is 744 amino acids in length. Referring to SEQ ID NO:43, position 46 is leucine.

The amino acid sequence of another SLC26A5 reference polypeptide is set forth in SEQ ID NO:44. Referring to SEQ ID NO:44, the SLC26A5 reference polypeptide is 685 amino acids in length. Referring to SEQ ID NO:44, position 46 is leucine.

The amino acid sequence of another SLC26A5 reference polypeptide is set forth in SEQ ID NO:45. Referring to SEQ ID NO:45, the SLC26A5 reference polypeptide is 516 amino acids in length. Referring to SEQ ID NO:45, position 46 is leucine.

The amino acid sequence of another SLC26A5 reference polypeptide is set forth in SEQ ID NO:46. Referring to SEQ ID NO:46, the SLC26A5 reference polypeptide is 335 amino acids in length. Referring to SEQ ID NO:46, position 46 is leucine.

The amino acid sequence of another SLC26A5 reference polypeptide is set forth in SEQ ID NO:47. Referring to SEQ ID NO:47, the SLC26A5 reference polypeptide is 712 amino acids in length. Referring to SEQ ID NO:47, position 46 is leucine.

The amino acid sequence of another SLC26A5 reference polypeptide is set forth in SEQ ID NO:48. Referring to SEQ ID NO:48, the SLC26A5 reference polypeptide is 714 amino acids in length. Referring to SEQ ID NO:48, position 46 is leucine.

The amino acid sequence of another SLC26A5 reference polypeptide is set forth in SEQ ID NO:49. Referring to SEQ ID NO:49, the SLC26A5 reference polypeptide is 473 amino acids in length. Referring to SEQ ID NO:49, position 46 is leucine.

The amino acid sequence of another SLC26A5 reference polypeptide is set forth in SEQ ID NO:50. Referring to SEQ ID NO:50, the SLC26A5 reference polypeptide is 447 amino acids in length. Referring to SEQ ID NO:50, position 46 is leucine.

The amino acid sequence of another SLC26A5 reference polypeptide is set forth in SEQ ID NO:51. Referring to SEQ ID NO:51, the SLC26A5 reference polypeptide is 746 amino acids in length. Referring to SEQ ID NO:51, position 46 is leucine.

An SLC26A5 variant polypeptide exists (Leu46Pro Isoform 1 or L46P-1), the amino acid sequence of which is set forth in SEQ ID NO:52. Referring to SEQ ID NO:52, the SLC26A5 variant polypeptide is 744 amino acids in length. Referring to SEQ ID NO:52, position 46 is proline.

Another SLC26A5 variant polypeptide exists (Leu46Pro Isoform 2 or L46P-2), the amino acid sequence of which is set forth in SEQ ID NO:53. Referring to SEQ ID NO:53, the SLC26A5 variant polypeptide is 685 amino acids in length. Referring to SEQ ID NO:53, position 46 is proline.

Another SLC26A5 variant polypeptide exists (Leu46Pro Isoform 3 or L46P-3), the amino acid sequence of which is set forth in SEQ ID NO:54. Referring to SEQ ID NO:54, the SLC26A5 variant polypeptide is 516 amino acids in length. Referring to SEQ ID NO:54, position 46 is proline.

Another SLC26A5 variant polypeptide exists (Leu46Pro Isoform 4 or L46P-4), the amino acid sequence of which is set forth in SEQ ID NO:55. Referring to SEQ ID NO:55, the SLC26A5 variant polypeptide is 355 amino acids in length. Referring to SEQ ID NO:55, position 46 is proline.

Another SLC26A5 variant polypeptide exists (Leu46Pro Isoform 5 or L46P-5), the amino acid sequence of which is set forth in SEQ ID NO:56. Referring to SEQ ID NO:56, the SLC26A5 variant polypeptide is 712 amino acids in length. Referring to SEQ ID NO:56, position 46 is proline.

Another SLC26A5 variant polypeptide exists (Leu46Pro Isoform 6 or L46P-6), the amino acid sequence of which is set forth in SEQ ID NO:57. Referring to SEQ ID NO:57, the SLC26A5 variant polypeptide is 714 amino acids in length. Referring to SEQ ID NO:57, position 46 is proline.

Another SLC26A5 variant polypeptide exists (Leu46Pro Isoform 7 or L46P-7), the amino acid sequence of which is set forth in SEQ ID NO:58. Referring to SEQ ID NO:58, the SLC26A5 variant polypeptide is 473 amino acids in length. Referring to SEQ ID NO:58, position 46 is proline.

Another SLC26A5 variant polypeptide exists (Leu46Pro Isoform 8 or L46P-8), the amino acid sequence of which is set forth in SEQ ID NO:59. Referring to SEQ ID NO:59, the SLC26A5 variant polypeptide is 447 amino acids in length. Referring to SEQ ID NO:59, position 46 is proline.

Another SLC26A5 variant polypeptide exists (Leu46Pro Isoform 9 or L46P-9), the amino acid sequence of which is set forth in SEQ ID NO:60. Referring to SEQ ID NO:60, the SLC26A5 variant polypeptide is 746 amino acids in length. Referring to SEQ ID NO:60, position 46 is proline.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit hearing loss for use in the treatment of hearing loss (or for use in the preparation of a medicament for treating hearing loss) in a subject, wherein the subject has any of the genomic nucleic acid molecules, mRNA molecules, and/or cDNA molecules encoding an SLC26A5 polypeptide described herein. The therapeutic agents that treat or inhibit hearing loss can be any of the therapeutic agents that treat or inhibit hearing loss described herein.

In some embodiments, the subject comprises: a genomic nucleic acid molecule having a nucleotide sequence encoding an SLC26A5 polypeptide, wherein the nucleotide sequence comprises a cytosine at a position corresponding to position 24,774 according to SEQ ID NO:2, or the complement thereof; an mRNA molecule having a nucleotide sequence encoding an SLC26A5 polypeptide, wherein the nucleotide sequence comprises a cytosine at a position corresponding to position 373 according to SEQ ID NO:13, or the complement thereof; a cDNA molecule having a nucleotide sequence encoding an SLC26A5 polypeptide, wherein the nucleotide sequence comprises a cytosine at a position corresponding to position 373 according to SEQ ID NO:33, or the complement thereof; or an SLC26A5 polypeptide that comprises a proline at a position corresponding to position 46 according to SEQ ID NO:52. The therapeutic agents that treat or inhibit hearing loss can be any of the therapeutic agents that treat or inhibit hearing loss described herein.

In some embodiments, the subject comprises: an mRNA molecule having a nucleotide sequence encoding an SLC26A5 polypeptide, wherein the nucleotide sequence comprises a cytosine at a position corresponding to position 373 according to SEQ ID NO:14, or the complement thereof; a cDNA molecule having a nucleotide sequence encoding an SLC26A5 polypeptide, wherein the nucleotide sequence comprises a cytosine at a position corresponding to position 373 according to SEQ ID NO:34, or the complement thereof; or an SLC26A5 polypeptide that comprises a proline at a position corresponding to position 46 according to SEQ ID NO:53. The therapeutic agents that treat or inhibit hearing loss can be any of the therapeutic agents that treat or inhibit hearing loss described herein.

In some embodiments, the subject comprises: an mRNA molecule having a nucleotide sequence encoding an SLC26A5 polypeptide, wherein the nucleotide sequence comprises a cytosine at a position corresponding to position 373 according to SEQ ID NO:15, or the complement thereof; a cDNA molecule having a nucleotide sequence encoding an SLC26A5 polypeptide, wherein the nucleotide sequence comprises a cytosine at a position corresponding to position 373 according to SEQ ID NO:35, or the complement thereof; or an SLC26A5 polypeptide that comprises a proline at a position corresponding to position 46 according to SEQ ID NO:54. The therapeutic agents that treat or inhibit hearing loss can be any of the therapeutic agents that treat or inhibit hearing loss described herein.

In some embodiments, the subject comprises: an mRNA molecule having a nucleotide sequence encoding an SLC26A5 polypeptide, wherein the nucleotide sequence comprises a cytosine at a position corresponding to position 373 according to SEQ ID NO:16, or the complement thereof; a cDNA molecule having a nucleotide sequence encoding an SLC26A5 polypeptide, wherein the nucleotide sequence comprises a cytosine at a position corresponding to position 373 according to SEQ ID NO:36, or the complement thereof; or an SLC26A5 polypeptide that comprises a proline at a position corresponding to position 46 according to SEQ ID NO:55. The therapeutic agents that treat or inhibit hearing loss can be any of the therapeutic agents that treat or inhibit hearing loss described herein.

In some embodiments, the subject comprises: an mRNA molecule having a nucleotide sequence encoding an SLC26A5 polypeptide, wherein the nucleotide sequence comprises a cytosine at a position corresponding to position 304 according to SEQ ID NO:17, or the complement thereof; a cDNA molecule having a nucleotide sequence encoding an SLC26A5 polypeptide, wherein the nucleotide sequence comprises a cytosine at a position corresponding to position 304 according to SEQ ID NO:37, or the complement thereof; or an SLC26A5 polypeptide that comprises a proline at a position corresponding to position 46 according to SEQ ID NO:56. The therapeutic agents that treat or inhibit hearing loss can be any of the therapeutic agents that treat or inhibit hearing loss described herein.

In some embodiments, the subject comprises: an mRNA molecule having a nucleotide sequence encoding an SLC26A5 polypeptide, wherein the nucleotide sequence comprises a cytosine at a position corresponding to position 304 according to SEQ ID NO:18, or the complement thereof; a cDNA molecule having a nucleotide sequence encoding an SLC26A5 polypeptide, wherein the nucleotide sequence comprises a cytosine at a position corresponding to position 304 according to SEQ ID NO:38, or the complement thereof; or an SLC26A5 polypeptide that comprises a proline at a position corresponding to position 46 according to SEQ ID NO:57. The therapeutic agents that treat or inhibit hearing loss can be any of the therapeutic agents that treat or inhibit hearing loss described herein.

In some embodiments, the subject comprises: an mRNA molecule having a nucleotide sequence encoding an SLC26A5 polypeptide, wherein the nucleotide sequence comprises a cytosine at a position corresponding to position 304 according to SEQ ID NO:19, or the complement thereof; a cDNA molecule having a nucleotide sequence encoding an SLC26A5 polypeptide, wherein the nucleotide sequence comprises a cytosine at a position corresponding to position 304 according to SEQ ID NO:39, or the complement thereof; or an SLC26A5 polypeptide that comprises a proline at a position corresponding to position 46 according to SEQ ID NO:58. The therapeutic agents that treat or inhibit hearing loss can be any of the therapeutic agents that treat or inhibit hearing loss described herein.

In some embodiments, the subject comprises: an mRNA molecule having a nucleotide sequence encoding an SLC26A5 polypeptide, wherein the nucleotide sequence comprises a cytosine at a position corresponding to position 145 according to SEQ ID NO:20, or the complement thereof; a cDNA molecule having a nucleotide sequence encoding an SLC26A5 polypeptide, wherein the nucleotide sequence comprises a cytosine at a position corresponding to position 145 according to SEQ ID NO:40, or the complement thereof; or an SLC26A5 polypeptide that comprises a proline at a position corresponding to position 46 according to SEQ ID NO:59. The therapeutic agents that treat or inhibit hearing loss can be any of the therapeutic agents that treat or inhibit hearing loss described herein.

In some embodiments, the subject comprises: an mRNA molecule having a nucleotide sequence encoding an SLC26A5 polypeptide, wherein the nucleotide sequence comprises a cytosine at a position corresponding to position 145 according to SEQ ID NO:21, or the complement thereof; a cDNA molecule having a nucleotide sequence encoding an SLC26A5 polypeptide, wherein the nucleotide sequence comprises a cytosine at a position corresponding to position 145 according to SEQ ID NO:41, or the complement thereof; or an SLC26A5 polypeptide that comprises a proline at a position corresponding to position 46 according to SEQ ID NO:60. The therapeutic agents that treat or inhibit hearing loss can be any of the therapeutic agents that treat or inhibit hearing loss described herein.

In some embodiments, the subject comprises an mRNA molecule having a nucleotide sequence encoding an SLC26A5 polypeptide, wherein the nucleotide sequence comprises a cytosine at a position corresponding to position 205 according to SEQ ID NO:22, or the complement thereof, or a cDNA molecule having a nucleotide sequence encoding an SLC26A5 polypeptide, wherein the nucleotide sequence comprises a cytosine at a position corresponding to position 205 according to SEQ ID NO:42, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: SLC26A5 Missense Variant is Associated with Hearing Loss

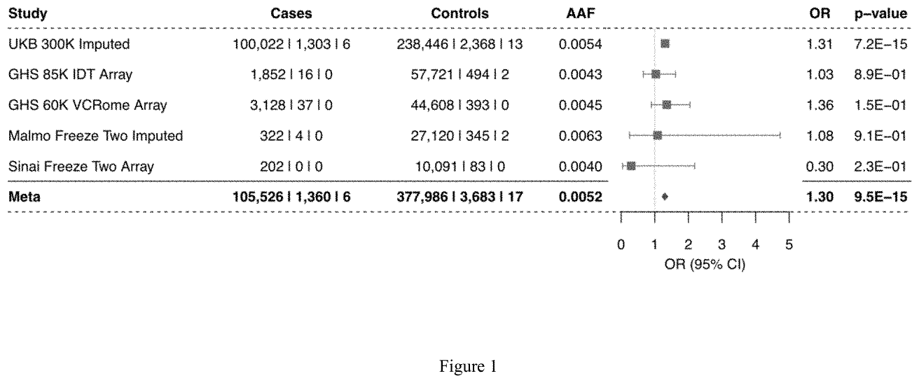

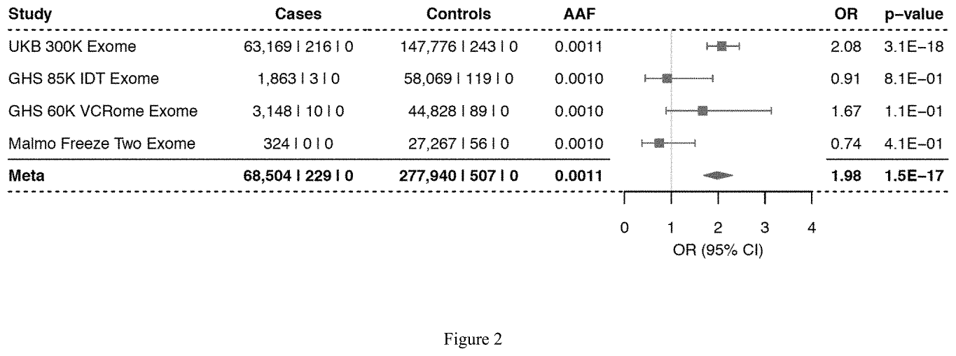

A genome-wide and exome-wide analysis of hearing loss was carried out in UK Biobank, Geisinger (GHS) and other datasets. A missense variant in SLC26A5 was associated with increased risk for hearing loss (OR=1.30, p=9.5E-15) in a meta-analysis of UK Biobank and 3 other cohorts (see, FIG. 1). In addition, an aggregate of rare (minor allele frequency of less than 1%), deleterious missense and predicted loss of function variants in SLC26A5 also show an association (in gene burden tests) with increased risk for hearing loss in the meta-analysis (FIG. 2). The variants in individuals with hearing loss that were aggregated in the above gene burden analysis are provided in Table 1.

Variant 7:103410474:C:T (hgvsc=c.646G>A:c.646G>A:c.646G>A:c.646G>A: c.646G>A:c.646G>A:c.646G>A:c.646G>A:c.646G>A:c.535G>A; hgvsp=p.Ala216Thr: p.Ala216Thr:p.Ala216Thr:p.Ala216Thr:p.Ala216Thr:p.Ala216Thr:p.Ala216Thr:p.Ala216Thr:p. Ala216Thr:p.Ala179Thr);

Variant 7:103389007:C:T (hgvsc=c.1514+1G>A:c.1514+1G>A:c.1418+1G>A: c.1514+1G>A:c.1418+1G>A:c.1514+1G>A:c.1514+1G>A:c.1418+1G>A:c.1403+1G>A);

Variant 7:103407915:C:T (hgvsc=c.824G>A:c.824G>A:c.824G>A:c.824G>A: c.824G>A:c.824G>A:c.824G>A:c.824G>A:c.824G>A:c.713G>A; hgvsp=p.Gly275Asp: p.Gly275Asp:p.Gly275Asp:p.Gly275Asp:p.Gly275Asp:p.Gly275Asp:p.Gly275Asp:p.Gly275Asp:p.Gly275Asp:p.Gly238Asp);

Variant 7:103389358:AG:A (hgvsc=c.1377delC:c.1377delC:c.1377delC:c.1377delC: c.1377delC:c.1266delC; hgvsp=p.Phe461fs:p.Phe461fs:p.Phe461fs:p.Phe461fs:p.Phe461fs: p.Phe424fs);

Variant 7:103420806:T:A (hgvsc=c.224A>T:c.224A>T:c.224A>T:c.224A>T:c.224A>T: c.224A>T:c.224A>T:c.224A>T:c.224A>T:c.224A>T; hgvsp=p.Lys75Ile:p.Lys75Ile:p.Lys75Ile: p.Lys75Ile:p.Lys75Ile:p.Lys75Ile:p.Lys75Ile:p.Lys75Ile:p.Lys75Ile:p.Lys75Ile);

Variant 7:103390495:AC:A (hgvsc=c.1244delG:c.1244delG:c.1244delG:c.1244delG: c.1244delG:c.1244delG:c.1244delG:c.1244delG:c.1133delG; hgvsp=p.Cys415fs:p.Cys415fs: p.Cys415fs:p.Cys415fs:p.Cys415fs:p.Cys415fs:p.Cys415fs:p.Cys415fs:p.Cys378fs);

Variant 7:103391636:C:T (hgvsc=c.1219G>A:c.1219G>A:c.1219G>A:c.1219G>A: c.1219G>A:c.1219G>A:c.1219G>A:c.1219G>A:c.1108G>A; hgvsp=p.Gly407Ser:p.Gly407Ser: p.Gly407Ser: p.Gly407Ser: p.Gly407Ser: p.Gly407Se r: p.Gly407Se r: p.Gly407Se r: p.Gly370Ser); and

Variant 7:103393007:A:G (hgvsc=c.1031T>C:c.1031T>C:c.1031T>C:c.1031T>C: c.1031T>C:c.1031T>C:c.1031T>C:c.1031T>C:c.920T>C; hgvsp=p.Ile344Thr:p.Ile344Thr: p.Ile344Thr:p.Ile344Thr:p.Ile344Thr:p.Ile344Thr:p.Ile344Thr:p.Ile344Thr:p.Ile307Thr).

In the case of chromosomal positions, the reference sequence is the December 2013 (GRCh38/hg38) human genome assembly. The chromosomal positions correspond to the genomic coordinates according to the annotation of the December 2013 (GRCh38/hg38) human genome assembly. For example, the position corresponding to position 7:103410474 or chromosomal position 7:103410474 and grammatic equivalents thereof mean a nucleotide residue located at position 103,410,474 of the human chromosome 7 as annotated in the December 2013 (GRCh38/hg38) human genome assembly.

TABLE 1
Variants in individuals with hearing loss that were aggregated for the analysis

| Chromosome:position: | | |
|---|---|---|
| reference:alternate | hgvsc | hgvsp |
| 7:103410474:C:T | c.646G>A:c.646G>A:c.646G>A:c.646G>A: | p.Ala216Thr:p.Ala216Thr:p.Ala216Thr: |
| | c.646G>A:c.646G>A:c.646G>A:c.646G>A: | p.Ala216Thr:p.Ala216Thr:p.Ala216Thr: |
| | c.646G>A:c.535G>A | p.Ala216Thr:p.Ala216Thr:p.Ala216Thr: |
| | | p.Ala179Thr |
| 7:103389007:C:T | c.1514+1G>A:c.1514+1G>A:c.1418+1G>A: | |
| | c.1514+1G>A:c.1418+1G>A:c.1514+1G>A: | |
| | c.1514+1G>A:c.1418+1G>A:c.1403+1G>A | |
| 7:103407915:C:T | c.824G>A:c.824G>A:c.824G>A:c.824G>A: | p.Gly275Asp:p.Gly275Asp:p.Gly275Asp: |
| | c.824G>A:c.824G>A:c.824G>A:c.824G>A: | p.Gly275Asp:p.Gly275Asp:p.Gly275Asp: |
| | c.824G>A:c.713G>A | p.Gly275Asp:p.Gly275Asp:p.Gly275Asp: |
| | | p.Gly238Asp |
| 7:103389358:AG:A | c.1377delC:c.1377delC:c.1377delC:c.1377delC: | p.Phe461fs:p.Phe461fs:p.Phe461fs: |
| | c.1377delC:c.1266delC | p.Phe461fs:p.Phe461fs:p.Phe424fs |
| 7:103420806:T:A | c.224A>T:c.224A>T:c.224A>T:c.224A>T: | p.Lys75Ile:p.Lys75Ile:p.Lys75Ile:p.Lys75Ile: |
| | c.224A>T:c.224A>T:c.224A>T:c.224A>T: | p.Lys75Ile:p.Lys75Ile:p.Lys75Ile:p.Lys75Ile: |
| | c.224A>T:c.224A>T | p.Lys75Ile:p.Lys75Ile |
| 7:103390495:AC:A | c.1244delG:c.1244delG:c.1244delG:c.1244delG: | p.Cys415fs:p.Cys415fs:p.Cys415fs:p.Cys415fs: |
| | c.1244delG:c.1244delG:c.1244delG:c.1244delG: | p.Cys415fs:p.Cys415fs:p.Cys415fs:p.Cys415fs: |
| | c.1133delG | p.Cys378fs |
| 7:103391636:C:T | c.1219G>A:c.1219G>A:c.1219G>A:c.1219G>A: | p.Gly407Ser:p.Gly407Ser:p.Gly407Ser: |
| | c.1219G>A:c.1219G>A:c.1219G>A:c.1219G>A: | p.Gly407Ser:p.Gly407Ser:p.Gly407Ser: |
| | c.1108G>A | p.Gly407Ser:p.Gly407Ser:p.Gly370Ser |
| 7:103393007:A:G | c.1031T>C:c.1031T>C:c.1031T>C:c.1031T>C: | p.Ile344Thr:p.Ile344Thr:p.Ile344Thr: |
| | c.1031T>C:c.1031T>C:c.1031T>C:c.1031T>C: | p.Ile344Thr:p.Ile344Thr:p.Ile344Thr: |
| | c.920T>C: | p.Ile344Thr:p.Ile344Thr:p.Ile307Thr |

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject with a therapeutic agent that treats or inhibits hearing loss, wherein the subject has hearing loss, the method comprising the steps of:
determining whether the subject has a Solute Carrier Family 26 Member 5 (SLC26A5) missense variant nucleic acid molecule encoding an SLC26A5 predicted loss-of-function polypeptide by:
obtaining or having obtained a biological sample from the subject; and
performing or having performed a sequence analysis on the biological sample to determine if the subject has a genotype comprising the SLC26A5 missense variant nucleic acid molecule encoding an SLC26A5 predicted loss-of-function polypeptide; and
administering or continuing to administer the therapeutic agent that treats or inhibits hearing loss in a standard dosage amount to a subject that is SLC26A5 reference; and
administering or continuing to administer the therapeutic agent that treats or inhibits hearing loss in an amount that is the same as or greater than a standard dosage amount to a subject that is heterozygous or homozygous for the SLC26A5 missense variant nucleic acid molecule;
wherein the presence of a genotype having the SLC26A5 missense variant nucleic acid molecule encoding an SLC26A5 predicted loss-of-function polypeptide indicates the subject has an increased risk of developing hearing loss.

2. The method according to claim 1, wherein the SLC26A5 missense variant nucleic acid molecule encodes SLC26A5 Leu46Pro.

3. The method according to claim 1, wherein the nucleic acid molecule encoding SLC26A5 Leu46Pro is SLC26A5 Leu46Pro Isoform 1.

4. The method according to claim 2, wherein the SLC26A5 missense variant nucleic acid molecule encoding an SLC26A5 predicted loss-of-function polypeptide is:
a genomic nucleic acid molecule having a nucleotide sequence comprising a cytosine at a position corresponding to position 24,774 according to SEQ ID NO:2;
an mRNA molecule having a nucleotide sequence comprising: a cytosine at a position corresponding to position 373 according to SEQ ID NO:13, a cytosine at a position corresponding to position 373 according to SEQ ID NO:14, a cytosine at a position corresponding to position 373 according to SEQ ID NO:15, a cytosine at a position corresponding to position 373 according to SEQ ID NO:16, a cytosine at a position corresponding to position 304 according to SEQ ID NO:17, a cytosine at a position corresponding to position 304 according to SEQ ID NO:18, a cytosine at a position corresponding to position 304 according to SEQ ID NO:19, a cytosine at a position corresponding to position 145 according to SEQ ID NO:20, a cytosine at a position corresponding to position 145 according to SEQ ID NO:21, or a cytosine at a position corresponding to position 205 according to SEQ ID NO:22; or
a cDNA molecule produced from an mRNA molecule, wherein the cDNA molecule has a nucleotide sequence comprising: a cytosine at a position corresponding to position 373 according to SEQ ID NO:33, a cytosine at a position corresponding to position 373 according to SEQ ID NO:34, a cytosine at a position corresponding to position 373 according to SEQ ID NO:35, a cytosine at a position corresponding to position 373 according to SEQ ID NO:36, a cytosine at a position corresponding to position 304 according to SEQ ID NO:37, a cytosine at a position corresponding to position 304 according to SEQ ID NO:38, a cytosine at a position corresponding to position 304 according to SEQ ID NO:39, a cytosine at a position corresponding to position 145 according to SEQ ID NO:40, a cytosine at a position corresponding to position 145 according to SEQ ID NO:41, or a cytosine at a position corresponding to position 205 according to SEQ ID NO:42.

5. The method according to claim 1, wherein the sequence analysis comprises sequencing at least a portion of the nucleotide sequence of the SLC26A5 genomic nucleic acid molecule in the biological sample, wherein the sequenced portion comprises a position corresponding to position 24,774 according to SEQ ID NO:2, or the complement thereof;
wherein when the sequenced portion of the SLC26A5 genomic nucleic acid molecule in the biological sample comprises a cytosine at a position corresponding to position 24,774 according to SEQ ID NO:2, then the SLC26A5 genomic nucleic acid molecule in the biological sample is an SLC26A5 missense variant nucleic acid molecule encoding an SLC26A5 predicted loss-of-function polypeptide.

6. The method according to claim 1, wherein the sequence analysis comprises sequencing at least a portion of the nucleotide sequence of the SLC26A5 mRNA molecule in the biological sample, wherein the sequenced portion comprises a position corresponding to: position 373 according to SEQ ID NO:13, or the complement thereof, position 373 according to SEQ ID NO:14, or the complement thereof, position 373 according to SEQ ID NO:15, or the complement thereof, position 373 according to SEQ ID NO:16, or the complement thereof, position 304 according to SEQ ID NO:17, or the complement thereof, position 304 according to SEQ ID NO:18, or the complement thereof, position 304 according to SEQ ID NO:19 the complement thereof, position 145 according to SEQ ID NO:20, the complement thereof, position 145 according to SEQ ID NO:21, or the complement thereof, or position 205 according to SEQ ID NO:22, or the complement thereof,
wherein when the sequenced portion of the SLC26A5 mRNA molecule in the biological sample comprises: a cytosine at a position corresponding to position 373 according to SEQ ID NO:13, a cytosine at a position corresponding to position 373 according to SEQ ID NO:14, a cytosine at a position corresponding to position 373 according to SEQ ID NO:15, a cytosine at a position corresponding to position 373 according to SEQ ID NO:16, a cytosine at a position corresponding to position 304 according to SEQ ID NO:17, a cytosine at a position corresponding to position 304 according to SEQ ID NO:18, a cytosine at a position corresponding to position 304 according to SEQ ID NO:19, a cytosine at a position corresponding to position 145 according to SEQ ID NO:20, a cytosine at a position corresponding to position 145 according to SEQ ID NO:21, or a cytosine at a position corresponding to position 205 according to SEQ ID NO:22, then the SLC26A5 mRNA molecule in the biological sample is an SLC26A5 missense variant nucleic acid molecule encoding an SLC26A5 predicted loss-of-function polypeptide.

7. The method according to claim 1, wherein the sequence analysis comprises sequencing at least a portion of the nucleotide sequence of the SLC26A5 cDNA molecule, wherein the sequenced portion comprises a position corresponding to: position 373 according to SEQ ID NO:33, or the complement thereof, position 373 according to SEQ ID NO:34, or the complement thereof, position 373 according to SEQ ID NO:35, or the complement thereof, position 373 according to SEQ ID NO:36, or the complement thereof, position 304 according to SEQ ID NO:37, or the complement thereof, position 304 according to SEQ ID NO:38, or the complement thereof, or position 304 according to SEQ ID NO:39 the complement thereof, position 145 according to SEQ ID NO:40, the complement thereof, position 145 according to SEQ ID NO:41, or the complement thereof, or position 205 according to SEQ ID NO:42, or the complement thereof,
wherein when the sequenced portion of the SLC26A5 cDNA molecule in the biological sample comprises: a cytosine at a position corresponding to position 373 according to SEQ ID NO:33, a cytosine at a position corresponding to position 373 according to SEQ ID NO:34, a cytosine at a position corresponding to position 373 according to SEQ ID NO:35, a cytosine at a position corresponding to position 373 according to SEQ ID NO:36, a cytosine at a position corresponding to position 304 according to SEQ ID NO:37, a cytosine at a position corresponding to position 304 according to SEQ ID NO:38, a cytosine at a position corresponding to position 304 according to SEQ ID NO:39, a cytosine at a position corresponding to position 145 according to SEQ ID NO:40, a cytosine at a position corresponding to position 145 according to SEQ ID NO:41, or a cytosine at a position corresponding to position 205 according to SEQ ID NO:42, then the SLC26A5 cDNA molecule in the biological sample is an SLC26A5 missense variant cDNA molecule encoding an SLC26A5 predicted loss-of-function polypeptide.

8-19. (canceled)

20. A method of identifying a subject having an increased risk for developing hearing loss, the method comprising:
determining or having determined the presence or absence of a Solute Carrier Family 26 Member 5 (SLC26A5) missense variant nucleic acid molecule encoding an SLC26A5 predicted loss-of-function polypeptide in a biological sample obtained from the subject;
wherein:
when the subject is SLC26A5 reference, then the subject does not have an increased risk for developing hearing loss; and
when the subject is heterozygous or homozygous for an SLC26A5 missense variant nucleic acid molecule encoding an SLC26A5 predicted loss-of-function polypeptide, then the subject has an increased risk for developing hearing loss.

21. The method according to claim 20, wherein the SLC26A5 missense variant nucleic acid molecule encodes SLC26A5 Leu46Pro.

22. The method according to claim 21, wherein the nucleic acid molecule encoding SLC26A5 Leu46Pro is SLC26A5 Leu46Pro Isoform 1.

23. The method according to claim 21, wherein the SLC26A5 missense variant nucleic acid molecule encoding an SLC26A5 predicted loss-of-function polypeptide is:
a genomic nucleic acid molecule having a nucleotide sequence comprising a cytosine at a position corresponding to position 24,774 according to SEQ ID NO:2;
an mRNA molecule having a nucleotide sequence comprising: a cytosine at a position corresponding to position 373 according to SEQ ID NO:13, a cytosine at a position corresponding to position 373 according to SEQ ID NO:14, a cytosine at a position corresponding to position 373 according to SEQ ID NO:15, a cytosine at a position corresponding to position 373 according to SEQ ID NO:16, a cytosine at a position corresponding to position 304 according to SEQ ID NO:17, a cytosine at a position corresponding to position 304 according to SEQ ID NO:18, a cytosine at a position corresponding to position 304 according to SEQ ID NO:19, a cytosine at a position corresponding to position 145 according to SEQ ID NO:20, a cytosine at a position corresponding to position 145 according to SEQ ID NO:21, or a cytosine at a position corresponding to position 205 according to SEQ ID NO:22; or
a cDNA molecule produced from an mRNA molecule, wherein the cDNA molecule has a nucleotide sequence comprising: a cytosine at a position corresponding to position 373 according to SEQ ID NO:33, a cytosine at a position corresponding to position 373 according to SEQ ID NO:34, a cytosine at a position corresponding to position 373 according to SEQ ID NO:35, a cytosine at a position corresponding to position 373 according to SEQ ID NO:36, a cytosine at a position corresponding to position 304 according to SEQ ID NO:37, a cytosine at a position corresponding to position 304 according to SEQ ID NO:38, a cytosine at a position corresponding to position 304 according to SEQ ID NO:39, a cytosine at a position corresponding to position 145 according to SEQ ID NO:40, a cytosine at a position corresponding to position 145 according to SEQ ID NO:41, or a cytosine at a position corresponding to position 205 according to SEQ ID NO:42.

24. (canceled)

25. The method according to claim 20, wherein the determining step comprises sequencing at least a portion of the nucleotide sequence of the SLC26A5 genomic nucleic acid molecule in the biological sample, wherein the sequenced portion comprises a position corresponding to position 24,774 according to SEQ ID NO:2, or the complement thereof;
wherein when the sequenced portion of the SLC26A5 genomic nucleic acid molecule in the biological sample comprises a cytosine at a position corresponding to position 24,774 according to SEQ ID NO:2, then the SLC26A5 genomic nucleic acid molecule in the biological sample is an SLC26A5 missense variant nucleic acid molecule encoding an SLC26A5 predicted loss-of-function polypeptide.

26. The method according to claim 20, wherein the determining step comprises sequencing at least a portion of the nucleotide sequence of the SLC26A5 mRNA molecule in the biological sample, wherein the sequenced portion comprises a position corresponding to: position 373 according to SEQ ID NO:13, or the complement thereof, position 373 according to SEQ ID NO:14, or the complement thereof, position 373 according to SEQ ID NO:15, or the complement thereof, position 373 according to SEQ ID NO:16, or the complement thereof, position 304 according to SEQ ID NO:17, or the complement thereof, position 304 according to SEQ ID NO:18, or the complement thereof, position 304 according to SEQ ID NO:19 the complement thereof, position 145 according to SEQ ID NO:20, the complement thereof, position 145 according to SEQ ID NO:21, or the complement thereof, or position 205 according to SEQ ID NO:22, or the complement thereof,
wherein when the sequenced portion of the SLC26A5 mRNA molecule in the biological sample comprises: a cytosine at a position corresponding to position 373 according to SEQ ID NO:13, a cytosine at a position corresponding to position 373 according to SEQ ID NO:14, a cytosine at a position corresponding to position 373 according to SEQ ID NO:15, a cytosine at a position corresponding to position 373 according to SEQ ID NO:16, a cytosine at a position corresponding to position 304 according to SEQ ID NO:17, a cytosine at a position corresponding to position 304 according to SEQ ID NO:18, a cytosine at a position corresponding to position 304 according to SEQ ID NO:19, a cytosine at a position corresponding to position 145 according to SEQ ID NO:20, a cytosine at a position corresponding to position 145 according to SEQ ID NO:21, or a cytosine at a position corresponding to position 205 according to SEQ ID NO:22, then the SLC26A5 mRNA molecule in the biological sample is an SLC26A5 missense variant nucleic acid molecule encoding an SLC26A5 predicted loss-of-function polypeptide.

27. The method according to claim 20, wherein the determining step comprises sequencing at least a portion of the nucleotide sequence of the SLC26A5 cDNA molecule, wherein the sequenced portion comprises a position corresponding to: position 373 according to SEQ ID NO:33, or the complement thereof, position 373 according to SEQ ID NO:34, or the complement thereof, position 373 according to SEQ ID NO:35, or the complement thereof, position 373 according to SEQ ID NO:36, or the complement thereof, position 304 according to SEQ ID NO:37, or the complement thereof, position 304 according to SEQ ID NO:38, or the complement thereof, or position 304 according to SEQ ID NO:39 the complement thereof, position 145 according to SEQ ID NO:40, the complement thereof, position 145 according to SEQ ID NO:41, or the complement thereof, or position 205 according to SEQ ID NO:42, or the complement thereof,
wherein when the sequenced portion of the SLC26A5 cDNA molecule in the biological sample comprises: a cytosine at a position corresponding to position 373 according to SEQ ID NO:33, a cytosine at a position corresponding to position 373 according to SEQ ID NO:34, a cytosine at a position corresponding to position 373 according to SEQ ID NO:35, a cytosine at a position corresponding to position 373 according to SEQ ID NO:36, a cytosine at a position corresponding to position 304 according to SEQ ID NO:37, a cytosine at a position corresponding to position 304 according to SEQ ID NO:38, a cytosine at a position corresponding to position 304 according to SEQ ID NO:39, a cytosine at a position corresponding to position 145 according to SEQ ID NO:40, a cytosine at a position corresponding to position 145 according to SEQ ID NO:41, or a cytosine at a position corresponding to position 205 according to SEQ ID NO:42, then the SLC26A5 cDNA molecule in the biological sample is an SLC26A5 missense variant cDNA molecule encoding an SLC26A5 predicted loss-of-function polypeptide.

28-38. (canceled)

39. The method according to claim 20, wherein the subject is heterozygous or homozygous for an SLC26A5 missense variant nucleic acid molecule encoding an SLC26A5 predicted loss-of-function polypeptide, and the subject is further administered a therapeutic agent that treats or inhibits hearing loss.

40. A method of detecting a Solute Carrier Family 26 Member 5 (SLC26A5) missense variant nucleic acid molecule in a subject comprising assaying a sample obtained from the subject to determine whether a nucleic acid molecule in the sample is:
a genomic nucleic acid molecule comprising a nucleotide sequence comprising a cytosine at a position corresponding to position 24,774 according to SEQ ID NO:2, or the complement thereof;
an mRNA molecule comprising a nucleotide sequence comprising: a cytosine at a position corresponding to position 373 according to SEQ ID NO:13, or the complement thereof, a cytosine at a position corresponding to position 373 according to SEQ ID NO:14, or the complement thereof, a cytosine at a position corresponding to position 373 according to SEQ ID NO:15, or the complement thereof; a cytosine at a position corresponding to position 373 according to SEQ ID NO:16, or the complement thereof, a cytosine at a position corresponding to position 304 according to SEQ ID NO:17, or the complement thereof, a cytosine at a position corresponding to position 304 according to SEQ ID NO:18, or the complement thereof, a cytosine at a position corresponding to position 304 according to SEQ ID NO:19, or the complement thereof, a cytosine at a position corresponding to position 145 according to SEQ ID NO:20, or the complement thereof, a cytosine at a position corresponding to position 145 according to SEQ ID NO:21, or the complement thereof, or a cytosine at a position corresponding to position 205 according to SEQ ID NO:22, or the complement thereof, or
a cDNA molecule comprising a nucleotide sequence comprising: a cytosine at a position corresponding to position 373 according to SEQ ID NO:33, or the complement thereof, a cytosine at a position corresponding to position 373 according to SEQ ID NO:34, or the complement thereof, a cytosine at a position corresponding to position 373 according to SEQ ID NO:35, or the complement thereof; a cytosine at a position corresponding to position 373 according to SEQ ID NO:36, or the complement thereof, a cytosine at a position corresponding to position 304 according to SEQ ID NO:37, a cytosine at a position corresponding to position 304 according to SEQ ID NO:38, or the complement thereof, a cytosine at a position corresponding to position 304 according to SEQ ID NO:39, or the complement thereof, a cytosine at a position corresponding to position 145 according to SEQ ID NO:40, or the complement thereof, a cytosine at a position corresponding to position 145 according to SEQ ID NO:41, or the complement thereof, or a cytosine at a position corresponding to position 205 according to SEQ ID NO:42, or the complement thereof.

41. (canceled)

42. The method according to claim 40, wherein the assay comprises sequencing at least a portion of the nucleic acid molecule, wherein the sequenced portion comprises a cytosine at a position corresponding to position 24,774 according to SEQ ID NO:2, or the complement thereof.

43. The method according to claim 40, wherein the assay comprises sequencing at least a portion of the nucleic acid molecule, wherein the sequenced portion comprises: a cytosine at a position corresponding to position 373 according to SEQ ID NO:13, or the complement thereof; a cytosine at a position corresponding to position 373 according to SEQ ID NO:14, or the complement thereof, a cytosine at a position corresponding to position 373 according to SEQ ID NO:15, or the complement thereof; a cytosine at a position corresponding to position 373 according to SEQ ID NO:16, or the complement thereof, a cytosine at a position corresponding to position 304 according to SEQ ID NO:17, or the complement thereof, a cytosine at a position corresponding to position 304 according to SEQ ID NO:18, or the complement thereof; a cytosine at a position corresponding to position 304 according to SEQ ID NO:19, or the complement thereof, a cytosine at a position corresponding to position 145 according to SEQ ID NO:20, or the complement thereof, a cytosine at a position corresponding to position 145 according to SEQ ID NO:21, or the complement thereof, or a cytosine at a position corresponding to position 205 according to SEQ ID NO:22, or the complement thereof.

44. The method according to claim 40, wherein the assay comprises sequencing at least a portion of the nucleic acid molecule, wherein the sequenced portion comprises: a cytosine at a position corresponding to position 373 according to SEQ ID NO:33, or the complement thereof; a cytosine at a position corresponding to position 373 according to SEQ ID NO:34, or the complement thereof, a cytosine at a position corresponding to position 373 according to SEQ ID NO:35, or the complement thereof; a cytosine at a position corresponding to position 373 according to SEQ ID NO:36, or the complement thereof, a cytosine at a position corresponding to position 304 according to SEQ ID NO:37, a cytosine at a position corresponding to position 304 according to SEQ ID NO:38, or the complement thereof, a cytosine at a position corresponding to position 304 according to SEQ ID NO:39, or the complement thereof, a cytosine at a position corresponding to position 145 according to SEQ ID NO:40, or the complement thereof, a cytosine at a position corresponding to position 145 according to SEQ ID NO:41, or the complement thereof, or a cytosine at a position corresponding to position 205 according to SEQ ID NO:42, or the complement thereof.

45-56. (canceled)

57. A method of detecting the presence of a Solute Carrier Family 26 Member 5 (SLC26A5) variant polypeptide, comprising performing an assay on a sample obtained from a subject to determine whether an SLC26A5 protein in the sample comprises: a proline at a position corresponding to position 46 according to SEQ ID NO:52, a proline at a position corresponding to position 46 according to SEQ ID NO:53, a proline at a position corresponding to position 46 according to SEQ ID NO:54, a proline at a position corresponding to position 46 according to SEQ ID NO:55, a proline at a position corresponding to position 46 according to SEQ ID NO:56, a proline at a position corresponding to position 46 according to SEQ ID NO:57, a proline at a position corresponding to position 46 according to SEQ ID NO:58, a proline at a position corresponding to position 46 according to SEQ ID NO:59, or a proline at a position corresponding to position 46 according to SEQ ID NO:60.

58. The method according to claim 57, wherein the assay comprises sequencing the polypeptide.

59. The method according to claim 57, wherein the assay is an immunoassay.

60. (canceled)


SEQUENCE LISTING (60 sequences)

SEQ ID NO: 1 - DNA, 93422 bp, Homo sapien
aggcagcggctgtggagcgcggcggggcggctccgcccagggcagcccgggctggtgagtgcgcgcgggcggccgcggggccgggggtcgcggtccctcgggaggggactgagctgaggcccgcgggcccacccgagcaggggccctcgctgtcaccgcctctgcccctgtctcccaggccccatgtcgcgctttttccctaaacgaaccggtgacagggctgacctgcccgcactctgctggtccctacgccgggaaggagaaagtctcacgaagctctttcctttgcaacccctgtccccttgccctctgcagctccacacagcatttttacttcccgtttgcctcctcgaatcccttcggtgctaatccagcccttcgccgtttccagtttgggtctggcctgtttgtatccaaatcgaattacacaaaaccaggccctcagttctgcctctggccctctttccggagacgggccctgcctcctcctgtgccgggctgggcttgcccggcattggccgcccggagctcccagggagcaaggctgtggtcccgaaactgggccctccgctcagcgccgggcaaggctcaaaccccgggtctctgacttatccggtcacgaagggactgctcagagcctaacagccctaaaatttggtattggccccctcccgggaaatccttctccctaccaatcaggcatcgcggcaggtgctctgccatcatagaggctctgacacgctgaactctcgaagctcgcggcagtgtttatttttaggagagatttagttgatcgggaaccaattgacttggttgactgcctcatctagtgcgcacctgggccgggccccgccgggaaggcgtgctggacgttctcagtgagctccgagacctacagaagaaacagctgcaaggatccgatcagctagctgtgaagaaagcctcagatacgctgtggggtgggcaaaattcttttcctagcttcgatgagtactgtggattctagaatttaaacaacattcatggagactcttaaaacaacaaaaaatatcaactttttgcgggcacagactgagatttagctttttcctatctgaaaactgaacaatgtgcactagatataaatgtattatctaaagtttaggaatttacagcatagaaatgcaggggaattcggagtatgcaatacgaagtatcaacctttttgaaaatgaaatttaaaatgtatttttgtcaggcattccttctaattacggtagtattgctttgtatcagaagactttcaggcaaaggacaggagggaagagagagaaattcaaactaatttgcattagaaagaaacttagggtgaagaattgcctaggtaaggttgctatagctcatatcttaagtgcactcttggaaagctttctagtctattaggtaacacctgaagccccaattctcttatgtagcatttctttttttctctctttaactactctgaaactatacagagttacacgttcctcattaatttatgagtgaaaccaaaagaattgactcaatccaacaagtatttgagtgcttttcgcatagaggcgctttgctaagaacagaagaatggaagactgaggaagtttctaacaatcaatggattgccttgtgaggtgtagatgcaggtgtgcactgcctatttaattgacgatcaaagtcagttgagttttcatctcattctaaatatcatgtgttgccccctggtatcttaatttttctaccgttgcaggtagtctgcattttagtaacccaagtgaaacagttttctagggaaatcatgataaataggaagcacgtccgtattcccaggtgttctcattatcattaagttaataaagttaagaaaaataagatgatgattttcctttcttttggtttcaaactagtgaaatcttgcacaaaagacacagcatggataaatttaatcattttgtgactcagagtacattttatgtaagtccattctgttggaggatagatccaatttcatgaactcttttaatccttcttttgaagactagttccaggaccaacttcataaaatgttttattgtaggtaccaacaactatcaacatcattatcttctccccaccttcacccttgaaaacaattgtattctagaggattatgaaggaggggacaggactctttcaaaatctggaggacactagggacttaccccagaaaaaaaataaaaaagctcacccattcaaaatcttgtacacctttcagaggattcctggtgcccaggttatgaaagctcacttcagaacgggtcagcgaattagtcagaggataggtagatggagggaaagggaaatgaagcaatgtgggtaaattcacaaactgagtggatatgacttaatttactttcaaatagaataaatacatggccccttgagtaagtattctgtgaatgggaaggaataattgtattctttgtaaaagactgaagacagattagcctaattaagtttttaattgatcaagatcaactcttatcttgaacattatgagtctctcatttttctacttttcttgctcttttcaaggcaactgctctaagaggatttgctcccactactgatgatgccccattctggacacaatttctcatatgcaaacttgtattcatgaattgctacatatgggaatggctgagttcaaaaattgaactatatctttctcacatcctagcagttcacccttaggaagaagatataattatatataattagtctatcgcatgtaagccactgtgatagcagtagctgtgccttggaagggagtgggttggtattaggaatgaaaggagagcgttaagaaatattttggacatagcttctttcaaaaaacaaaccaacaactgctgaattaacttccttttcttcttctatacatagggccaaggagcgagctctcccttctcctgctctcagcctcagtgatcaaggcttcagtgaactgcactggagctcccagcgggggatcttgtcccctgtcccgacttttgtgctgcacattggatctgggtaagtagacggccccctggaactagtaaataggagagtgggtcagaggtagtaatggattttatccagatcaaaggattcctcaaaatttccagaggtctaaatcacacaccactatttggaagaaaagtataagacagaatgccagaaccatcacaccttacacttaccttttaacttggatcaggatcagcctaagatatccttagctatcaaccagctttgacttacagtattcttgagtggcaactaggtggtgggccaacaaccctagaatgtacttaatagatgcttactggatttgtaaattaatggagatcatggttttaagccaagctctgccactaaccaggcatatgatcttgggcaagtcacaggttagttaaattctttggaccttaatttttctacgtataaaatgagggtgttttatgggaccaatggttctcagcaaccaatgtataattccattgcatttgcattgcctgggagtttatttagaaaaaacccaacagagattctgattaattattaatcagatttattctgaggataaagtccttagtgttccctagattttgaaaaagacctgtcccctccctcatggccctccagaatacaatcgttttcaatagtgaaagtgtgggccaggctcagtggctcacacctgtaatcccagcactttgggagaccgaggagggtgtaccatttgaggccaggagttcgagaccagcttggccaacatagcaaaaccctgtctctactaaaaatataaaaattagccaggcgtggtggcacatgcctgtaatcccaactactcgggaggctgaaacagaagaatcaaacccaggaggtggaggttgcagtgagccgagattgcgccactatactccagcctgggcaacaagagcgagactcagtctcaaagaaaattaaaaaaaaaagagtgatgaaggtggggaggagacaatggactggcataggcccaggcatctttattttttaaaagtaaccccagcaaatactaatgtccagtcaggattgagaatcactgggttaagtgattgctaagaactgattctaagattgtgtcatcttaggatggaaagcaggcccacagagtccctgttaaacatagcctggcccagcattgcgcctcctctgtggtaaccaaacctaaacatgcctttctcagaagcagtccagatgaagccatttcaggttattcggggggagctgctgtgcctgctcacctttttgatattcccacaatgctttgctgactttgcagcacccagtagtttttgatggactgaattaggtgtaattctgcctatccaggatgatcagctgttcctaaaggtccccagagacgataacatctctgctctgagtatccatgccagtgtttaatagcctcttgaaattttctccttgaagtgatctgtgacttgttgaggtggggaagaaggtgtcagcaggggataagcatgatcaacttgatcgtagctgaaatgtgaactttagtggccaaactgagataaaaaatgatcctcacaccttctcaagtagttgaagactattgagttaccagaactgggagaggagaaaaagagcacattgtttaccttaattatttcttgggacctaatcatgttgccattacattgtaaattctgtagattttcacacttggaaaattcttcttaaatttagagttaggacttactaaattgtaatccatgaaaggtatttgtttaccctactcttcttttaatctcctactacctttcatttcccaaaatacctagaagacaaaaaacagtgtatatatgtgactgtcaaatcggtaagagagacagacatgagttgagcaaaaatggaaaggcattaaggaagaaaaactgacaagggtaagaggcacgtcggttaccaggagctgcacttaatttctctcttttctttctcgattagtcttatctctgctataagcctattctcaagcttgctaaaatgcaggcagatcaccatatcccacctgctccatgttgacaaatgtgggcttatggcccttgattccctcatgttccctgcagccttccatacacagtgcatgcagtgctgtctccaggggtgaactggcagcctggacacacttggtctaatccagcattggccaatcccagggcctgacttaattctaaatcacagcttaggttgaagcatattttttcctagagtggaaagctactggcagtaaagttctttacctctgtatctcagaggagtaggctttacctgatgtcaaaaatccaatgcagacctacaaaactagctcagcccccacctctccagccacctagagctggttgtactcattgccagtaaacaaacaaaacaaaattctaggattggagtcattttatatgtttattgctgatcgttgttcccaaagatttagtagtattgggcacttgctgttttgttatttcataatttattcctgacctacttcttaaaatgaattaaagtaggaagaagtgtaccaggaaaagatagatttgtcataatttcaactctaatggaaacttaattattactaaactcaatggcccatttatgtgtttataaataaaaatgtatccttccatacatctagtttcttaagataataagaatagtgttgtgctagtccttaggattattattagtttgctttttaaatctttacttccaaaaagcattacaattttgcaaaataattagaaatggtaaaaatttcttagccagtgcctgtttattgaacacttttaacaacaatctcaaaggtaacaagggttttcattgcttcctttcttgctttattatttccttcctttcttcctagagtcactcattatccaacaaatatctaatgagttccccctatgcccctgacttcatgtagcttacattcttatgaagaggggaagagagacagaaaataaagaagtaaaccatcatgtaggttagatagtgttaagtgctatggataaaaatatatctagaaagaagatggtagaggtgttggctgtgaaatttttgaaagaaggtggtcagaaaaagcttcccctgagaaagtgatattgagcatagaccggaaggtggtgaaggaggaagttacgagtctatctgggagtagagcatcctggccagtgcaaaggccctgaggcagacaagtgcttggcatgttcataaaacagcaaggaggccaactgcggtggctcacgtctatcatccaagcacttttgggaggccaaagcgggcagatcgcttgaggccaggagtccgagaccagcctgaccaacatggtgaaatcccatttctactaaaaacacaaaaattaggtgggcatggttgcgcatgcctgtaatcccagctgctcggtaggctgaggtgccagaatcacttgaacctgggaagcagatgttgcagcgagctgtgatcgcaccactgcactccagcctgggcaacagagtgagactctgtctcaaaacaaacaaacaaacaaacaacaacaacagacagcaaggaagtccatgtgactggagcagagtgacaaggtgggaaggtaaagaaaatgaccacagcaaggaaaggagcagtggacaagagtttgagagggtgctaaagatcacacagggccccttttcagtgggatacttgtaaggtgatgtgaagatggagtttcttactgtttagttgaaacagcagcagcctgtgttctctttcttccttgttatacctcctggatagtccccccagttttcctttcctctccaaattagcctcttcccattcctccccttgtgcttcctttcccccaaggctggctgcagagagggcttcggtttccctttctccttgttgccctgaagataagtctgcccctcctgttctaagaaatgtgctgctagttgctaaagaataaaaactgtcaggtagcacccaaaccaatagtcatgaaataaatcataataaacttatgcaaaatgtattcttagtttatactgcagaccagacacaaagtggttgtgttgctgttgagcatagttgaacctatgtgttttgttgggtctgcacattgtttcatgaaaatgtggattggtttccaacatttaaaaattgagaaattttttattaaaatgtattttcaagttttccttccaaattcatgttcctgatgaagttctagaataggcaaaactgatctatggtgatagaaatcagaactatagttgccgacacatcagagattgactgggtagggacagaagggaaatttctagataatgaaagattctatttctagtagtctgaaatgcacagtaaactcattaaaccatatactgatctgtatagttcactatgtgtacatgaaatctcaatatagctgggcacggtggctcacacttgtaatcccagcactttgggaggctgaggtgggtggatcgcttgaagtcaggagttcaagaccagccaggccaacatggtgaaaccctgtctctactaaaattatgaaaattagctgggtgtggtggcacatgcctgtaatcccagctacttgggaggctgagggaggagaattgcttgaacctgggaggcagaggttgcagtgagtcgagagcgagactccatctcaaaaaaaaaaaaagaaaagaaatctcaatataaagaaatttttttaaaattatgacgaatattttaactgataaattataattttatatatttatgggtacaatgtgatgttttgatatatgtatacaatggagaattattaaatcaagctaacatatccactaacctgcttacttacctggtttttataatttgaaatttactcttagttttttttaaaatatacagttgaccctttgtatccacaggttctgcagatagagagggccaactagggacttgaacatctgtggattttgatatccttgtggcgtcctggcaccactctcctatggatatagagggctgaagtcacagttatgatttccatcaccatagattagttttgcctattctagaacttcatatagaacatgagtttggaaggagaacttgaaactacattttaataaaaactttctcaatttttaaatgttggaaaccaatgcaaattttcatgaagcaatgtgcagacccaacaaaacacacagtcctcctgctgtgcaatcgctctcaaaacctattcttgtctatcttaaactttgtacccttcagtcaacaattccccattccttccctcccacccatgacctagcctctggtcaacaccattctactctctatttctgtgagttttttagattccacatgtaagtgagatgatgtggtatttgtctttctgtacctggcttttttcacatagcatcatatcctccagattcatccatattatcacaaataacatgatttctttcttttttaaggctgaatcatatttcactgtgtctatgtaccacattttctttattcattcatccatcaatgaacacttaaattggttccatgtcttagctattgtgaataatgctgtaataaacatgggaatgcagatatccctttgacgtattgatttcaactcctttggatatatacccagaagtgggattgttggatcatatggtagttctattttcagttttctgaggaactgtcatagcattttccataatggttgtactaatatacactcccaccaacaatgtgtaagagttctgttttctctgcatgctcaccaacacttgttatcattcatctttttgatgaaggccattctaacaggtgacattatgtatcattgtgtttttaaagtgcatttccctaaatattggtaatgctggacattttttcatgtacctcttggccacttctgcgtcttttgagaaatatctattctggtcctttgcccattttttaattgtgttatttgttttcttgctatcaggttgtttgagttccttatatattttagatattaagcacttgtcagatgtgtggtttgcaaatacttactcccattctgtgggttgtctcctcattttgttgtttcctttgctgtgcagagccttagtttgatgccattccatttgtctgtttttgcttttgttgtctgtgactttggagccatatccagaaaatcattgcctagaccagcatcatggagctttcccccattttcttttagtagatttacagttttaggtctcaacttttaagtctttaatccattttgagttggtttttgcatatagtatgagaaaaggatccaatttcactcttctgcatgtggatagccagttttcccagcaccatttattgaagagactgtctaaagaaaagttttaaatggaagccctacccaacattcagcaatacctggatagcagctgaccactttgggcttggcacatgctttctagattggcttagacaccttgtccacttctgtcattttcattaactacctggctccttgtattggtattggtggtattggtttatgactccttcagtagatgcttagcagtttgggtttcaactatcagtggggagcaaatctgaaggggcctgatacgtttaattggtagtttagaaaatagtacctgcctcttagggttgtatgtgaagcatgtaacaaaatcataagcctgattttgttaaatcagtccagaaactcgcctattcccctttcacttaatgctggtctcaaatctcaggattctcaggggtctctggacctaacttgcgctttcgtctcaaatgccaagggtctcctgaattttcagacttctttcctgagacgataacagaaaatatcttaactattactcctattatcaacacttaacacttaggcttataatgtgtgttgctaaaataatagtgcctactatggaaattttagaaaatacagataagcaaaaaaaataaaattactcataattctaccatcaaacataacctctgtttaccatgttatggtaaatactttcaaaactttctccatacacctatatattttatattaaaatgtgttttataaattttagcgtacaaatgcccagtttacaaaatggtcaataataacaagtcaaatgttaaagatgacaggccaagcataagtacttgcttactctctatcctaaaactccattaaaatgaccgtaaaagatacaaagggggaagaactctgaagataatgggatatgaaaatgcttggaagcaaattactgattaaacagaaccaagaatttgtacaaaaaggaccaccctcggagtgcaagtgatcccctagaaagaaccccctgtatggagttagcagcaatggggagcatgcatgagcaacgagattaatggaaagtcttcatggactagttatagtgaagtagggcttctctagagtattggcaccatagagtaaatcttttctctttctggcatttagaaagagggtgggccctggctaaaaggctagctcttgcctcttcacccaaagtataactagctatcaacaaagcccactccggtacacaaaggctgttaatgttcatttcgttcagggaagagtcctatcctggagacacttatatacatgacagtggaagaaagtggcacaaatcaaccagaaaaacaaacgaaatccttaatttataaagaaattatataaccaagaataacctgacatatgaagaagcatgaaaaagaaacatcaagataaacaattactattgccagaggagagatcattcagggaaaagaaaatcgccttttttaaaaatctgagcactcttagcatgattagagttgttgtttccacacaacaaaagcgtgatgttattcagaaagagcaatcagaaaataaaaaagatgttgaacatttttttaaataactaaaataaaaataatttactagaaggctaataatggaaattcttcctggtccaagtccagtggtgtttacaactaattgatcacaaccagttactgatttctttgttccttcatttccactgcttcacttgactagcctaaataataatattaataacaataatgatttttaaatctcctgtaatgggccaggtgcagtggctcacgcctgtaatcccatcactttgggaggctgaggtgggcagatcacgaggtcaggagttcgagaccagcctggccaaaacggtgaaaccccgtctttactaaaaatacaaaaattggctgggcgtggtggcgcactcctgtactcccagctactcagaaggctgaggcaggagaatcacttgaacccaggaggcagaggtttcagtgagctgaggtcgcgccactgcactccagcctgggcaatagagtgagactttgtctcaataaataaataaataaataaataaaaataatctcttggaatgcacaaccaaattttgaagaaagaaagaatatgctagaaaagaggagagacagaagcaatccagtaagaaaatctttgttttctgagatagaaaacaaagaaaataaaaggtaagaaattgacaattaaatgctaggaaaaaaagttcctgtagttgaggaaaagtgtgaggttaaaaggcccatcaagtgaatgaaaaaacatgcagagatacaattgttacaaaatttccaagtcaagaatacacagagattcctaaaactttctgaaaggaaaaataaagtcgtttaagattaacataaggcttctcatgaggaaactggattttaatagagcaatggctttaaatttgtaaggaaaaatgattttgaacttagaatcatattgtcagtaacttatcaagcgtgagggtgaaatgaagacattttcagacattcaaagactcaggccgtttacttctgagtaccttttctaaagcagttgcctaaggatgcactccagcaaaatgagggtataaagcaaggaggaggaagctgtggaattagaaagagtggagctaatccacaattgcaatgaaataaactgtaggttgacagatgttcaccaggcttagaaaaaattaatacaggccaggcgcagtggctcacgcctgtaatcccagcactttgggaggctgagatgggcagatcacgaggtcaggagatcaagaccatcctggctaacacggtgaaaccctgtctctactaaaaatacaaaaattagccgggtgtggtggcgggcacctgtagtcccagctactcaggagactgaggcaggagaatggcatgaacccgggagatggagcttgcagtgagcccagatcgcatcactgcacaccagcctgggcgacagagtgagactctgtctcaaaaaaaaaaaaaaaaagaaagaaaaaaaaattaatacaaattagggcagatagtgagctcaagaagaatgactttaagaagaattaaatgcatttccttttataggtagaagtagcagtgaacaatattcacatagttatagaaataatggaaattcttcctggtccaagtgcagtggtatttacaactgatttatacaaccagttactgatttctttgttccttctacattcccactgcttcacttgactagcctaaaaaaatgatgatgctgatgatgatggcacttctagcaacacagagggcgaagctgactcattcagatctccctccaggacaaacacacaaaaatgaaggataaaacataacaaatgttcaaataaccttgaaataaagactgtcagaagaatgcagagaaaaatgtttaaaaaagaacaataataataagaaatcctacatgcagcccaagccataggaatttttctgtggaatatagaccctagagtgttgaatgatgaagttgtatcattcaaacctggaaagaaaatttggcttcaggcccacaggagaccaggaccatggaattgaaacactggataagcctggagctctaaagacctgtctagtccattaatagagtattaggcaaactctaaccactacttagggaaacaaggaagtcttgtaggccttgagtgaaagttaaaaaaagaaagtcacccttgaacaaacaaaaccctgggcctgtgcctcattcagaaattaggttcaaaattatacttcctctgtggttcagaagcctcaaaccatattaaacaaacaccaacagtagtgaaaatgtccaggacccactaaaaccaccataaccctagcagagtagacatgaaagtgtctaaaaacacatcacgacttaagtgtacatggaacatccacagaaccactcctccccttccacaattaaatatgactcagaattttaaaaacctacaaatcactgcaagggagtgtcagcaaacagaacaagcagaagaatcagatccccaagaatttaaaacaatggaaatagatgaacaagatcagaagtgtgttaaaaattattgaaagaataaagagaattgaaaacaaaagggaaaatgcaacactatgaaattagaacaagttgatttcaaaaagtatagaaatgaaaaatataggctttaaatatttttaaaaaattaatgtcctacttaatgtattagagccaaaagtagattattaaactggaagttaaatctgagaagactatctgtaatcaacccaatgagataaaaagctgtaaaatgtaagaggttaaagcacacaatgacaggagaagctccaacaaatgtcaaatagaaattccagaaggaaagagaaaagggagaataaatcagtatatattggttttataggtaaagaaattaaaaatagtaatataatttagcaaaaatagaagtgagtaaggagacttaaatgttaatatttcggctgagtgtagtggctcatgcctgtaatcccagcactttggaaggccaaagtgggtggatcacttgagctcaggagtttgagaccagcctaggcaacatggtgataccccgtttctacaaaaaatacaaaaattaggtgagtgtggtggcatgcacctgtagtgttagctactcaagaggttgagacacaacaatctattgaacccaggaagtggaagttgcagtgaaccgagctcatgccactgcactccagcctgggtgacagagccagaccctgtctcaaaaaagttaatttttcatggtaagcattcgtgaataatgtctaaacttgataagtaaaaaaaaaaagccatataattttattacttagagcagtagttctcgattccaggaaagattgggaagtgtatcctgtggatggagaatgctactagcatttaatgtcaagaagagagatgctaaatgcacagaactatctcatacaaatcccacctgaaataccagtttctctttctgagaattgctgatttagagttaataataaactcaccagaagaattaatattaaaaatgattttaagaaaatggtggctggctacagtggctcacacctgtaatcccagcactttgggaagccaaggtgggaggatcacttgagcccatgagttcaagatcggcctgggcaccataatgagaccctttctctatgaagaaaaatttaaaaattagccagctgtggtggcacatgcctgtgatcccagttactcaagaggctgaggcaagagaacagctcgagtgcaggaggttgaggctgcagtgagccatgatcacaccactgcactccagcctgggtgacagagtgacacctgtctcagaaaggaaaaaaaaaaagaaagaaagaaaatggctgcttcaggaaatagaactaaagttggggagaatgggatggaaaagtattgcttttcattattagttatttcataatacttgcaattttatcatgtgtatattatgttggaaaaagaaatgactagtcaaccagtagcttcttctttcatttttgcttagagaactcttttctattgaagtatctgtcattcaaacctaagcaccactttgaattactgagctttttacctcaaagaaaattctaaaggcatatcttatgagcgttaaagatctttcaagtgtttctggctggaaaataaatcttgctatttattaatttagggagtcacagcaaattagtgctggaagggattcaacttatttagtggtatggaataatttatccgtggtagagtgattttgagcagtagctctgtataggacaaaggaggactggggtatggtcaaataacaagagcccatggctgcaaattggaacctgcatcctgcctttcatggacttaaaagatctgatcctacttgacctctccagttttatttcctgtaaccacccaaagcacgatttctaccagtacctttggtattccatgaacatgtcttattagcgtttaccttttcacatctctatgcaaaatcacgtgcctgcctttttgccttctatccattatttgagagccacttcaaatccagtatcctctccaaagccatcctctctggaacagcaggcagaagccaggaccttggtcaccctacttagatcaagtaactcaccagatgtcccaccatggtggcagaaccaggacttgaattgaggtctatgtcattccatagcctgtactcccagctgccatttacagataaggaaattagggtacagaaagcttgtatatcttgcaaaaaaacatgaagctctaaagtgccagttctggctgggcgcgttggctcacgcctgtaatcccagcactttgggaggccgaggcgggcggctcacctgaggtcaggagtttgagaccagcctggccagcatagtaaaactccatctctactaaaaataaaaaaattagccaggtatggtggcatgtgcctgtagttccagctacttgggaggctgaggcaggagaatcgcttgaacccaggaggcagaggttgcagtgagccgagatcacaccatcgcactttagggtgggttacaagagtgaaactctatttcaaaaaaaaaaaaaaaaaaagtgccatttccagccttcaaacacagtcctgaaccctaggctctgcatgtggtattgcctctacggatattaacgtgtaagagtcctttggcagaggaatttaaggggagtggataagtattgtttttgcctctgaaagtaatggcaaaaaccacaatcacttttgcaccaacctaatataaaagaagagggaaaggatgggcacagcacaagactacatgctatggcaaccttggcccctgccactcactcaagatcattcagccaacaaatgtctgctagccagctgcatgccaggctgtgcacttgatgctcagaaataacagtcctgccctcatcgagcttgcagtctacattcaaaaccagcttgcataggtcttagggccttttgctctttctacatattgagccactataattaatcaatgatagttaataagggaaaacagatagataatatattctggttattgctaacagtccaaaaagtagtaagaatagaaaacagcttttcaaattccttttaggaaaggctgataaagccataagtgaaaatatgttttttctgggttctaattcatgagacttttgctatttgctgtgtacattatagaaaagttcacatcctgttgctcagggcaatgttgaaaaaattcatagtaaaatttatttttgttgtttatattgaggcccatagagaagtcatgtgaaagacgtagtaagcttgggcatgtgcttgcatatttgagattaaaaggttaatatatgcattattaaagttataaatgtgtaaattattttgagtagatggatgaatgcccttactcactcttttctgatacctccggttcacagagaaaaatacaatttcaaaacttaaagaaaagcattactttactgatgatggctcttagtttcagacaactgaatccattctagctagtttaagcaccaagagatttaattggcattttgaaacttgccagatactgttcccagaatgttgtcactggaaacatttaaagataaggaaacatagaagcagagaatagtaatttccagggactgggaggagagaaaaatggggagaaattaatcaaaggaaaccaagtgaaaattatgcaagatggagaaatcctagagatctacagtacagcatagtacctgcagttaaccacccactgctgtacacttaaaaatgcactaacaggccaggcacaggggtttacacctgtaatcccagtactttgggagaccaaggcagatggatcacttgaggtcaagagttcaagaccagccttgccaacatggtgaaaccccatctctagtaaaaatattatacaaaaattagccaggcgtagtggcacacacctgtagtcccagctactgaggagactgaggcaggagaatcgcttgaacctgggaggtggaggttgcagtgagccaagatggtgccactgcactacagcctgggcaacaagagcaggtgtctgtttcaaaaaaaaaaaaaaaaaaaaagtactggcagggtagaccttgtgttcttatcacaaaataataataaattaagagaaaaagcaaacttttggaggtgatggatatatttatggcatagattcctccaaacttatcaacttgtatacactaagtatatacagctttttgtatgtcagtcatatctcaataaagtggtttaaaagttaataaagatgaggaaactaagactcaggcaaattatttttatatgttcattcagcaagcattttttagccctaagtaccaatcactgtacagaaattagtattgcgtttctcctttcatgaggctcacaatctattttcccaataggttgtgcatcagtctcactccatgagctttaaaaaaatacagattctagaattattaaattagaatctctagacggggccaggcatagtggcttatgcctgtaatcccagcactttgggaggctaaggtgggcagatcacctgaggtcaagagttggagaccagcctggccaatatggtgaaaccccatctctactaaaaatacagaaattacctggccatggtggtgggtgcctgtaatcccagctacttgggaagctgaggcaggagaatcgcttgaacccaggaggcataggttgcagtgagccaagatcgcaccactgcactccagcctgggcgacagagtgagactccatctcaaaagaaaaaaaaaaaaaagaatctctagaaggcctggaaattgtggttttaatctccccaggtgattgtgacatgccagcaaggcatagaaaaccctggtgtagttgaccctgccataacaacgtagaacaactgagacacttaaggtacagaaccagggctctacactgatcatgacctccaattcagagcatctaccacaaaaccacagtgaatcttcattattggcatgcagctgactttggctcttagtctgaagtttaaaatgttgttaccattttgttatttttttatttataaatatttatcatggcaatattttataagtatcagagatgccgtttaaagactactttgaataggagacagggtttgtgttttgcaaatatttttggataagcctcttacccccaagtgagtgtagatggatccattttctcatctacaaagtgagggagttacgataaatttagattttaaaatctatgatgaggccaggcacagtggttcacacccataattccagcactttgggaggccaaggtgggcaaatcacctgagggtcaagagtttgagaacagcctggccaacaaggggaaacctcaatctctaccaaaaaaatacaaaaagtagttgggcgtggtggcgcacacctgtaatcccagctactcgggaggctgaggcaggaaaatcgcttgaacccaggaggcagaggttgcagtgagccaagatcacgccactgcactccagcctaggcaacagagtgagaatgtgtctcaaaaaaaaaaaaaaaatctatgatgaaaaatgttctaagttaccttcaaattcctcccttcctgcctgtggcgaaatggaccttgaagctctaggtcaatttgctttgctgtcattagtattttgtgtttgaggacctgccagaagatgtgtccctttaggtgatcttagtaagtgacttttctcatgtaaggatttgtgtacttttacaatttggtgatttataatttcaatcagcacactggaagctcattatctgtcatataagagtctggttatttgatctcctagtgttggttccttggttactttgcctctctgcctctgataagatggttattgatagccttcgaggctttaattatttaatcattgttgatgcaaataaaaagtcccccgaaaggacagcctttcattacaacatcttctcctttcctttcccagttgaaatgtccttgaactaccaaccagctatctcaaaaaggacatagattatagccagggtgccagtatctcaaaaatatttgagaatcgtctcctctctgtgtctcttcctaacctacagagagttaaggttgccagatttagcaaagaaaaatacaagatacccagttaaagttgagacaaagaatactttttaatataattatatcccatgcaatatttgagacatactaaaagattatttcttgattaatatgcaaatttaactgagccttctatattttctctggcaaccctacagatagtgcatatcttaatgctttagaaaatccatcttatgttttgagatctaaatttgtttgagatttgctcctaattgaaacccacaatgaaatggttgtgacagttaagactgcatttgcctgaagtatagtggatgttgcactgcccacctccgcacaactctcttttgttgtgagaatataagcatagaaacaagactttgcttgagaaacctaagctaaatagtactcttttgtaatttgtattaaatattttgcagacacacataagtaaagatagtgctataatgcattgcacagaatttatttacaccctatttagtttttttctttttgtttgggtgctgggtacatggatgggtgggtgagggtgtttgttttgccatctcctatgtcagtggttttgagcctttgctatacaatagaatcacctgagagcttcttaagctctcaatccctgtctgctttgcagaccaattaaatcagaatcactggaaatagactcgggcatcagtatttaaagctgcccaggtgactccaatgtgcagccatggctgagaaccaatgccctaaatgaaaatggcaaatttgaaaaagctcttttaactactaaattatgaccttatattttaatcctcttcaaaactgatttcatgtacttttcagtaagcatcttatggcaattacatttattattttgtattatatattctatatatgatgtataatataaagtttactttaaaaaatgatcttatgaggctttaatcttgtcactgattcagtattaatttcttcaacatctataattggttttcattttcaaatatttctttgtgatttttcttatgtatttttcatatgtatttctctctctctcagattctaagccagtgcttctcaacttgaactgctcgttaaaatcatcttaggatgctataaagaaatgcccaagcctgcctcctggaggctgtgctctagttggtcaggtgtggatcctaagcattagtgttttataaaagtacctaaggaagttctaggtttagccagagttgaaagccagtgttttcaatatgtcaagtacctatctagctgatcaactgcatgtcctggggaacttttaatacataaatataattgctctgcttaatgaaatagaaggaattttgaggttctgggagaaggagaaataagggagtggtaagtgaccaccctcttgtgtttatactctcagattcacgggtgtcacgaggaattcctgatgttggggatagccgaaggactcttaagactaaagataaagatacctagtgttttaagtgttttctctgctatgctacagtgtaaaccattctgttgccctgaaatctccagagaaggttgccacatacatgggtaagagacacacgcttacatccacaagtagtgaatacccattgtttaggcctcacccagtggaaatcctaactgggtaggttcaggttaaggccctgaacccagtaattcaagccctccccagaccattgtggtgattggccacattttgtcaccactgccttgtaggtgataaaacatccctagacttgatcatcataattcacctgattgacagctgagcaaactcctgtggggtaacaggaaaatcaatggtaaaactgagtttctgcaacaggagaaaaactagagtctgattcttattagaccttcctcaggtctacctgttccttttctcttcaaggggccatatgtaggaagaaattacctttctgaactaggacaaaagactaagaagatgatgtctacaaaaaataataaaagttcaaagcagacataatctataagatgaaagagaggaagcatggcatgcattatcaataatcataacatacccattcaaagatttcattcacctcccagttactgagcacctgctaagatacaagtactgagctaggtcctgggaattcagagtatgatattgtctcaaagaatttacagtctagaagaggagatcaatgattctattagaatagaagaatggaagcgtataccaagtaacatgcataggcccatttcataaaggattataagtgaggggagacctcacagaaaagcagaagacagaacaagggtttggaagatacaatgattaggctgtggggaaaatggaaggagtgaagggaaagcaacaacatctcttattagaatgaattgaaactctggatgggcacagaggcttaaagcagcatggtatgttgcctggattacaaatggttttataatgctgtcatgtaatgatctggaggagttagggtaggtggaagccagtggtagggtttgtgatgtgtcaaactccactcatacatcagcccagtcattcttgtattttgatgagttcctagagtggccctaaagagctagatgagaggtggccaacccaccttactagtctgaaacaaaaaacatatcatctttatttctcaggttctgctgcaaaattcttcctccagatttaccatttcttagttcatatgagctgctataacaaaataccacagacagaaatttataaacaatagaaatttatttctcacagttctggaggctgggaggtccaagaacaatgcaccagctggtaagggccaggtgtctctgcttccaagatggtgtcttattgcatcatcctctagagaggaggaacactgtatcctcacatggcggaagggacagaagggatgaaagggggtgaactccctgtgctaagtctttttataacttaatcccattcgtgagtgctccagagcccacatgatcaatcacttcctaaggaccacacctcgtaatactcttgtattgggggttaagtttcaacttgaattttgaaagagacaaaaacagtcaaaccataacattttaatatcatccttggtggtacttggttgcatttgctgccagaatttctttttattataggctcttgaattttcaacctctttggacataggtatttattttgcacaatattttgtggtagatcatacacaaccctgatttcaatttgttttgttgcctttgagacggagtttcactcttgtcgcccaggctggagtacaatggcgcgatctcagctcactgcaacctctgcctcccgggttcaagtgattttcctgtctcagcctcccaagtatctgggattacacgtgtgcatcaccactcccagctaatttttttttttttttttttttttagtagaggcaggttttctccatgttggccaggttggtttcgaactcctgacctcaggtgatccacccaccttagcctcccaaagtgctgggattacaggtgtgagcctccatgcctggccttgatttcaatttgaatgtgatttcattttgatgaaaacctgagttttcactgtgaaggagttgtattgaccaacattagtagccctcacaattccacagtcaccacacaagcacttaaccatgcattcatgttaacctctgcctgctctgccattaacttccttcatttgggaatactggctttagttgttgtgcatttaatattttattatgaggtatatagttcaaatgtacagacaagtacaatcatgtaacaagcaccagtttacttactaagcagcataatcagatcttcacattttgctgtctttgtgccagttcttttttttacaaactaaaacattgcagatacagttagtagaagccctctactatcccttcttagtctcatttccctctctctagaggtaacaccatcctgatctcagtatttattgttgtcatgagtggttttatacttttactacaaatatatgtaataacatatactattgttttatgtttttaaactttaaataattgaatcctctcagtattcaactgtgccttacatttttgttgaaaattgcctctgagatgtattgatgatgacacagaaatttagtgcatttcttttaattactacatagaattaaatttatgaatataccaaaatgtattttttactgcatacaagtacttttcactgttataaggctgcagaaaacattcttgtacacaactccatatgcacaaatatgttgtatttttattctttaatcattgttttactcatataagtggtatcactgtgtagcaatgaaaaaaactatcagtcattctaataatcaaaaacatgacaatgtaatcaacttcaaaacaatattagaatagtgtgctgatgttcttaacttactgcctttcacaaaggtcaccagcaacttgcctcaaacagacatgtaagcctaaactctgaaccatagaataatttaaagagtaatcaaccataaatatagaattggaaatgaggctaatatagctttaagaaacattgagtcaatttaaggactgatcgtactctttttttctgtttccttgacatatagctggtcaaaattgacctaatttgcaattcctcttatgtggacctagagggaactgactagtatttagccattgcacttaatatgagcttttgttaaaagtacacacattaaccatttattactctgggagggaataagtcacgtttttttaattgattttgatttcattaattactgcctcctatatacagatcaatacattaggtcctctggggcgtccaaagaaagcctcagaagaacttggaacaccacaaagaagagatgcttaggaaaagtcatcagtaggattttggcaagtaaaatggagtttttcttttttcccctagtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacgtacgttgccttttaacctgtttctgttattgtatcattcagttatgagaggcgcaaggtcaaatgtgtaaagaaaatctgtttggtgaagtggagagaaaaatgaatgctgagccatcatggtttgcaaagctgttcacgaggttcagtcagcgttagatatttgcacacaggcttcatcatgaatttcaccatatttattgaattcctactatgccttttttctgtgtaaatgttaacataaccaacatgctttctaattctccacccaccttgaaaatatcttttgtctcccaggaatattccacccactctctttttttccctatccctgactcctaccctgtttggcaacatcttgaatcaccaattggaaatttttccagattgctcctgggaataaggaagggaaaatgctttggtttgaattctacagagatgtttctcatttataactttatctctgtctgtctttgtcctttcagatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggtcagtagatctttctttctttccccccttgctgtcctcaaacaattgcttgaccttctgtttatttcatcatgattgcaaattcataatttttatcatatctttctagtaacctttacctatctaaaggtattcttcagaaacatgtaatccttaaacctgtaatctcagcactttggaaggccaaggtgggaggatcacttgagactgggagttggagaccagcctgggcaacaaagtgagacactgtctctaccaaaacaaaaacaaacaaaaacaaaaaacatagctgggcatgcctgtagtctcagctactcaagaggctgacataggaggatcatttgagccccaaagttcaaggctgtaatgagctatgatcatactacttcactccggcctgggagacagagtgagtccctgtctcaaaaaaaaaaaaaaaaaaaaaaaagctccagggatattaatagtagaaaatggtgttactactttgcagatcagtacactgcttgggcattatcttaccacttgctaagtcagtcactcaagaaacattttgttgttatggttgttgttaaggtttgtggcttctgagtccatattctaattacaagggaccatgatatgctaaattcctgtagtatattgcagcaactctgtggttttatatgtgatataagaacttaatattcattaacttattcattaatttattcatgtattctaaacagtacatgtctattaggcacctatctggctcactgtcctcatggagctcactaaccatccaatcaaggcatgagattgctaagcagaaaaaagcacatataaaaattttaattataatgataagcaaaatggaggaaaatataaggtattgtgaaataaaaatagtggatctagggccggctgtggtggcttacgcctgtaatcccagcactttggaaggctgaggtgggtggatcacctgaggtcaggagttcgagatcagcctggccaacatggtgaaaccccatatctacaaaaaatataaaaattagccaggcatggtggcatgtgcctgtagtctcagatactcaggaggctgaagcaggataatcacttgaacctgggaggtggaggttgcggtgagctgagatcgtgccactggactccagcctgggcaacagagcaagactctattggaaaaaaaaaaaaatagtggatccaggaaatgaggtttgagctgagacctgactcaggtcaagaatgagagaaaatgcatttcaggcagaaggaatagcatgtgtgaaggcactgagcaggaaaagaattgggtatggtacaaagaatgaatggaaaagcagtgttccagcagtgtgagggagcagggggcagtggcaggcgaggtgggcagaagtaggcctgcagagtcttcttaggccatttttgggtttcattctgagtacagtgaaaagctatcagcattactttttatttaggttttctttgtgcataagtcttctcacagtgctaacaaagacatacctgagacttggtaatttataaagaaaaagaggtttaatagactcacagttccatgtggctggggaggcctcacaatcatggcagaaggcaaacgagcaaagtcacatcttacatggtggcaggcaagagagagcttgtgtggggaactgcccctcgtaaaatcatcagatctcgtgagacatattcactatcatgaggacagcatgggaaagacccgcccccatgattcaattacctcctatccggtccctctcacaacacatgggaattatgggagctacaattcaagatgagatttgcgtagggacacagccaaaccatatcactttgtaagaggtcactgaagactgctgtggggggaatggatttgggtagtgggagaggcaccagaatggatgtgaggagaccaggtagaggtgacagcagctcatggtaggcaaaggtgataatggcttgaacagctgtaggtcagctggccaaaaatcaattcccttctagccagtttccaaaaaaataacttgtcaaaaacaaatcacttcccctaatgatcaattagctgagttttttattttccaagtgactgatttaccatatttattaaatctattatacaaaatcttaaagcagatttgggacagattcccacaatcattttggtgccatcaccccaagaacagagaaagggacagattcaaaggcaggggcaagggcagggtgaggagcggataagggcccagtatctgaaacaggaagcagaagtcaaactgacattcagtcaagctggactgacggagctgcaggaatgactgctgacaaccacgcaagctcacactcaaagaactcctcaggattctgcctagttgaattctcacagtcattctgcaaacttattttcaaccagctgactttcttccatcttgaactagaatggtggctgtaagaatggaaagactttggaaagacatatgatacattttgtaggtgagatcagcaaaactggctgttggattaatttagaaagaaggaggagggtgtgtcaacaacaactttaaggctgatgggttggatgtgtggggatggtgaagaggagggatggtcaagaatgacttccaggttgctgtttcaaccacctgagggatggaggtgccaattgcagacataataaagatgacagggctgggtgtggtggctcacacctgtaatcccaacactggaaggccaaggtgggtggataacctgaggtcaggggttcaagactgaccaacatggtgaaacccatatctactaaaaatacaaaataagccaggcatagtggcacatgcctgtagtcccagctactcgggaggctgaggcaggagaatcgcttgaacctgggagacagaggttgcagtgagctgagatcatgccattgcactccagcctgggtgacaagagcaaaactccatctcaaaaaataaaaataataagatgacaggaataggattgaagagtaagtttgattatgttaaatttgagatgcttgtgagatagacaagtagatatttcatgtttgtatttaatatatggtgagcccaaaagagaggtaggggctagagatatcaactgtggaatcagacctatagaaagcatttaaaccatggcaatagaccacggcacctagggaaagaatgtggaatgaaaaaagaagggagctaagatcaagctgtgtccaacattaaaggccaaatacagaaggaggaaccagcagaagagatagaaaagtatctggaaaggaaaactaggagtgtatattattacagaaacaataaattatttctatttttttttctttttttttttttttttgagacagagtctcgctctattggcaagctggagtgcagtggcgtgatctcggctcactgcaacctccgcctcccggattcaagcaattctcctgcctcagcctcctgagtagctgggactacaggtgcgtgccaccacgcccagctaatttttgtattttcagtagagacagggtttcaccatgttggccaggatggtctcgatctcttgaccttgtgatccacccgcctcggcctcccaaagtgctgggattacaggggtgagccactgtgcccagcctaggaagaaattatttcaaatcagggaataattcagtaggtggaacactgctgagaggcacagatcatcccaccatcaaatttgcattggaaaggtatttttctgctcattattaatgaagatagataaatatgaaaaacaaaatgtagcaaagatagagatctaagagaaagtaaatacatttgttgtgattggtaaataatttaaattaccaaaaagattttgtgttaagcatgtcagagtgaaagtatgctcttatctctaattgtcttaaaaatccctctaaaatgacaataaaagagggggaaatggtataaactagcaaggaggaagaaaatgagagaggagacacgtggaacaggaaaaatatatatatggcaagcagatggacaaggggcagctgaaatattagagtagaggaagctgaaatgtaggtacgtacactacaaaacactgggaaattggaggctccaagtattgtgaaagctggaagtcaggcaaaaggctaaaagcaagggaactggttcaaagtctttgcaatcagtagcatcaatacactcaggctcccacatttacacgatgcagtggatggtaggataaaaataccaagagatgtcaagaaggttaactaaaagtctgcacactgaactgtgagccagcaaaggctgttcccttctctactttgtttacttcaaagtgcctgcaaaaaaacttatagcccaaagcagatggtgacctataaaagacctatagtgacaagtgggggtcccccagtgaagatgctggctggccacccagttgtcccacaataaggcccatcagtaaacgaagagccaatacacatgaagcttcaagaaagagaccaaatcacacaaaaaagaattcataaggaacagaaataacagggcaaaataaaaaactttaaaactatagtatcctaaaagagatacaaaaacacattgaatctatgaaacaaaaacaggatgctattttgaaaagagcattcagagaacaagaaagagctcatagaaattaaaaatgtgatagctggaataaatatttagtcagggttggaagataaagtagaggaaatctcctggaagtagaagaaaaagacagtaagagagaaaacataagaacaccagaggatcaagcctgatgtcccatcatacatctaataggacttccagaaagaaagaagagagtaaacagaaggaagaaatgatcaaagagtaatccccaggaatgaaatacatgtgttttcagactgagagaaatcattaaatacccagcagaataaatgaaaaataaaacaaggcacaccatagtaaaatagaataccagggataaatagaggatctcagactagaaagaaaaaaagtcacataatgaaaagctgaaaggtaaacacaatggaaaattaagtttgcatctaaaattgataaactgagaaatcatgatacaaacatatttagaacgagttttctcaacctgatgccatttactctgttgttgggggcctgtcttgtgcagtgaagggcacttagcagcacccctggccttcacccactagatgccagtaatactcccacccggtacccacatcatgataatcaaaaatgtctccagacatccccaaatgtctcctgccagaagtgggggtgggggagcgctctcctttgagaatgatgatttaggaaaatggaggtaaatagagaaaaacagctaaacgagttggaagtgattggctccgaggagcaaaacctggaggaagaggggtgagcagaaaagagctggctcttccagagagcctttgctgcttggctttttaaatcgtatgcatacatttctttgctagaaataaatttttttaacaacattcgagtgaccatgttattatacatgaagttaggatgctttcccaactcttcttttgctaaagctgttttctccacagcataacagtgcattaaatgagttttctaattgtcccagaaatttggctgagcccccaaagctgagaataaaaagggcttattgcctacacattctgaagagggatgccagtcaatctatcccaagctatgaatgaatgacccaggccctcttgagtgtgtcactttaacagaatatatgagggaacgaggagactgatttggaatgagaattttccagggagaggttaaaaccctatccatttcccctccacctgtagtcaagtaagaaaggactacaagaaagctgctcagcgtgctgaacagtaccccctggagtttgtgaagactgctgcctaattcttgcatgaaaaagtccagctgctctaaaaactagtttttaaaaaactttcaagtgaacctgcacatggatgtttatagcagctttattcataattgccaaaccttggaagcaatcaagatggccttcagtagatgaatggataaactgtggtacatccaaacaatgaaatattcaatattaaaaaggaatgagctatcaagccatgaaaagacatgaaggaaccttaaatgcttattacttagtgaaagaagtcagcctggtgtagcttaatactgtatgatcccaactgaatgacattctaaaaaaggccaaactggccaggcacagtgacttatgcctgtaatcccagcactttgggaggctgaggtgggcagatcgcctgagcctaggagttcaagaccagcctgggaaacatggggaaactctgtctctacaaaaattagccaggtgtgatggtacacacctgtagtcccaactactagggaggctgaggtgggaggattgcttgagcccgggaggttgtggctgcattgagccatgaacatgcccctgtactccagcctgggtgacagagcaagaccctgtcttaaaaaaaaaaaaaggggggggcaaaacttcaaagacagtaaaaatatcattagtgtccaggggatggggggagggaagtatgaatagagcacagagaatttctgagcagtgaaactactttatatcacactatcatgatggctacctgtcattgtacatttgcatagactgtaccacaccaagagtaaacctcatgtaaactatggactttgggtgatagtgataggtcaatgtaggttcatcacttgtaacagatgtgccactctggtgggggatgttgatagtgggggatgttgatagtgggggaggctgtgcatacataagggcagggaatgtatggaaaacctctgtatcttttgctcaatttgctgtgaacctaaactgctctaaaaattctatttaaaaaaaaatgtctaaaggatgatgtgaactgcctaggaccgcagagtgagggaaagagtcccctagagggtgtgacctgcacttccaaagtttcttgggagtaaaatgtcaagctttcccattgagtaactggaaggtaatgagacttggcccatttgataggtacctgcctaagaagactgcctacaggatgagcatacaccagccaaaagaacgtggaaggctgtaccttctgataagagtcggaggagggatatctaagagtcaggaggagggagcattacataggttagggcatagtaactgagagtcccttattgcaaaggggtctccagagaacccacaaaaacaccccattggagaacgagctcgcatttgtgagtcaacaccaaattacaatgacaaccactaagataagacttttttctgcttttatctaatttgtgaaagaataaagcagaaaaaaaaatcacccactggcaaaaaggagatgacaatgcaaagtttctcagcttggactctatcagcaccaaggtaagatgatgtgcaggactggggtagggcaggttgcagcttaaattaagatgggcttcactcgttcatcagaaataaagaaccaagagaaaaacacatgtaacacctctgtcttctaatgatcccccatttctgtttggtttttaatcccacaggcttagcctttgcaatgctggcagctgtgcctccaatatttggcctgtactcttcattttaccctgttatcatgtattgttttcttggaacctccagacacatatccataggtaaaagcttacatttgatattctattacctttccttgtgtatttccaaccttgcaatgtgccaaagaaatagcatggtcattattaaaaattgtcttaataacaacttttgttactgcatacacaataggtgctttaaaaacctacactcggccaggtgcggtggctcatccctgtaatcccagcactctgggaggccgaggcgggcagatcgtctgaggtcagtagttcgagaccagccagaccaacatagtgaaaccccatctctttgtaaaaatacaaaaatttgccaggcttggtgaggggtgactgtaatcccagctactcaggaggctgaggcaggagaatcgcttcaacctgggaggcagagattgcagtgagctgagatcacaccactgcactccaacctgggcaacagaacgagactctgtctcaaaaaaaaaaaaaacaaaaaaaaaactacactcctcccaagatttctagaacttacaattttcttataatcttgggataataaaaaagacaattttcttgtgtaaaagggcaattgttgaaatctgtgaaatgagataaatgcttgatttcataatgtaatttgccagcttctggtttttatttttatactaatggtagtacttcaagttcctaatcgctatgcatgtatttcatttttgtcatattttgggctctgaattgtacatattttcagtttatcaggggcatatggatttcataatagttctggtgctgcataaggctactacctaacacacacatactttccagatatctactgtttggtaacaaatacccccaaaatgtagcagctttacaacaacaatcatgtttgtatcttccatagtgcctgcagttcaggacttgggaagggctggtctgggtaattcttcctctctccctttttcctccctcccttccctcttccctgccttcctttctccatgcagtcccaggaattctccacttggcctagttggagctttcttacacatggtgacttagggcttcagcagaaggtggaaactgcagcccttttattacctagcctgagaagtcacacaacgtcacttctgctgcattcttttgattactaacaagtcaaagccaagccagattcaaggagagagaaattagactccacctcttgatggggaaatgtcaagaagactctgtggatggaagatattgctatgtctatttttggaaaatacagtctgcatgcgcacacacgcacacacacggttgctcatacaccaggggcaggacactaaagaagggagcgtagccttccttcaagcatgatttcttgacctcacctttgtctcttggtgtactggcaaaaaccatatcagatactctgaaccctgaacagggatcttcatgcttcattatttcaggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcaggtaaggctcaaagatggggaaatggactgtctcgtttgttaacctaacaagtatctactgagtgctcttggctggtcttactacacggtggatggggtcagccagaaaaaccccagagctctgggagtaactgcagaccaactgcatgccatattcacaactatgagtgacgagcaagcaagcttcaggtgggtgttttaaagatcccaaaagcaagacaagtttgacacggcaggattggttagtgctgggagaggacattcctctctgcttggaagaaacagccagtgatcatgatcatgcactttagtctacatgatgaagcatgcccccagctcaatgagagacagggaaggcattagtgtggtcagcgcactgtggggcaccaagccactccagaccttccattgttttctgtataaccagtggttgattttctagctacttacccacttgaggtacaagtttgcagtgttagaaaatggtccagaggctgggcgcggtggctcacacctttaatcccagcactttgggaggctgaggcaggcgaatcacaaggtcaggagttcgagaccaccctgggcaacatagtgaaaccccgtgtctactaaaaatacaaaaattagccggacatggtggcacacacctttagtcccagctactcaggaggctgaggcaggagaatcgcttgaacccgggaggcagaggttgcagtgagctgagactgtgccactgcctgggtgacagagcaagactccatctcaaaaaaaaaagaaaatggtcaagaaagaacttgtatatttgctgtgggtcataacttcctgacctatcatgtgattcatttgtttctttgtcattaaaaaaaaatagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatgtaagtaagaaactacctgacgttctggtacttttctctttattccccccaaccttctcttctgtatctctttttactttcaagactcatttttctttttctccagtaatttccataaaaagggaaaaattcatttattctaaggttttctgaaaagagtgttacaaagagaactaaaccttaaaatctctgttattaatatgtgatactgcctacttcacaaggaattttttgctaggtctttatttggtgactgcaccatcactctaggtaaagagagagctttttcttacaactaaatcatgatctttttggaaactaacaaataatttaaaggcatgaaggtagaacatttctctctcaaaaactgctaattaaggccgggcgtggtggcttatgcctgtaatcccagcactttgaaagggctagatgggcggatcacgaggtcaggagatcgagactatcctggctaacaagtgaaaccccgtctctactaaaaatagaaaaaaattagccaggtgtggtggcgggcacctgtgatcccagctactcaggaggctgagacaggagaatggcgtgaacccgggaggtggagcttgtagtgacccgagatcccatcactgcactccagcctgagcgacagagcaagactctgtctcaaaaaaaaaacaaaacctgctagttcaaactggcaaaaaataagtaaggctataaaagatttaactaacatgaatcttttaattatatagaccccttaccaaacacagagaaaatataaattatttgcaagcacttaaggaacaatggtaataactgatcatgttgaaatcgatcatgtgaaactgatcacaaattctaaaaattcaatgttttttcacacagtgcaatataataaaaaaatcaaaaactaaacatagctttaaaactcccatatatttagaaaccaaaaaacatttctaaatgacccatgaataaagagggaattaggaaataattagaattaattgacagtagaagtactacatatcaaaacctctgagatgctgttaaagctatccttgaaaagagaggcctgttgcctttgtaggtatttatgtgccagacactgctaggtgccaagatgcagctgtgagctaagaagatacaggccttgccctctggagctttcagtacagtgggaagattgtaaaccattaatcgcatacatgtgtagtcaattacaatttgtgttgagtgctatgaagaagaagtaccagctgctctgaaagttaaatagggagacctttagattaggggtctaggaaaggcactttgacatttaagttgagtccctgactgataaaaatggaatcaggctttaggaatattctctaaataacaaaattcttagtttcccataatagctattatcatacttcttactctgagacttgtcgaaatgccttgtcacaaaggaaataaaacatactgagtactctaggcatcgataggaaagggtgcatgttgtgggctgagtgggaagtctactttattgggtttcaaatgagaactgactatatttgaggtgcccagttggtaaccctaggatataattaagtggatttgttctctagttctcctaggttcctttcccaaacatcagcactcttgacttcaaagtcctgctaacaataatttctctctctttgagctcctaaaagcttttcaatgtatttacctttggagtactttgctatcaacccttaattctttccaattgctcaggactcagcttcagggttccttttctggattttataatattacattgagacatcagcatatgataagaataattgtttgataagaataactataaggctgggcacagtggctcacacctgtaatcccagaactttgggaggccgaggcaggcagatgacttgaggtcaggagttcgagaccagcctgaccaacatggtgaaactccgtgtctactaaaaatacaaaaattagctgggtgtggcggcaggcacctgtaatcccagctactcaggaggctgaggcaggagaattgcttgaacctgggaggcagaggttgcagtgagccaagatcatgccactgcactgcagcctgggcaacagagcaagactgtgtctcaaaaataaataaataaataaataacaactataaaaagaaaagagtaacaactataaagttgtagactgggtaaaaatgcctcctaattggtagaagctacaaagagagatttcagccttggaactttccactagaaatatccaataacggaatggctggtgtgaattagtgcgctagagtgtctctgtctctctcaggggcgatttcttgatgtaaacatccagcattcactgtgttacagagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtaagtcaccttcggtcagtgacctactcggcttctacaactgcatttacttatttctgtgagggattttgaagtggatgaaaggaattttttgctctcaattctgccaaaagaaggttacaatttgtcaaatttgacaaattcattgacaagaaagtaaaaagaaaaagccttaaaacatagacacagatgattttttaaattatatgtaagactctgcatgaaaattttaacattaattacaagtgatgggattatggaggattttctcctcaaatttttattggctaattgttttgtaatgagtacatattactttaataacctaaaaaagaaatccatttccattttgacaaagcaaaggttcccaaactttctccatcccttgacactcctgagccaaaagaaatacctcacagttccacttattaagtagttaggtccactttaataatatttatgtttaacaacttcgtagttgtttataaaaatatgcacataaattggaataaaaatattttattatgttcttaagtaaccacaattatgtaatcatggaatgcatatgcctgatgggaactgcttagatcctcaaacctctgaatcaaaaaggacaatgctaccttcatttcctgttcacatgaatatgtatgtggttcctgctttttcatcacagcaaccactgaacacccagctttgcaaagatatggcattattgaaaggaacacagtgttcaaactgtgaactattgccaactagaagtttgcacagtgcccaatagatgtcactgtttcactggggaccttttaatatccaaagtgtcccacagagtgcccttagcaccctggggcttgtttgcacgtgtgtgggagcggcaaggataagaaatgaagagtcgtctaggtgtggtgactccacatctgtaatcctagcactttgggaggccaaggcgggcagatcacttgaggccaggagttcgaggccagcctggctaacatggtgaaaccccgtctctactaaaaatacaaaaaattagatgtggtgaaatgcacctgtaatcccagctacttgggagactgaggcatgagaattgcttgaacctgggaggcagaggttgcagtgagccaagatcacacctctgcactccagcctgggtgacaaagcaagattttgtctcaaaagaaatgaagagtacatttgggggtaaagggctggcaagatggccgaataggaacagctctggtctgcagctcccttcgagatcaacacagaaggtgggtgatttctgcatttccacctgaggaatccggctcacctcattgggcatggttagacaatggatgcagcccacagagggcaagctgaaccagggtggggcatcacttcacccaggaagcgcaaggggtcagggaattccctcccttagccaagggaagctgtgaggtactgtgctgtgaggaacagtgcattctggcccagacactatgcttttctcatggtcttcacaacccacagactaggagactcccctgggtgcctacaccaccaagattcccttggatttcaagcacaaaactgggcagctatttgggcagacactgagctagctgcaggagtttttttcataccccagtggtgcctggaacaccagtgagacagagccattcactcccttggaaagggggctaaaggcagggagccaagtggtctagctcagcggatcccacccctgcagagcccagaaagctaagctccattggcttgaaattctcactgccaccacagcagtctgaagtcaacctgggacgcttgagcttggtggggggaggggcatctgccattactgaggcttcagtaggcggttttcccctcacagtgtaaacaaagccacctggaaggtccaactgggtggattccaccgcagctcagcaaagctgctgtagccagactgcttctctagattcctcctctctgggcagggcatctctgaaagaaaggcagcagccacaatcaggggcttatagataaaactcccatctccctgggacagagaacctgggggaaggggcagctgtgggcgcagcttcagcaaacttaaacattccggtctgccagctctgaagagagcaccagatctcctagcacagcattcgagctctgctaagggtcagactgccttctcaagtgggacccccgtaactcctgactgggagacacctcccagtaggggccaaagacacctcatccaggggatctctggctggcatatggcgggtgcccctctgggacaaagcttctggaggaaggaacaggtagcaatcattgctgttctgcagccttcgctggtgatacccaggcaaacagggtctggaatggacgtcagcaaactccagcacacctgtagcagaggggcctgactgttagaaggaaaactaacaaacagaaaggaatagcatcaacatcaacagaaaggacgtcaacataaaaaccccatccgaaggtcaccaagagcaaagaccaaaggtagataaatccatgaagatgaggaaaaaccaatgcaaaaagctgaaaattccaaaagccagaacacctcttctccttcaaaggatcacaactcttcaccagcaagggaacaaaagtggatggagaatgagtttgacgaatggacagaagtaagcttcagaaggtgggtaataacaaactcctccgagctaaaggaacatattctaacccaatgcaaggaagctaagaaccttgaaaaaaggttagaggaattgctaactagaataaccagtttagagaagaacataaatgacttgatggagctgaaaaacacagcacaagaatttcatgaagcatacacaagtataaatagtcaaatcaaacagaagaaagggtatcagagattgaagatcaacttaatgaaatatagtgtaaagacaagattagaggaaaaaaaaagaatgaaaaggaatgaacaaagcctccaggaaatatgggactgtgtgaaaagaccaaacctacgtttgactggtgtacctgaaagtgacggggagaatggaaccaagttggaaaacactcttcaggacattatccaggagaacttccacaacctagcaagacaggccaacagtcaaattcaggaaatacagagaacaccacaaagatactcctcaagaagagcaaccccaagacacataatcatcagattcaccaaggttgaaatgaaggaaaaaatgttaagggcagccagagagaaaggtcgggttacccacaaagggaaccccatcagactaatagcggatctatctgcagacaccctacaagccagaagacagtgggagccaatattcaacattcttaaagaaaattttcaaaccagaatttcacatccagccaaactaagcttcataagcaaaggataaatgaaatcctttacagacaagcaaatgctgagagattttgtcaccaccaggcctgccttacaagagctcctgagaaagcactaaatgtggaaaggaaaacccggtaccagccactgcaaaaacaaatgaaaatgtaaaaagcatcgatactgtgaagaaactgcatcaactaatgggcaaaataaccagctagcatcataatgacaggatcaaattcacacataacaatattttttttttttgagacaaagtcttgctcttgtcccccaggctggagtgcaatggtgcgatctcggttcactgcaacttccgcttcccgggttcaagcaattctcctgcttcagcctcctgagtagctgggattacaggtgcctgccaccacgcccagctagtttttgtatttttagcagagactgggtttcaccatgttggccaggctggtctcgaactcctgacctcaggtgatctgcctgccttagcctcccaaagtgctgggattacaggcgtgagccaccgcacctggcccacgcataacaatattaaccttaaatgtaagagggctaaatgccccaactgaaagacacagattggcaaattagataaagagtcaaaacccatcagtgtgctgtattcgggagactcatctcatatacaaagacacacatagtctcaaaataaagggatggaggaagatataccaagcaaatgaaaagcaaaaaaaaaaaaaaaagcaggggattgcagttctagtctctgataaaacagactttaaaccaacaaagatcaaaaaagacaagggcattacataatgataaagagatcaatgcagcaagaagaggtaactaccctaaatatatatgcacccaatacaggagcacccagattcataaaccaagtccttagagacctacaaagagacttagactcccacacaataatagtgggagactttaacaccccactgtcaatatgagacaggtcaatgacacagaaaattagcaagcatattcaggacttgaactcagctctggaccaagcaaacctaatagacatctacagaactcttcaccccaaatcaacagaatattcattcttctcagcaccacatcactctgattctaaaattgacctcataattggaagtaaaacacttagcaaatacaaaagaatggaaatcataacaaacagtctgtcagaccacagtgcagtcaaattagaactcacgatcaagaaactcactcaaaactgcataactacatggaaactgaaaaacctgctcctgaatgactactgggtaaataacaatattaaggcagaaataaataagttatttgaaaccaatgagaacaaagacacaacgaccagaatctctgggacacagctaaaacagtgtttagagggaaatttatagcactaaatgcccacaggagaaagtgggaaaatctaaaatcaacaccctaacatcacaattaaaataacttgagaaggaagagcaaacaaatccaaaagctggcagaagacaagaaataactaagatcagagcagaactgaaggagatagagacaagaaaaacccttcaaaaaaatcaatgaatccaggagctggttttctgaaaagattaacaaaatagatagactattagccagactaataagaagaatcaaataggcacagtaaaaaatgagaaaggggatatcaccactgctcccacagaaatacaaactactggctgggcacggtggctcacgcctgtaatccgagcactttgggaggccgaggcaggcaaatcagaaagtcaagagatcaagaccatattggccaacatggtgaaatcccctctctattaaaaataaaaaaaattagccagacatggtggcacatgcctgtagtcccagctacttgggaggttgaggcaggagaatcacttgaatctgggaggcagaggttgcagtgagccaagatcacactagtgcactccagcctgggcaacagagcgagaatgagactctgtctcaaaaaaaaaaaaaaaaaagagcaatacgtacaaactaccatcagagaatactataaacacctctatgcaaattaactagaaaatctagaagaaatggataaattcctggacacatacaccctcccaagactaaaccagggggaacttgaatccctgaatagaccaataacaggctctgaaattgaggcagcaattaatagcctaccaccaaaaaaaagtccaggaccaaatggattcacagccgaattctaccagaggtacaaagagaagctggtaccattccttctgaaactattccaaacaatagaaaaagagggaatcctccctaactgattttatgatgccagcatcatcctgctaccaaaacctggcagagacacaacaacaaaaaaaagaaaatttcaggccagcatccctgatgaatatcgatgcgaaaatcctcaataaaatactggcaaactgaatccagcagcacattaaaaagcttgtccaccatgatcaagtcagcttcatccctgggatgcaaggctggttcaacatacacaaatcaataaacatagtccatcacataaacagaaccaatgacaaaaaccacttgattatctcaatagatgcagaaaaggccttcaaggaaattcaacagcccttcaagctaaaaactctcaaatgaggtattgatggaacatatcaaaataataagagctatttatgacaaacccacagccaatatcatactgaataggcaaaaactggaagcattccctttgaaaaccagcacaagaaaaggatgccctctctcaccactcctattcaacatagtattggaagttctggccaggacaatcaggcaagagaaagaaataaagggtattcaaataggaagagaggaagtcaaattgtctgtttgcaggtgacattattttatatttagaaaaacccttcgtatcagccccaaatctccttaagctgataagcaacttcagcaaagtatcgggatacaaaatcaatgtgcaaaaatcacaagcattcctatacatcaaaaatagagagccaaatcatgagcaaactcccattcacaattgctccaaagagaagaaaatgtctatgaatacaacttataagggatgtgaaggacctcttcaaggagaactacaaaccactgctcaaggaaatcagagaggacacaaacaaatggaaaaacattccatgctcatggataggaagaatcaaaatcgtgaaaatggtcatactgcccaaagtaatttatagaatttatagattcaatgctatccccatcaagctaccactgactttcttcacagaattagaaaaaactactttaaatttcacatgggaccaaaaaagggcccgcatagccaagacaatcctaagcaaaaagaacaaagctggatgcaacacgttacctgacttcaaactatactacaaggctacagtaaccaaaacagcatggtactggtaccaaaacagatatatagacctgtggaacagaacagaggcctcagaaataacaccacacatctacaaccatctgatctttgacaaacttggcaaaaacaagcatggggaaaggattccctatttaataaatggtgttgggaaaactggctagccatatgtggaaaactgcacccctttcttacaccttatacaaaaattaactcaagatggattaaagacttaaatgttaaggcctaaaaccgtaaaaaccctggaagaaaacctaggcagtaccattcaggacataggcatgggcaaagacttcatgactaaaacaccaaaagcaatggcaacaaaagccaaattgacaaatgggatctaattaaactaaagagcttctgcacagcaaaagaaactaccgtcagagtgaacaggcaacctacagaatgggagaaaatttttgcaatctgtccatctgacaaagggctaatatccagaatctacaaggaacttaaacaaatttacaagaaaaaaacaaccctatgaaaaagtgggcaaaggctatgaacagacacttttcaaaagaagacgtttatgcagccaacaaacatatggaaaaaagctcatcatcactggccattagagaaatgcaaatcaaaaccacaacgggataccatctcatgccagttagactggtgatcatttaaaaatcaggaaacaacagatgctggagagggtgtggagaaataggaatgcttttacactgttggtgggagtgtaaattagttcaaccattgtggaagacagtgtggtgattcctcaaggatttagaactagaaataccatttgacccagcaatcccgttactgggtatatacccaaaggattataaatcatgccactataaagacacatccacacatgtgtttattgcagcactattcacaatagcaaagacttggaaccaacccaaatgcccatcaatgatagactggataaagaaaatgtggcacatatacaccatggaatactatgcagccataaaaaaggatgagttcatgtcctttgcagggacatggatgaagctggaaaccatcattctcagcaaactaacacaggaacagaaaaccaaacaccacatgttctcacttgcaagtgggagttgaacaatgagaacacatggacacaggcaggggaacatcacacactggggcctgtccatgggtcaggggctgaggagggatagcattaggagaaatacctaatgtagatgatggttgatgggtgcagcaaaccaccatggcacttgtgtatgtatgtaacctgcacattctgcacatgtatcccagaacttaaagtataataaatttaaaaatttaaaaaagaagtgaagagtataggtgtctgatccaaattataacaacaagttctagctgtggaaacaactggctggcgtagctcacaaggaagcaataccttcaaaccttttcttttcctcagtcttgtctacatggcattagaaccttttaacaggcaaaacaaaacgaaatgtatctctaacagtttcaaaacctcttctctcactggggctaggcacagagttcccattttgacttggagtacagcaagaggttctgagcacctttgcgtgggaaagaagtcaggcaagccccaattatgaagtaaatgaccagcttgcctctcctgctttgggttactaattctgtttcccagattccctttaaagactcttccatgtttcccaggggaatggtttcccagaattctagaaagtttagggacagcatctttatgactccctttaagtgtcaaatttattttttattataaaaatattttttgagttttgcattcaattttataatacatctgtttgtcttgataagaaaacaatggtttaaattacttaaccactgtataaaattatagctccaggagcttgagccatatatattctgtggatttgaatactctaggcactcccctagttagagatgcacaaaaaatgcaattaagggtgatgaaaataattgtcatcaacagaaaaggatcctcccatcaaaagagccagaatcacccatgcccctggtctcccaggctgctgtttctgataataagtttattatttgtttcctgcagtggccctcctcaggaggggctggcttccttccctggccagctctaatttcttctctgttgggtaactcactcagaaagctctataggcaatagttcctgggcgtggagcaaattgctatctcaccttttgttttgtttctcttgcaactcattagggattgtactttacaccccaagtaaatctatgccctgccaatccaggatgggcgtatgtaaagtgaccctctgtttgatgtacccttctaccctgactttgtagctgaaatgagaggagacagctatgggttgctgctggggccctatcaccacacttagggctgctgtcccagaaacaggatcaccaggaggattctaaatccagatgtacattttgtaatcatacagtgtcattgtaccaccctcgaagtacagagttaaatatcctttattcataacattgtggccagagactcatttcaccaccctaaaggtaaggtcaacgtttgaaaggctttatggcctttttctgtagcagtcaactagccagacgccttagtatagaaaaggacaaagcatgaagaaaatgccaggtcataaactctgtagagttcattttaattatgtggggtagggtcattaagagaagttagaattgttgaagctaatggtaaatcttctttttaaatctgaatgttaaaagtgagaatagatttgacttgactaacatattatttgaagctgatttttgcagtatttgaacattcatctctaaaggtgcatttggatttgtgtctttttaggtcgtaatgggaactggcatttcagctgggtttaacttgaaagaatcatacaatgtggatgtcgttggaacacttcctctagggtaggaaagtggttttaagtaactgtgtgactgaaataatcaatggatctattgcctctaactcttacattagttctcttgcaatgaaaaataatcttttttctatacatcaaatagctgagggctttagggggattgggtgcattttatttttatttttattgtagtaaaaaacacataacaaatttttgtcttaaccttttttttttttttttgagacgaagtctcactctgtcgcctgggctggagtgcagtggcgccttctctgctcactgcaacctccgtctcccaggttgaagcgattctgctgcctcagcctcccaagtagctgggattacaggcgtatgctaccacacctggctaatttttttttttttttttttttttttttgagatggagtcttgctctgtcccccaggctggagtgcagtggcatgatctcggctcactgcaagctccgcctcctgggttcatgccattctcctccctcagcctcccgagcagctgggactacaggcacccgccaccacgcccggctgattttttgtatttttaatagagacggggtttcaccatgttagccaggatggtctcaatttcctgacctcgtgatccacccgccttggccttccaaagtgctgggattacaggcatgagccaccacacccggctaatttttatatttttagtagagacagggtttctccatgttggccaggctggtctcaaactcctgacctcaggtgatccgcccgcctcagcttcccaaagtgctggagtgctgggattacaggtgtgagccactgtgcttggctttttttttttttctttttttttgagaccgagtctcactctgttgcccaggctggagtgcaatggcacaatctcggctcactgcaacctctgcctctgaggttcaagtgattctcctgcctgagcctcctgagtagctgggattacaggtgtgtgccaccacatccaactaatttttatatttttagtagagacagggtttcactatgttggccaggctggtctcgaactcctgacctcatgtgatccgcccgcctcagcttcccaaagtgctgggattataggcatgagccaccacgcccagcaaatcttaatcatttttaagtgtacagttcagtagtgttaaatatatccccattattgtaaagcgtatctccaggactttttcaccttgcaaatctgtaactctccaccccttaagcaagagcttcctcttttcttcttcccccagcccccagtaaccaccgttcatctttctgtttttatgaatttgacttctctagatgcctcatgtaagtggaatcatacagcatttgtcttttcgtgactggcgtatttcacttagcataatgtcttcagagtttgtctatattgtagcatatgacagtattttcttcccttcttaaggccgaataatattccattgtggatatacactgtattttatttctccatgcatccactgatgagcacgtgggttgcctttacctcttggctattgtgaatagtgcagctgtggatatgggtgtgcaaatatgtctttgagacctttctttcagttcttttgggtatatacctgtaagtgggattgctggatctgtttttacttttttgagagcctttcatactgtttcccatagtagttatgccattttacagtcccacccatagtggcaagtggttttctgttttcttttgttttcctaaatttaatagttttaaaaatctctccttgaatgacatgggatttaagagcaaaacaacctaaatcctagcctgccacgtactagctgtgtgatcttaagcaggccacttatcctctcaaagcctccatttccttacctgccaggtggccatggtaattaatacttatctcaagggcagttgtgagagctacaggagataatgcatggaaggcaatgagtgaacacctgaagacacagtaagcattcaagtcgtgttaactacttattagtaatgggttctaaggctgttatttcagtgagtttttagtttctaaccatgttacttctttgcttcatctatattatctaactgtacatttttaaaacatattattggccgggcacggtggcagactcctgtaatcccagcactttaggaggctgaggctggtggatcacaaggtcaggagttccagaccagcctggcaaacatggtgaaaccccatctctagtaaaaatacaaaaattagccgggcatggtggcgggtgcctgtaatcccactattggaaggccaagcaggagaatcgcttgaaaccagaaggcggaggttgcagtgagtcaagatcacgccatggcattctagcctgggcaacaagagcgaaacaccatctcaaaaaaaaaaaaaaaaatttatatatatatatatatatacatacatatatatgcgtatatatatatgtatatatatatataaatctacttgtcactataatttctacttaatatcttcctttccctattgcattgtactcaaacatactgcttcccttctgtatgtgacagcttaactaaaaaaaaaaaaaaaaaaagaccttacgtttctggaaacaggctatccagaggagaccttatctttctggaacattgcccaaaaccaagtaggtaacaaatacctacctacctatctacatacaatcataccatttcagttccctgtgagtcttattgctccactgataacaggctaacccaaaacagtgacttaaaacagccaccattaatttgctcataattctttagtgtaggttaggctcagttgggtggttctttggcaggtctcacttggggtttgttatgcatctgcagatgtctgcaggtgtgactagtgttggaggatccaagagggcttcactcacgtgtctgacacttcatctggagtcactggaacaggtgagggctagcaggcatcttctctacatgtggtatctccagcagcattgccagacctctttacatggtggctcagagcaacaggaaagaaaagcagagaatttaccaggcctcttaaggcactcatacagcatcacttccaccacattctgttgctcgaagcacatcacgaggccagcccaaatatagctggaggggaaacagattccccatctagatttggggagtgacatgcacatacaagtatgggaggaatgactggtggcctatttggaggcaaactaccatgcttctatatacacgtacctacacatataaatgcatctgctgtagacaagcaggttcttcatggttttgtacttggtaaatgagcagtgcattttgcggtgcccagaaagtgacagttgactattaaagatcccaatcatagcactatatcctgttttcttgtttgtttgtttactttccttaacttgttccaaaatatgtccttttcttatattcttattcccttaggcccaaacctgctgctaggccttccctcttagttcatgcctgtgcagcaaagagaagggaaggacaacaggttttatagccagaccccccaggtttgactccctgctacagtttaggaaaatcacttaacatctgtcaaaatccattttttcacctacaaattgggataatagtagaacctatgtaatgggagtgctgtgagggaaaatgagacgacttatgtaaagcatgtagcaggtaaactcaccttaacaggcattaggttcctttccctttcttcctctgtttccttacttcacgatgtgaaacatcccttccgtcactgtgctatgacatagtaagagaaatagctgtggccaagcaggttctttatggttttgttgtacttggtagatgagctgcttgtccttggacaagttgctgaaccttgctatacctcattttcttcatctgtaaaatgggaattacagtggtgcctagttcacagggccattgaaaacaattaaatgagcatagaacctggcacagagaaagcatataataaatgcttgttattgtaataattataattattagtatcagttaagttattgttattattgtttagcccaatgccttgaattggaggagaatgtctgcaaatccctattgccccttcccccataacattctagggaatccctattctgtttctttaggataaggatcccagactacaagtatgtaccacaaaagctgggttctccattgtgggattccaggagtcacacttttatctcctccagattcacaagattcccaggtgacacccaaggagtatagtcttctgctgctcctttcccatgttacaacctcagctaatgggataaaacacacacacacacacacacacacacacaggaagaggtagcacactcagagtccttacatcagatgacaagggggagaagtatggggtgcctagcattgctaggacattaattctcctctaaggtagcccttagactactctcctccccaccccaagacttgggttcagatgcctggtggcatctgagcactttgctgaccctcgcttcttcctggtttctgtgtcttacttacacttgccatcagggtgccatgaggtatgaggttactccttttataaataaaacggcatctgatctcttctgataacccatccaggtagaaagctgcactttgcagtttgagcctgaaaagaatttctctgcgtcaaggagggccctcctttttgccaaccagagcaaataagtatccaagcattgattacttcatttgcagataatgcccccctaaaatgcttccctgcagtcgcaaggttttttttcttttcccttgtggtcacaacacaagttaaaggcagcttgacttttcaggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggtaaagataatcagtttcctgcacatgtttcatagcagtacaatcaccaatgtctggctcacgcctgtaatcccagcacttcgggaggccaaggcaggcggatcatgaggtcaggagattgagaccatcctggctaacatggtgaaaccccatctctactgaaaatacaaaaaattagccgggcatggtggcaggcccctgtagtcccagctactcgggaggctgaggcaggagaatggcatgaacccaggaggcggagcttgcagtgagccgaggtcgcgccgctgcactccagcctgggcgacagagcgagactctgcctcagaaaaaaaataaataaataaccaaggtcggttatggaaggcacaagaactaatctatcttggtagaaaaactttttgtactggtgattgatgtatctgaagcatgtacttattcagtctgcaatgttactcctgtttttaaagctcacagcttaggattaaatgtttttccagggcgtttttgctccctaggaaggtttatctttgttcattaaatattgggtatctgattcatcactaactccacaatattctctttgtcacctacttttataacgcagccacagataaggtatttaatccaaacactgtgacctataatgttgacataacaatgtctatttcaatccttatgtattctaaggagaaactagatgctccacattcatctgagatgcagttgtcaaactagaaatatgcaaagtaaacctaagagcaggactcacttgccccaggaagacactggcatgtactcatttgtgagcaatagcccaggcattaaaaagatgccaagagtcctatacaacaataactttctcttcagcagtaaaaatagttgatcccttttagtgcagttttcttcttaagcgttatttatgtagagggtgtgtttggaaaggaggctgagcagggagcccagcgtttatctctgacaaaataaaaattcaaaatgttcccccctctcactataaaatatgccataattcctattttgaactcatgctgaacaaattcttacatctgaaaggaaaatgaagagataggctgtagtgctgacaatgtgtattaaatgggctattatgcttttttttcctgaagaatgacctatctcttttgtgttttgtttctacatgggaaaggagctcattgccctgggactgtgcaattccattggctcactcttccagaccttttcaatttcatgctccttgtctcgaagccttgttcaggagggaaccggtgggaagacacaggtatgtacataacaggcctctaagacctgatgatatgggtggtaattaaattttaatcttttgattatattctgaacccaaacaaagctactctggcctgcagatccatgctccagtgagctgtgtagggaatcagagaaaggcatgattggttgaagtgaatcacaaggaactgaatctataggaaagaggtatattctccagttacattggaaagttatgcaacactcggctaccagtaaacttcaaagttcagacatagaatattgttatgaaagcctgagactgagtatgggcagtatgccatcaccatccagtagaactttccgcaatgatggaaatgttcaatatctgcactgtccaatatggcagccactagcaacatgtggctcttgagcacttgaaatgtggttagttcgaccaagacctgacttttaatttttacaaatgtaaatggccggccaagtgcggtagctcatgcctgtaatcgcagcactctgggaggccaaggcgggcagatcatgaggtcaagagatcgagaccatcctgtccaacctggtgaaccctgtctctactaaaaatacaaaaattagctgggcatggtggcggatgcctgtagtcccagctacttgggaggctgaggcgggagaatcgcttgaacccaggaggcagaggttgcagtgagccagggtcgcgccactgcactccatcctggcgacagagtgagagtctgtctcaaaaaaaaaaaaaaaaaaagtaaatggccacatgtggctactggctcccatattggacagcagacacttatctgaactcttctggtggcccttgtaggtctcaggttggcaccagacatctttcctccacctctggaaagggcattttaattaggatattacaacatttcagcttccatttaaaagccataagaagaaccaaaacttcagagagggagtgtgatggagctataggttatgtagtggtttagttgacttatttgaatccatattttcttgatcttcccatagtaattatcaaaagaataaaaccaacttatgccctgcctcttattcaagtctcctaaaagccccttccatgtgtcttctctcattcagcttgcaggttgtttggcctcattaatgattctgctggtcatattagcaactggattcctctttgaatcattgccccaggtgtgtgtaatgtggacttgggtgaagtttttttcatgtgtagagattttgctattttatttgtttatgttcttatggctatattagggttatttactgtaaccctcacaggatggtatgagaaagggccaaggctaggaccaagatcctgctctataacctactgacagctgcctgcaggacctggggaaagccatcttaactcctctgagtctcggtttccttatctgcaaagtcggggtttggactggctgatcttgaacatcccttctctctttgaccttctatgaccttgtactttcataaaaaacctgagatggtctacattcataagacatttacatgatggtatatttaatctagtagagatcaggaaccatatagagtagagaagatcataatgtagaaagaccataggtgaaataattccttgagtaccaagtttaattctgaacctcttggccaccatgcaggtttgctattaggatataagaatgatttccagccgggtgtggtggctcacacctgtaattggcactttgggaggctgaggcgggcagataacctgtggttaggagttcgagaccagcctgggcaacatggtgaaatcccgtctctactagaaatacaaaaattaactgagtgtggtagcacccacctgtagtctcagctattctagtgggaaggttgaggcaggagaattgcttgaacctgggaggcagaggttgcagttagccaagattacgctactgcactccagcctgggcgacagagcaagacttcatctcaaaaaaagaaaaaagaaagactgattttccaaggccccttttagctaaccaggtcctgaaaatccaggtgttcatggcttcaaagctaccatcggtgccatataggaagtgagtagggagaaggcatggggaggacagcaggaggacagtgaggaaccagcaaaggacactctgtggacacaagaggagaggcatgggggatgtgctctgtttcaggctgtgctgtcggccattgtgattgtcaacctgaagggaatgtttatgcagttctcagatctcccctttttctggagaaccagcaaaatagagctggtaagtaacaaaggtgatggctctgttaatatgtcatagagcagatatgattagtgctaagaaaaatgtgagaaagataaattaatgcagttgttatgtgcagtatccagttagacaaggtaaaagtagcatgtgtgtgtgtgcacacttgtgtgtttaactcttgtgggatgtttaacatggttctccatcaagttttctgtcttttgcttagaccatctggcttaccacttttgtgtcctccttgttcctgggattggactatggtttgatcactgctgtgatcattgctctgctgactgtgatttacagaacacagaggtgagtgcccagattggaatgggtgtgaatgtcccggcagagatgacaatgttgactttaggtgtagaccaaagtttaagttggtagaagtggagccctttgatgatttctagttagcgtgagagggagctataacactcatgtagcctgttgactagatgaacaaaatgccaatttaaaaattccatataattttgccaaatgctcttctatgtcacaatttatgctcccatcaatggttatgttaaaagagcctaatttccatcattgtttctgccattcctggtctagtgctatgctggtttatttatcctcttgtgatttgttttggcaccaagtactgacatgagtttcaatgacatgaagcaaactctgacaccaagttatcgtatgcattccttccactgtcatttcctccaccctgaaccactttcccttgttatctcttctccctagtaggaagctgagcccactagggaaagtataaaataaattaaaaaaatatatggttgggtgcagtggctcacgcctgtaatcccagcactttcagaggccaaggcaggcggaacacttgaggtcaggagtttgaaaccagcctggccaacatggtgaaaccccatctccgctaaaaatacaaaaaaaaattagccaggcttggtggcacacacctgtaatcccagctacttgggaggctaaggcaggagaattgcttgaactcaggaggtggaggttgcagtaagccaagatcacaccactacgctccagcctgggcaacagagcaagactccatctcaaaaaaaaaaaaaaaaaaaaactttatgggctgggcacagtagctaacacctataatcccaattccagccctttggaaggctgaggtgggaggatcacttgaggccaagaccagcctgggcaacatagcaaaactccatccctaacaaaaaacaaacaaacaaaaaaacacctttttgtgataaatcaccataaagatgaaaaggaaacattgtcatagaaatagcctggactggccaggcacagtgactcttgtctggaatcccagcactttgggaggccaaggcaggtggatcacaaagtcaggagttcaagagcagcctggccaacatggtgaaaccctgtctttactaaaaatacaaaaaaattagctgggcatggtggcgtgcacctgtaatcccagctacttaggaggctgaggcaggagaatcgcttgaacctgggaggcagaggttgcagtgagccgaggtcgtgccattgcactccagcttgggcaacaagagcaaaactccatctcaaaaaattttttaaaataaaataaagaaatagcctggactttagtcaggtcctggcggcaccttctgtaagctctgtgattttgcatgcctctcagagcctgtttctgcatctataaaatgagactaatacttcctacctcatatagttacaataaggcttaaatgacaaggcctgcaaacagccagcacagtgcctgacaagtgctacccactctaccaatgccagtgcccttcccgctctaacacagagggaaatgaaaatgaagctagtgtggttttgcctatggttgcaaatcacatgaaaccttaaagttgtgactgtgatgtgttgaaagggcactttatgaggatgttgaccccttatctgcaggaggtcaggatgctccccaccctcaagggtgggaaatgagcattttcatcatcacaaatgacataacagaaaaatgttaagaatatgtgggattctaaactcaggataagaatttaactacctttaataacaagttccaacactgttgcccgtatttatctaaaagaagtgtttcccatttttataaagaactttattagcatatgtaagagttctttatctcagaactttggtgaacattatggttttaaaatatctataacttttggccaggtgtggtgtctcacacctgtaatgccaggaattggaagactgaggcaggcagatcacttgaggtcaggagttcaagaccagcctgggcaacatggcaaaccctcatctctactaaaattacaaaagcgagccaggtgtggtggcgagtgcctgtaatcccagctactcaggagggtgaggcagaaaattgctggaacccaggaggtggaggttgcagtgagctgagattgagccattgcactccagcctgggcgacagagtgacacacgtctcaaaaaaaagaaaaagaaaaaaaaatctataacttttaacaagctacagtatagaatttgagaatttttttgctggtggtggtgttttttgttttttttactgaataggattttatggtatcttatcataaattttatttatttttatttacttagtctttatttttctaacactagagaactcacttgagttataaaatttttttgtttgttttcttttgagacagagtcttgctctgtcacccaggctggagtgcagtggcatgatctcggctcactgcaacctccacctcctgggttcaagtcttctgcctcagcctcctgagtagctgagactacaggcgcgtgccaccacgcccggccaattttttttatttttagtaaagacagagtttcatcgtgtcagccaggatggtgtcgacctcctgacctcgcaatctgcccacctcggactcccaaagtgctgggattacaggcatgagccatcgtgcccggccggttatcataaattttaaagtccagcaagttttttttttttaatgctccagtttgtgctgggtgcagtggctcactcctgtaatccaagcactttgggagactgaggcaagcggatcatttgaggtcaggtgttcaagactagcctgaccaacatggtaaaaccctgtctctactaaaaatacaaaaaaaaatagctgggtgtggtggtgcacacctgtaatcccagctactgaggaggctgaggcaggagaatcgcttaaacctgatagactgaggttgcagtgagccaagatcacaccactacactccagcctgggtgaaagagtgagagcctatctcaaaaaaaaaagaaagaaagaaaaaagctcccgtttgggcaacctggtaaaaccccatccataccagaaatacaaaatattaactgggcatggtggtgcacacctgtggtcccagctacttggaaggctgaggtgggaggatcacttgagcctaggaggcagaggttcaagtgagctgagatggcactcctgcgctccagcctgggtgacagagtgagaccctgtctcaaaaaaaaaaaaaaaagaaaagaaaagaaaaaaataaaagctctacttctctttcttcctcaaataatagttaactgaagttgatattttttatgttctatcctttgttggtgattttgctgggtctttgtatgttagagtttaggagatcagcactcagggtatgtgtttattttcagctggcttcttctgtaattagggcacacttctggagaggggtagaggtgcactgggtttggccgtttgtttgaaacactcattttccctcagagcaggcatagttaagagacaatcatggcccggccagctgtaaacccagcactttgggaggccaaagcaggtggatcacgaggtcaggagatcgagaccatcctggctcgatcaaaaaattagccaggcgtggtggcgggcgcctgtagttccagctactggggaggctgaggcaggagaatggtgtgaacccggggaggcggagcttgtagtgagccgagatcacgccactgcactccagcccaggcgacagagcgagactctgtctcaacaacaacaacaacaacaacaaagagacaatcacatcattctgcagataattcctgaattcaggcacttgcatgtgttataaatatgacagaggttcagcttctgccccccaaagctcacaatttggttagcattataaaattttaaaaattataatttctctaaataggtatttgttgagggcctattatatttccagggagatgctagactctagggacagtgcagtatataagacatggtccttcccctcaaaggttgcgttgtcctaaggatgacagacacgtaatcatgatgatgatgttaataaggacaatgaagtaatgatggcacaaattattataattacttatgtgcctggctctgtgcaaagatcaatgctctatgccatttcatataaccttataaacctatgacaatcagcaaccccatttcacagatgtggaagctgaagcctagaatgattgggtaacttgcacaagatcacagtaagtggtgaggcaatgactcagatgtgagtttttgcaaatccagaagctgacaaatcagcacttttttttttaagacagagtcatgctctgtcgcccaggctggagtgcagtggcatgatctcagctcactgcaacctccacctcctgggttcaagcagttctctgcctctgcctcatcctcttgagtagctgggattacaggtgcccgccaccacacccagctaattttttgtattttttgtagagatggggtttcaccatcttggccaggctggtcttgaactcctcacctcgtgatccacctgcctcagcctcccaaagtgctgggattagaggcatgagccaccgcgcccagcctcaatcagcattttataatccatcttcaaaaaattagaatataggccaaacacagtgtctcatctcatgcccataatcccagcaatttaggaggccaaggcaggaggattgctgaggcgaggagtttgagaccagcctggacaacataataagaccatgcctctaccaaaaaaaaaattttttttattttattgtttttagagacagagttttactttgccacatgggctgcagtgcacttgtacggtcacagctcactgccaccttgaattcctgggctcaaaggatcctcccacctcagcctcccatgatgggcacacaccatcacacctggctaatttttaaattttttgtagagatggggtctcgccatcatgctctggctggtcttgaactcctgggctcaagtgttcctcccacctcagcctcccaaagtgctgggattacagttgtgagacattgcattgtgagacctacaaaattttttagttggcaaggtgtagtggtgcacccctatagtcccagctactcaggaagctgaggcacaaggatcactagagcctaggagttcaaggctacagtgagccatgatcaggccactgcactccagcgtgggccacagaataagatcccatctctaaaaaaataaataagtaaataataaaacaaaaataaatttttaaagacttagaatacacagttataagtgcctcaacaggtgtaagcaaggtacagtgtgagaagcataagagatgccaaataaaactgcatgggatttcattaaaagagactttcttgctcttggtgtcattagggaagtagtaaggacttggccttcactgggagcaatagtatttagacttgcagaaatggtgtgccagggcactgcaggtcgtcaggaaggcctgttcatcatctgcatttgaaatcctcatcctttgaggcccagctcacccagtcagggacctcaacttcccctgtgtacccacaacaatgctctaatccttccctagcaccttgcgtgtgtccatctcagctactcctgtacttgtgatccatgtgcccatcccttactctgcctcccctgtggcactggattgcaacacatggacagcactttgagtctcttagaggttttctcataattgcaaactcagctgagagccttagccccttatctacaatcagtcctcagagccatatttgtccacagatatcactagattgtgagcatcttgttggcagaagtgatgttcctggagtctagtaccaggcacatcatttgtgttctctgaatgcttatggaggaaatttacccagcaaaacagctaacacttgtgtagtatattctaatttacaaagtcctttgacattcatgctaagaagcaccaggacatagtagcaaaactgtggaatgtcaaatcaaagaccttgagccatattctgcctctggcaagctgtctgacattgaacaacttacctaaccccactgagcctcaatttctcatctacgtgatggcaggattgttaataatgttaactattattataatattattaatgtgcccagcagcattgttcaaaggatataataacaggtgttcaagaagtagtatctgtcattattacagtatctaatttgagccagatggtcctttacaaagagttaggaattaggggcaggcacagtggctcatgcctgtctgtaatcctggcactttgggaggccgaggtgggtagatgacttgaggtcaggagtttgagaccagcctggccaacatggtgaaaccctgtctctattaaaaatacaaaaattagctgggcatggtggcgcacaactgtaatcccagctgcttgggaggctgaggcaggagaattgcttgaacccgggaggcggaggttgcaatgagccaagattgcaccattgcactccagcctgggcaacaggataagactccgcctcaaaaaaaaaaaaaaaaaaaaaggaaatagggctctcaaaaaataatgtttaaaatctattcagactaaattaagttaacatctatgctaatttgagattttcattttctccccacgccctcaattgactactcccctactatgagtcagtgattcttgctaacaacaaacaaaaattccctctcctactttagagttagagcagaaagacagttctgtctactgagtggcttgaggagaacttgtgttacatatatccctggcataacttgaactctgcgaaattcagcgttgcctccacctctaaatgcctgaaccttttctcctgaccagatcgtgtttgtggtatggtgttggggggaggggaggtatagggtgtgtgatgtgcatcgtgaatgtggtgtgaggtatacgtgatgtgtgtgtgtgtgtgtgatgtgaagtgtgaggtgtatgtgatgtatgtatatgtgtgtgatgtgtatgtatgtgtattatgtgtgtgttgtgtgtgtggtgtatatgcatgtgtgtgttgtgtgtattgtgtcttggatgaaggttgtatatgcatgtgtatatgtatatgggtgtgtgtgtgtgtgtggtatgaggggtatgtggtgtgtgtattgtgtatggtattgtgtgtggcatgtagtgggggtatgtatgcatgtgtctgcacgtatgtgtattatgtgtgtagtgtgtgcatatatgtggtatgtacgcatgtgtggtgtgtgtattgtatattgcatgtgtggtgtgtatgcatgtgtgtgtgtatatgtatgtaggggtgtgtgtggtgtgaggtatatggtatgtgtgtattgtgtatggtattgtgtgtggcatgtagtgtgtgtgctgtgtatgcatgtgtgggtgtgtatgcatggtatgtggtgtgattgtgtatattgtgtatgtgtggtgtgtgtgtattgtttgtgtttgtgtgtagtgtgtatgcattgtgtgtatgtatgtgatatgtggggtgatgtgtgtgtatattgtgtttatatgtgtgtgtggtgtacatgcattatgtgtagatgtgtgtattgtgtatgtgtgtgtggtatgtgggtgtgtatacattgtatgtgtgtgtattgcgtatgtgtgtgggagtgtgtatatgtgtggtatgaggtgtatatggtatgtgtgtattgcatatgtgtggtatgtatgtgcatggtgtgtatgcatgtgaggggtgtgcattgcatagtatatatgcagtgtgtatgatgtgtatatgtgtggtatgtagtgtagtgtgtgtatattgtgtgtgatatgaggtatgtatgtatggtgtgtgtgcattgtgtgtgtatgcatgtgtgtgggggtatgtattgtgcattatacatgtggtatgtatgcatgtgtgtatgtatatgtgtggtatatcgtataatgtgtgtgtattctgtatgtgtggtgtgtatgcattgtgtgtaggtatgtatattgtgtatgtgtgtggtgtgtgaggtaggtattgggatgtgtatgtgtagtatgaggtgtacatggtgtgtgcgtatcatgtgtggtatgtatgtgtgtgtgtggcatgtgatgtgtatgtggcatgaggtgtgtggggtgtattaggcttggaatggcactgggaacgtgatatgtgtggagcccttctgcaaaggcaccatgtaaggatcaagcattcccaaatgcaccagcaaaccccaacctgacataaacatacacaacctcctgttgaaatcacacgctgtgcctcaacacaatggtatttaacactctttatcttcagtccaagctacaaagtccttggaaagcttcctgaaactgatgtgtatattgatatagacgcatatgaggaggtaggacctttttctatcacttaaaaaggttgtaagcatgtgatgtgcttggctaagtactgtttttacccaggataaaaagtaggggtaagatgggaggtatagtggtcaaagtgaaaagtctgagctttgatctactatatggattcaattttataatttatactatgcattaactactcaatttccaatatttatttttcctcatttttaatggggataaatatatattgttgaaattaaagtatacatttatacagtattgaaactgaaaggatggtttgtgtgtgcatggtttttttctgatgaatgtataatggtaaaataattttaaccatgtaaaatgtagatggtggggctgtggaattctcccacttcatttgaatggttaaaattacagcttcagatttccttgattttgagtgttcttttcttctgtggaatatcgaagatttttacaaaatcttttaaaactagttttgtctgtcggtcttgtcttatgcttgggaagaaattctcagactcactttaaggtattaggatatgaggaaacaaaattcactctaaggtaaaggtgttagaattgctatatttttcctcgatccaaagaagtatatgcaacattgcttgtttccagcctgcctcctagagttccacaagggttcttgataaaatattcactcagtttcacttgtgtccatttaagctccttcctgcttctgtgaatcaaaccagtgtaaacttctgttcactttatttgcctgcctctcacagtctgggcagcagccttccatctcctgtccattttcttttctgtttttgagtatttttcagtttcttctttgtgtttaatggtaaaaataaaacgaagactgacttttaaaacaaatggaaagatgtgcatgttaaaaaggtctctgtcagttctcttccattcagagcatgagtagataagagtgactttttttttcttttcttatctttttttttttttttggtctgtgtgtgacatcattttagctaagccctgaaaaggggaccttagcaacttctgcattcattttctatttggggtggggaagctatgcagttttgattctgaaatatttccatgtgttaaattcagcaatattttactcttcaggtgaaagaaattcctggaataaaaatatttcaaataaatgcaccaatttactatgcaaatagcgacttgtatagcaatgcattaaaacgaaaggtgagtcatgagaaattgattatttctgaggatgggatttgtgggttgacagggatatgaggatcactagtatacctttgtatatactatttcattctcgctttgtttctctctcttttattgtagtaaaatatacataacataaaatatactattttaagcattttcaaagtatacagtccagtgggtcgcattaagtacataaacattgtgcagccatcaccattgttcattctctcttatcgtatatataatattacttttcaaacaaaattttaagaaaacatgagaaaataattttaggtttcttattcttgggatcatgtagattcctttggtcatgtttagaactcaaccctacaccacgctgtatataggaaaagctccctgtcaacccacagagtctcttaaatataacaacctgacagatctgaacttgaaaaagactgctctaaaaattgtctttaatattttagcttctggttgtcttgaataaagactgagaataagaaaggctactaatgtaagggagtaaccagttcagggcctctgagggaaaggcagtgcaagggttggggtgataatctgacccagaaagcagttaaatggggagatttgaaggggagtggcattggggtgggtccctgagagccatgaagtgattttgcatatggtgctcttctgtaaagactggagtgaacccagcagtcatcatgggagcaaggagaaaggccatgcggaagtacgctaaggaagtcggaaatgcaaatatggccaacgcaactgttgtcaaagcagtgagtggtcttttcattgaaataatccgttctgtcttgttctgcaatgccctccatctttactcatgactgaagtttaaacacagcacgaagttactaaaagaggaaagtttagaaaccaccttagtatcctctatattttacaagcacctgataaggtcagttatgaggctgtttctgaaaaaagtaaggaaaacatcattttaaaaaataaaaattacacaataccttaacagattttaccattagcactttgtcatagatttattcagctgatagtttaaaaagctcaaaggtggcttttgctgttcaggcatgctaatgtgtgacccatcatactaaagttaatggaactattttagactttagcagaaaatggataggataactgaggagagtggattttgttggacaagaaaaaaaaggaggagaaggaaaaagagagaagttgacttaaaatggaatgttatacatacatcctttttttgttcagttacaaggctagaatatgacaaatattttacaaggggtctggtagagtcagtagcttaagaacacaagagcagattttccattataaatctttctcacaaccagaaatgtgagttgttcataaaattctggtttggtttaatttttgacacaggatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcaggggtaagcatcctcttgaacgagtcatttaatacctctatgcctggtgtcgtactatcagcataatcagggaggtgggctaggctctcttttcaacaaaagatttatccccttttgattatggaaaatttcaaaaatatatagaagtaaagattttagtatattgaacccccgtgaactatcactcagcttcaacaatgatcaatttatggtcaatctggtttcctttatagctgacattcactttctcttgttttattttgaaacacatcccagtcataacatcatctgtaaatattttaatgtgtattgctacaagaagattcgttttagaaaacattactgtaatatgctaggctactcaggaggctgaagcaggaggattgctgagctcaggagttcaagaccagcatgagtaaactagtgagatgctgtctctaaaaaaaatcgttttttattttgaaattataatatcctttaagttaatataatagttttattgtacttaagtaagatttaacagtatttttgctacccctaattatcactgtccacataaaaagcaaaagagatacaaaattatttttaactgattagctcaaaaatacatcattattgaagcaaagtagctcaagtgaatgaattagagtgtcttctatcacgaaaaacattggtaaaaagagactttcatctggcacagagtaaagagaaactaaattttatttcaacattattacagattgtaaaagaatatggagacgtcggtatatatgtatacttagcaggatgcagtggtgagtatacctctaagatggggtgaagcttaatattttagttttgtttctcttatttaaataattgcacttggaaatgtccttaattttaattattttataaaaatgaaaattgttcttctgaaaaatcactctaaagccagctttgaaattagccacatggaggcattcatattacaagttacttgtgacaaccctcctgaactggagttggaggaaaggagccaataagccatcatttattataatattaatggctgctataaatatagtaattataatatatacatgaccacagtagcaattatctaatggctgccataaacagttacatataatcattcaggttgcttgtttctctatccaatcatttgggaatactttgcctgctatttttgttttatattacattttggttaaaacagtgcttgaggccgggtgttgtgactcacacctgtaatcccagcacttcgggaggccaaggcagatggattgcctgagcctaggagttcaagaccagcctgggcaacatggcaaaaccccatctctactaaaaatacaaaaattagccagacatggtggcgcatgcctgtaatcccagctactcgggaggctgaggtgggaggatgtcttgaatctgggaggcagaagttgcagtgagccaacatcacgccactacactccagcctgggcaacagaatgagaccttgtttccaaaaaaaaaagaaagaaaatgattttttagaaaaggagcccggcgtggtggctcacacctgtaatcccagcactttgggaggctgaggcaggcggatcacctgaggtcagaagatcgagaccatcttggctaacacggtgaaaccccatctctactaaaaatacaaaaaattagcccggcttggtggcgcatgactttagtcccagctactcggaaggctgaggcaggagaatcgcttgaacccaggaggcggaggttgcagtgagccgagattgcaccactgctctccaggctgggtgacagagtaagactccgtctcaaaaaagaaaaggaaaaaaaaaaagtaaaacagtgcctgaggctgggtgtggtggctcacacctataatcccaacactttgggaggccaaggtaggaggatcgcttgagcctaagagttcaagaccagcctaagcaacatagtgagatcccatctttatttaaaagaaaaaataataataattttaaaaagtgcttgaaaatgaaatcttcataaagatgctacttttcacctgtaagattgccaaaaatcaaaaataaatgacaccatgctccataggcatatgatgggaaaacaggctctctcagacattactgacgggagcacaaaatgcaacagcccctatggagggcagactaacattagctttcaaaattataagtgcatttacctttcaacacagaaaatccagcaatatgctgcatatgttcaaaatgacacttgtatgtcgttgatcatcacagcattgttataaaacaaaaaactcaagtaccaaacaaaagggactagttaaataaactagggacatctatacaatagaatacgatgtagtggtaaaaaaattaagcaaaaagggttcagaattatctatatatagtttatatatatatataacattaatctatatatttcccacttttcttatccccaaaacagcattttatatatatatgtatatatatgtgtgtgtatatatatatttatatatgtgtgtatatatgtgtgtgtgtgtgtgtgtttgtgtatatattttttccccacttttcttatccccaaatatataaaatatgctgttttggggataagaaaagtgggaaataagaatacatacttatatttatttatatttacatagagaaatcctggaagaacacacaagaaagtagtttcctgggattgaggttatgaagggtggagacatgaattggagtgaaacttttactatagcattttaatatttaaactatatgtgcttgaacgcaggagttggaggttgcagtgagctgagatcacaccactactctccagcctgggtgacagagtgagactctgtctcaaaaaaaaaaaagaaacaaaaaacaataacaaaaaaaaaaacactatatggaagtgttacttttttttaattgttgaagactgatatccagagtgtggactaattttcttttcttcttttaatagcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacgcctagcaagatgtattgtacttgtgtttttttaattgaatacttcaaagataaattggcctgttgcctgcatttattatggagagacatttctgctacattctagtattatctttgtaagctaagcatcgaaaaatttatttcagtcaagtattttacccacaaataagatattacatagtggttctttgtttttgtacttgttgatctacagataagtagtggttctttgtttttgtacttgttggtctacagactgtctataatcagttatctaacagaagacaggaactgcaattttctagaagttcttgttttaaaataaccttttatttattatagaagcagtataaatgcattgaggacattttaaaaaagtagacaagcaaaaataagaacataaaacatatttccaccactcagagattaccactgttaatatccttccagatctttttccatacatatacatttacatttttaaccaaagtaagaccacatctatagtgttttataaccgatttttctttaatcaacaatctccactttctcattccagcacatttttgatcttagaccatatttaaatttccccaattgtccccagatttgcagttggtttgtgcaaactgatattcattctatgaccttacattgcaactggtcatgtcctttaaagtcccttacatctattttaatctagcacagttccttttttatgacattgacttgttaaagagactggggcagcggtcctgtagaatcctgtcttgtgaatttgcctagtttctttttcatggtgttgtttaacttgtttttatattgcctgtactacctgcaatctggaagttatatctaaagcctttataggttcaagttaaaacattttgtgctagattatatcatagataagatacatgcttcatatcgcatcacttcagaaaacgtaatatctagttgatctaccattaactaatctattgatctaagtttgattactggatcagcatgacagtctgatcatccagttagatttttcccctttgtgactacagacttatctgtgtggtattcctttggcattgaatgaattaccatttacccaggagttttaacattaggtggtaattcttgcctaaatcattaactttgttaaagtttgtgagttggttgtctagttctttcatttcttctccgtttgttagttggttattctatgtgaactaagctttcccttctccacttgagctatttggtcatccttacctacagttctaccagaagggcaggttgaatgctttattattttcatttaattatcaacttcaaagtaaataactggtttattagatggtgacaaattagtgtttttttttttttttagatttcccttttctggtgtgatggtggtcttgtgaatttttatagttttaatgggtttcaatccattatgttcttttcgatgctcaaattgtcacaactgtggccagtataagtccttagaaactttgctattgttgctaatcatgaagtagcattaaagaccatgtttttaagaaaactaacccccacatataacttaaatcttccttagaagttcataacagctatcagttcattatacatcaaactcagtgttcagcctaggagtaagcataatgctcatcttactcattccacagaaacatggaatgtcaaaggttggaggaggatacctttaaaccaatttgtcactttcagttgtttgtaattaccagctcactacaaatcaaagcacaggcattagccaacctgtctaaagcctcctctgctaagagttcctgctgatagtgagtttgtcttttctttaatatgctgtcttattgacatcttgttatagctttcatgaataagatactgccaggagaaagtagtcagatcccagtagaattgttagcatggcctacttgcaaccaaagcctgtggtgtatgtgttgatgttaactcctatcagatttctcactctctttactaatttaattcaaaataaactcctccttttaatcaaaactttaactaattctagagtgaaatttagattggcttagttatttcctttgcaatcatcacgagatcagatacatctgcattctgatacggactgccttctgaaagagcatgtagttgcagaacctcagcttacactagaaacttttgtaaacgagctaatatcatttcagccatttaagtagaatttaaggattttaaactatgttataaaatacttcggccaggtgcggtggctcacgcctgtaatcccagtactttgggaggccaaggcgggcagatcacaaggtcaggaattcaagaccagcctggccatcatggtgaaaccctgtctctatttaaaaatacaaaagtgagctgggcatggtggcgcgcacctgtagtcccagctactcgggaggctgaggcaggagaatggaatgaacccaggaggcggaggttgcagtgagccgagaccacgccattgcactccagcctgggtgtcagagcaagactccatctcaaaaaaaaaaaaaaaaaattattgcacattctttagcaccagccttccacttagcagatactatggttgatagattgacagtagtgtacaggccaggcgcagtggctcacgcctgtaatccagtactttgggaggccgaggcaggcagatcacgaggtcaggagattgagatcatcctggctaacatggtgaaaccccgtctctgctaaaaatacaaaaaaattagccgggcgtagtggcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaagtagtgtataaaaaaaggtatttgtcttatacatttgtatttaacttttctgttgaacttttaaactgttttgaatagaattgttatgtatattaaaagcacaagtcttaaggtgggaaaatatatttttgttttatcactttgcttagcatctgagaacgagtgcatttcaaaatacatttcatccatatgtaaatacgtcagaatgttttactcaattatttcatacagcaaaaatttcctgacttttttgtcttacagtctcgtctctggctttcaaggaaataatatataagcccacctgttctttctgggccttaatagctgctactgtatcttctatgtttttccaccctgattcaaattcaggttcatattgtcctatcaaaatgggcataatctacataatctaagcactagccttattttatggcaatatatgggaaagcactgttttgaagaaggtcccataaagaaaagtcctttgaccttctgaaatctcgtgcagtatgacagtggttgatctatgtctcatgaatcgttggaatatgtttgaaaacatttgtgaaccagggcccaggaaactccaatctatgtcttagacactgactctgtattagtatatggcagctgataatgggatcccaattgccaatctcatctgcagtggaaccccaagaaactgttaagggtatggatcctcttcaagccctggaatagctcattttgtagttgcaggaaacatctaaaacatccactctgtgtggtttgatcctgtgtccttacctgtttagcagagaaagacagtaccagcctctgagtgtacactttgagaaaatgagtctgctctcttgcgtggatgaagaaaagagagggtagcaaacctagtgaatctgtcctggggtgggtgggtgtgtttttgtgtgtgtgttgggggggtgggggtgtgcccttgtgtggggatgagggagggaagagcaggtggtggggaaggctcttttatgtctgcccctttaaagatagtaaatcaatttaaattctaaaatgaaaccgaacttccttggtgtgagcctgtatgctttactgagtgcaaaaaaatgctattggtcctttttagtgcaccttacctttcaggagtgcctttaggattcacgatgtttagattcttactgttttgaatgctcataaaagtttgagaactataaaagtattactgaattcttcaaacatacagctaaccaaaatgcatgcaatggttttgaaacccctccaatcccagcaacacaacggtaaaagttagcaccacaccaggtaacgtgcctgcttagtgacttagattttatcaagtttgagcatcttaaaactccacctggcctttgagaaagctgtaccaggcttttcattatactggtgtattgcatttcactttacgtaacagataaacctcagaagcatcatgaaaacggagttttttaatacacctaatcacatttttaacactaagaagtttttaaacaggattctacggtatatttttctaaagtcataaatcctttgtattctaacttatcagcaccctgacacatgtatatatatatatgtgcatctacacacatgtatatagacacatctattttaggtggaggaagttactggtaaatgtctgtttcatctctttgagtacaaacatgctgacctaggcaatgttgatgctacctgcataaatatgccttgacagaaaaacccaaatttttaaagtagttgattttacatttgatgtgtggccgattagattttccttatggacctaaattatcacgatagtcactctgtatcgttagaacagagtgtgctcaacattatctaattcgagttgtctttaattggtatatatgagcacgtggccatgacaaagtggaaagacttcagagtgtgttctaaatgtgtacatttatttaatcaaaacattcaaaaacatacatcttcagtcattttcttaagtatattttattgtaagcataacctgaaaatagtttgctctatttaattcctcaagaaaaattagggcagggttaaaaaagatgaatttttctgatcaagttcataaaaatgaaaaatcaagaatgttggttggattttataaaaggctttaatttctttacttgaaaactttcataattttaagtatttcagtgcaaagcagcattttctatgttgtcaactttttctattgtgaattaaaaatatatccaactcttattttgcaacttatttatgaatatcgaacttcaataaaaatattcattattgtgctttggtcttggcattgggggaaggggtaattgtggtagaagataggatttggtggtggttacacaggaggtaattttaaagagatgttgaggattctaatataacctttatttttaaaatacctcactgtagaccagaagcctaacagaaactttaccaaaattttaaagtaatttgtaaaatgaaagtaatcacttttctgtatgaagccaattctggactttctaaattagatcatatgtacaaatatttttttttctttttttactagtctttcacttctggctgaacatttttctttctgctttattgcaccactttactgctcattaaaatcactaattcttcctgattaatttgaaatcaaatggcaatgcaaaacatatacatccaatgtttctcctactttaaaaaagctttggccaaagcttttattattcaatatggtggtgggaaactatcataatgcatgtggtcatacgcctaagtgagctactttgttgcgggacagaagcggataataggagcataggtaatggagccaacaaacttgtacttgaaagccagctttgccactacaagtaagtaggaaagtttatgcatccattggatgaagatccatttgataatcttcctcagtggacagatgggaggatacaatgagaatttatatgtagagcacccggccagcatatgcctaggatagaagccatctaagcaagcgctttgtatgatgtgaaatccttaaacgggtgtgggtcaaaaaaatagtcaaaatatattctaacaagcttttctgcctaataacattagaaatcaccttttattatattacaagaagagatatggaaaaaagcatacaattttgaagccatttatttgtttatgaattggttattaacaagtctatataaggttaggattccaatttaaagttttcacttgaaagccttcagggttcagttgtatgtcatgtaacgaggagtagcactgaatttggcataagacttaatgaccttatttacagcttccaagaaatccttctcggtagcaatttttcgccgtgctctgatggcaaacataccagcctctgtgcagacgcttctaatctcagcacctttcaaacaaaaagaaaataaacgggcttaattaaagcagcctgagcagacagaccagcaaatataagcaagaactttctacttaccagtgctatttggacacagtcgtgctaacagttcaaatctgatatctctttcaacactcattgaacgagcgtgaatcttaaatatgtgggtccgaccctaaaaatgagaaaattgacaaatattaaaatggaaaaaaatcacaggaagaaaaaacttcaaaaatccctttcctaaaatgaaatggttttcttacctctagatcgggcaagctaaattcaatttttctatccaatctccctggcctcatcagtgctggatccaaagtatcaggtctgttagtggccatcagcactttaatattgcctctaggatcaaaaccatcaagctgattgatcagttccaacattgttctctgcacttcattgtcacctccagcaccatcatcaaaacgagcccctgagaagacaaaaggaaagataagctcttcaagtccactcaagagtagcactatgcatgacctgaaagtaccaaaaatctcagctcaaatcatgtcctcaggcacaagtgacatcccactttgaagcaaatcctgtaagaacagcttgctttatataaaactaattagaaagtgaatctttgctttcaaaagtattcatcacagatttacttaacgttttttaaactgcctatggggcagtttagtgttttcaaagtctaccacatttattacttaatttggacctctcaaaaactcatgaagtacacaagacaacaattattccctttataagatgagcaaactgaggctcagagattagttaaaataacttgtcctcaggagttcgagaccagcctggccaacatggtgaaaccacgtctctactaaaaatacaaaaattagccaggcatggtggtaggcatctgtaattgcagctactcaggaggctgagacatgagaattgcttgaacccaggaggcagaggttgcagtgagccaagatcatgccactgaactccaacctgagcaacagagtgagactgtttcttaaaacaaaaaaaaaagtctagatttcatggctaatactgtatttcatcaaatctgtgaagccaacaagaacattattttatgtgcactaagaaaaaccaccatcaattaaattatgccccagtgctttcttatgacattagatgatacttctcaatttacgagttgtttaaagtatggaaataggtgttgtatgaatgaaatcagatatagactgagcatcccaaatttgcaaatgcaaaatccaaaatgcttcaaaatttgaaagtttgagcaccaacatgacaaaagcaatactcattggagcactttagatttcaggttttcagatttgggatactcaatcagtataatgcaagtacttcaaaagccaaaatccaaaatatgtgaattccaaaacacttctcaaacatttcggctaagggatattcaacccatagcacaaaaataaaggctttgaaaaaatgacaatggtatataatgtgtcttcttgcctttatagtttagcactactacttattagatctgtggcgccactgtttcagagctcatagaagagttggagttaaaattctgtacaagattaacatatcacgacatatcactttacagctttcatgaaactgaatcctaaagcagttctctaacgtatcattctcacctccaatagcatcaatttcatcaaagaagataaggcaggctttttttgttctggccatttcaaagagttcacgaaccattcgagccccctaatgagaaaaatgatttattaattcaaaattggtttcaaaatattttaaaataagtaaaaagttatgcttcaaacttttcaatatttattcctaacagtattctcacccattattacctaaacgttttttatttttttatttttttgagatggagtcttcctctgtcgcccaggctggagtgcagtggtgcgagctcggctcactgcaacctctgccgcctgggttcaagcgattcccctacctcagcctcccgggtagctgggattacaggcatgtgtcaccacaccccgctaatttttgtatttttagtagagacggggtttcagcatcttggccaggctggcattgaactcctgacctcatgttccacccgtctcagcctcccaaagtgctgggattacagacgtgagccactgcgcgcagctattttaattttattttttgagacagagtttcactcttgttgcccaggctggtgtgcaatggcacgatcttggctcagtgcaaccttcgccttctgggttcaagcgattctcatgcctcggcctcccgaggagctgggattacagacatgcgccaccacacccggctttttctattttcaatggagacggggtttctccatgttggtcaggctggtttccaactcctgaccttaggtgatccgcctgcctctgcctcccaaagtgctaggattacaggcatgagccaccgtgcctggcctatctaaactttttttaaaaaagactgaagtttgctccccagtccaccaaattaatcttgccagccttaaaatattaagtcattttcatgattctgtatctattaaatagcaataggtttcagatctgaagaggtcaaattaatagttaaataaaggaaccattctaattagtaaaacttgagtaaataaaagcatctgatctatcacttggaattcctgatcattgaatttgacttgcaagttttagttagggaataaaaggatacctcaactcacgcagctccgtagctaattcttattgtaaatatacgaatcatcaacaggtttcagatacaatacatatagccctagacttattctctaaatatatatatatatatatatatatatgtatgtctacaaacataaaaacaacctatgttctcactttctcattaggactgaaaacactgcccctgaagtaaaagtatactttttaattaatttaccaggcatttccgtagaaaagccacatctgtagaaaatgtcacccttttgaggagggggcatactggttaaaattttaagtggttgcctatctccaaatgaccacaaaggcagcttctaaactactaattacaatcaagaaatttcaggtctcaggccgggcgtggtggttcttgcctatactaccagcactctgggaggccaaagcagatggatcacttgagcccaggagtttgagaccagctgggccacatggtaaaacccctctctacaaaaaatagaaaaattagctgggtgtggtggcatgtagccatggtctcagctacctgagaggctgagatgggaggatcgcctaagcccagagaggtagagactgcagtgagctgagatcatgtcactgcactccagcctgggcaacacaatgagactctgtctcaacaaaagaaaaaagaaatttcaggtctggatccctcaatgcagaagtgccatttttaatttcattcatactgtaaaatctttcacaaggctatatattctttgataaatttactttacctcaccgacgtatttctgtacaagctcagatccaataactcgaatgaagcacgcatcagtccgattagcaactgcccgcgcacagagtgtcttgcctgtaccgggtggaccaaagagcagcacgcccttgggaggctcaatgccaaggttcacaaacctctctggctgtgatagagacacaattttcacacttaccacttctgtataacaaaaatcagactacttctaaatcaacaaagtgcttttatgtttagtattttatcctgaaaaccatactttaatatttaaatcaaaattattaagtccacaatatacatacttgaactaaataagaaattttacttattctaagacataagttctaaactggacttctatcaatacataagactaaatggcttttaaaaagcacttccattatccagtcttaaaatattttcctaatttaaaaaaaattatggtgtttaaggaacaaaattatctggtattcagtatcctagttttccattatcctcccaagtttaacctgcttcaatttaagcccctttcttctcgatgtcttttgaagaaccagtttcttctataatagcctgtaacatatttaacatattgctaaaggttacactgtgcctcctctgtcactgtaaacagcagatctttcttttttaaatatacctgaccactcaatcctttttgcttgctctcccctagaacttaccaactctccacacctcctctctccaccctctcaaaaaactctcattagggaaaatattaaaagccatctaatatacaagcaattttagtgctcaatacacaaaagacatctttatacaaagaaatttaaatacaacactcagctacttacatgaagtaatggggtttcaactacttctcgcagtttctcaatctgttccttacagccaccaacatcactgtatgtgacatcaggtttctcttccacctaagagagggacagaaatgtacaacatacagtcacaggctttttggatactgtccaaattcaatagaatagacctaataccttaaaagaaaaactcacaagtgagaattttaataaaaattgaatacattgcccagtgccctggaactacttgctgtattaacttgttatgacactaatgaatgaaaaacaaactagtaaaataaagacaggctgagactctgtctcaaaaaatatatatataaaatactgccgggctcagtggctcacgcctggaatcccagcactttgggaggctgaggcaggccaaacacttgaggtcatgagttcgaaaccagtctggccaacatggtgaaacaccgtctctactaaaaatacaaaaattagctgggcgtggtggcgggtgcctgtaatcccagctactcgggaggctgaggcaggagaattgcttgaacccgggaggcagaggttgcagtgagccaagatcacaccattctactccagcctggacaagagtgaaactccgcctcaaaaaaaaaaaaaaaaaaagacatagccttccttttccctcccatagtttcttacctgcatcatggtaactgttgggtcaatcttaggaggcaatggaatgtgaatttgatatttatttctatccacgctaaggaagtaaaaaagatagtcattaatacattctttaagctttcaaagtgcttttatgtttagtcctttatccccaaaaccatactttaagaaaacaagagagagacagagtccctaattatataaagagaaataaagagagcatgaaatctgaggatactaaactacatgtctaatgtcgcaaacaacctatccaacagcaatcaaacctttgaggagggaggggagatttttttttaatttactaagtgagttgttatgtattctatagacattaacacacttagcatcacaactatcctaaagcaagtactcccccaacctataccatgttaccttaaatggactggtaacctcaactctgaaatttgaagccattcataataaacaacagtgggagtaggggagtcagcgaagccgatgggaagttaggaagcagatgcagaagacagaattttcaactatagtacattactagatcatcctttggtattccaaggaactcagtccatttattcactacaaagccaaggatatgaatgtgagacagtatccttagtgaaagaaaagtatttattgtaaatagaaatcttaccccactctcatcccttcttcaatgtcagtaggtgccacctgatcactaaggtccaccacaaacttggcaaactgctttacgttgataatgtattttgggtcctccgaatcagcattgattatctttgtacacctaacacagcaaaaagcttgattagaataaggaacctaagccactaataatgaattcaactaactagattatatcctgagtgtgcaatatacaggtataaactagaactacagaaactgggacactgttaaagatccttttctatacccaactccaaaggtccattcgactttagccaagtcataaaattttcaaatacattctcctggaaaattaaacccaaatatctcatcattaagaagttctctgatttcttgatttccatagtttgtattcctttattattagtgatgctcctgtatatcttaagttgctctcgtttttattttccctacaaaaagatggtcatcttgtcttacaccaaattcagtaataaatgggttttcattcttttcctgagtttatactttaaatccgtttttctttttttttttttttttttttttgagatggagtttcactcttgttgcccaggctgaaacacaatggtgtgatcttggctcactgcaacctccacctcttgggttcatgcaattctcctgcctcagcctcccgagtagctgggattacaggcatgcaccaccatgcctagctaattttgtatttttttttttttttttagtagagagggggtttctcttgaactcccaacctcaggctagtcaggctggtctcgaaatcccaacctcaggtgatccgcccgcctcagcttcccaaagtgctgggattacaggcgtgagccaccacacccggactttaaatccatttttattgtttttgctacttttttctttttttaactgaggcagtagaagcttgtttttttttctttttcttttttttgagacagagtcttgctctggagcccagactggagtgcagtggtgcgatctcggctcattacaatctccgcctcctgggttcaagcagttctcctgcctcagcctcccaagtagctggcattacaggaatgcgccagcacgcccggctaattgttttgtatttttaatatatacggggtttcaccatgttggccaggctggtttcaaactcccgacctcaagtgatccacctgcttcggcctcccaaagtgctgggattacaggcgtgagccattgcacccggccagtagaagctatttttaacttaattatattcatttccatatggcctggtaaaaatgaatttataatgccatgcacatcacagtataaaatagtcaaccgcctttccccttctatagtatctatctactaaagaaaaacaaaaacaaaaaacagaactcacattagaaagctgctacataagccacagcaagaattacagagaaatacaaaaatagcctgtggagtgcgcacataggtagagctaaaagtaaggtacagaatgctttggcttagccaggtgtggtgggatgcacctgtagtcccagctactgaggaggctgaggctggaggatcacttgagcccaggaggtcaaggctaaaaaagaatgctttgtcctgatagcataattgtgtgaaggactctgttttctgttaccaaccatgtttgctagggaagaagaattatacccattctctaccaaaaaaaaagaggctgaccaaaaaacctgaaaggaaaaacaagaacaacaaacagcaataataccaaaccctggggaaaggaagaatttgatttccagaggtgacacattacattattttaaatgtctagttatcaccaaaaagttatgagatgcaaaacattatgaaatgcattttcccttatgtatgacattatggctcatacataaggggaaaaaaatcagtcaatagaaattgtcccttaagaagcctagatgttggtgttattaaacaaaactttttttttttttttgagacggaatctcactgctgcccaggctggagtgcagtggcgcgatcttgactcactgcaacttccgcctcccacgttcgagcaattctcctgcgtcagcctcctaagtgtctgggattacaggcgcctgccaccacgcccagctaatatttttatatttttagtagagacaggtttcaccatgttggccaggctgatctcaaactcccaacctcaagtgatccacctgcctcagcctcccaaagtgctgggattacaggcatgagccactgcgcccagtcaaacaaaaaccttaaatcagtacaagaaatattttcaaaaagctaaaggaaaccatatctaaagagtttataggaatgatgagaagaatgtctcaccaatagtgaatacagatagagaaattagactggaatattatttggccataaaaagttccatctattttagaatacggatgaaccttgaaatatgctaagtgaaggaagccatgtactatatgattccatttagatgatatgtccaggatagacaaattcatagaaacagaaagtagataaatggttgccaggggctggaaagagacgggaatgaagactgctaatgggtattgggtttgtttttggggtgacaaaaatattctattctggaattagatagtgatgatcaatgcacaattttctagatattctaaaaatcactaaattgtatactttaaaagagtgaacctcatgatatctgaattttatatcagtaagattgttattaaaaaaattcataagctggctgggcacggtggctcacacctgtaatcccagaactttgggaggccaaggtgggcagatggcttgagcctaggagttcaagagcagcccaggcaacatggcaagaccctgtctctactaaaaattaaaaaaaaaaaaaaaaaaaaaaaaaaaaaaaagccagacatggggtggtatgcacctgtggtcccagccacttgggaggctgaggtgggagcccaggaagcagaggctgcagcgagctgagatcatgttactgcactccagccttggtaacaaagtgagaccttgtctcaaaaaaaaaaaaaaattcacaagctaaaaattacacatacacaaattttatcaagagttttaagcaaagagtatgagactaaaatcaatggaaactttttcaaaataatctttgctttctttaaggcctgggatcttgtaggggtatagggctctttagctagtctgtctgttatgaaaaatctatatgaaaagtttccctctaactgctttacagaaaaaagaaaattcccctcatacggcctagaaaaatgtacctagattttttaatttaccatagagagataagaacatattacaaccatgatataagagcagaacattcaacacacacacaaaaatgaaagaataaagataagagccttgaaataaaaaacctgagtttttttacctgagaaattaaaaacctacgagaagaaaaatgggaggaaaaaaacatcaacagaaaaattcaaagataaagaaaagctcatagaaagtaaagtaaaaaggcaaaaagaagaaaaacaggaaggaaagattagcaaattagaggagcaatcctggaggccccatatccaaataagagaattcaggaataagggtccaagaaaacggaaggaaagaaattaacaaatagtttcaagaagatttgccttactaaaggacaagagagcccaggacaatggatgaaaacagaccaacaccaaggcacatcaccatgaaatttcaaaatagaacaagagaagactctacaattttcagagaaaaattaggccacatatacaggactgaggatcagaatggcttcagagtaacactgggaggcaaaggacaacagaacgacgccttcaaaattttgaagggaaaagaactgtatactcagctaagacactttcagacatatgcagtctcaaaatattacctcccaactgacccttttccaagaagctaccagaggatgtattagaccaagcaacaggagagaggtaaagggcaatctccagggtagagaagtcagacaacaattgtcattaaacaggcataaaggacaactggtccaaaccaaagcaggtcagaaggtgctgggaaaaacttaaggaaaatgaaaccgagagaacaccagatgcaactaaggtataactgagagaaaacgaacagatctaatgcatctgaagagcagtttagacaatcaagagaaagtgggaggttgagttcataatatatgcacagaaaaccaggcaaatgaaaaaacaagacgttataacaaccaccactaaaagaagtggtacaagaaaggaaaagcaatcacagcttaccacttggctctttgtgaatagtgtttacatagccataatcatataaacactgaatactgatctaaccaaaattataacagaattacataggaaggatgaggggataggaaaggtgtgccttcaaggtgaaggcagaggaaacaaaacaaaatcctcatcttccatgatggaaagccaaaagataatacctaaaactaaaaaatcaagaagtaggaatctataatacacaagatatagcaatatccattagaaccatgaagacgaaaaccaaaataatcagctaaaaaaggtgaaaatgagtgtcattagggaagagaaattgggaaatacagttgttattatttaactgtttaactcttttcttttttttttttttttgagacagagtatcactctgtcaccccaggctgaagtgcagtggcacaatctcagcttactgcaacctctgcctcccaggttcaagcgattctcctgcctcagcctctccagtagctgggattacaagcgcccgccaccacacccagttaatttttgtatttttagtagagacggggtttcactatgttgaccaggctggtcttgaactcctgacctcaagtcatctgcctgcctgggcctcccaaagtgctgggattacaggcgtgagctgccgtgcccagcctgaactatttaacccttcaaaaaccatgataaaactaaaaagtgagtgaataaataaaagtgagaaaagatggaaattgtgaagtagggagggtgagtagatgggaaataaaaataatctttagcttttaggaactaaaaataaaaacaaaatttttaaaaggattataaacagaggagaatatgtttatagccttgaattgggctaggtttttctaagcaacattaacacccaaaaggtataatggaaatattggtatattttatctcctaaaaatgtaaaacattagtacaacaaaaggtaccaagaagttaaaagtgactagggaaaaatacttgtagcatacttaagaaaagacaatatgtacaatatatgaactgtttctaaaattaacaagaaacaacccaagaacaaaacatgagtagtcaaagaaatacagatgtttagtaaacaaaagaagtagctatcatactagtaatcaacacagtacaaattaaaattattttccctcaaatggcaaaagtgtaaaaattgaaaactgcgtatcttacattgatcagacaaatggacatttgcctcaaagcctcttggtaaaagtataaactggccaaagtaattttcacaggattttaaaactataatatctttgacccctagaattccacttgtagtaagctagcctacagaaatgtatgcacatacactgagaaacatttaacacgaatattcactacagcactgtgtatagtaacagaaattgttacagcctaacatctacacattatgcaatttttgcaattttttaaaaacacaagaaaaaagaaataagtatacctatattaaagacaaagattcccaaaacatgtagttatatctaaaaaaaaaaaaatcaggatatgtttctttgcatgcaaatacaggaagatacacagaggattacgagggccaaactatttcagtagttacgtttgaggaaagggcaacgggaaagcaagtatgagaagggagtttttcatttcttgctctacatctttttatatcatttaaatcttttaaatgattatgccttcatatatttttgaagaattgtttaaggcataaaattttaaccccagttttgtagaaagctttatttacttggaatttggaatcaaaattatttatgaactattgcattatccctgagactctaacattaagtgcttgaaaatacagacaaaggtaagttgccacagagcacccgtgcatacctggcaacctgtaaaggctgttcactctggagtgtctgcttatctgcagccaaatcccagagtgctggtggggccaggccagtgtcagattctttaatacctacatagagaaagatgacaaaatttactaaaatgtaatgcatcaccctataataagctttctaaacctcagcactactgatattttgcactgaatcattcattgtgaggactatcatgtgcattatagactgtttagtagcatccctggcctctcaccactagacgccagcagcaccaccctctctcaaagtcatgacaatcaaggatgtctccagacattgcccagtgtcctctgggaggggttgggagggtggggcaaaatcaccccagctgaaaactacaccctgccctaaagtaattgtcaagccaaaaatataatgccacatttgacatttttgtgtaggtcaaatattataaaacttcatcattcaagttccccccaaaaagttccaggaaagagcccaattacttgaagcaaaactatgttgtgagatcttatttttatccaaaaaagttttttgacttgttaactgaagtttactgaactgggttttgaccaacagaaatattactggcttagaagatggtactatttcttatttcttaactgcataagcattaaacctggttgtaaaaagcagtgaaagatatggttaaatgagatctaatttcttaacagaaaggaagaataatgattttttttaaatctgtaatcatgaaggagtaaaatctgtatttaaactcttgatattattcacacattacatttaaaggaaggaaatgggaatttaaaaatacataccagtgagctcattaattttcttgagaagttgctgaatgtcatcttcaacttgcttgatctgcctagagtaagtgctctgaccctaggtgatgaaggtcagtttagttagaatatcaggatatcaaccatttattaaaactacaggtaaaaattatttttaagttacaatttcaatagctatcattatcccaaagcttgtgaagtagagaggtattcctgacccaatatttccccctaaatacacaatccactattatgcaagataacaaatgaaaatcatggccaggcatggtggctcatgcctataatccctgcactttagggggcggaggcgggtggatcacctgaggtcaataattcaagaccagcctggccaacatgatgaaaccctgtctctaccaaaaatacaaaaaattagccagacgtggtggtgggcgctcataatcccagctactcaggaggctgaggcaggagaatcgcttgaacccaggaggcggaggttgcagtgaaccaagatcgcgccattgcactccagcctgggtgacaagagcaacactccgtttcaaaaaaaaaaaaaaaaaaaatcgtgtgtgaaattatgcaaaactatcgatttctatgcattaaaacttcatttcagaactaattcagtaatacttgttaaatatagtaaacttgtgtaaatgatttcaaatatctttcaatatagcatcacattttaatcacgaatagtattaaaataatacataaactgagacatgccaaatactttgatctattctataaactttaggtaggtaacttcatttagtgaagattactagaagctgccagacattcttttctaattgtttctttttattttattttttgtttggttttttaatttttggtttcttaacttcttctaattattttggaaggacaataccaaaacagtttttatttcaacataaaacatctatagtttgtagacactgaaaggacttacataagttttcaacaaggcaatatccccctcatccagagctgaaagaagagagacaatacgtgagattcaaaaagaaactagaattaaaatttttaaaaaagaaactagagctttttttctgttcgattcaaattgttttaagtgagtgtcaaaaatcaagcaagattatgatgatacatattcaatcaaatagttcctggactcaaggaatttttatctaactgggaggaaaataggtttttaaaagtacataatatacgtgatgacctattttatatagtacatacataaaacacatccattaaggatcttggttaaattataccatagtcatacaatgaaatacaatgcaggccccgtgcggtggctcctgcctataatcccagtactttgggaggcccaggcaggcagatcacctgaggtcaggagtttgagaccagcctagccaacctggcgaaaccccgtctctaccaaaaatacaaaaattggcctggcatggtggcgcatgtctgtagtcccagctactcaggaggctgaagctggagaattgcttgaacccaggaggcggaggttgcagtgagcccaaatcacatcaccgcactccagcctgggtgaccgagagagactccatctcaaaaagaaaaagaaaaggaaatacaatgcagcaatttaaaatgataacagaacatagacatagaaagcaatccaacaaatgctattcagtgaatcaggtaggtttaaaaaaaaggatataatgtgcatatattttgtttttatataaaaatcacttaaaacacatgtggagagtaacctgaaatggtgttctacggtggttatctgggatttcaagtgatttacagttaatcttttctattcacaaattctgcatttctgaatttgcttagctgctcaaatttatttgtaatccccaaatcaatactcatgtgctttcatggtcatttgcaaatagacagagtggtaaaaaatttaagttcacaactaagaacaagtcactctaccctcttgtttcagctttcatacagagatgaccagaagatggagaccgtaggggaaatgccgtgtaatccaagcagttcaggctttggggccaggtggacagggtttgaatgccagctctggaatctgttagtggggtgacctcagccaagtcacttaacacttctgaactttgttttctcttttgtgaaataaagaaaaaatgaatctac

SEQ ID NO: 2 - DNA, 93422 bp, Homo sapien
aggcagcggctgtggagcgcggcggggcggctccgcccagggcagcccgggctggtgagtgcgcgcgggcggccgcggggccgggggtcgcggtccctcgggaggggactgagctgaggcccgcgggcccacccgagcaggggccctcgctgtcaccgcctctgcccctgtctcccaggccccatgtcgcgctttttccctaaacgaaccggtgacagggctgacctgcccgcactctgctggtccctacgccgggaaggagaaagtctcacgaagctctttcctttgcaacccctgtccccttgccctctgcagctccacacagcatttttacttcccgtttgcctcctcgaatcccttcggtgctaatccagcccttcgccgtttccagtttgggtctggcctgtttgtatccaaatcgaattacacaaaaccaggccctcagttctgcctctggccctctttccggagacgggccctgcctcctcctgtgccgggctgggcttgcccggcattggccgcccggagctcccagggagcaaggctgtggtcccgaaactgggccctccgctcagcgccgggcaaggctcaaaccccgggtctctgacttatccggtcacgaagggactgctcagagcctaacagccctaaaatttggtattggccccctcccgggaaatccttctccctaccaatcaggcatcgcggcaggtgctctgccatcatagaggctctgacacgctgaactctcgaagctcgcggcagtgtttatttttaggagagatttagttgatcgggaaccaattgacttggttgactgcctcatctagtgcgcacctgggccgggccccgccgggaaggcgtgctggacgttctcagtgagctccgagacctacagaagaaacagctgcaaggatccgatcagctagctgtgaagaaagcctcagatacgctgtggggtgggcaaaattcttttcctagcttcgatgagtactgtggattctagaatttaaacaacattcatggagactcttaaaacaacaaaaaatatcaactttttgcgggcacagactgagatttagctttttcctatctgaaaactgaacaatgtgcactagatataaatgtattatctaaagtttaggaatttacagcatagaaatgcaggggaattcggagtatgcaatacgaagtatcaacctttttgaaaatgaaatttaaaatgtatttttgtcaggcattccttctaattacggtagtattgctttgtatcagaagactttcaggcaaaggacaggagggaagagagagaaattcaaactaatttgcattagaaagaaacttagggtgaagaattgcctaggtaaggttgctatagctcatatcttaagtgcactcttggaaagctttctagtctattaggtaacacctgaagccccaattctcttatgtagcatttctttttttctctctttaactactctgaaactatacagagttacacgttcctcattaatttatgagtgaaaccaaaagaattgactcaatccaacaagtatttgagtgcttttcgcatagaggcgctttgctaagaacagaagaatggaagactgaggaagtttctaacaatcaatggattgccttgtgaggtgtagatgcaggtgtgcactgcctatttaattgacgatcaaagtcagttgagttttcatctcattctaaatatcatgtgttgccccctggtatcttaatttttctaccgttgcaggtagtctgcattttagtaacccaagtgaaacagttttctagggaaatcatgataaataggaagcacgtccgtattcccaggtgttctcattatcattaagttaataaagttaagaaaaataagatgatgattttcctttcttttggtttcaaactagtgaaatcttgcacaaaagacacagcatggataaatttaatcattttgtgactcagagtacattttatgtaagtccattctgttggaggatagatccaatttcatgaactcttttaatccttcttttgaagactagttccaggaccaacttcataaaatgttttattgtaggtaccaacaactatcaacatcattatcttctccccaccttcacccttgaaaacaattgtattctagaggattatgaaggaggggacaggactctttcaaaatctggaggacactagggacttaccccagaaaaaaaataaaaaagctcacccattcaaaatcttgtacacctttcagaggattcctggtgcccaggttatgaaagctcacttcagaacgggtcagcgaattagtcagaggataggtagatggagggaaagggaaatgaagcaatgtgggtaaattcacaaactgagtggatatgacttaatttactttcaaatagaataaatacatggccccttgagtaagtattctgtgaatgggaaggaataattgtattctttgtaaaagactgaagacagattagcctaattaagtttttaattgatcaagatcaactcttatcttgaacattatgagtctctcatttttctacttttcttgctcttttcaaggcaactgctctaagaggatttgctcccactactgatgatgccccattctggacacaatttctcatatgcaaacttgtattcatgaattgctacatatgggaatggctgagttcaaaaattgaactatatctttctcacatcctagcagttcacccttaggaagaagatataattatatataattagtctatcgcatgtaagccactgtgatagcagtagctgtgccttggaagggagtgggttggtattaggaatgaaaggagagcgttaagaaatattttggacatagcttctttcaaaaaacaaaccaacaactgctgaattaacttccttttcttcttctatacatagggccaaggagcgagctctcccttctcctgctctcagcctcagtgatcaaggcttcagtgaactgcactggagctcccagcgggggatcttgtcccctgtcccgacttttgtgctgcacattggatctgggtaagtagacggccccctggaactagtaaataggagagtgggtcagaggtagtaatggattttatccagatcaaaggattcctcaaaatttccagaggtctaaatcacacaccactatttggaagaaaagtataagacagaatgccagaaccatcacaccttacacttaccttttaacttggatcaggatcagcctaagatatccttagctatcaaccagctttgacttacagtattcttgagtggcaactaggtggtgggccaacaaccctagaatgtacttaatagatgcttactggatttgtaaattaatggagatcatggttttaagccaagctctgccactaaccaggcatatgatcttgggcaagtcacaggttagttaaattctttggaccttaatttttctacgtataaaatgagggtgttttatgggaccaatggttctcagcaaccaatgtataattccattgcatttgcattgcctgggagtttatttagaaaaaacccaacagagattctgattaattattaatcagatttattctgaggataaagtccttagtgttccctagattttgaaaaagacctgtcccctccctcatggccctccagaatacaatcgttttcaatagtgaaagtgtgggccaggctcagtggctcacacctgtaatcccagcactttgggagaccgaggagggtgtaccatttgaggccaggagttcgagaccagcttggccaacatagcaaaaccctgtctctactaaaaatataaaaattagccaggcgtggtggcacatgcctgtaatcccaactactcgggaggctgaaacagaagaatcaaacccaggaggtggaggttgcagtgagccgagattgcgccactatactccagcctgggcaacaagagcgagactcagtctcaaagaaaattaaaaaaaaaagagtgatgaaggtggggaggagacaatggactggcataggcccaggcatctttattttttaaaagtaaccccagcaaatactaatgtccagtcaggattgagaatcactgggttaagtgattgctaagaactgattctaagattgtgtcatcttaggatggaaagcaggcccacagagtccctgttaaacatagcctggcccagcattgcgcctcctctgtggtaaccaaacctaaacatgcctttctcagaagcagtccagatgaagccatttcaggttattcggggggagctgctgtgcctgctcacctttttgatattcccacaatgctttgctgactttgcagcacccagtagtttttgatggactgaattaggtgtaattctgcctatccaggatgatcagctgttcctaaaggtccccagagacgataacatctctgctctgagtatccatgccagtgtttaatagcctcttgaaattttctccttgaagtgatctgtgacttgttgaggtggggaagaaggtgtcagcaggggataagcatgatcaacttgatcgtagctgaaatgtgaactttagtggccaaactgagataaaaaatgatcctcacaccttctcaagtagttgaagactattgagttaccagaactgggagaggagaaaaagagcacattgtttaccttaattatttcttgggacctaatcatgttgccattacattgtaaattctgtagattttcacacttggaaaattcttcttaaatttagagttaggacttactaaattgtaatccatgaaaggtatttgtttaccctactcttcttttaatctcctactacctttcatttcccaaaatacctagaagacaaaaaacagtgtatatatgtgactgtcaaatcggtaagagagacagacatgagttgagcaaaaatggaaaggcattaaggaagaaaaactgacaagggtaagaggcacgtcggttaccaggagctgcacttaatttctctcttttctttctcgattagtcttatctctgctataagcctattctcaagcttgctaaaatgcaggcagatcaccatatcccacctgctccatgttgacaaatgtgggcttatggcccttgattccctcatgttccctgcagccttccatacacagtgcatgcagtgctgtctccaggggtgaactggcagcctggacacacttggtctaatccagcattggccaatcccagggcctgacttaattctaaatcacagcttaggttgaagcatattttttcctagagtggaaagctactggcagtaaagttctttacctctgtatctcagaggagtaggctttacctgatgtcaaaaatccaatgcagacctacaaaactagctcagcccccacctctccagccacctagagctggttgtactcattgccagtaaacaaacaaaacaaaattctaggattggagtcattttatatgtttattgctgatcgttgttcccaaagatttagtagtattgggcacttgctgttttgttatttcataatttattcctgacctacttcttaaaatgaattaaagtaggaagaagtgtaccaggaaaagatagatttgtcataatttcaactctaatggaaacttaattattactaaactcaatggcccatttatgtgtttataaataaaaatgtatccttccatacatctagtttcttaagataataagaatagtgttgtgctagtccttaggattattattagtttgctttttaaatctttacttccaaaaagcattacaattttgcaaaataattagaaatggtaaaaatttcttagccagtgcctgtttattgaacacttttaacaacaatctcaaaggtaacaagggttttcattgcttcctttcttgctttattatttccttcctttcttcctagagtcactcattatccaacaaatatctaatgagttccccctatgcccctgacttcatgtagcttacattcttatgaagaggggaagagagacagaaaataaagaagtaaaccatcatgtaggttagatagtgttaagtgctatggataaaaatatatctagaaagaagatggtagaggtgttggctgtgaaatttttgaaagaaggtggtcagaaaaagcttcccctgagaaagtgatattgagcatagaccggaaggtggtgaaggaggaagttacgagtctatctgggagtagagcatcctggccagtgcaaaggccctgaggcagacaagtgcttggcatgttcataaaacagcaaggaggccaactgcggtggctcacgtctatcatccaagcacttttgggaggccaaagcgggcagatcgcttgaggccaggagtccgagaccagcctgaccaacatggtgaaatcccatttctactaaaaacacaaaaattaggtgggcatggttgcgcatgcctgtaatcccagctgctcggtaggctgaggtgccagaatcacttgaacctgggaagcagatgttgcagcgagctgtgatcgcaccactgcactccagcctgggcaacagagtgagactctgtctcaaaacaaacaaacaaacaaacaacaacaacagacagcaaggaagtccatgtgactggagcagagtgacaaggtgggaaggtaaagaaaatgaccacagcaaggaaaggagcagtggacaagagtttgagagggtgctaaagatcacacagggccccttttcagtgggatacttgtaaggtgatgtgaagatggagtttcttactgtttagttgaaacagcagcagcctgtgttctctttcttccttgttatacctcctggatagtccccccagttttcctttcctctccaaattagcctcttcccattcctccccttgtgcttcctttcccccaaggctggctgcagagagggcttcggtttccctttctccttgttgccctgaagataagtctgcccctcctgttctaagaaatgtgctgctagttgctaaagaataaaaactgtcaggtagcacccaaaccaatagtcatgaaataaatcataataaacttatgcaaaatgtattcttagtttatactgcagaccagacacaaagtggttgtgttgctgttgagcatagttgaacctatgtgttttgttgggtctgcacattgtttcatgaaaatgtggattggtttccaacatttaaaaattgagaaattttttattaaaatgtattttcaagttttccttccaaattcatgttcctgatgaagttctagaataggcaaaactgatctatggtgatagaaatcagaactatagttgccgacacatcagagattgactgggtagggacagaagggaaatttctagataatgaaagattctatttctagtagtctgaaatgcacagtaaactcattaaaccatatactgatctgtatagttcactatgtgtacatgaaatctcaatatagctgggcacggtggctcacacttgtaatcccagcactttgggaggctgaggtgggtggatcgcttgaagtcaggagttcaagaccagccaggccaacatggtgaaaccctgtctctactaaaattatgaaaattagctgggtgtggtggcacatgcctgtaatcccagctacttgggaggctgagggaggagaattgcttgaacctgggaggcagaggttgcagtgagtcgagagcgagactccatctcaaaaaaaaaaaaagaaaagaaatctcaatataaagaaatttttttaaaattatgacgaatattttaactgataaattataattttatatatttatgggtacaatgtgatgttttgatatatgtatacaatggagaattattaaatcaagctaacatatccactaacctgcttacttacctggtttttataatttgaaatttactcttagttttttttaaaatatacagttgaccctttgtatccacaggttctgcagatagagagggccaactagggacttgaacatctgtggattttgatatccttgtggcgtcctggcaccactctcctatggatatagagggctgaagtcacagttatgatttccatcaccatagattagttttgcctattctagaacttcatatagaacatgagtttggaaggagaacttgaaactacattttaataaaaactttctcaatttttaaatgttggaaaccaatgcaaattttcatgaagcaatgtgcagacccaacaaaacacacagtcctcctgctgtgcaatcgctctcaaaacctattcttgtctatcttaaactttgtacccttcagtcaacaattccccattccttccctcccacccatgacctagcctctggtcaacaccattctactctctatttctgtgagttttttagattccacatgtaagtgagatgatgtggtatttgtctttctgtacctggcttttttcacatagcatcatatcctccagattcatccatattatcacaaataacatgatttctttcttttttaaggctgaatcatatttcactgtgtctatgtaccacattttctttattcattcatccatcaatgaacacttaaattggttccatgtcttagctattgtgaataatgctgtaataaacatgggaatgcagatatccctttgacgtattgatttcaactcctttggatatatacccagaagtgggattgttggatcatatggtagttctattttcagttttctgaggaactgtcatagcattttccataatggttgtactaatatacactcccaccaacaatgtgtaagagttctgttttctctgcatgctcaccaacacttgttatcattcatctttttgatgaaggccattctaacaggtgacattatgtatcattgtgtttttaaagtgcatttccctaaatattggtaatgctggacattttttcatgtacctcttggccacttctgcgtcttttgagaaatatctattctggtcctttgcccattttttaattgtgttatttgttttcttgctatcaggttgtttgagttccttatatattttagatattaagcacttgtcagatgtgtggtttgcaaatacttactcccattctgtgggttgtctcctcattttgttgtttcctttgctgtgcagagccttagtttgatgccattccatttgtctgtttttgcttttgttgtctgtgactttggagccatatccagaaaatcattgcctagaccagcatcatggagctttcccccattttcttttagtagatttacagttttaggtctcaacttttaagtctttaatccattttgagttggtttttgcatatagtatgagaaaaggatccaatttcactcttctgcatgtggatagccagttttcccagcaccatttattgaagagactgtctaaagaaaagttttaaatggaagccctacccaacattcagcaatacctggatagcagctgaccactttgggcttggcacatgctttctagattggcttagacaccttgtccacttctgtcattttcattaactacctggctccttgtattggtattggtggtattggtttatgactccttcagtagatgcttagcagtttgggtttcaactatcagtggggagcaaatctgaaggggcctgatacgtttaattggtagtttagaaaatagtacctgcctcttagggttgtatgtgaagcatgtaacaaaatcataagcctgattttgttaaatcagtccagaaactcgcctattcccctttcacttaatgctggtctcaaatctcaggattctcaggggtctctggacctaacttgcgctttcgtctcaaatgccaagggtctcctgaattttcagacttctttcctgagacgataacagaaaatatcttaactattactcctattatcaacacttaacacttaggcttataatgtgtgttgctaaaataatagtgcctactatggaaattttagaaaatacagataagcaaaaaaaataaaattactcataattctaccatcaaacataacctctgtttaccatgttatggtaaatactttcaaaactttctccatacacctatatattttatattaaaatgtgttttataaattttagcgtacaaatgcccagtttacaaaatggtcaataataacaagtcaaatgttaaagatgacaggccaagcataagtacttgcttactctctatcctaaaactccattaaaatgaccgtaaaagatacaaagggggaagaactctgaagataatgggatatgaaaatgcttggaagcaaattactgattaaacagaaccaagaatttgtacaaaaaggaccaccctcggagtgcaagtgatcccctagaaagaaccccctgtatggagttagcagcaatggggagcatgcatgagcaacgagattaatggaaagtcttcatggactagttatagtgaagtagggcttctctagagtattggcaccatagagtaaatcttttctctttctggcatttagaaagagggtgggccctggctaaaaggctagctcttgcctcttcacccaaagtataactagctatcaacaaagcccactccggtacacaaaggctgttaatgttcatttcgttcagggaagagtcctatcctggagacacttatatacatgacagtggaagaaagtggcacaaatcaaccagaaaaacaaacgaaatccttaatttataaagaaattatataaccaagaataacctgacatatgaagaagcatgaaaaagaaacatcaagataaacaattactattgccagaggagagatcattcagggaaaagaaaatcgccttttttaaaaatctgagcactcttagcatgattagagttgttgtttccacacaacaaaagcgtgatgttattcagaaagagcaatcagaaaataaaaaagatgttgaacatttttttaaataactaaaataaaaataatttactagaaggctaataatggaaattcttcctggtccaagtccagtggtgtttacaactaattgatcacaaccagttactgatttctttgttccttcatttccactgcttcacttgactagcctaaataataatattaataacaataatgatttttaaatctcctgtaatgggccaggtgcagtggctcacgcctgtaatcccatcactttgggaggctgaggtgggcagatcacgaggtcaggagttcgagaccagcctggccaaaacggtgaaaccccgtctttactaaaaatacaaaaattggctgggcgtggtggcgcactcctgtactcccagctactcagaaggctgaggcaggagaatcacttgaacccaggaggcagaggtttcagtgagctgaggtcgcgccactgcactccagcctgggcaatagagtgagactttgtctcaataaataaataaataaataaataaaaataatctcttggaatgcacaaccaaattttgaagaaagaaagaatatgctagaaaagaggagagacagaagcaatccagtaagaaaatctttgttttctgagatagaaaacaaagaaaataaaaggtaagaaattgacaattaaatgctaggaaaaaaagttcctgtagttgaggaaaagtgtgaggttaaaaggcccatcaagtgaatgaaaaaacatgcagagatacaattgttacaaaatttccaagtcaagaatacacagagattcctaaaactttctgaaaggaaaaataaagtcgtttaagattaacataaggcttctcatgaggaaactggattttaatagagcaatggctttaaatttgtaaggaaaaatgattttgaacttagaatcatattgtcagtaacttatcaagcgtgagggtgaaatgaagacattttcagacattcaaagactcaggccgtttacttctgagtaccttttctaaagcagttgcctaaggatgcactccagcaaaatgagggtataaagcaaggaggaggaagctgtggaattagaaagagtggagctaatccacaattgcaatgaaataaactgtaggttgacagatgttcaccaggcttagaaaaaattaatacaggccaggcgcagtggctcacgcctgtaatcccagcactttgggaggctgagatgggcagatcacgaggtcaggagatcaagaccatcctggctaacacggtgaaaccctgtctctactaaaaatacaaaaattagccgggtgtggtggcgggcacctgtagtcccagctactcaggagactgaggcaggagaatggcatgaacccgggagatggagcttgcagtgagcccagatcgcatcactgcacaccagcctgggcgacagagtgagactctgtctcaaaaaaaaaaaaaaaaagaaagaaaaaaaaattaatacaaattagggcagatagtgagctcaagaagaatgactttaagaagaattaaatgcatttccttttataggtagaagtagcagtgaacaatattcacatagttatagaaataatggaaattcttcctggtccaagtgcagtggtatttacaactgatttatacaaccagttactgatttctttgttccttctacattcccactgcttcacttgactagcctaaaaaaatgatgatgctgatgatgatggcacttctagcaacacagagggcgaagctgactcattcagatctccctccaggacaaacacacaaaaatgaaggataaaacataacaaatgttcaaataaccttgaaataaagactgtcagaagaatgcagagaaaaatgtttaaaaaagaacaataataataagaaatcctacatgcagcccaagccataggaatttttctgtggaatatagaccctagagtgttgaatgatgaagttgtatcattcaaacctggaaagaaaatttggcttcaggcccacaggagaccaggaccatggaattgaaacactggataagcctggagctctaaagacctgtctagtccattaatagagtattaggcaaactctaaccactacttagggaaacaaggaagtcttgtaggccttgagtgaaagttaaaaaaagaaagtcacccttgaacaaacaaaaccctgggcctgtgcctcattcagaaattaggttcaaaattatacttcctctgtggttcagaagcctcaaaccatattaaacaaacaccaacagtagtgaaaatgtccaggacccactaaaaccaccataaccctagcagagtagacatgaaagtgtctaaaaacacatcacgacttaagtgtacatggaacatccacagaaccactcctccccttccacaattaaatatgactcagaattttaaaaacctacaaatcactgcaagggagtgtcagcaaacagaacaagcagaagaatcagatccccaagaatttaaaacaatggaaatagatgaacaagatcagaagtgtgttaaaaattattgaaagaataaagagaattgaaaacaaaagggaaaatgcaacactatgaaattagaacaagttgatttcaaaaagtatagaaatgaaaaatataggctttaaatatttttaaaaaattaatgtcctacttaatgtattagagccaaaagtagattattaaactggaagttaaatctgagaagactatctgtaatcaacccaatgagataaaaagctgtaaaatgtaagaggttaaagcacacaatgacaggagaagctccaacaaatgtcaaatagaaattccagaaggaaagagaaaagggagaataaatcagtatatattggttttataggtaaagaaattaaaaatagtaatataatttagcaaaaatagaagtgagtaaggagacttaaatgttaatatttcggctgagtgtagtggctcatgcctgtaatcccagcactttggaaggccaaagtgggtggatcacttgagctcaggagtttgagaccagcctaggcaacatggtgataccccgtttctacaaaaaatacaaaaattaggtgagtgtggtggcatgcacctgtagtgttagctactcaagaggttgagacacaacaatctattgaacccaggaagtggaagttgcagtgaaccgagctcatgccactgcactccagcctgggtgacagagccagaccctgtctcaaaaaagttaatttttcatggtaagcattcgtgaataatgtctaaacttgataagtaaaaaaaaaaagccatataattttattacttagagcagtagttctcgattccaggaaagattgggaagtgtatcctgtggatggagaatgctactagcatttaatgtcaagaagagagatgctaaatgcacagaactatctcatacaaatcccacctgaaataccagtttctctttctgagaattgctgatttagagttaataataaactcaccagaagaattaatattaaaaatgattttaagaaaatggtggctggctacagtggctcacacctgtaatcccagcactttgggaagccaaggtgggaggatcacttgagcccatgagttcaagatcggcctgggcaccataatgagaccctttctctatgaagaaaaatttaaaaattagccagctgtggtggcacatgcctgtgatcccagttactcaagaggctgaggcaagagaacagctcgagtgcaggaggttgaggctgcagtgagccatgatcacaccactgcactccagcctgggtgacagagtgacacctgtctcagaaaggaaaaaaaaaaagaaagaaagaaaatggctgcttcaggaaatagaactaaagttggggagaatgggatggaaaagtattgcttttcattattagttatttcataatacttgcaattttatcatgtgtatattatgttggaaaaagaaatgactagtcaaccagtagcttcttctttcatttttgcttagagaactcttttctattgaagtatctgtcattcaaacctaagcaccactttgaattactgagctttttacctcaaagaaaattctaaaggcatatcttatgagcgttaaagatctttcaagtgtttctggctggaaaataaatcttgctatttattaatttagggagtcacagcaaattagtgctggaagggattcaacttatttagtggtatggaataatttatccgtggtagagtgattttgagcagtagctctgtataggacaaaggaggactggggtatggtcaaataacaagagcccatggctgcaaattggaacctgcatcctgcctttcatggacttaaaagatctgatcctacttgacctctccagttttatttcctgtaaccacccaaagcacgatttctaccagtacctttggtattccatgaacatgtcttattagcgtttaccttttcacatctctatgcaaaatcacgtgcctgcctttttgccttctatccattatttgagagccacttcaaatccagtatcctctccaaagccatcctctctggaacagcaggcagaagccaggaccttggtcaccctacttagatcaagtaactcaccagatgtcccaccatggtggcagaaccaggacttgaattgaggtctatgtcattccatagcctgtactcccagctgccatttacagataaggaaattagggtacagaaagcttgtatatcttgcaaaaaaacatgaagctctaaagtgccagttctggctgggcgcgttggctcacgcctgtaatcccagcactttgggaggccgaggcgggcggctcacctgaggtcaggagtttgagaccagcctggccagcatagtaaaactccatctctactaaaaataaaaaaattagccaggtatggtggcatgtgcctgtagttccagctacttgggaggctgaggcaggagaatcgcttgaacccaggaggcagaggttgcagtgagccgagatcacaccatcgcactttagggtgggttacaagagtgaaactctatttcaaaaaaaaaaaaaaaaaaagtgccatttccagccttcaaacacagtcctgaaccctaggctctgcatgtggtattgcctctacggatattaacgtgtaagagtcctttggcagaggaatttaaggggagtggataagtattgtttttgcctctgaaagtaatggcaaaaaccacaatcacttttgcaccaacctaatataaaagaagagggaaaggatgggcacagcacaagactacatgctatggcaaccttggcccctgccactcactcaagatcattcagccaacaaatgtctgctagccagctgcatgccaggctgtgcacttgatgctcagaaataacagtcctgccctcatcgagcttgcagtctacattcaaaaccagcttgcataggtcttagggccttttgctctttctacatattgagccactataattaatcaatgatagttaataagggaaaacagatagataatatattctggttattgctaacagtccaaaaagtagtaagaatagaaaacagcttttcaaattccttttaggaaaggctgataaagccataagtgaaaatatgttttttctgggttctaattcatgagacttttgctatttgctgtgtacattatagaaaagttcacatcctgttgctcagggcaatgttgaaaaaattcatagtaaaatttatttttgttgtttatattgaggcccatagagaagtcatgtgaaagacgtagtaagcttgggcatgtgcttgcatatttgagattaaaaggttaatatatgcattattaaagttataaatgtgtaaattattttgagtagatggatgaatgcccttactcactcttttctgatacctccggttcacagagaaaaatacaatttcaaaacttaaagaaaagcattactttactgatgatggctcttagtttcagacaactgaatccattctagctagtttaagcaccaagagatttaattggcattttgaaacttgccagatactgttcccagaatgttgtcactggaaacatttaaagataaggaaacatagaagcagagaatagtaatttccagggactgggaggagagaaaaatggggagaaattaatcaaaggaaaccaagtgaaaattatgcaagatggagaaatcctagagatctacagtacagcatagtacctgcagttaaccacccactgctgtacacttaaaaatgcactaacaggccaggcacaggggtttacacctgtaatcccagtactttgggagaccaaggcagatggatcacttgaggtcaagagttcaagaccagccttgccaacatggtgaaaccccatctctagtaaaaatattatacaaaaattagccaggcgtagtggcacacacctgtagtcccagctactgaggagactgaggcaggagaatcgcttgaacctgggaggtggaggttgcagtgagccaagatggtgccactgcactacagcctgggcaacaagagcaggtgtctgtttcaaaaaaaaaaaaaaaaaaaaagtactggcagggtagaccttgtgttcttatcacaaaataataataaattaagagaaaaagcaaacttttggaggtgatggatatatttatggcatagattcctccaaacttatcaacttgtatacactaagtatatacagctttttgtatgtcagtcatatctcaataaagtggtttaaaagttaataaagatgaggaaactaagactcaggcaaattatttttatatgttcattcagcaagcattttttagccctaagtaccaatcactgtacagaaattagtattgcgtttctcctttcatgaggctcacaatctattttcccaataggttgtgcatcagtctcactccatgagctttaaaaaaatacagattctagaattattaaattagaatctctagacggggccaggcatagtggcttatgcctgtaatcccagcactttgggaggctaaggtgggcagatcacctgaggtcaagagttggagaccagcctggccaatatggtgaaaccccatctctactaaaaatacagaaattacctggccatggtggtgggtgcctgtaatcccagctacttgggaagctgaggcaggagaatcgcttgaacccaggaggcataggttgcagtgagccaagatcgcaccactgcactccagcctgggcgacagagtgagactccatctcaaaagaaaaaaaaaaaaaagaatctctagaaggcctggaaattgtggttttaatctccccaggtgattgtgacatgccagcaaggcatagaaaaccctggtgtagttgaccctgccataacaacgtagaacaactgagacacttaaggtacagaaccagggctctacactgatcatgacctccaattcagagcatctaccacaaaaccacagtgaatcttcattattggcatgcagctgactttggctcttagtctgaagtttaaaatgttgttaccattttgttatttttttatttataaatatttatcatggcaatattttataagtatcagagatgccgtttaaagactactttgaataggagacagggtttgtgttttgcaaatatttttggataagcctcttacccccaagtgagtgtagatggatccattttctcatctacaaagtgagggagttacgataaatttagattttaaaatctatgatgaggccaggcacagtggttcacacccataattccagcactttgggaggccaaggtgggcaaatcacctgagggtcaagagtttgagaacagcctggccaacaaggggaaacctcaatctctaccaaaaaaatacaaaaagtagttgggcgtggtggcgcacacctgtaatcccagctactcgggaggctgaggcaggaaaatcgcttgaacccaggaggcagaggttgcagtgagccaagatcacgccactgcactccagcctaggcaacagagtgagaatgtgtctcaaaaaaaaaaaaaaaatctatgatgaaaaatgttctaagttaccttcaaattcctcccttcctgcctgtggcgaaatggaccttgaagctctaggtcaatttgctttgctgtcattagtattttgtgtttgaggacctgccagaagatgtgtccctttaggtgatcttagtaagtgacttttctcatgtaaggatttgtgtacttttacaatttggtgatttataatttcaatcagcacactggaagctcattatctgtcatataagagtctggttatttgatctcctagtgttggttccttggttactttgcctctctgcctctgataagatggttattgatagccttcgaggctttaattatttaatcattgttgatgcaaataaaaagtcccccgaaaggacagcctttcattacaacatcttctcctttcctttcccagttgaaatgtccttgaactaccaaccagctatctcaaaaaggacatagattatagccagggtgccagtatctcaaaaatatttgagaatcgtctcctctctgtgtctcttcctaacctacagagagttaaggttgccagatttagcaaagaaaaatacaagatacccagttaaagttgagacaaagaatactttttaatataattatatcccatgcaatatttgagacatactaaaagattatttcttgattaatatgcaaatttaactgagccttctatattttctctggcaaccctacagatagtgcatatcttaatgctttagaaaatccatcttatgttttgagatctaaatttgtttgagatttgctcctaattgaaacccacaatgaaatggttgtgacagttaagactgcatttgcctgaagtatagtggatgttgcactgcccacctccgcacaactctcttttgttgtgagaatataagcatagaaacaagactttgcttgagaaacctaagctaaatagtactcttttgtaatttgtattaaatattttgcagacacacataagtaaagatagtgctataatgcattgcacagaatttatttacaccctatttagtttttttctttttgtttgggtgctgggtacatggatgggtgggtgagggtgtttgttttgccatctcctatgtcagtggttttgagcctttgctatacaatagaatcacctgagagcttcttaagctctcaatccctgtctgctttgcagaccaattaaatcagaatcactggaaatagactcgggcatcagtatttaaagctgcccaggtgactccaatgtgcagccatggctgagaaccaatgccctaaatgaaaatggcaaatttgaaaaagctcttttaactactaaattatgaccttatattttaatcctcttcaaaactgatttcatgtacttttcagtaagcatcttatggcaattacatttattattttgtattatatattctatatatgatgtataatataaagtttactttaaaaaatgatcttatgaggctttaatcttgtcactgattcagtattaatttcttcaacatctataattggttttcattttcaaatatttctttgtgatttttcttatgtatttttcatatgtatttctctctctctcagattctaagccagtgcttctcaacttgaactgctcgttaaaatcatcttaggatgctataaagaaatgcccaagcctgcctcctggaggctgtgctctagttggtcaggtgtggatcctaagcattagtgttttataaaagtacctaaggaagttctaggtttagccagagttgaaagccagtgttttcaatatgtcaagtacctatctagctgatcaactgcatgtcctggggaacttttaatacataaatataattgctctgcttaatgaaatagaaggaattttgaggttctgggagaaggagaaataagggagtggtaagtgaccaccctcttgtgtttatactctcagattcacgggtgtcacgaggaattcctgatgttggggatagccgaaggactcttaagactaaagataaagatacctagtgttttaagtgttttctctgctatgctacagtgtaaaccattctgttgccctgaaatctccagagaaggttgccacatacatgggtaagagacacacgcttacatccacaagtagtgaatacccattgtttaggcctcacccagtggaaatcctaactgggtaggttcaggttaaggccctgaacccagtaattcaagccctccccagaccattgtggtgattggccacattttgtcaccactgccttgtaggtgataaaacatccctagacttgatcatcataattcacctgattgacagctgagcaaactcctgtggggtaacaggaaaatcaatggtaaaactgagtttctgcaacaggagaaaaactagagtctgattcttattagaccttcctcaggtctacctgttccttttctcttcaaggggccatatgtaggaagaaattacctttctgaactaggacaaaagactaagaagatgatgtctacaaaaaataataaaagttcaaagcagacataatctataagatgaaagagaggaagcatggcatgcattatcaataatcataacatacccattcaaagatttcattcacctcccagttactgagcacctgctaagatacaagtactgagctaggtcctgggaattcagagtatgatattgtctcaaagaatttacagtctagaagaggagatcaatgattctattagaatagaagaatggaagcgtataccaagtaacatgcataggcccatttcataaaggattataagtgaggggagacctcacagaaaagcagaagacagaacaagggtttggaagatacaatgattaggctgtggggaaaatggaaggagtgaagggaaagcaacaacatctcttattagaatgaattgaaactctggatgggcacagaggcttaaagcagcatggtatgttgcctggattacaaatggttttataatgctgtcatgtaatgatctggaggagttagggtaggtggaagccagtggtagggtttgtgatgtgtcaaactccactcatacatcagcccagtcattcttgtattttgatgagttcctagagtggccctaaagagctagatgagaggtggccaacccaccttactagtctgaaacaaaaaacatatcatctttatttctcaggttctgctgcaaaattcttcctccagatttaccatttcttagttcatatgagctgctataacaaaataccacagacagaaatttataaacaatagaaatttatttctcacagttctggaggctgggaggtccaagaacaatgcaccagctggtaagggccaggtgtctctgcttccaagatggtgtcttattgcatcatcctctagagaggaggaacactgtatcctcacatggcggaagggacagaagggatgaaagggggtgaactccctgtgctaagtctttttataacttaatcccattcgtgagtgctccagagcccacatgatcaatcacttcctaaggaccacacctcgtaatactcttgtattgggggttaagtttcaacttgaattttgaaagagacaaaaacagtcaaaccataacattttaatatcatccttggtggtacttggttgcatttgctgccagaatttctttttattataggctcttgaattttcaacctctttggacataggtatttattttgcacaatattttgtggtagatcatacacaaccctgatttcaatttgttttgttgcctttgagacggagtttcactcttgtcgcccaggctggagtacaatggcgcgatctcagctcactgcaacctctgcctcccgggttcaagtgattttcctgtctcagcctcccaagtatctgggattacacgtgtgcatcaccactcccagctaatttttttttttttttttttttttagtagaggcaggttttctccatgttggccaggttggtttcgaactcctgacctcaggtgatccacccaccttagcctcccaaagtgctgggattacaggtgtgagcctccatgcctggccttgatttcaatttgaatgtgatttcattttgatgaaaacctgagttttcactgtgaaggagttgtattgaccaacattagtagccctcacaattccacagtcaccacacaagcacttaaccatgcattcatgttaacctctgcctgctctgccattaacttccttcatttgggaatactggctttagttgttgtgcatttaatattttattatgaggtatatagttcaaatgtacagacaagtacaatcatgtaacaagcaccagtttacttactaagcagcataatcagatcttcacattttgctgtctttgtgccagttcttttttttacaaactaaaacattgcagatacagttagtagaagccctctactatcccttcttagtctcatttccctctctctagaggtaacaccatcctgatctcagtatttattgttgtcatgagtggttttatacttttactacaaatatatgtaataacatatactattgttttatgtttttaaactttaaataattgaatcctctcagtattcaactgtgccttacatttttgttgaaaattgcctctgagatgtattgatgatgacacagaaatttagtgcatttcttttaattactacatagaattaaatttatgaatataccaaaatgtattttttactgcatacaagtacttttcactgttataaggctgcagaaaacattcttgtacacaactccatatgcacaaatatgttgtatttttattctttaatcattgttttactcatataagtggtatcactgtgtagcaatgaaaaaaactatcagtcattctaataatcaaaaacatgacaatgtaatcaacttcaaaacaatattagaatagtgtgctgatgttcttaacttactgcctttcacaaaggtcaccagcaacttgcctcaaacagacatgtaagcctaaactctgaaccatagaataatttaaagagtaatcaaccataaatatagaattggaaatgaggctaatatagctttaagaaacattgagtcaatttaaggactgatcgtactctttttttctgtttccttgacatatagctggtcaaaattgacctaatttgcaattcctcttatgtggacctagagggaactgactagtatttagccattgcacttaatatgagcttttgttaaaagtacacacattaaccatttattactctgggagggaataagtcacgtttttttaattgattttgatttcattaattactgcctcctatatacagatcaatacattaggtcctctggggcgtccaaagaaagcctcagaagaacttggaacaccacaaagaagagatgcttaggaaaagtcatcagtaggattttggcaagtaaaatggagtttttcttttttcccctagtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacgtacgttgccttttaacctgtttctgttattgtatcattcagttatgagaggcgcaaggtcaaatgtgtaaagaaaatctgtttggtgaagtggagagaaaaatgaatgctgagccatcatggtttgcaaagctgttcacgaggttcagtcagcgttagatatttgcacacaggcttcatcatgaatttcaccatatttattgaattcctactatgccttttttctgtgtaaatgttaacataaccaacatgctttctaattctccacccaccttgaaaatatcttttgtctcccaggaatattccacccactctctttttttccctatccctgactcctaccctgtttggcaacatcttgaatcaccaattggaaatttttccagattgctcctgggaataaggaagggaaaatgctttggtttgaattctacagagatgtttctcatttataactttatctctgtctgtctttgtcctttcagatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggtcagtagatctttctttctttccccccttgctgtcctcaaacaattgcttgaccttctgtttatttcatcatgattgcaaattcataatttttatcatatctttctagtaacctttacctatctaaaggtattcttcagaaacatgtaatccttaaacctgtaatctcagcactttggaaggccaaggtgggaggatcacttgagactgggagttggagaccagcctgggcaacaaagtgagacactgtctctaccaaaacaaaaacaaacaaaaacaaaaaacatagctgggcatgcctgtagtctcagctactcaagaggctgacataggaggatcatttgagccccaaagttcaaggctgtaatgagctatgatcatactacttcactccggcctgggagacagagtgagtccctgtctcaaaaaaaaaaaaaaaaaaaaaaaagctccagggatattaatagtagaaaatggtgttactactttgcagatcagtacactgcttgggcattatcttaccacttgctaagtcagtcactcaagaaacattttgttgttatggttgttgttaaggtttgtggcttctgagtccatattctaattacaagggaccatgatatgctaaattcctgtagtatattgcagcaactctgtggttttatatgtgatataagaacttaatattcattaacttattcattaatttattcatgtattctaaacagtacatgtctattaggcacctatctggctcactgtcctcatggagctcactaaccatccaatcaaggcatgagattgctaagcagaaaaaagcacatataaaaattttaattataatgataagcaaaatggaggaaaatataaggtattgtgaaataaaaatagtggatctagggccggctgtggtggcttacgcctgtaatcccagcactttggaaggctgaggtgggtggatcacctgaggtcaggagttcgagatcagcctggccaacatggtgaaaccccatatctacaaaaaatataaaaattagccaggcatggtggcatgtgcctgtagtctcagatactcaggaggctgaagcaggataatcacttgaacctgggaggtggaggttgcggtgagctgagatcgtgccactggactccagcctgggcaacagagcaagactctattggaaaaaaaaaaaaatagtggatccaggaaatgaggtttgagctgagacctgactcaggtcaagaatgagagaaaatgcatttcaggcagaaggaatagcatgtgtgaaggcactgagcaggaaaagaattgggtatggtacaaagaatgaatggaaaagcagtgttccagcagtgtgagggagcagggggcagtggcaggcgaggtgggcagaagtaggcctgcagagtcttcttaggccatttttgggtttcattctgagtacagtgaaaagctatcagcattactttttatttaggttttctttgtgcataagtcttctcacagtgctaacaaagacatacctgagacttggtaatttataaagaaaaagaggtttaatagactcacagttccatgtggctggggaggcctcacaatcatggcagaaggcaaacgagcaaagtcacatcttacatggtggcaggcaagagagagcttgtgtggggaactgcccctcgtaaaatcatcagatctcgtgagacatattcactatcatgaggacagcatgggaaagacccgcccccatgattcaattacctcctatccggtccctctcacaacacatgggaattatgggagctacaattcaagatgagatttgcgtagggacacagccaaaccatatcactttgtaagaggtcactgaagactgctgtggggggaatggatttgggtagtgggagaggcaccagaatggatgtgaggagaccaggtagaggtgacagcagctcatggtaggcaaaggtgataatggcttgaacagctgtaggtcagctggccaaaaatcaattcccttctagccagtttccaaaaaaataacttgtcaaaaacaaatcacttcccctaatgatcaattagctgagttttttattttccaagtgactgatttaccatatttattaaatctattatacaaaatcttaaagcagatttgggacagattcccacaatcattttggtgccatcaccccaagaacagagaaagggacagattcaaaggcaggggcaagggcagggtgaggagcggataagggcccagtatctgaaacaggaagcagaagtcaaactgacattcagtcaagctggactgacggagctgcaggaatgactgctgacaaccacgcaagctcacactcaaagaactcctcaggattctgcctagttgaattctcacagtcattctgcaaacttattttcaaccagctgactttcttccatcttgaactagaatggtggctgtaagaatggaaagactttggaaagacatatgatacattttgtaggtgagatcagcaaaactggctgttggattaatttagaaagaaggaggagggtgtgtcaacaacaactttaaggctgatgggttggatgtgtggggatggtgaagaggagggatggtcaagaatgacttccaggttgctgtttcaaccacctgagggatggaggtgccaattgcagacataataaagatgacagggctgggtgtggtggctcacacctgtaatcccaacactggaaggccaaggtgggtggataacctgaggtcaggggttcaagactgaccaacatggtgaaacccatatctactaaaaatacaaaataagccaggcatagtggcacatgcctgtagtcccagctactcgggaggctgaggcaggagaatcgcttgaacctgggagacagaggttgcagtgagctgagatcatgccattgcactccagcctgggtgacaagagcaaaactccatctcaaaaaataaaaataataagatgacaggaataggattgaagagtaagtttgattatgttaaatttgagatgcttgtgagatagacaagtagatatttcatgtttgtatttaatatatggtgagcccaaaagagaggtaggggctagagatatcaactgtggaatcagacctatagaaagcatttaaaccatggcaatagaccacggcacctagggaaagaatgtggaatgaaaaaagaagggagctaagatcaagctgtgtccaacattaaaggccaaatacagaaggaggaaccagcagaagagatagaaaagtatctggaaaggaaaactaggagtgtatattattacagaaacaataaattatttctatttttttttctttttttttttttttttgagacagagtctcgctctattggcaagctggagtgcagtggcgtgatctcggctcactgcaacctccgcctcccggattcaagcaattctcctgcctcagcctcctgagtagctgggactacaggtgcgtgccaccacgcccagctaatttttgtattttcagtagagacagggtttcaccatgttggccaggatggtctcgatctcttgaccttgtgatccacccgcctcggcctcccaaagtgctgggattacaggggtgagccactgtgcccagcctaggaagaaattatttcaaatcagggaataattcagtaggtggaacactgctgagaggcacagatcatcccaccatcaaatttgcattggaaaggtatttttctgctcattattaatgaagatagataaatatgaaaaacaaaatgtagcaaagatagagatctaagagaaagtaaatacatttgttgtgattggtaaataatttaaattaccaaaaagattttgtgttaagcatgtcagagtgaaagtatgctcttatctctaattgtcttaaaaatccctctaaaatgacaataaaagagggggaaatggtataaactagcaaggaggaagaaaatgagagaggagacacgtggaacaggaaaaatatatatatggcaagcagatggacaaggggcagctgaaatattagagtagaggaagctgaaatgtaggtacgtacactacaaaacactgggaaattggaggctccaagtattgtgaaagctggaagtcaggcaaaaggctaaaagcaagggaactggttcaaagtctttgcaatcagtagcatcaatacactcaggctcccacatttacacgatgcagtggatggtaggataaaaataccaagagatgtcaagaaggttaactaaaagtctgcacactgaactgtgagccagcaaaggctgttcccttctctactttgtttacttcaaagtgcctgcaaaaaaacttatagcccaaagcagatggtgacctataaaagacctatagtgacaagtgggggtcccccagtgaagatgctggctggccacccagttgtcccacaataaggcccatcagtaaacgaagagccaatacacatgaagcttcaagaaagagaccaaatcacacaaaaaagaattcataaggaacagaaataacagggcaaaataaaaaactttaaaactatagtatcctaaaagagatacaaaaacacattgaatctatgaaacaaaaacaggatgctattttgaaaagagcattcagagaacaagaaagagctcatagaaattaaaaatgtgatagctggaataaatatttagtcagggttggaagataaagtagaggaaatctcctggaagtagaagaaaaagacagtaagagagaaaacataagaacaccagaggatcaagcctgatgtcccatcatacatctaataggacttccagaaagaaagaagagagtaaacagaaggaagaaatgatcaaagagtaatccccaggaatgaaatacatgtgttttcagactgagagaaatcattaaatacccagcagaataaatgaaaaataaaacaaggcacaccatagtaaaatagaataccagggataaatagaggatctcagactagaaagaaaaaaagtcacataatgaaaagctgaaaggtaaacacaatggaaaattaagtttgcatctaaaattgataaactgagaaatcatgatacaaacatatttagaacgagttttctcaacctgatgccatttactctgttgttgggggcctgtcttgtgcagtgaagggcacttagcagcacccctggccttcacccactagatgccagtaatactcccacccggtacccacatcatgataatcaaaaatgtctccagacatccccaaatgtctcctgccagaagtgggggtgggggagcgctctcctttgagaatgatgatttaggaaaatggaggtaaatagagaaaaacagctaaacgagttggaagtgattggctccgaggagcaaaacctggaggaagaggggtgagcagaaaagagctggctcttccagagagcctttgctgcttggctttttaaatcgtatgcatacatttctttgctagaaataaatttttttaacaacattcgagtgaccatgttattatacatgaagttaggatgctttcccaactcttcttttgctaaagctgttttctccacagcataacagtgcattaaatgagttttctaattgtcccagaaatttggctgagcccccaaagctgagaataaaaagggcttattgcctacacattctgaagagggatgccagtcaatctatcccaagctatgaatgaatgacccaggccctcttgagtgtgtcactttaacagaatatatgagggaacgaggagactgatttggaatgagaattttccagggagaggttaaaaccctatccatttcccctccacctgtagtcaagtaagaaaggactacaagaaagctgctcagcgtgctgaacagtaccccctggagtttgtgaagactgctgcctaattcttgcatgaaaaagtccagctgctctaaaaactagtttttaaaaaactttcaagtgaacctgcacatggatgtttatagcagctttattcataattgccaaaccttggaagcaatcaagatggccttcagtagatgaatggataaactgtggtacatccaaacaatgaaatattcaatattaaaaaggaatgagctatcaagccatgaaaagacatgaaggaaccttaaatgcttattacttagtgaaagaagtcagcctggtgtagcttaatactgtatgatcccaactgaatgacattctaaaaaaggccaaactggccaggcacagtgacttatgcctgtaatcccagcactttgggaggctgaggtgggcagatcgcctgagcctaggagttcaagaccagcctgggaaacatggggaaactctgtctctacaaaaattagccaggtgtgatggtacacacctgtagtcccaactactagggaggctgaggtgggaggattgcttgagcccgggaggttgtggctgcattgagccatgaacatgcccctgtactccagcctgggtgacagagcaagaccctgtcttaaaaaaaaaaaaaggggggggcaaaacttcaaagacagtaaaaatatcattagtgtccaggggatggggggagggaagtatgaatagagcacagagaatttctgagcagtgaaactactttatatcacactatcatgatggctacctgtcattgtacatttgcatagactgtaccacaccaagagtaaacctcatgtaaactatggactttgggtgatagtgataggtcaatgtaggttcatcacttgtaacagatgtgccactctggtgggggatgttgatagtgggggatgttgatagtgggggaggctgtgcatacataagggcagggaatgtatggaaaacctctgtatcttttgctcaatttgctgtgaacctaaactgctctaaaaattctatttaaaaaaaaatgtctaaaggatgatgtgaactgcctaggaccgcagagtgagggaaagagtcccctagagggtgtgacctgcacttccaaagtttcttgggagtaaaatgtcaagctttcccattgagtaactggaaggtaatgagacttggcccatttgataggtacctgcctaagaagactgcctacaggatgagcatacaccagccaaaagaacgtggaaggctgtaccttctgataagagtcggaggagggatatctaagagtcaggaggagggagcattacataggttagggcatagtaactgagagtcccttattgcaaaggggtctccagagaacccacaaaaacaccccattggagaacgagctcgcatttgtgagtcaacaccaaattacaatgacaaccactaagataagacttttttctgcttttatctaatttgtgaaagaataaagcagaaaaaaaaatcacccactggcaaaaaggagatgacaatgcaaagtttctcagcttggactctatcagcaccaaggtaagatgatgtgcaggactggggtagggcaggttgcagcttaaattaagatgggcttcactcgttcatcagaaataaagaaccaagagaaaaacacatgtaacacctctgtcttctaatgatcccccatttctgtttggtttttaatcccacaggcttagcctttgcaatgctggcagctgtgcctccaatatttggcctgtactcttcattttaccctgttatcatgtattgttttcttggaacctccagacacatatccataggtaaaagcttacatttgatattctattacctttccttgtgtatttccaaccttgcaatgtgccaaagaaatagcatggtcattattaaaaattgtcttaataacaacttttgttactgcatacacaataggtgctttaaaaacctacactcggccaggtgcggtggctcatccctgtaatcccagcactctgggaggccgaggcgggcagatcgtctgaggtcagtagttcgagaccagccagaccaacatagtgaaaccccatctctttgtaaaaatacaaaaatttgccaggcttggtgaggggtgactgtaatcccagctactcaggaggctgaggcaggagaatcgcttcaacctgggaggcagagattgcagtgagctgagatcacaccactgcactccaacctgggcaacagaacgagactctgtctcaaaaaaaaaaaaaacaaaaaaaaaactacactcctcccaagatttctagaacttacaattttcttataatcttgggataataaaaaagacaattttcttgtgtaaaagggcaattgttgaaatctgtgaaatgagataaatgcttgatttcataatgtaatttgccagcttctggtttttatttttatactaatggtagtacttcaagttcctaatcgctatgcatgtatttcatttttgtcatattttgggctctgaattgtacatattttcagtttatcaggggcatatggatttcataatagttctggtgctgcataaggctactacctaacacacacatactttccagatatctactgtttggtaacaaatacccccaaaatgtagcagctttacaacaacaatcatgtttgtatcttccatagtgcctgcagttcaggacttgggaagggctggtctgggtaattcttcctctctccctttttcctccctcccttccctcttccctgccttcctttctccatgcagtcccaggaattctccacttggcctagttggagctttcttacacatggtgacttagggcttcagcagaaggtggaaactgcagcccttttattacctagcctgagaagtcacacaacgtcacttctgctgcattcttttgattactaacaagtcaaagccaagccagattcaaggagagagaaattagactccacctcttgatggggaaatgtcaagaagactctgtggatggaagatattgctatgtctatttttggaaaatacagtctgcatgcgcacacacgcacacacacggttgctcatacaccaggggcaggacactaaagaagggagcgtagccttccttcaagcatgatttcttgacctcacctttgtctcttggtgtactggcaaaaaccatatcagatactctgaaccctgaacagggatcttcatgcttcattatttcaggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcaggtaaggctcaaagatggggaaatggactgtctcgtttgttaacctaacaagtatctactgagtgctcttggctggtcttactacacggtggatggggtcagccagaaaaaccccagagctctgggagtaactgcagaccaactgcatgccatattcacaactatgagtgacgagcaagcaagcttcaggtgggtgttttaaagatcccaaaagcaagacaagtttgacacggcaggattggttagtgctgggagaggacattcctctctgcttggaagaaacagccagtgatcatgatcatgcactttagtctacatgatgaagcatgcccccagctcaatgagagacagggaaggcattagtgtggtcagcgcactgtggggcaccaagccactccagaccttccattgttttctgtataaccagtggttgattttctagctacttacccacttgaggtacaagtttgcagtgttagaaaatggtccagaggctgggcgcggtggctcacacctttaatcccagcactttgggaggctgaggcaggcgaatcacaaggtcaggagttcgagaccaccctgggcaacatagtgaaaccccgtgtctactaaaaatacaaaaattagccggacatggtggcacacacctttagtcccagctactcaggaggctgaggcaggagaatcgcttgaacccgggaggcagaggttgcagtgagctgagactgtgccactgcctgggtgacagagcaagactccatctcaaaaaaaaaagaaaatggtcaagaaagaacttgtatatttgctgtgggtcataacttcctgacctatcatgtgattcatttgtttctttgtcattaaaaaaaaatagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatgtaagtaagaaactacctgacgttctggtacttttctctttattccccccaaccttctcttctgtatctctttttactttcaagactcatttttctttttctccagtaatttccataaaaagggaaaaattcatttattctaaggttttctgaaaagagtgttacaaagagaactaaaccttaaaatctctgttattaatatgtgatactgcctacttcacaaggaattttttgctaggtctttatttggtgactgcaccatcactctaggtaaagagagagctttttcttacaactaaatcatgatctttttggaaactaacaaataatttaaaggcatgaaggtagaacatttctctctcaaaaactgctaattaaggccgggcgtggtggcttatgcctgtaatcccagcactttgaaagggctagatgggcggatcacgaggtcaggagatcgagactatcctggctaacaagtgaaaccccgtctctactaaaaatagaaaaaaattagccaggtgtggtggcgggcacctgtgatcccagctactcaggaggctgagacaggagaatggcgtgaacccgggaggtggagcttgtagtgacccgagatcccatcactgcactccagcctgagcgacagagcaagactctgtctcaaaaaaaaaacaaaacctgctagttcaaactggcaaaaaataagtaaggctataaaagatttaactaacatgaatcttttaattatatagaccccttaccaaacacagagaaaatataaattatttgcaagcacttaaggaacaatggtaataactgatcatgttgaaatcgatcatgtgaaactgatcacaaattctaaaaattcaatgttttttcacacagtgcaatataataaaaaaatcaaaaactaaacatagctttaaaactcccatatatttagaaaccaaaaaacatttctaaatgacccatgaataaagagggaattaggaaataattagaattaattgacagtagaagtactacatatcaaaacctctgagatgctgttaaagctatccttgaaaagagaggcctgttgcctttgtaggtatttatgtgccagacactgctaggtgccaagatgcagctgtgagctaagaagatacaggccttgccctctggagctttcagtacagtgggaagattgtaaaccattaatcgcatacatgtgtagtcaattacaatttgtgttgagtgctatgaagaagaagtaccagctgctctgaaagttaaatagggagacctttagattaggggtctaggaaaggcactttgacatttaagttgagtccctgactgataaaaatggaatcaggctttaggaatattctctaaataacaaaattcttagtttcccataatagctattatcatacttcttactctgagacttgtcgaaatgccttgtcacaaaggaaataaaacatactgagtactctaggcatcgataggaaagggtgcatgttgtgggctgagtgggaagtctactttattgggtttcaaatgagaactgactatatttgaggtgcccagttggtaaccctaggatataattaagtggatttgttctctagttctcctaggttcctttcccaaacatcagcactcttgacttcaaagtcctgctaacaataatttctctctctttgagctcctaaaagcttttcaatgtatttacctttggagtactttgctatcaacccttaattctttccaattgctcaggactcagcttcagggttccttttctggattttataatattacattgagacatcagcatatgataagaataattgtttgataagaataactataaggctgggcacagtggctcacacctgtaatcccagaactttgggaggccgaggcaggcagatgacttgaggtcaggagttcgagaccagcctgaccaacatggtgaaactccgtgtctactaaaaatacaaaaattagctgggtgtggcggcaggcacctgtaatcccagctactcaggaggctgaggcaggagaattgcttgaacctgggaggcagaggttgcagtgagccaagatcatgccactgcactgcagcctgggcaacagagcaagactgtgtctcaaaaataaataaataaataaataacaactataaaaagaaaagagtaacaactataaagttgtagactgggtaaaaatgcctcctaattggtagaagctacaaagagagatttcagccttggaactttccactagaaatatccaataacggaatggctggtgtgaattagtgcgctagagtgtctctgtctctctcaggggcgatttcttgatgtaaacatccagcattcactgtgttacagagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtaagtcaccttcggtcagtgacctactcggcttctacaactgcatttacttatttctgtgagggattttgaagtggatgaaaggaattttttgctctcaattctgccaaaagaaggttacaatttgtcaaatttgacaaattcattgacaagaaagtaaaaagaaaaagccttaaaacatagacacagatgattttttaaattatatgtaagactctgcatgaaaattttaacattaattacaagtgatgggattatggaggattttctcctcaaatttttattggctaattgttttgtaatgagtacatattactttaataacctaaaaaagaaatccatttccattttgacaaagcaaaggttcccaaactttctccatcccttgacactcctgagccaaaagaaatacctcacagttccacttattaagtagttaggtccactttaataatatttatgtttaacaacttcgtagttgtttataaaaatatgcacataaattggaataaaaatattttattatgttcttaagtaaccacaattatgtaatcatggaatgcatatgcctgatgggaactgcttagatcctcaaacctctgaatcaaaaaggacaatgctaccttcatttcctgttcacatgaatatgtatgtggttcctgctttttcatcacagcaaccactgaacacccagctttgcaaagatatggcattattgaaaggaacacagtgttcaaactgtgaactattgccaactagaagtttgcacagtgcccaatagatgtcactgtttcactggggaccttttaatatccaaagtgtcccacagagtgcccttagcaccctggggcttgtttgcacgtgtgtgggagcggcaaggataagaaatgaagagtcgtctaggtgtggtgactccacatctgtaatcctagcactttgggaggccaaggcgggcagatcacttgaggccaggagttcgaggccagcctggctaacatggtgaaaccccgtctctactaaaaatacaaaaaattagatgtggtgaaatgcacctgtaatcccagctacttgggagactgaggcatgagaattgcttgaacctgggaggcagaggttgcagtgagccaagatcacacctctgcactccagcctgggtgacaaagcaagattttgtctcaaaagaaatgaagagtacatttgggggtaaagggctggcaagatggccgaataggaacagctctggtctgcagctcccttcgagatcaacacagaaggtgggtgatttctgcatttccacctgaggaatccggctcacctcattgggcatggttagacaatggatgcagcccacagagggcaagctgaaccagggtggggcatcacttcacccaggaagcgcaaggggtcagggaattccctcccttagccaagggaagctgtgaggtactgtgctgtgaggaacagtgcattctggcccagacactatgcttttctcatggtcttcacaacccacagactaggagactcccctgggtgcctacaccaccaagattcccttggatttcaagcacaaaactgggcagctatttgggcagacactgagctagctgcaggagtttttttcataccccagtggtgcctggaacaccagtgagacagagccattcactcccttggaaagggggctaaaggcagggagccaagtggtctagctcagcggatcccacccctgcagagcccagaaagctaagctccattggcttgaaattctcactgccaccacagcagtctgaagtcaacctgggacgcttgagcttggtggggggaggggcatctgccattactgaggcttcagtaggcggttttcccctcacagtgtaaacaaagccacctggaaggtccaactgggtggattccaccgcagctcagcaaagctgctgtagccagactgcttctctagattcctcctctctgggcagggcatctctgaaagaaaggcagcagccacaatcaggggcttatagataaaactcccatctccctgggacagagaacctgggggaaggggcagctgtgggcgcagcttcagcaaacttaaacattccggtctgccagctctgaagagagcaccagatctcctagcacagcattcgagctctgctaagggtcagactgccttctcaagtgggacccccgtaactcctgactgggagacacctcccagtaggggccaaagacacctcatccaggggatctctggctggcatatggcgggtgcccctctgggacaaagcttctggaggaaggaacaggtagcaatcattgctgttctgcagccttcgctggtgatacccaggcaaacagggtctggaatggacgtcagcaaactccagcacacctgtagcagaggggcctgactgttagaaggaaaactaacaaacagaaaggaatagcatcaacatcaacagaaaggacgtcaacataaaaaccccatccgaaggtcaccaagagcaaagaccaaaggtagataaatccatgaagatgaggaaaaaccaatgcaaaaagctgaaaattccaaaagccagaacacctcttctccttcaaaggatcacaactcttcaccagcaagggaacaaaagtggatggagaatgagtttgacgaatggacagaagtaagcttcagaaggtgggtaataacaaactcctccgagctaaaggaacatattctaacccaatgcaaggaagctaagaaccttgaaaaaaggttagaggaattgctaactagaataaccagtttagagaagaacataaatgacttgatggagctgaaaaacacagcacaagaatttcatgaagcatacacaagtataaatagtcaaatcaaacagaagaaagggtatcagagattgaagatcaacttaatgaaatatagtgtaaagacaagattagaggaaaaaaaaagaatgaaaaggaatgaacaaagcctccaggaaatatgggactgtgtgaaaagaccaaacctacgtttgactggtgtacctgaaagtgacggggagaatggaaccaagttggaaaacactcttcaggacattatccaggagaacttccacaacctagcaagacaggccaacagtcaaattcaggaaatacagagaacaccacaaagatactcctcaagaagagcaaccccaagacacataatcatcagattcaccaaggttgaaatgaaggaaaaaatgttaagggcagccagagagaaaggtcgggttacccacaaagggaaccccatcagactaatagcggatctatctgcagacaccctacaagccagaagacagtgggagccaatattcaacattcttaaagaaaattttcaaaccagaatttcacatccagccaaactaagcttcataagcaaaggataaatgaaatcctttacagacaagcaaatgctgagagattttgtcaccaccaggcctgccttacaagagctcctgagaaagcactaaatgtggaaaggaaaacccggtaccagccactgcaaaaacaaatgaaaatgtaaaaagcatcgatactgtgaagaaactgcatcaactaatgggcaaaataaccagctagcatcataatgacaggatcaaattcacacataacaatattttttttttttgagacaaagtcttgctcttgtcccccaggctggagtgcaatggtgcgatctcggttcactgcaacttccgcttcccgggttcaagcaattctcctgcttcagcctcctgagtagctgggattacaggtgcctgccaccacgcccagctagtttttgtatttttagcagagactgggtttcaccatgttggccaggctggtctcgaactcctgacctcaggtgatctgcctgccttagcctcccaaagtgctgggattacaggcgtgagccaccgcacctggcccacgcataacaatattaaccttaaatgtaagagggctaaatgccccaactgaaagacacagattggcaaattagataaagagtcaaaacccatcagtgtgctgtattcgggagactcatctcatatacaaagacacacatagtctcaaaataaagggatggaggaagatataccaagcaaatgaaaagcaaaaaaaaaaaaaaaagcaggggattgcagttctagtctctgataaaacagactttaaaccaacaaagatcaaaaaagacaagggcattacataatgataaagagatcaatgcagcaagaagaggtaactaccctaaatatatatgcacccaatacaggagcacccagattcataaaccaagtccttagagacctacaaagagacttagactcccacacaataatagtgggagactttaacaccccactgtcaatatgagacaggtcaatgacacagaaaattagcaagcatattcaggacttgaactcagctctggaccaagcaaacctaatagacatctacagaactcttcaccccaaatcaacagaatattcattcttctcagcaccacatcactctgattctaaaattgacctcataattggaagtaaaacacttagcaaatacaaaagaatggaaatcataacaaacagtctgtcagaccacagtgcagtcaaattagaactcacgatcaagaaactcactcaaaactgcataactacatggaaactgaaaaacctgctcctgaatgactactgggtaaataacaatattaaggcagaaataaataagttatttgaaaccaatgagaacaaagacacaacgaccagaatctctgggacacagctaaaacagtgtttagagggaaatttatagcactaaatgcccacaggagaaagtgggaaaatctaaaatcaacaccctaacatcacaattaaaataacttgagaaggaagagcaaacaaatccaaaagctggcagaagacaagaaataactaagatcagagcagaactgaaggagatagagacaagaaaaacccttcaaaaaaatcaatgaatccaggagctggttttctgaaaagattaacaaaatagatagactattagccagactaataagaagaatcaaataggcacagtaaaaaatgagaaaggggatatcaccactgctcccacagaaatacaaactactggctgggcacggtggctcacgcctgtaatccgagcactttgggaggccgaggcaggcaaatcagaaagtcaagagatcaagaccatattggccaacatggtgaaatcccctctctattaaaaataaaaaaaattagccagacatggtggcacatgcctgtagtcccagctacttgggaggttgaggcaggagaatcacttgaatctgggaggcagaggttgcagtgagccaagatcacactagtgcactccagcctgggcaacagagcgagaatgagactctgtctcaaaaaaaaaaaaaaaaaagagcaatacgtacaaactaccatcagagaatactataaacacctctatgcaaattaactagaaaatctagaagaaatggataaattcctggacacatacaccctcccaagactaaaccagggggaacttgaatccctgaatagaccaataacaggctctgaaattgaggcagcaattaatagcctaccaccaaaaaaaagtccaggaccaaatggattcacagccgaattctaccagaggtacaaagagaagctggtaccattccttctgaaactattccaaacaatagaaaaagagggaatcctccctaactgattttatgatgccagcatcatcctgctaccaaaacctggcagagacacaacaacaaaaaaaagaaaatttcaggccagcatccctgatgaatatcgatgcgaaaatcctcaataaaatactggcaaactgaatccagcagcacattaaaaagcttgtccaccatgatcaagtcagcttcatccctgggatgcaaggctggttcaacatacacaaatcaataaacatagtccatcacataaacagaaccaatgacaaaaaccacttgattatctcaatagatgcagaaaaggccttcaaggaaattcaacagcccttcaagctaaaaactctcaaatgaggtattgatggaacatatcaaaataataagagctatttatgacaaacccacagccaatatcatactgaataggcaaaaactggaagcattccctttgaaaaccagcacaagaaaaggatgccctctctcaccactcctattcaacatagtattggaagttctggccaggacaatcaggcaagagaaagaaataaagggtattcaaataggaagagaggaagtcaaattgtctgtttgcaggtgacattattttatatttagaaaaacccttcgtatcagccccaaatctccttaagctgataagcaacttcagcaaagtatcgggatacaaaatcaatgtgcaaaaatcacaagcattcctatacatcaaaaatagagagccaaatcatgagcaaactcccattcacaattgctccaaagagaagaaaatgtctatgaatacaacttataagggatgtgaaggacctcttcaaggagaactacaaaccactgctcaaggaaatcagagaggacacaaacaaatggaaaaacattccatgctcatggataggaagaatcaaaatcgtgaaaatggtcatactgcccaaagtaatttatagaatttatagattcaatgctatccccatcaagctaccactgactttcttcacagaattagaaaaaactactttaaatttcacatgggaccaaaaaagggcccgcatagccaagacaatcctaagcaaaaagaacaaagctggatgcaacacgttacctgacttcaaactatactacaaggctacagtaaccaaaacagcatggtactggtaccaaaacagatatatagacctgtggaacagaacagaggcctcagaaataacaccacacatctacaaccatctgatctttgacaaacttggcaaaaacaagcatggggaaaggattccctatttaataaatggtgttgggaaaactggctagccatatgtggaaaactgcacccctttcttacaccttatacaaaaattaactcaagatggattaaagacttaaatgttaaggcctaaaaccgtaaaaaccctggaagaaaacctaggcagtaccattcaggacataggcatgggcaaagacttcatgactaaaacaccaaaagcaatggcaacaaaagccaaattgacaaatgggatctaattaaactaaagagcttctgcacagcaaaagaaactaccgtcagagtgaacaggcaacctacagaatgggagaaaatttttgcaatctgtccatctgacaaagggctaatatccagaatctacaaggaacttaaacaaatttacaagaaaaaaacaaccctatgaaaaagtgggcaaaggctatgaacagacacttttcaaaagaagacgtttatgcagccaacaaacatatggaaaaaagctcatcatcactggccattagagaaatgcaaatcaaaaccacaacgggataccatctcatgccagttagactggtgatcatttaaaaatcaggaaacaacagatgctggagagggtgtggagaaataggaatgcttttacactgttggtgggagtgtaaattagttcaaccattgtggaagacagtgtggtgattcctcaaggatttagaactagaaataccatttgacccagcaatcccgttactgggtatatacccaaaggattataaatcatgccactataaagacacatccacacatgtgtttattgcagcactattcacaatagcaaagacttggaaccaacccaaatgcccatcaatgatagactggataaagaaaatgtggcacatatacaccatggaatactatgcagccataaaaaaggatgagttcatgtcctttgcagggacatggatgaagctggaaaccatcattctcagcaaactaacacaggaacagaaaaccaaacaccacatgttctcacttgcaagtgggagttgaacaatgagaacacatggacacaggcaggggaacatcacacactggggcctgtccatgggtcaggggctgaggagggatagcattaggagaaatacctaatgtagatgatggttgatgggtgcagcaaaccaccatggcacttgtgtatgtatgtaacctgcacattctgcacatgtatcccagaacttaaagtataataaatttaaaaatttaaaaaagaagtgaagagtataggtgtctgatccaaattataacaacaagttctagctgtggaaacaactggctggcgtagctcacaaggaagcaataccttcaaaccttttcttttcctcagtcttgtctacatggcattagaaccttttaacaggcaaaacaaaacgaaatgtatctctaacagtttcaaaacctcttctctcactggggctaggcacagagttcccattttgacttggagtacagcaagaggttctgagcacctttgcgtgggaaagaagtcaggcaagccccaattatgaagtaaatgaccagcttgcctctcctgctttgggttactaattctgtttcccagattccctttaaagactcttccatgtttcccaggggaatggtttcccagaattctagaaagtttagggacagcatctttatgactccctttaagtgtcaaatttattttttattataaaaatattttttgagttttgcattcaattttataatacatctgtttgtcttgataagaaaacaatggtttaaattacttaaccactgtataaaattatagctccaggagcttgagccatatatattctgtggatttgaatactctaggcactcccctagttagagatgcacaaaaaatgcaattaagggtgatgaaaataattgtcatcaacagaaaaggatcctcccatcaaaagagccagaatcacccatgcccctggtctcccaggctgctgtttctgataataagtttattatttgtttcctgcagtggccctcctcaggaggggctggcttccttccctggccagctctaatttcttctctgttgggtaactcactcagaaagctctataggcaatagttcctgggcgtggagcaaattgctatctcaccttttgttttgtttctcttgcaactcattagggattgtactttacaccccaagtaaatctatgccctgccaatccaggatgggcgtatgtaaagtgaccctctgtttgatgtacccttctaccctgactttgtagctgaaatgagaggagacagctatgggttgctgctggggccctatcaccacacttagggctgctgtcccagaaacaggatcaccaggaggattctaaatccagatgtacattttgtaatcatacagtgtcattgtaccaccctcgaagtacagagttaaatatcctttattcataacattgtggccagagactcatttcaccaccctaaaggtaaggtcaacgtttgaaaggctttatggcctttttctgtagcagtcaactagccagacgccttagtatagaaaaggacaaagcatgaagaaaatgccaggtcataaactctgtagagttcattttaattatgtggggtagggtcattaagagaagttagaattgttgaagctaatggtaaatcttctttttaaatctgaatgttaaaagtgagaatagatttgacttgactaacatattatttgaagctgatttttgcagtatttgaacattcatctctaaaggtgcatttggatttgtgtctttttaggtcgtaatgggaactggcatttcagctgggtttaacttgaaagaatcatacaatgtggatgtcgttggaacacttcctctagggtaggaaagtggttttaagtaactgtgtgactgaaataatcaatggatctattgcctctaactcttacattagttctcttgcaatgaaaaataatcttttttctatacatcaaatagctgagggctttagggggattgggtgcattttatttttatttttattgtagtaaaaaacacataacaaatttttgtcttaaccttttttttttttttttgagacgaagtctcactctgtcgcctgggctggagtgcagtggcgccttctctgctcactgcaacctccgtctcccaggttgaagcgattctgctgcctcagcctcccaagtagctgggattacaggcgtatgctaccacacctggctaatttttttttttttttttttttttttttgagatggagtcttgctctgtcccccaggctggagtgcagtggcatgatctcggctcactgcaagctccgcctcctgggttcatgccattctcctccctcagcctcccgagcagctgggactacaggcacccgccaccacgcccggctgattttttgtatttttaatagagacggggtttcaccatgttagccaggatggtctcaatttcctgacctcgtgatccacccgccttggccttccaaagtgctgggattacaggcatgagccaccacacccggctaatttttatatttttagtagagacagggtttctccatgttggccaggctggtctcaaactcctgacctcaggtgatccgcccgcctcagcttcccaaagtgctggagtgctgggattacaggtgtgagccactgtgcttggctttttttttttttctttttttttgagaccgagtctcactctgttgcccaggctggagtgcaatggcacaatctcggctcactgcaacctctgcctctgaggttcaagtgattctcctgcctgagcctcctgagtagctgggattacaggtgtgtgccaccacatccaactaatttttatatttttagtagagacagggtttcactatgttggccaggctggtctcgaactcctgacctcatgtgatccgcccgcctcagcttcccaaagtgctgggattataggcatgagccaccacgcccagcaaatcttaatcatttttaagtgtacagttcagtagtgttaaatatatccccattattgtaaagcgtatctccaggactttttcaccttgcaaatctgtaactctccaccccttaagcaagagcttcctcttttcttcttcccccagcccccagtaaccaccgttcatctttctgtttttatgaatttgacttctctagatgcctcatgtaagtggaatcatacagcatttgtcttttcgtgactggcgtatttcacttagcataatgtcttcagagtttgtctatattgtagcatatgacagtattttcttcccttcttaaggccgaataatattccattgtggatatacactgtattttatttctccatgcatccactgatgagcacgtgggttgcctttacctcttggctattgtgaatagtgcagctgtggatatgggtgtgcaaatatgtctttgagacctttctttcagttcttttgggtatatacctgtaagtgggattgctggatctgtttttacttttttgagagcctttcatactgtttcccatagtagttatgccattttacagtcccacccatagtggcaagtggttttctgttttcttttgttttcctaaatttaatagttttaaaaatctctccttgaatgacatgggatttaagagcaaaacaacctaaatcctagcctgccacgtactagctgtgtgatcttaagcaggccacttatcctctcaaagcctccatttccttacctgccaggtggccatggtaattaatacttatctcaagggcagttgtgagagctacaggagataatgcatggaaggcaatgagtgaacacctgaagacacagtaagcattcaagtcgtgttaactacttattagtaatgggttctaaggctgttatttcagtgagtttttagtttctaaccatgttacttctttgcttcatctatattatctaactgtacatttttaaaacatattattggccgggcacggtggcagactcctgtaatcccagcactttaggaggctgaggctggtggatcacaaggtcaggagttccagaccagcctggcaaacatggtgaaaccccatctctagtaaaaatacaaaaattagccgggcatggtggcgggtgcctgtaatcccactattggaaggccaagcaggagaatcgcttgaaaccagaaggcggaggttgcagtgagtcaagatcacgccatggcattctagcctgggcaacaagagcgaaacaccatctcaaaaaaaaaaaaaaaaatttatatatatatatatatatacatacatatatatgcgtatatatatatgtatatatatatataaatctacttgtcactataatttctacttaatatcttcctttccctattgcattgtactcaaacatactgcttcccttctgtatgtgacagcttaactaaaaaaaaaaaaaaaaaaagaccttacgtttctggaaacaggctatccagaggagaccttatctttctggaacattgcccaaaaccaagtaggtaacaaatacctacctacctatctacatacaatcataccatttcagttccctgtgagtcttattgctccactgataacaggctaacccaaaacagtgacttaaaacagccaccattaatttgctcataattctttagtgtaggttaggctcagttgggtggttctttggcaggtctcacttggggtttgttatgcatctgcagatgtctgcaggtgtgactagtgttggaggatccaagagggcttcactcacgtgtctgacacttcatctggagtcactggaacaggtgagggctagcaggcatcttctctacatgtggtatctccagcagcattgccagacctctttacatggtggctcagagcaacaggaaagaaaagcagagaatttaccaggcctcttaaggcactcatacagcatcacttccaccacattctgttgctcgaagcacatcacgaggccagcccaaatatagctggaggggaaacagattccccatctagatttggggagtgacatgcacatacaagtatgggaggaatgactggtggcctatttggaggcaaactaccatgcttctatatacacgtacctacacatataaatgcatctgctgtagacaagcaggttcttcatggttttgtacttggtaaatgagcagtgcattttgcggtgcccagaaagtgacagttgactattaaagatcccaatcatagcactatatcctgttttcttgtttgtttgtttactttccttaacttgttccaaaatatgtccttttcttatattcttattcccttaggcccaaacctgctgctaggccttccctcttagttcatgcctgtgcagcaaagagaagggaaggacaacaggttttatagccagaccccccaggtttgactccctgctacagtttaggaaaatcacttaacatctgtcaaaatccattttttcacctacaaattgggataatagtagaacctatgtaatgggagtgctgtgagggaaaatgagacgacttatgtaaagcatgtagcaggtaaactcaccttaacaggcattaggttcctttccctttcttcctctgtttccttacttcacgatgtgaaacatcccttccgtcactgtgctatgacatagtaagagaaatagctgtggccaagcaggttctttatggttttgttgtacttggtagatgagctgcttgtccttggacaagttgctgaaccttgctatacctcattttcttcatctgtaaaatgggaattacagtggtgcctagttcacagggccattgaaaacaattaaatgagcatagaacctggcacagagaaagcatataataaatgcttgttattgtaataattataattattagtatcagttaagttattgttattattgtttagcccaatgccttgaattggaggagaatgtctgcaaatccctattgccccttcccccataacattctagggaatccctattctgtttctttaggataaggatcccagactacaagtatgtaccacaaaagctgggttctccattgtgggattccaggagtcacacttttatctcctccagattcacaagattcccaggtgacacccaaggagtatagtcttctgctgctcctttcccatgttacaacctcagctaatgggataaaacacacacacacacacacacacacacacaggaagaggtagcacactcagagtccttacatcagatgacaagggggagaagtatggggtgcctagcattgctaggacattaattctcctctaaggtagcccttagactactctcctccccaccccaagacttgggttcagatgcctggtggcatctgagcactttgctgaccctcgcttcttcctggtttctgtgtcttacttacacttgccatcagggtgccatgaggtatgaggttactccttttataaataaaacggcatctgatctcttctgataacccatccaggtagaaagctgcactttgcagtttgagcctgaaaagaatttctctgcgtcaaggagggccctcctttttgccaaccagagcaaataagtatccaagcattgattacttcatttgcagataatgcccccctaaaatgcttccctgcagtcgcaaggttttttttcttttcccttgtggtcacaacacaagttaaaggcagcttgacttttcaggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggtaaagataatcagtttcctgcacatgtttcatagcagtacaatcaccaatgtctggctcacgcctgtaatcccagcacttcgggaggccaaggcaggcggatcatgaggtcaggagattgagaccatcctggctaacatggtgaaaccccatctctactgaaaatacaaaaaattagccgggcatggtggcaggcccctgtagtcccagctactcgggaggctgaggcaggagaatggcatgaacccaggaggcggagcttgcagtgagccgaggtcgcgccgctgcactccagcctgggcgacagagcgagactctgcctcagaaaaaaaataaataaataaccaaggtcggttatggaaggcacaagaactaatctatcttggtagaaaaactttttgtactggtgattgatgtatctgaagcatgtacttattcagtctgcaatgttactcctgtttttaaagctcacagcttaggattaaatgtttttccagggcgtttttgctccctaggaaggtttatctttgttcattaaatattgggtatctgattcatcactaactccacaatattctctttgtcacctacttttataacgcagccacagataaggtatttaatccaaacactgtgacctataatgttgacataacaatgtctatttcaatccttatgtattctaaggagaaactagatgctccacattcatctgagatgcagttgtcaaactagaaatatgcaaagtaaacctaagagcaggactcacttgccccaggaagacactggcatgtactcatttgtgagcaatagcccaggcattaaaaagatgccaagagtcctatacaacaataactttctcttcagcagtaaaaatagttgatcccttttagtgcagttttcttcttaagcgttatttatgtagagggtgtgtttggaaaggaggctgagcagggagcccagcgtttatctctgacaaaataaaaattcaaaatgttcccccctctcactataaaatatgccataattcctattttgaactcatgctgaacaaattcttacatctgaaaggaaaatgaagagataggctgtagtgctgacaatgtgtattaaatgggctattatgcttttttttcctgaagaatgacctatctcttttgtgttttgtttctacatgggaaaggagctcattgccctgggactgtgcaattccattggctcactcttccagaccttttcaatttcatgctccttgtctcgaagccttgttcaggagggaaccggtgggaagacacaggtatgtacataacaggcctctaagacctgatgatatgggtggtaattaaattttaatcttttgattatattctgaacccaaacaaagctactctggcctgcagatccatgctccagtgagctgtgtagggaatcagagaaaggcatgattggttgaagtgaatcacaaggaactgaatctataggaaagaggtatattctccagttacattggaaagttatgcaacactcggctaccagtaaacttcaaagttcagacatagaatattgttatgaaagcctgagactgagtatgggcagtatgccatcaccatccagtagaactttccgcaatgatggaaatgttcaatatctgcactgtccaatatggcagccactagcaacatgtggctcttgagcacttgaaatgtggttagttcgaccaagacctgacttttaatttttacaaatgtaaatggccggccaagtgcggtagctcatgcctgtaatcgcagcactctgggaggccaaggcgggcagatcatgaggtcaagagatcgagaccatcctgtccaacctggtgaaccctgtctctactaaaaatacaaaaattagctgggcatggtggcggatgcctgtagtcccagctacttgggaggctgaggcgggagaatcgcttgaacccaggaggcagaggttgcagtgagccagggtcgcgccactgcactccatcctggcgacagagtgagagtctgtctcaaaaaaaaaaaaaaaaaaagtaaatggccacatgtggctactggctcccatattggacagcagacacttatctgaactcttctggtggcccttgtaggtctcaggttggcaccagacatctttcctccacctctggaaagggcattttaattaggatattacaacatttcagcttccatttaaaagccataagaagaaccaaaacttcagagagggagtgtgatggagctataggttatgtagtggtttagttgacttatttgaatccatattttcttgatcttcccatagtaattatcaaaagaataaaaccaacttatgccctgcctcttattcaagtctcctaaaagccccttccatgtgtcttctctcattcagcttgcaggttgtttggcctcattaatgattctgctggtcatattagcaactggattcctctttgaatcattgccccaggtgtgtgtaatgtggacttgggtgaagtttttttcatgtgtagagattttgctattttatttgtttatgttcttatggctatattagggttatttactgtaaccctcacaggatggtatgagaaagggccaaggctaggaccaagatcctgctctataacctactgacagctgcctgcaggacctggggaaagccatcttaactcctctgagtctcggtttccttatctgcaaagtcggggtttggactggctgatcttgaacatcccttctctctttgaccttctatgaccttgtactttcataaaaaacctgagatggtctacattcataagacatttacatgatggtatatttaatctagtagagatcaggaaccatatagagtagagaagatcataatgtagaaagaccataggtgaaataattccttgagtaccaagtttaattctgaacctcttggccaccatgcaggtttgctattaggatataagaatgatttccagccgggtgtggtggctcacacctgtaattggcactttgggaggctgaggcgggcagataacctgtggttaggagttcgagaccagcctgggcaacatggtgaaatcccgtctctactagaaatacaaaaattaactgagtgtggtagcacccacctgtagtctcagctattctagtgggaaggttgaggcaggagaattgcttgaacctgggaggcagaggttgcagttagccaagattacgctactgcactccagcctgggcgacagagcaagacttcatctcaaaaaaagaaaaaagaaagactgattttccaaggccccttttagctaaccaggtcctgaaaatccaggtgttcatggcttcaaagctaccatcggtgccatataggaagtgagtagggagaaggcatggggaggacagcaggaggacagtgaggaaccagcaaaggacactctgtggacacaagaggagaggcatgggggatgtgctctgtttcaggctgtgctgtcggccattgtgattgtcaacctgaagggaatgtttatgcagttctcagatctcccctttttctggagaaccagcaaaatagagctggtaagtaacaaaggtgatggctctgttaatatgtcatagagcagatatgattagtgctaagaaaaatgtgagaaagataaattaatgcagttgttatgtgcagtatccagttagacaaggtaaaagtagcatgtgtgtgtgtgcacacttgtgtgtttaactcttgtgggatgtttaacatggttctccatcaagttttctgtcttttgcttagaccatctggcttaccacttttgtgtcctccttgttcctgggattggactatggtttgatcactgctgtgatcattgctctgctgactgtgatttacagaacacagaggtgagtgcccagattggaatgggtgtgaatgtcccggcagagatgacaatgttgactttaggtgtagaccaaagtttaagttggtagaagtggagccctttgatgatttctagttagcgtgagagggagctataacactcatgtagcctgttgactagatgaacaaaatgccaatttaaaaattccatataattttgccaaatgctcttctatgtcacaatttatgctcccatcaatggttatgttaaaagagcctaatttccatcattgtttctgccattcctggtctagtgctatgctggtttatttatcctcttgtgatttgttttggcaccaagtactgacatgagtttcaatgacatgaagcaaactctgacaccaagttatcgtatgcattccttccactgtcatttcctccaccctgaaccactttcccttgttatctcttctccctagtaggaagctgagcccactagggaaagtataaaataaattaaaaaaatatatggttgggtgcagtggctcacgcctgtaatcccagcactttcagaggccaaggcaggcggaacacttgaggtcaggagtttgaaaccagcctggccaacatggtgaaaccccatctccgctaaaaatacaaaaaaaaattagccaggcttggtggcacacacctgtaatcccagctacttgggaggctaaggcaggagaattgcttgaactcaggaggtggaggttgcagtaagccaagatcacaccactacgctccagcctgggcaacagagcaagactccatctcaaaaaaaaaaaaaaaaaaaaactttatgggctgggcacagtagctaacacctataatcccaattccagccctttggaaggctgaggtgggaggatcacttgaggccaagaccagcctgggcaacatagcaaaactccatccctaacaaaaaacaaacaaacaaaaaaacacctttttgtgataaatcaccataaagatgaaaaggaaacattgtcatagaaatagcctggactggccaggcacagtgactcttgtctggaatcccagcactttgggaggccaaggcaggtggatcacaaagtcaggagttcaagagcagcctggccaacatggtgaaaccctgtctttactaaaaatacaaaaaaattagctgggcatggtggcgtgcacctgtaatcccagctacttaggaggctgaggcaggagaatcgcttgaacctgggaggcagaggttgcagtgagccgaggtcgtgccattgcactccagcttgggcaacaagagcaaaactccatctcaaaaaattttttaaaataaaataaagaaatagcctggactttagtcaggtcctggcggcaccttctgtaagctctgtgattttgcatgcctctcagagcctgtttctgcatctataaaatgagactaatacttcctacctcatatagttacaataaggcttaaatgacaaggcctgcaaacagccagcacagtgcctgacaagtgctacccactctaccaatgccagtgcccttcccgctctaacacagagggaaatgaaaatgaagctagtgtggttttgcctatggttgcaaatcacatgaaaccttaaagttgtgactgtgatgtgttgaaagggcactttatgaggatgttgaccccttatctgcaggaggtcaggatgctccccaccctcaagggtgggaaatgagcattttcatcatcacaaatgacataacagaaaaatgttaagaatatgtgggattctaaactcaggataagaatttaactacctttaataacaagttccaacactgttgcccgtatttatctaaaagaagtgtttcccatttttataaagaactttattagcatatgtaagagttctttatctcagaactttggtgaacattatggttttaaaatatctataacttttggccaggtgtggtgtctcacacctgtaatgccaggaattggaagactgaggcaggcagatcacttgaggtcaggagttcaagaccagcctgggcaacatggcaaaccctcatctctactaaaattacaaaagcgagccaggtgtggtggcgagtgcctgtaatcccagctactcaggagggtgaggcagaaaattgctggaacccaggaggtggaggttgcagtgagctgagattgagccattgcactccagcctgggcgacagagtgacacacgtctcaaaaaaaagaaaaagaaaaaaaaatctataacttttaacaagctacagtatagaatttgagaatttttttgctggtggtggtgttttttgttttttttactgaataggattttatggtatcttatcataaattttatttatttttatttacttagtctttatttttctaacactagagaactcacttgagttataaaatttttttgtttgttttcttttgagacagagtcttgctctgtcacccaggctggagtgcagtggcatgatctcggctcactgcaacctccacctcctgggttcaagtcttctgcctcagcctcctgagtagctgagactacaggcgcgtgccaccacgcccggccaattttttttatttttagtaaagacagagtttcatcgtgtcagccaggatggtgtcgacctcctgacctcgcaatctgcccacctcggactcccaaagtgctgggattacaggcatgagccatcgtgcccggccggttatcataaattttaaagtccagcaagttttttttttttaatgctccagtttgtgctgggtgcagtggctcactcctgtaatccaagcactttgggagactgaggcaagcggatcatttgaggtcaggtgttcaagactagcctgaccaacatggtaaaaccctgtctctactaaaaatacaaaaaaaaatagctgggtgtggtggtgcacacctgtaatcccagctactgaggaggctgaggcaggagaatcgcttaaacctgatagactgaggttgcagtgagccaagatcacaccactacactccagcctgggtgaaagagtgagagcctatctcaaaaaaaaaagaaagaaagaaaaaagctcccgtttgggcaacctggtaaaaccccatccataccagaaatacaaaatattaactgggcatggtggtgcacacctgtggtcccagctacttggaaggctgaggtgggaggatcacttgagcctaggaggcagaggttcaagtgagctgagatggcactcctgcgctccagcctgggtgacagagtgagaccctgtctcaaaaaaaaaaaaaaaagaaaagaaaagaaaaaaataaaagctctacttctctttcttcctcaaataatagttaactgaagttgatattttttatgttctatcctttgttggtgattttgctgggtctttgtatgttagagtttaggagatcagcactcagggtatgtgtttattttcagctggcttcttctgtaattagggcacacttctggagaggggtagaggtgcactgggtttggccgtttgtttgaaacactcattttccctcagagcaggcatagttaagagacaatcatggcccggccagctgtaaacccagcactttgggaggccaaagcaggtggatcacgaggtcaggagatcgagaccatcctggctcgatcaaaaaattagccaggcgtggtggcgggcgcctgtagttccagctactggggaggctgaggcaggagaatggtgtgaacccggggaggcggagcttgtagtgagccgagatcacgccactgcactccagcccaggcgacagagcgagactctgtctcaacaacaacaacaacaacaacaaagagacaatcacatcattctgcagataattcctgaattcaggcacttgcatgtgttataaatatgacagaggttcagcttctgccccccaaagctcacaatttggttagcattataaaattttaaaaattataatttctctaaataggtatttgttgagggcctattatatttccagggagatgctagactctagggacagtgcagtatataagacatggtccttcccctcaaaggttgcgttgtcctaaggatgacagacacgtaatcatgatgatgatgttaataaggacaatgaagtaatgatggcacaaattattataattacttatgtgcctggctctgtgcaaagatcaatgctctatgccatttcatataaccttataaacctatgacaatcagcaaccccatttcacagatgtggaagctgaagcctagaatgattgggtaacttgcacaagatcacagtaagtggtgaggcaatgactcagatgtgagtttttgcaaatccagaagctgacaaatcagcacttttttttttaagacagagtcatgctctgtcgcccaggctggagtgcagtggcatgatctcagctcactgcaacctccacctcctgggttcaagcagttctctgcctctgcctcatcctcttgagtagctgggattacaggtgcccgccaccacacccagctaattttttgtattttttgtagagatggggtttcaccatcttggccaggctggtcttgaactcctcacctcgtgatccacctgcctcagcctcccaaagtgctgggattagaggcatgagccaccgcgcccagcctcaatcagcattttataatccatcttcaaaaaattagaatataggccaaacacagtgtctcatctcatgcccataatcccagcaatttaggaggccaaggcaggaggattgctgaggcgaggagtttgagaccagcctggacaacataataagaccatgcctctaccaaaaaaaaaattttttttattttattgtttttagagacagagttttactttgccacatgggctgcagtgcacttgtacggtcacagctcactgccaccttgaattcctgggctcaaaggatcctcccacctcagcctcccatgatgggcacacaccatcacacctggctaatttttaaattttttgtagagatggggtctcgccatcatgctctggctggtcttgaactcctgggctcaagtgttcctcccacctcagcctcccaaagtgctgggattacagttgtgagacattgcattgtgagacctacaaaattttttagttggcaaggtgtagtggtgcacccctatagtcccagctactcaggaagctgaggcacaaggatcactagagcctaggagttcaaggctacagtgagccatgatcaggccactgcactccagcgtgggccacagaataagatcccatctctaaaaaaataaataagtaaataataaaacaaaaataaatttttaaagacttagaatacacagttataagtgcctcaacaggtgtaagcaaggtacagtgtgagaagcataagagatgccaaataaaactgcatgggatttcattaaaagagactttcttgctcttggtgtcattagggaagtagtaaggacttggccttcactgggagcaatagtatttagacttgcagaaatggtgtgccagggcactgcaggtcgtcaggaaggcctgttcatcatctgcatttgaaatcctcatcctttgaggcccagctcacccagtcagggacctcaacttcccctgtgtacccacaacaatgctctaatccttccctagcaccttgcgtgtgtccatctcagctactcctgtacttgtgatccatgtgcccatcccttactctgcctcccctgtggcactggattgcaacacatggacagcactttgagtctcttagaggttttctcataattgcaaactcagctgagagccttagccccttatctacaatcagtcctcagagccatatttgtccacagatatcactagattgtgagcatcttgttggcagaagtgatgttcctggagtctagtaccaggcacatcatttgtgttctctgaatgcttatggaggaaatttacccagcaaaacagctaacacttgtgtagtatattctaatttacaaagtcctttgacattcatgctaagaagcaccaggacatagtagcaaaactgtggaatgtcaaatcaaagaccttgagccatattctgcctctggcaagctgtctgacattgaacaacttacctaaccccactgagcctcaatttctcatctacgtgatggcaggattgttaataatgttaactattattataatattattaatgtgcccagcagcattgttcaaaggatataataacaggtgttcaagaagtagtatctgtcattattacagtatctaatttgagccagatggtcctttacaaagagttaggaattaggggcaggcacagtggctcatgcctgtctgtaatcctggcactttgggaggccgaggtgggtagatgacttgaggtcaggagtttgagaccagcctggccaacatggtgaaaccctgtctctattaaaaatacaaaaattagctgggcatggtggcgcacaactgtaatcccagctgcttgggaggctgaggcaggagaattgcttgaacccgggaggcggaggttgcaatgagccaagattgcaccattgcactccagcctgggcaacaggataagactccgcctcaaaaaaaaaaaaaaaaaaaaaggaaatagggctctcaaaaaataatgtttaaaatctattcagactaaattaagttaacatctatgctaatttgagattttcattttctccccacgccctcaattgactactcccctactatgagtcagtgattcttgctaacaacaaacaaaaattccctctcctactttagagttagagcagaaagacagttctgtctactgagtggcttgaggagaacttgtgttacatatatccctggcataacttgaactctgcgaaattcagcgttgcctccacctctaaatgcctgaaccttttctcctgaccagatcgtgtttgtggtatggtgttggggggaggggaggtatagggtgtgtgatgtgcatcgtgaatgtggtgtgaggtatacgtgatgtgtgtgtgtgtgtgtgatgtgaagtgtgaggtgtatgtgatgtatgtatatgtgtgtgatgtgtatgtatgtgtattatgtgtgtgttgtgtgtgtggtgtatatgcatgtgtgtgttgtgtgtattgtgtcttggatgaaggttgtatatgcatgtgtatatgtatatgggtgtgtgtgtgtgtgtggtatgaggggtatgtggtgtgtgtattgtgtatggtattgtgtgtggcatgtagtgggggtatgtatgcatgtgtctgcacgtatgtgtattatgtgtgtagtgtgtgcatatatgtggtatgtacgcatgtgtggtgtgtgtattgtatattgcatgtgtggtgtgtatgcatgtgtgtgtgtatatgtatgtaggggtgtgtgtggtgtgaggtatatggtatgtgtgtattgtgtatggtattgtgtgtggcatgtagtgtgtgtgctgtgtatgcatgtgtgggtgtgtatgcatggtatgtggtgtgattgtgtatattgtgtatgtgtggtgtgtgtgtattgtttgtgtttgtgtgtagtgtgtatgcattgtgtgtatgtatgtgatatgtggggtgatgtgtgtgtatattgtgtttatatgtgtgtgtggtgtacatgcattatgtgtagatgtgtgtattgtgtatgtgtgtgtggtatgtgggtgtgtatacattgtatgtgtgtgtattgcgtatgtgtgtgggagtgtgtatatgtgtggtatgaggtgtatatggtatgtgtgtattgcatatgtgtggtatgtatgtgcatggtgtgtatgcatgtgaggggtgtgcattgcatagtatatatgcagtgtgtatgatgtgtatatgtgtggtatgtagtgtagtgtgtgtatattgtgtgtgatatgaggtatgtatgtatggtgtgtgtgcattgtgtgtgtatgcatgtgtgtgggggtatgtattgtgcattatacatgtggtatgtatgcatgtgtgtatgtatatgtgtggtatatcgtataatgtgtgtgtattctgtatgtgtggtgtgtatgcattgtgtgtaggtatgtatattgtgtatgtgtgtggtgtgtgaggtaggtattgggatgtgtatgtgtagtatgaggtgtacatggtgtgtgcgtatcatgtgtggtatgtatgtgtgtgtgtggcatgtgatgtgtatgtggcatgaggtgtgtggggtgtattaggcttggaatggcactgggaacgtgatatgtgtggagcccttctgcaaaggcaccatgtaaggatcaagcattcccaaatgcaccagcaaaccccaacctgacataaacatacacaacctcctgttgaaatcacacgctgtgcctcaacacaatggtatttaacactctttatcttcagtccaagctacaaagtccttggaaagcttcctgaaactgatgtgtatattgatatagacgcatatgaggaggtaggacctttttctatcacttaaaaaggttgtaagcatgtgatgtgcttggctaagtactgtttttacccaggataaaaagtaggggtaagatgggaggtatagtggtcaaagtgaaaagtctgagctttgatctactatatggattcaattttataatttatactatgcattaactactcaatttccaatatttatttttcctcatttttaatggggataaatatatattgttgaaattaaagtatacatttatacagtattgaaactgaaaggatggtttgtgtgtgcatggtttttttctgatgaatgtataatggtaaaataattttaaccatgtaaaatgtagatggtggggctgtggaattctcccacttcatttgaatggttaaaattacagcttcagatttccttgattttgagtgttcttttcttctgtggaatatcgaagatttttacaaaatcttttaaaactagttttgtctgtcggtcttgtcttatgcttgggaagaaattctcagactcactttaaggtattaggatatgaggaaacaaaattcactctaaggtaaaggtgttagaattgctatatttttcctcgatccaaagaagtatatgcaacattgcttgtttccagcctgcctcctagagttccacaagggttcttgataaaatattcactcagtttcacttgtgtccatttaagctccttcctgcttctgtgaatcaaaccagtgtaaacttctgttcactttatttgcctgcctctcacagtctgggcagcagccttccatctcctgtccattttcttttctgtttttgagtatttttcagtttcttctttgtgtttaatggtaaaaataaaacgaagactgacttttaaaacaaatggaaagatgtgcatgttaaaaaggtctctgtcagttctcttccattcagagcatgagtagataagagtgactttttttttcttttcttatctttttttttttttttggtctgtgtgtgacatcattttagctaagccctgaaaaggggaccttagcaacttctgcattcattttctatttggggtggggaagctatgcagttttgattctgaaatatttccatgtgttaaattcagcaatattttactcttcaggtgaaagaaattcctggaataaaaatatttcaaataaatgcaccaatttactatgcaaatagcgacttgtatagcaatgcattaaaacgaaaggtgagtcatgagaaattgattatttctgaggatgggatttgtgggttgacagggatatgaggatcactagtatacctttgtatatactatttcattctcgctttgtttctctctcttttattgtagtaaaatatacataacataaaatatactattttaagcattttcaaagtatacagtccagtgggtcgcattaagtacataaacattgtgcagccatcaccattgttcattctctcttatcgtatatataatattacttttcaaacaaaattttaagaaaacatgagaaaataattttaggtttcttattcttgggatcatgtagattcctttggtcatgtttagaactcaaccctacaccacgctgtatataggaaaagctccctgtcaacccacagagtctcttaaatataacaacctgacagatctgaacttgaaaaagactgctctaaaaattgtctttaatattttagcttctggttgtcttgaataaagactgagaataagaaaggctactaatgtaagggagtaaccagttcagggcctctgagggaaaggcagtgcaagggttggggtgataatctgacccagaaagcagttaaatggggagatttgaaggggagtggcattggggtgggtccctgagagccatgaagtgattttgcatatggtgctcttctgtaaagactggagtgaacccagcagtcatcatgggagcaaggagaaaggccatgcggaagtacgctaaggaagtcggaaatgcaaatatggccaacgcaactgttgtcaaagcagtgagtggtcttttcattgaaataatccgttctgtcttgttctgcaatgccctccatctttactcatgactgaagtttaaacacagcacgaagttactaaaagaggaaagtttagaaaccaccttagtatcctctatattttacaagcacctgataaggtcagttatgaggctgtttctgaaaaaagtaaggaaaacatcattttaaaaaataaaaattacacaataccttaacagattttaccattagcactttgtcatagatttattcagctgatagtttaaaaagctcaaaggtggcttttgctgttcaggcatgctaatgtgtgacccatcatactaaagttaatggaactattttagactttagcagaaaatggataggataactgaggagagtggattttgttggacaagaaaaaaaaggaggagaaggaaaaagagagaagttgacttaaaatggaatgttatacatacatcctttttttgttcagttacaaggctagaatatgacaaatattttacaaggggtctggtagagtcagtagcttaagaacacaagagcagattttccattataaatctttctcacaaccagaaatgtgagttgttcataaaattctggtttggtttaatttttgacacaggatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcaggggtaagcatcctcttgaacgagtcatttaatacctctatgcctggtgtcgtactatcagcataatcagggaggtgggctaggctctcttttcaacaaaagatttatccccttttgattatggaaaatttcaaaaatatatagaagtaaagattttagtatattgaacccccgtgaactatcactcagcttcaacaatgatcaatttatggtcaatctggtttcctttatagctgacattcactttctcttgttttattttgaaacacatcccagtcataacatcatctgtaaatattttaatgtgtattgctacaagaagattcgttttagaaaacattactgtaatatgctaggctactcaggaggctgaagcaggaggattgctgagctcaggagttcaagaccagcatgagtaaactagtgagatgctgtctctaaaaaaaatcgttttttattttgaaattataatatcctttaagttaatataatagttttattgtacttaagtaagatttaacagtatttttgctacccctaattatcactgtccacataaaaagcaaaagagatacaaaattatttttaactgattagctcaaaaatacatcattattgaagcaaagtagctcaagtgaatgaattagagtgtcttctatcacgaaaaacattggtaaaaagagactttcatctggcacagagtaaagagaaactaaattttatttcaacattattacagattgtaaaagaatatggagacgtcggtatatatgtatacttagcaggatgcagtggtgagtatacctctaagatggggtgaagcttaatattttagttttgtttctcttatttaaataattgcacttggaaatgtccttaattttaattattttataaaaatgaaaattgttcttctgaaaaatcactctaaagccagctttgaaattagccacatggaggcattcatattacaagttacttgtgacaaccctcctgaactggagttggaggaaaggagccaataagccatcatttattataatattaatggctgctataaatatagtaattataatatatacatgaccacagtagcaattatctaatggctgccataaacagttacatataatcattcaggttgcttgtttctctatccaatcatttgggaatactttgcctgctatttttgttttatattacattttggttaaaacagtgcttgaggccgggtgttgtgactcacacctgtaatcccagcacttcgggaggccaaggcagatggattgcctgagcctaggagttcaagaccagcctgggcaacatggcaaaaccccatctctactaaaaatacaaaaattagccagacatggtggcgcatgcctgtaatcccagctactcgggaggctgaggtgggaggatgtcttgaatctgggaggcagaagttgcagtgagccaacatcacgccactacactccagcctgggcaacagaatgagaccttgtttccaaaaaaaaaagaaagaaaatgattttttagaaaaggagcccggcgtggtggctcacacctgtaatcccagcactttgggaggctgaggcaggcggatcacctgaggtcagaagatcgagaccatcttggctaacacggtgaaaccccatctctactaaaaatacaaaaaattagcccggcttggtggcgcatgactttagtcccagctactcggaaggctgaggcaggagaatcgcttgaacccaggaggcggaggttgcagtgagccgagattgcaccactgctctccaggctgggtgacagagtaagactccgtctcaaaaaagaaaaggaaaaaaaaaaagtaaaacagtgcctgaggctgggtgtggtggctcacacctataatcccaacactttgggaggccaaggtaggaggatcgcttgagcctaagagttcaagaccagcctaagcaacatagtgagatcccatctttatttaaaagaaaaaataataataattttaaaaagtgcttgaaaatgaaatcttcataaagatgctacttttcacctgtaagattgccaaaaatcaaaaataaatgacaccatgctccataggcatatgatgggaaaacaggctctctcagacattactgacgggagcacaaaatgcaacagcccctatggagggcagactaacattagctttcaaaattataagtgcatttacctttcaacacagaaaatccagcaatatgctgcatatgttcaaaatgacacttgtatgtcgttgatcatcacagcattgttataaaacaaaaaactcaagtaccaaacaaaagggactagttaaataaactagggacatctatacaatagaatacgatgtagtggtaaaaaaattaagcaaaaagggttcagaattatctatatatagtttatatatatatataacattaatctatatatttcccacttttcttatccccaaaacagcattttatatatatatgtatatatatgtgtgtgtatatatatatttatatatgtgtgtatatatgtgtgtgtgtgtgtgtgtttgtgtatatattttttccccacttttcttatccccaaatatataaaatatgctgttttggggataagaaaagtgggaaataagaatacatacttatatttatttatatttacatagagaaatcctggaagaacacacaagaaagtagtttcctgggattgaggttatgaagggtggagacatgaattggagtgaaacttttactatagcattttaatatttaaactatatgtgcttgaacgcaggagttggaggttgcagtgagctgagatcacaccactactctccagcctgggtgacagagtgagactctgtctcaaaaaaaaaaaagaaacaaaaaacaataacaaaaaaaaaaacactatatggaagtgttacttttttttaattgttgaagactgatatccagagtgtggactaattttcttttcttcttttaatagcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacgcctagcaagatgtattgtacttgtgtttttttaattgaatacttcaaagataaattggcctgttgcctgcatttattatggagagacatttctgctacattctagtattatctttgtaagctaagcatcgaaaaatttatttcagtcaagtattttacccacaaataagatattacatagtggttctttgtttttgtacttgttgatctacagataagtagtggttctttgtttttgtacttgttggtctacagactgtctataatcagttatctaacagaagacaggaactgcaattttctagaagttcttgttttaaaataaccttttatttattatagaagcagtataaatgcattgaggacattttaaaaaagtagacaagcaaaaataagaacataaaacatatttccaccactcagagattaccactgttaatatccttccagatctttttccatacatatacatttacatttttaaccaaagtaagaccacatctatagtgttttataaccgatttttctttaatcaacaatctccactttctcattccagcacatttttgatcttagaccatatttaaatttccccaattgtccccagatttgcagttggtttgtgcaaactgatattcattctatgaccttacattgcaactggtcatgtcctttaaagtcccttacatctattttaatctagcacagttccttttttatgacattgacttgttaaagagactggggcagcggtcctgtagaatcctgtcttgtgaatttgcctagtttctttttcatggtgttgtttaacttgtttttatattgcctgtactacctgcaatctggaagttatatctaaagcctttataggttcaagttaaaacattttgtgctagattatatcatagataagatacatgcttcatatcgcatcacttcagaaaacgtaatatctagttgatctaccattaactaatctattgatctaagtttgattactggatcagcatgacagtctgatcatccagttagatttttcccctttgtgactacagacttatctgtgtggtattcctttggcattgaatgaattaccatttacccaggagttttaacattaggtggtaattcttgcctaaatcattaactttgttaaagtttgtgagttggttgtctagttctttcatttcttctccgtttgttagttggttattctatgtgaactaagctttcccttctccacttgagctatttggtcatccttacctacagttctaccagaagggcaggttgaatgctttattattttcatttaattatcaacttcaaagtaaataactggtttattagatggtgacaaattagtgtttttttttttttttagatttcccttttctggtgtgatggtggtcttgtgaatttttatagttttaatgggtttcaatccattatgttcttttcgatgctcaaattgtcacaactgtggccagtataagtccttagaaactttgctattgttgctaatcatgaagtagcattaaagaccatgtttttaagaaaactaacccccacatataacttaaatcttccttagaagttcataacagctatcagttcattatacatcaaactcagtgttcagcctaggagtaagcataatgctcatcttactcattccacagaaacatggaatgtcaaaggttggaggaggatacctttaaaccaatttgtcactttcagttgtttgtaattaccagctcactacaaatcaaagcacaggcattagccaacctgtctaaagcctcctctgctaagagttcctgctgatagtgagtttgtcttttctttaatatgctgtcttattgacatcttgttatagctttcatgaataagatactgccaggagaaagtagtcagatcccagtagaattgttagcatggcctacttgcaaccaaagcctgtggtgtatgtgttgatgttaactcctatcagatttctcactctctttactaatttaattcaaaataaactcctccttttaatcaaaactttaactaattctagagtgaaatttagattggcttagttatttcctttgcaatcatcacgagatcagatacatctgcattctgatacggactgccttctgaaagagcatgtagttgcagaacctcagcttacactagaaacttttgtaaacgagctaatatcatttcagccatttaagtagaatttaaggattttaaactatgttataaaatacttcggccaggtgcggtggctcacgcctgtaatcccagtactttgggaggccaaggcgggcagatcacaaggtcaggaattcaagaccagcctggccatcatggtgaaaccctgtctctatttaaaaatacaaaagtgagctgggcatggtggcgcgcacctgtagtcccagctactcgggaggctgaggcaggagaatggaatgaacccaggaggcggaggttgcagtgagccgagaccacgccattgcactccagcctgggtgtcagagcaagactccatctcaaaaaaaaaaaaaaaaaattattgcacattctttagcaccagccttccacttagcagatactatggttgatagattgacagtagtgtacaggccaggcgcagtggctcacgcctgtaatccagtactttgggaggccgaggcaggcagatcacgaggtcaggagattgagatcatcctggctaacatggtgaaaccccgtctctgctaaaaatacaaaaaaattagccgggcgtagtggcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaagtagtgtataaaaaaaggtatttgtcttatacatttgtatttaacttttctgttgaacttttaaactgttttgaatagaattgttatgtatattaaaagcacaagtcttaaggtgggaaaatatatttttgttttatcactttgcttagcatctgagaacgagtgcatttcaaaatacatttcatccatatgtaaatacgtcagaatgttttactcaattatttcatacagcaaaaatttcctgacttttttgtcttacagtctcgtctctggctttcaaggaaataatatataagcccacctgttctttctgggccttaatagctgctactgtatcttctatgtttttccaccctgattcaaattcaggttcatattgtcctatcaaaatgggcataatctacataatctaagcactagccttattttatggcaatatatgggaaagcactgttttgaagaaggtcccataaagaaaagtcctttgaccttctgaaatctcgtgcagtatgacagtggttgatctatgtctcatgaatcgttggaatatgtttgaaaacatttgtgaaccagggcccaggaaactccaatctatgtcttagacactgactctgtattagtatatggcagctgataatgggatcccaattgccaatctcatctgcagtggaaccccaagaaactgttaagggtatggatcctcttcaagccctggaatagctcattttgtagttgcaggaaacatctaaaacatccactctgtgtggtttgatcctgtgtccttacctgtttagcagagaaagacagtaccagcctctgagtgtacactttgagaaaatgagtctgctctcttgcgtggatgaagaaaagagagggtagcaaacctagtgaatctgtcctggggtgggtgggtgtgtttttgtgtgtgtgttgggggggtgggggtgtgcccttgtgtggggatgagggagggaagagcaggtggtggggaaggctcttttatgtctgcccctttaaagatagtaaatcaatttaaattctaaaatgaaaccgaacttccttggtgtgagcctgtatgctttactgagtgcaaaaaaatgctattggtcctttttagtgcaccttacctttcaggagtgcctttaggattcacgatgtttagattcttactgttttgaatgctcataaaagtttgagaactataaaagtattactgaattcttcaaacatacagctaaccaaaatgcatgcaatggttttgaaacccctccaatcccagcaacacaacggtaaaagttagcaccacaccaggtaacgtgcctgcttagtgacttagattttatcaagtttgagcatcttaaaactccacctggcctttgagaaagctgtaccaggcttttcattatactggtgtattgcatttcactttacgtaacagataaacctcagaagcatcatgaaaacggagttttttaatacacctaatcacatttttaacactaagaagtttttaaacaggattctacggtatatttttctaaagtcataaatcctttgtattctaacttatcagcaccctgacacatgtatatatatatatgtgcatctacacacatgtatatagacacatctattttaggtggaggaagttactggtaaatgtctgtttcatctctttgagtacaaacatgctgacctaggcaatgttgatgctacctgcataaatatgccttgacagaaaaacccaaatttttaaagtagttgattttacatttgatgtgtggccgattagattttccttatggacctaaattatcacgatagtcactctgtatcgttagaacagagtgtgctcaacattatctaattcgagttgtctttaattggtatatatgagcacgtggccatgacaaagtggaaagacttcagagtgtgttctaaatgtgtacatttatttaatcaaaacattcaaaaacatacatcttcagtcattttcttaagtatattttattgtaagcataacctgaaaatagtttgctctatttaattcctcaagaaaaattagggcagggttaaaaaagatgaatttttctgatcaagttcataaaaatgaaaaatcaagaatgttggttggattttataaaaggctttaatttctttacttgaaaactttcataattttaagtatttcagtgcaaagcagcattttctatgttgtcaactttttctattgtgaattaaaaatatatccaactcttattttgcaacttatttatgaatatcgaacttcaataaaaatattcattattgtgctttggtcttggcattgggggaaggggtaattgtggtagaagataggatttggtggtggttacacaggaggtaattttaaagagatgttgaggattctaatataacctttatttttaaaatacctcactgtagaccagaagcctaacagaaactttaccaaaattttaaagtaatttgtaaaatgaaagtaatcacttttctgtatgaagccaattctggactttctaaattagatcatatgtacaaatatttttttttctttttttactagtctttcacttctggctgaacatttttctttctgctttattgcaccactttactgctcattaaaatcactaattcttcctgattaatttgaaatcaaatggcaatgcaaaacatatacatccaatgtttctcctactttaaaaaagctttggccaaagcttttattattcaatatggtggtgggaaactatcataatgcatgtggtcatacgcctaagtgagctactttgttgcgggacagaagcggataataggagcataggtaatggagccaacaaacttgtacttgaaagccagctttgccactacaagtaagtaggaaagtttatgcatccattggatgaagatccatttgataatcttcctcagtggacagatgggaggatacaatgagaatttatatgtagagcacccggccagcatatgcctaggatagaagccatctaagcaagcgctttgtatgatgtgaaatccttaaacgggtgtgggtcaaaaaaatagtcaaaatatattctaacaagcttttctgcctaataacattagaaatcaccttttattatattacaagaagagatatggaaaaaagcatacaattttgaagccatttatttgtttatgaattggttattaacaagtctatataaggttaggattccaatttaaagttttcacttgaaagccttcagggttcagttgtatgtcatgtaacgaggagtagcactgaatttggcataagacttaatgaccttatttacagcttccaagaaatccttctcggtagcaatttttcgccgtgctctgatggcaaacataccagcctctgtgcagacgcttctaatctcagcacctttcaaacaaaaagaaaataaacgggcttaattaaagcagcctgagcagacagaccagcaaatataagcaagaactttctacttaccagtgctatttggacacagtcgtgctaacagttcaaatctgatatctctttcaacactcattgaacgagcgtgaatcttaaatatgtgggtccgaccctaaaaatgagaaaattgacaaatattaaaatggaaaaaaatcacaggaagaaaaaacttcaaaaatccctttcctaaaatgaaatggttttcttacctctagatcgggcaagctaaattcaatttttctatccaatctccctggcctcatcagtgctggatccaaagtatcaggtctgttagtggccatcagcactttaatattgcctctaggatcaaaaccatcaagctgattgatcagttccaacattgttctctgcacttcattgtcacctccagcaccatcatcaaaacgagcccctgagaagacaaaaggaaagataagctcttcaagtccactcaagagtagcactatgcatgacctgaaagtaccaaaaatctcagctcaaatcatgtcctcaggcacaagtgacatcccactttgaagcaaatcctgtaagaacagcttgctttatataaaactaattagaaagtgaatctttgctttcaaaagtattcatcacagatttacttaacgttttttaaactgcctatggggcagtttagtgttttcaaagtctaccacatttattacttaatttggacctctcaaaaactcatgaagtacacaagacaacaattattccctttataagatgagcaaactgaggctcagagattagttaaaataacttgtcctcaggagttcgagaccagcctggccaacatggtgaaaccacgtctctactaaaaatacaaaaattagccaggcatggtggtaggcatctgtaattgcagctactcaggaggctgagacatgagaattgcttgaacccaggaggcagaggttgcagtgagccaagatcatgccactgaactccaacctgagcaacagagtgagactgtttcttaaaacaaaaaaaaaagtctagatttcatggctaatactgtatttcatcaaatctgtgaagccaacaagaacattattttatgtgcactaagaaaaaccaccatcaattaaattatgccccagtgctttcttatgacattagatgatacttctcaatttacgagttgtttaaagtatggaaataggtgttgtatgaatgaaatcagatatagactgagcatcccaaatttgcaaatgcaaaatccaaaatgcttcaaaatttgaaagtttgagcaccaacatgacaaaagcaatactcattggagcactttagatttcaggttttcagatttgggatactcaatcagtataatgcaagtacttcaaaagccaaaatccaaaatatgtgaattccaaaacacttctcaaacatttcggctaagggatattcaacccatagcacaaaaataaaggctttgaaaaaatgacaatggtatataatgtgtcttcttgcctttatagtttagcactactacttattagatctgtggcgccactgtttcagagctcatagaagagttggagttaaaattctgtacaagattaacatatcacgacatatcactttacagctttcatgaaactgaatcctaaagcagttctctaacgtatcattctcacctccaatagcatcaatttcatcaaagaagataaggcaggctttttttgttctggccatttcaaagagttcacgaaccattcgagccccctaatgagaaaaatgatttattaattcaaaattggtttcaaaatattttaaaataagtaaaaagttatgcttcaaacttttcaatatttattcctaacagtattctcacccattattacctaaacgttttttatttttttatttttttgagatggagtcttcctctgtcgcccaggctggagtgcagtggtgcgagctcggctcactgcaacctctgccgcctgggttcaagcgattcccctacctcagcctcccgggtagctgggattacaggcatgtgtcaccacaccccgctaatttttgtatttttagtagagacggggtttcagcatcttggccaggctggcattgaactcctgacctcatgttccacccgtctcagcctcccaaagtgctgggattacagacgtgagccactgcgcgcagctattttaattttattttttgagacagagtttcactcttgttgcccaggctggtgtgcaatggcacgatcttggctcagtgcaaccttcgccttctgggttcaagcgattctcatgcctcggcctcccgaggagctgggattacagacatgcgccaccacacccggctttttctattttcaatggagacggggtttctccatgttggtcaggctggtttccaactcctgaccttaggtgatccgcctgcctctgcctcccaaagtgctaggattacaggcatgagccaccgtgcctggcctatctaaactttttttaaaaaagactgaagtttgctccccagtccaccaaattaatcttgccagccttaaaatattaagtcattttcatgattctgtatctattaaatagcaataggtttcagatctgaagaggtcaaattaatagttaaataaaggaaccattctaattagtaaaacttgagtaaataaaagcatctgatctatcacttggaattcctgatcattgaatttgacttgcaagttttagttagggaataaaaggatacctcaactcacgcagctccgtagctaattcttattgtaaatatacgaatcatcaacaggtttcagatacaatacatatagccctagacttattctctaaatatatatatatatatatatatatatgtatgtctacaaacataaaaacaacctatgttctcactttctcattaggactgaaaacactgcccctgaagtaaaagtatactttttaattaatttaccaggcatttccgtagaaaagccacatctgtagaaaatgtcacccttttgaggagggggcatactggttaaaattttaagtggttgcctatctccaaatgaccacaaaggcagcttctaaactactaattacaatcaagaaatttcaggtctcaggccgggcgtggtggttcttgcctatactaccagcactctgggaggccaaagcagatggatcacttgagcccaggagtttgagaccagctgggccacatggtaaaacccctctctacaaaaaatagaaaaattagctgggtgtggtggcatgtagccatggtctcagctacctgagaggctgagatgggaggatcgcctaagcccagagaggtagagactgcagtgagctgagatcatgtcactgcactccagcctgggcaacacaatgagactctgtctcaacaaaagaaaaaagaaatttcaggtctggatccctcaatgcagaagtgccatttttaatttcattcatactgtaaaatctttcacaaggctatatattctttgataaatttactttacctcaccgacgtatttctgtacaagctcagatccaataactcgaatgaagcacgcatcagtccgattagcaactgcccgcgcacagagtgtcttgcctgtaccgggtggaccaaagagcagcacgcccttgggaggctcaatgccaaggttcacaaacctctctggctgtgatagagacacaattttcacacttaccacttctgtataacaaaaatcagactacttctaaatcaacaaagtgcttttatgtttagtattttatcctgaaaaccatactttaatatttaaatcaaaattattaagtccacaatatacatacttgaactaaataagaaattttacttattctaagacataagttctaaactggacttctatcaatacataagactaaatggcttttaaaaagcacttccattatccagtcttaaaatattttcctaatttaaaaaaaattatggtgtttaaggaacaaaattatctggtattcagtatcctagttttccattatcctcccaagtttaacctgcttcaatttaagcccctttcttctcgatgtcttttgaagaaccagtttcttctataatagcctgtaacatatttaacatattgctaaaggttacactgtgcctcctctgtcactgtaaacagcagatctttcttttttaaatatacctgaccactcaatcctttttgcttgctctcccctagaacttaccaactctccacacctcctctctccaccctctcaaaaaactctcattagggaaaatattaaaagccatctaatatacaagcaattttagtgctcaatacacaaaagacatctttatacaaagaaatttaaatacaacactcagctacttacatgaagtaatggggtttcaactacttctcgcagtttctcaatctgttccttacagccaccaacatcactgtatgtgacatcaggtttctcttccacctaagagagggacagaaatgtacaacatacagtcacaggctttttggatactgtccaaattcaatagaatagacctaataccttaaaagaaaaactcacaagtgagaattttaataaaaattgaatacattgcccagtgccctggaactacttgctgtattaacttgttatgacactaatgaatgaaaaacaaactagtaaaataaagacaggctgagactctgtctcaaaaaatatatatataaaatactgccgggctcagtggctcacgcctggaatcccagcactttgggaggctgaggcaggccaaacacttgaggtcatgagttcgaaaccagtctggccaacatggtgaaacaccgtctctactaaaaatacaaaaattagctgggcgtggtggcgggtgcctgtaatcccagctactcgggaggctgaggcaggagaattgcttgaacccgggaggcagaggttgcagtgagccaagatcacaccattctactccagcctggacaagagtgaaactccgcctcaaaaaaaaaaaaaaaaaaagacatagccttccttttccctcccatagtttcttacctgcatcatggtaactgttgggtcaatcttaggaggcaatggaatgtgaatttgatatttatttctatccacgctaaggaagtaaaaaagatagtcattaatacattctttaagctttcaaagtgcttttatgtttagtcctttatccccaaaaccatactttaagaaaacaagagagagacagagtccctaattatataaagagaaataaagagagcatgaaatctgaggatactaaactacatgtctaatgtcgcaaacaacctatccaacagcaatcaaacctttgaggagggaggggagatttttttttaatttactaagtgagttgttatgtattctatagacattaacacacttagcatcacaactatcctaaagcaagtactcccccaacctataccatgttaccttaaatggactggtaacctcaactctgaaatttgaagccattcataataaacaacagtgggagtaggggagtcagcgaagccgatgggaagttaggaagcagatgcagaagacagaattttcaactatagtacattactagatcatcctttggtattccaaggaactcagtccatttattcactacaaagccaaggatatgaatgtgagacagtatccttagtgaaagaaaagtatttattgtaaatagaaatcttaccccactctcatcccttcttcaatgtcagtaggtgccacctgatcactaaggtccaccacaaacttggcaaactgctttacgttgataatgtattttgggtcctccgaatcagcattgattatctttgtacacctaacacagcaaaaagcttgattagaataaggaacctaagccactaataatgaattcaactaactagattatatcctgagtgtgcaatatacaggtataaactagaactacagaaactgggacactgttaaagatccttttctatacccaactccaaaggtccattcgactttagccaagtcataaaattttcaaatacattctcctggaaaattaaacccaaatatctcatcattaagaagttctctgatttcttgatttccatagtttgtattcctttattattagtgatgctcctgtatatcttaagttgctctcgtttttattttccctacaaaaagatggtcatcttgtcttacaccaaattcagtaataaatgggttttcattcttttcctgagtttatactttaaatccgtttttctttttttttttttttttttttttgagatggagtttcactcttgttgcccaggctgaaacacaatggtgtgatcttggctcactgcaacctccacctcttgggttcatgcaattctcctgcctcagcctcccgagtagctgggattacaggcatgcaccaccatgcctagctaattttgtatttttttttttttttttagtagagagggggtttctcttgaactcccaacctcaggctagtcaggctggtctcgaaatcccaacctcaggtgatccgcccgcctcagcttcccaaagtgctgggattacaggcgtgagccaccacacccggactttaaatccatttttattgtttttgctacttttttctttttttaactgaggcagtagaagcttgtttttttttctttttcttttttttgagacagagtcttgctctggagcccagactggagtgcagtggtgcgatctcggctcattacaatctccgcctcctgggttcaagcagttctcctgcctcagcctcccaagtagctggcattacaggaatgcgccagcacgcccggctaattgttttgtatttttaatatatacggggtttcaccatgttggccaggctggtttcaaactcccgacctcaagtgatccacctgcttcggcctcccaaagtgctgggattacaggcgtgagccattgcacccggccagtagaagctatttttaacttaattatattcatttccatatggcctggtaaaaatgaatttataatgccatgcacatcacagtataaaatagtcaaccgcctttccccttctatagtatctatctactaaagaaaaacaaaaacaaaaaacagaactcacattagaaagctgctacataagccacagcaagaattacagagaaatacaaaaatagcctgtggagtgcgcacataggtagagctaaaagtaaggtacagaatgctttggcttagccaggtgtggtgggatgcacctgtagtcccagctactgaggaggctgaggctggaggatcacttgagcccaggaggtcaaggctaaaaaagaatgctttgtcctgatagcataattgtgtgaaggactctgttttctgttaccaaccatgtttgctagggaagaagaattatacccattctctaccaaaaaaaaagaggctgaccaaaaaacctgaaaggaaaaacaagaacaacaaacagcaataataccaaaccctggggaaaggaagaatttgatttccagaggtgacacattacattattttaaatgtctagttatcaccaaaaagttatgagatgcaaaacattatgaaatgcattttcccttatgtatgacattatggctcatacataaggggaaaaaaatcagtcaatagaaattgtcccttaagaagcctagatgttggtgttattaaacaaaactttttttttttttttgagacggaatctcactgctgcccaggctggagtgcagtggcgcgatcttgactcactgcaacttccgcctcccacgttcgagcaattctcctgcgtcagcctcctaagtgtctgggattacaggcgcctgccaccacgcccagctaatatttttatatttttagtagagacaggtttcaccatgttggccaggctgatctcaaactcccaacctcaagtgatccacctgcctcagcctcccaaagtgctgggattacaggcatgagccactgcgcccagtcaaacaaaaaccttaaatcagtacaagaaatattttcaaaaagctaaaggaaaccatatctaaagagtttataggaatgatgagaagaatgtctcaccaatagtgaatacagatagagaaattagactggaatattatttggccataaaaagttccatctattttagaatacggatgaaccttgaaatatgctaagtgaaggaagccatgtactatatgattccatttagatgatatgtccaggatagacaaattcatagaaacagaaagtagataaatggttgccaggggctggaaagagacgggaatgaagactgctaatgggtattgggtttgtttttggggtgacaaaaatattctattctggaattagatagtgatgatcaatgcacaattttctagatattctaaaaatcactaaattgtatactttaaaagagtgaacctcatgatatctgaattttatatcagtaagattgttattaaaaaaattcataagctggctgggcacggtggctcacacctgtaatcccagaactttgggaggccaaggtgggcagatggcttgagcctaggagttcaagagcagcccaggcaacatggcaagaccctgtctctactaaaaattaaaaaaaaaaaaaaaaaaaaaaaaaaaaaaaagccagacatggggtggtatgcacctgtggtcccagccacttgggaggctgaggtgggagcccaggaagcagaggctgcagcgagctgagatcatgttactgcactccagccttggtaacaaagtgagaccttgtctcaaaaaaaaaaaaaaattcacaagctaaaaattacacatacacaaattttatcaagagttttaagcaaagagtatgagactaaaatcaatggaaactttttcaaaataatctttgctttctttaaggcctgggatcttgtaggggtatagggctctttagctagtctgtctgttatgaaaaatctatatgaaaagtttccctctaactgctttacagaaaaaagaaaattcccctcatacggcctagaaaaatgtacctagattttttaatttaccatagagagataagaacatattacaaccatgatataagagcagaacattcaacacacacacaaaaatgaaagaataaagataagagccttgaaataaaaaacctgagtttttttacctgagaaattaaaaacctacgagaagaaaaatgggaggaaaaaaacatcaacagaaaaattcaaagataaagaaaagctcatagaaagtaaagtaaaaaggcaaaaagaagaaaaacaggaaggaaagattagcaaattagaggagcaatcctggaggccccatatccaaataagagaattcaggaataagggtccaagaaaacggaaggaaagaaattaacaaatagtttcaagaagatttgccttactaaaggacaagagagcccaggacaatggatgaaaacagaccaacaccaaggcacatcaccatgaaatttcaaaatagaacaagagaagactctacaattttcagagaaaaattaggccacatatacaggactgaggatcagaatggcttcagagtaacactgggaggcaaaggacaacagaacgacgccttcaaaattttgaagggaaaagaactgtatactcagctaagacactttcagacatatgcagtctcaaaatattacctcccaactgacccttttccaagaagctaccagaggatgtattagaccaagcaacaggagagaggtaaagggcaatctccagggtagagaagtcagacaacaattgtcattaaacaggcataaaggacaactggtccaaaccaaagcaggtcagaaggtgctgggaaaaacttaaggaaaatgaaaccgagagaacaccagatgcaactaaggtataactgagagaaaacgaacagatctaatgcatctgaagagcagtttagacaatcaagagaaagtgggaggttgagttcataatatatgcacagaaaaccaggcaaatgaaaaaacaagacgttataacaaccaccactaaaagaagtggtacaagaaaggaaaagcaatcacagcttaccacttggctctttgtgaatagtgtttacatagccataatcatataaacactgaatactgatctaaccaaaattataacagaattacataggaaggatgaggggataggaaaggtgtgccttcaaggtgaaggcagaggaaacaaaacaaaatcctcatcttccatgatggaaagccaaaagataatacctaaaactaaaaaatcaagaagtaggaatctataatacacaagatatagcaatatccattagaaccatgaagacgaaaaccaaaataatcagctaaaaaaggtgaaaatgagtgtcattagggaagagaaattgggaaatacagttgttattatttaactgtttaactcttttcttttttttttttttttgagacagagtatcactctgtcaccccaggctgaagtgcagtggcacaatctcagcttactgcaacctctgcctcccaggttcaagcgattctcctgcctcagcctctccagtagctgggattacaagcgcccgccaccacacccagttaatttttgtatttttagtagagacggggtttcactatgttgaccaggctggtcttgaactcctgacctcaagtcatctgcctgcctgggcctcccaaagtgctgggattacaggcgtgagctgccgtgcccagcctgaactatttaacccttcaaaaaccatgataaaactaaaaagtgagtgaataaataaaagtgagaaaagatggaaattgtgaagtagggagggtgagtagatgggaaataaaaataatctttagcttttaggaactaaaaataaaaacaaaatttttaaaaggattataaacagaggagaatatgtttatagccttgaattgggctaggtttttctaagcaacattaacacccaaaaggtataatggaaatattggtatattttatctcctaaaaatgtaaaacattagtacaacaaaaggtaccaagaagttaaaagtgactagggaaaaatacttgtagcatacttaagaaaagacaatatgtacaatatatgaactgtttctaaaattaacaagaaacaacccaagaacaaaacatgagtagtcaaagaaatacagatgtttagtaaacaaaagaagtagctatcatactagtaatcaacacagtacaaattaaaattattttccctcaaatggcaaaagtgtaaaaattgaaaactgcgtatcttacattgatcagacaaatggacatttgcctcaaagcctcttggtaaaagtataaactggccaaagtaattttcacaggattttaaaactataatatctttgacccctagaattccacttgtagtaagctagcctacagaaatgtatgcacatacactgagaaacatttaacacgaatattcactacagcactgtgtatagtaacagaaattgttacagcctaacatctacacattatgcaatttttgcaattttttaaaaacacaagaaaaaagaaataagtatacctatattaaagacaaagattcccaaaacatgtagttatatctaaaaaaaaaaaaatcaggatatgtttctttgcatgcaaatacaggaagatacacagaggattacgagggccaaactatttcagtagttacgtttgaggaaagggcaacgggaaagcaagtatgagaagggagtttttcatttcttgctctacatctttttatatcatttaaatcttttaaatgattatgccttcatatatttttgaagaattgtttaaggcataaaattttaaccccagttttgtagaaagctttatttacttggaatttggaatcaaaattatttatgaactattgcattatccctgagactctaacattaagtgcttgaaaatacagacaaaggtaagttgccacagagcacccgtgcatacctggcaacctgtaaaggctgttcactctggagtgtctgcttatctgcagccaaatcccagagtgctggtggggccaggccagtgtcagattctttaatacctacatagagaaagatgacaaaatttactaaaatgtaatgcatcaccctataataagctttctaaacctcagcactactgatattttgcactgaatcattcattgtgaggactatcatgtgcattatagactgtttagtagcatccctggcctctcaccactagacgccagcagcaccaccctctctcaaagtcatgacaatcaaggatgtctccagacattgcccagtgtcctctgggaggggttgggagggtggggcaaaatcaccccagctgaaaactacaccctgccctaaagtaattgtcaagccaaaaatataatgccacatttgacatttttgtgtaggtcaaatattataaaacttcatcattcaagttccccccaaaaagttccaggaaagagcccaattacttgaagcaaaactatgttgtgagatcttatttttatccaaaaaagttttttgacttgttaactgaagtttactgaactgggttttgaccaacagaaatattactggcttagaagatggtactatttcttatttcttaactgcataagcattaaacctggttgtaaaaagcagtgaaagatatggttaaatgagatctaatttcttaacagaaaggaagaataatgattttttttaaatctgtaatcatgaaggagtaaaatctgtatttaaactcttgatattattcacacattacatttaaaggaaggaaatgggaatttaaaaatacataccagtgagctcattaattttcttgagaagttgctgaatgtcatcttcaacttgcttgatctgcctagagtaagtgctctgaccctaggtgatgaaggtcagtttagttagaatatcaggatatcaaccatttattaaaactacaggtaaaaattatttttaagttacaatttcaatagctatcattatcccaaagcttgtgaagtagagaggtattcctgacccaatatttccccctaaatacacaatccactattatgcaagataacaaatgaaaatcatggccaggcatggtggctcatgcctataatccctgcactttagggggcggaggcgggtggatcacctgaggtcaataattcaagaccagcctggccaacatgatgaaaccctgtctctaccaaaaatacaaaaaattagccagacgtggtggtgggcgctcataatcccagctactcaggaggctgaggcaggagaatcgcttgaacccaggaggcggaggttgcagtgaaccaagatcgcgccattgcactccagcctgggtgacaagagcaacactccgtttcaaaaaaaaaaaaaaaaaaaatcgtgtgtgaaattatgcaaaactatcgatttctatgcattaaaacttcatttcagaactaattcagtaatacttgttaaatatagtaaacttgtgtaaatgatttcaaatatctttcaatatagcatcacattttaatcacgaatagtattaaaataatacataaactgagacatgccaaatactttgatctattctataaactttaggtaggtaacttcatttagtgaagattactagaagctgccagacattcttttctaattgtttctttttattttattttttgtttggttttttaatttttggtttcttaacttcttctaattattttggaaggacaataccaaaacagtttttatttcaacataaaacatctatagtttgtagacactgaaaggacttacataagttttcaacaaggcaatatccccctcatccagagctgaaagaagagagacaatacgtgagattcaaaaagaaactagaattaaaatttttaaaaaagaaactagagctttttttctgttcgattcaaattgttttaagtgagtgtcaaaaatcaagcaagattatgatgatacatattcaatcaaatagttcctggactcaaggaatttttatctaactgggaggaaaataggtttttaaaagtacataatatacgtgatgacctattttatatagtacatacataaaacacatccattaaggatcttggttaaattataccatagtcatacaatgaaatacaatgcaggccccgtgcggtggctcctgcctataatcccagtactttgggaggcccaggcaggcagatcacctgaggtcaggagtttgagaccagcctagccaacctggcgaaaccccgtctctaccaaaaatacaaaaattggcctggcatggtggcgcatgtctgtagtcccagctactcaggaggctgaagctggagaattgcttgaacccaggaggcggaggttgcagtgagcccaaatcacatcaccgcactccagcctgggtgaccgagagagactccatctcaaaaagaaaaagaaaaggaaatacaatgcagcaatttaaaatgataacagaacatagacatagaaagcaatccaacaaatgctattcagtgaatcaggtaggtttaaaaaaaaggatataatgtgcatatattttgtttttatataaaaatcacttaaaacacatgtggagagtaacctgaaatggtgttctacggtggttatctgggatttcaagtgatttacagttaatcttttctattcacaaattctgcatttctgaatttgcttagctgctcaaatttatttgtaatccccaaatcaatactcatgtgctttcatggtcatttgcaaatagacagagtggtaaaaaatttaagttcacaactaagaacaagtcactctaccctcttgtttcagctttcatacagagatgaccagaagatggagaccgtaggggaaatgccgtgtaatccaagcagttcaggctttggggccaggtggacagggtttgaatgccagctctggaatctgttagtggggtgacctcagccaagtcacttaacacttctgaactttgttttctcttttgtgaaataaagaaaaaatgaatctac

SEQ ID NO: 3 - DNA, 2492 bp, Homo sapien
aggcagcggcuguggagcgcggcggggcggcuccgcccagggcagcccgggcugggccaaggagcgagcucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccaggcugugcugucggccauugugauugucaaccugaagggaauguuuaugcaguucucagaucuccccuuuuucuggagaaccagcaaaauagagcugaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcagaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcuuucauacagagaugaccagaagauggagaccguaggggaaaugccguguaauccaagcaguucaggcuuuggggccagguggacaggguuugaaugccagcucuggaaucuguuaguggggugaccucagccaagucacuuaacacuucugaacuuuguuuucucuuuugugaaauaaagaaaaaaugaaucuacaaaaaaaaaaaaaaaaaa

SEQ ID NO: 4 - DNA, 1965 bp, Homo sapien
aggcagcggcuguggagcgcggcggggcggcuccgcccagggcagcccgggcugggccaaggagcgagcucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccaggcugugcugucggccauugugauugucaaccugaagggaauguuuaugcaguucucagaucuccccuuuuucuggagaaccagcaaaauagagcugaccaucuggcuuaccacuuuuguguccuccuuguuccugggauuggacuaugguuugaucacugcugugaucauugcucugcugacugugauuuacagaacacagagcuuucauacagagaugaccagaagauggagaccguaggggaaaugccguguaauccaagcaguucaggcuuuggggccagguggacaggguuugaaugccagcucuggaaucuguuaguggggugaccucagccaagucacuuaacacuucugaacuuuguuuucucuuuugugaaauaaagaaaaaaugaaucuacaaaaaaaaaaaaaaaaaa

SEQ ID NO: 5 - DNA, 1422 bp, Homo sapien
aggcagcggcuguggagcgcggcggggcggcuccgcccagggcagcccgggcugggccaaggagcgagcucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuaggcuuucauacagagaugaccagaagauggagaccguaggggaaaugccguguaauccaagcaguucaggcuuuggggccagguggacaggguuugaaugccagcucuggaaucuguuaguggggugaccucagccaagucacuuaacacuucugaacuuuguuuucucuuuugugaaauaaagaaaaaaugaaucuacaaaaaaaaaaaaaaaaaa

SEQ ID NO: 6 - DNA, 2671 bp, Homo sapien
aggcagcggcuguggagcgcggcggggcggcuccgcccagggcagcccgggcugggccaaggagcgagcucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccaggcugugcugucggccauugugauugucaaccugaagggaauguuuaugcaguucucagaucuccccuuuuucuggagaaccagcaaaauagagcugaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcagaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggaugggguuauaagccucucaugaaguucauaauuuacacguuuuaaauacuagacgcuagauuuuuuuuucuaagggugaauacuaguaguccaggcuugauuuggagggugaaugacgccuagcaagauguauuguacuuguguuuuuuuaauugaauacuucaaagauaaaaaaaaaaa

SEQ ID NO: 7 - DNA, 2374 bp, Homo sapien
ucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccagaccaucuggcuuaccacuuuuguguccuccuuguuccugggauuggacuaugguuugaucacugcugugaucauugcucugcugacugugauuuacagaacacagaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcagaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggaugggguuauaagccucucaugaaguucauaauuuacacguuuuaaauacuagacgcuagauuuuuuuuucuaagggugaauacuaguaguccaggcuugauuuggagggugaaugacg

SEQ ID NO: 8 - DNA, 2444 bp, Homo sapien
ucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccagaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcagaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggaugggguuauaagccucucaugaaguucauaauuuacacguuuuaaauacuagacgcuagauuuuuuuuucuaagggugaauacuaguaguccaggcuugauuuggagggugaaugacg

SEQ ID NO: 9 - DNA, 2188 bp, Homo sapien
ucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagaccaucuggcuuaccacuuuuguguccuccuuguuccugggauuggacuaugguuugaucacugcugugaucauugcucugcugacugugauuuacagaacacagaggugaaagaaauuccuggaauaaaaauauuucaaauaaaugcaccaauuuacuaugcaaauagcgacuuguauagcaaugcauuaaaacgaaaggaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggaugggguuauaagccucucaugaaguucauaauuuacacguuuuaaauacuagacgcuagauuuuuuuuucuaagggugaauacuaguaguccaggcuugauuuggagggugaaugacg

SEQ ID NO: 10 - DNA, 2275 bp, Homo sapien
agaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccaggcugugcugucggccauugugauugucaaccugaagggaauguuuaugcaguucucagaucuccccuuuuucuggagaaccagcaaaauagagcugaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcaacacaggaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggauaagcc

SEQ ID NO: 11 - DNA, 2179 bp, Homo sapien
agaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccagaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcaacacaggaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggauaagcc

SEQ ID NO: 12 - DNA, 1554 bp, Homo sapien
guucguugcaacaaauugaugagcaaugcuuuuuuauaaugccaacuuuguacaaaaaaguuggcaccauggaucaugcugaagaaaaugaaauccuugcagcaacccagagguacuauguggaaaggccuaucuuuagucauccgguccuccaggaaagacuacacacaaaggacaagguuccugauuccauugcggauaagcugaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccagaccaucuggcuuaccacuuuuguguccuccuuguuccugggauuggacuaugguuugaucacugcugugaucauugcucugcugacugugauuuacagaacacagagguugccaacuuucuuguacaaaguuggcauuauaagaaagcauugcuuaucaauuuguugcaacgaac

SEQ ID NO: 13 - DNA, 2492 bp, Homo sapien
aggcagcggcuguggagcgcggcggggcggcuccgcccagggcagcccgggcugggccaaggagcgagcucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccaggcugugcugucggccauugugauugucaaccugaagggaauguuuaugcaguucucagaucuccccuuuuucuggagaaccagcaaaauagagcugaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcagaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcuuucauacagagaugaccagaagauggagaccguaggggaaaugccguguaauccaagcaguucaggcuuuggggccagguggacaggguuugaaugccagcucuggaaucuguuaguggggugaccucagccaagucacuuaacacuucugaacuuuguuuucucuuuugugaaauaaagaaaaaaugaaucuacaaaaaaaaaaaaaaaaaa

SEQ ID NO: 14 - DNA, 1965 bp, Homo sapien
aggcagcggcuguggagcgcggcggggcggcuccgcccagggcagcccgggcugggccaaggagcgagcucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccaggcugugcugucggccauugugauugucaaccugaagggaauguuuaugcaguucucagaucuccccuuuuucuggagaaccagcaaaauagagcugaccaucuggcuuaccacuuuuguguccuccuuguuccugggauuggacuaugguuugaucacugcugugaucauugcucugcugacugugauuuacagaacacagagcuuucauacagagaugaccagaagauggagaccguaggggaaaugccguguaauccaagcaguucaggcuuuggggccagguggacaggguuugaaugccagcucuggaaucuguuaguggggugaccucagccaagucacuuaacacuucugaacuuuguuuucucuuuugugaaauaaagaaaaaaugaaucuacaaaaaaaaaaaaaaaaaa

SEQ ID NO: 15 - DNA, 1422 bp, Homo sapien
aggcagcggcuguggagcgcggcggggcggcuccgcccagggcagcccgggcugggccaaggagcgagcucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuaggcuuucauacagagaugaccagaagauggagaccguaggggaaaugccguguaauccaagcaguucaggcuuuggggccagguggacaggguuugaaugccagcucuggaaucuguuaguggggugaccucagccaagucacuuaacacuucugaacuuuguuuucucuuuugugaaauaaagaaaaaaugaaucuacaaaaaaaaaaaaaaaaaa

SEQ ID NO: 16 - DNA, 2671 bp, Homo sapien
aggcagcggcuguggagcgcggcggggcggcuccgcccagggcagcccgggcugggccaaggagcgagcucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccaggcugugcugucggccauugugauugucaaccugaagggaauguuuaugcaguucucagaucuccccuuuuucuggagaaccagcaaaauagagcugaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcagaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggaugggguuauaagccucucaugaaguucauaauuuacacguuuuaaauacuagacgcuagauuuuuuuuucuaagggugaauacuaguaguccaggcuugauuuggagggugaaugacgccuagcaagauguauuguacuuguguuuuuuuaauugaauacuucaaagauaaaaaaaaaaa

SEQ ID NO: 17 - DNA, 2374 bp, Homo sapien
ucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccagaccaucuggcuuaccacuuuuguguccuccuuguuccugggauuggacuaugguuugaucacugcugugaucauugcucugcugacugugauuuacagaacacagaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcagaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggaugggguuauaagccucucaugaaguucauaauuuacacguuuuaaauacuagacgcuagauuuuuuuuucuaagggugaauacuaguaguccaggcuugauuuggagggugaaugacg

SEQ ID NO: 18 - DNA, 2444 bp, Homo sapien
ucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccagaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcagaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggaugggguuauaagccucucaugaaguucauaauuuacacguuuuaaauacuagacgcuagauuuuuuuuucuaagggugaauacuaguaguccaggcuugauuuggagggugaaugacg

SEQ ID NO: 19 - DNA, 2188 bp, Homo sapien
ucucccuucuccugcucucagccucagugaucaaggcuucagugaacugcacuggagcucccagcgggggaucuuguccccugucccgacuuuugugcugcacauuggaucuggugacacucaggaaaugcuugucuccggcuguuaaggaauaauuucagaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagaccaucuggcuuaccacuuuuguguccuccuuguuccugggauuggacuaugguuugaucacugcugugaucauugcucugcugacugugauuuacagaacacagaggugaaagaaauuccuggaauaaaaauauuucaaauaaaugcaccaauuuacuaugcaaauagcgacuuguauagcaaugcauuaaaacgaaaggaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggaugggguuauaagccucucaugaaguucauaauuuacacguuuuaaauacuagacgcuagauuuuuuuuucuaagggugaauacuaguaguccaggcuugauuuggagggugaaugacg

SEQ ID NO: 20 - DNA, 2275 bp, Homo sapien
agaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccaggcugugcugucggccauugugauugucaaccugaagggaauguuuaugcaguucucagaucuccccuuuuucuggagaaccagcaaaauagagcugaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcaacacaggaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggauaagcc

SEQ ID NO: 21 - DNA, 2179 bp, Homo sapien
agaguacuauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccagaccaucuggcuuaccacuuuuguguccuccuuguuccugggauuggacuaugguuugaucacugcugugaucauugcucugcugacugugauuuacagaacacagaguccaagcuacaaaguccuuggaaagcuuccugaaacugauguguauauugauauagacgcauaugaggaggugaaagaaauuccuggaauaaaaauauuucaaauaaaugcaccaauuuacuaugcaaauagcgacuuguauagcaaugcauuaaaacgaaagacuggagugaacccagcagucaucaugggagcaaggagaaaggccaugcggaaguacgcuaaggaagucggaaaugcaaauauggccaacgcaacuguugucaaagcaacacaggaugcagaaguagauggagaggaugcuaccaagccugaagaagaggauggugaaguaaaauaucccccaauagugaucaaaagcacauuuccugaggaaaugcaaagauuuaugcccccaggggauaacguccacacugucauuuuggauuucacucaagucaauuuuauugauucuguuggagugaaaacucuggcagggauuguaaaagaauauggagacgucgguauauauguauacuuagcaggaugcagugcacaaguugugaaugaccucacucggaauagauuuuuugaaaauccugcccuaugggagcugcuguuccacagcauucaugaugcaguuuuaggcagccaacuuagagaggcacuugcugaacaggaagccucggcucccccuucccaggaggacuuggagcccaaugccacuccugccacuccugaggcauagaugaggaccucacccuaggauaagcc

SEQ ID NO: 22 - DNA, 1554 bp, Homo sapien
guucguugcaacaaauugaugagcaaugcuuuuuuauaaugccaacuuuguacaaaaaaguuggcaccauggaucaugcugaagaaaaugaaauccuugcagcaacccagagguacuauguggaaaggccuaucuuuagucauccgguccuccaggaaagacuacacacaaaggacaagguuccugauuccauugcggauaagccgaaacaggcauucacauguacuccuaaaaaaauaagaaauaucauuuauauguuccuacccauaacuaaauggcugccagcauacaaauucaaggaauauguguugggugacuuggucucaggcauaagcacaggggugcuucagcuuccucaaggcuuagccuuugcaaugcuggcagcugugccuccaauauuuggccuguacucuucauuuuacccuguuaucauguauuguuuucuuggaaccuccagacacauauccauagguccuuuugcuguuauuagccugaugauuggugguguagcuguucgauuaguaccagaugauauagucauuccaggaggaguaaaugcaaccaauggcacagaggccagagaugccuugagagugaaagucgccaugucugugaccuuacuuucaggaaucauucaguuuugccuaggugucuguagguuuggauuuguggccauauaucucacagagccucugguccguggguuuaccaccgcagcagcugugcaugucuucaccuccauguuaaaauaucuguuuggaguuaaaacaaagcgguacaguggaaucuuuuccgugguguauaguacaguugcuguguugcagaauguuaaaaaccucaacguguguucccuaggcgucgggcugaugguuuuugguuugcuguuggguggcaaggaguuuaaugagagauuuaaagagaaauugccggcgccuauuccuuuagaguucuuugcggucguaaugggaacuggcauuucagcuggguuuaacuugaaagaaucauacaauguggaugucguuggaacacuuccucuagggcugcuaccuccagccaauccggacaccagccucuuccaccuuguguacguagaugccauugccauagccaucguuggauuuucagugaccaucuccauggccaagaccuuagcaaauaaacauggcuaccagguugacggcaaucaggagcucauugcccugggacugugcaauuccauuggcucacucuuccagaccuuuucaauuucaugcuccuugucucgaagccuuguucaggagggaaccggugggaagacacagcuugcagguuguuuggccucauuaaugauucugcuggucauauuagcaacuggauuccucuuugaaucauugccccagaccaucuggcuuaccacuuuuguguccuccuuguuccugggauuggacuaugguuugaucacugcugugaucauugcucugcugacugugauuuacagaacacagagguugccaacuuucuuguacaaaguuggcauuauaagaaagcauugcuuaucaauuuguugcaacgaac

SEQ ID NO: 23 - DNA, 2492 bp, Homo sapien
aggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccaggctgtgctgtcggccattgtgattgtcaacctgaagggaatgtttatgcagttctcagatctcccctttttctggagaaccagcaaaatagagctgaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcagatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgctttcatacagagatgaccagaagatggagaccgtaggggaaatgccgtgtaatccaagcagttcaggctttggggccaggtggacagggtttgaatgccagctctggaatctgttagtggggtgacctcagccaagtcacttaacacttctgaactttgttttctcttttgtgaaataaagaaaaaatgaatctacaaaaaaaaaaaaaaaaaa

SEQ ID NO: 24 - DNA, 1965 bp, Homo sapien
aggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccaggctgtgctgtcggccattgtgattgtcaacctgaagggaatgtttatgcagttctcagatctcccctttttctggagaaccagcaaaatagagctgaccatctggcttaccacttttgtgtcctccttgttcctgggattggactatggtttgatcactgctgtgatcattgctctgctgactgtgatttacagaacacagagctttcatacagagatgaccagaagatggagaccgtaggggaaatgccgtgtaatccaagcagttcaggctttggggccaggtggacagggtttgaatgccagctctggaatctgttagtggggtgacctcagccaagtcacttaacacttctgaactttgttttctcttttgtgaaataaagaaaaaatgaatctacaaaaaaaaaaaaaaaaaa

SEQ ID NO: 25 - DNA, 1422 bp, Homo sapien
aggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctaggctttcatacagagatgaccagaagatggagaccgtaggggaaatgccgtgtaatccaagcagttcaggctttggggccaggtggacagggtttgaatgccagctctggaatctgttagtggggtgacctcagccaagtcacttaacacttctgaactttgttttctcttttgtgaaataaagaaaaaatgaatctacaaaaaaaaaaaaaaaaaa

SEQ ID NO: 26 - DNA, 2671 bp, Homo sapien
aggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccaggctgtgctgtcggccattgtgattgtcaacctgaagggaatgtttatgcagttctcagatctcccctttttctggagaaccagcaaaatagagctgaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcagatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacgcctagcaagatgtattgtacttgtgtttttttaattgaatacttcaaagataaaaaaaaaaa

SEQ ID NO: 27 - DNA, 2374 bp, Homo sapien
tctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccagaccatctggcttaccacttttgtgtcctccttgttcctgggattggactatggtttgatcactgctgtgatcattgctctgctgactgtgatttacagaacacagaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcagatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacg

SEQ ID NO: 28 - DNA, 2444 bp, Homo sapien
tctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccagaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcagatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacg

SEQ ID NO: 29 - DNA, 2188 bp, Homo sapien
tctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagaccatctggcttaccacttttgtgtcctccttgttcctgggattggactatggtttgatcactgctgtgatcattgctctgctgactgtgatttacagaacacagaggtgaaagaaattcctggaataaaaatatttcaaataaatgcaccaatttactatgcaaatagcgacttgtatagcaatgcattaaaacgaaaggatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacg

SEQ ID NO: 30 - DNA, 2275 bp, Homo sapien
agagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccaggctgtgctgtcggccattgtgattgtcaacctgaagggaatgtttatgcagttctcagatctcccctttttctggagaaccagcaaaatagagctgaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcaacacaggatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggataagcc

SEQ ID NO: 31 - DNA, 2179 bp, Homo sapien
agagtactatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccagaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcaacacaggatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggataagcc

SEQ ID NO: 32 - DNA, 1554 bp, Homo sapien
gttcgttgcaacaaattgatgagcaatgcttttttataatgccaactttgtacaaaaaagttggcaccatggatcatgctgaagaaaatgaaatccttgcagcaacccagaggtactatgtggaaaggcctatctttagtcatccggtcctccaggaaagactacacacaaaggacaaggttcctgattccattgcggataagctgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccagaccatctggcttaccacttttgtgtcctccttgttcctgggattggactatggtttgatcactgctgtgatcattgctctgctgactgtgatttacagaacacagaggttgccaactttcttgtacaaagttggcattataagaaagcattgcttatcaatttgttgcaacgaac

SEQ ID NO: 33 - DNA, 2492 bp, Homo sapien
aggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccaggctgtgctgtcggccattgtgattgtcaacctgaagggaatgtttatgcagttctcagatctcccctttttctggagaaccagcaaaatagagctgaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcagatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgctttcatacagagatgaccagaagatggagaccgtaggggaaatgccgtgtaatccaagcagttcaggctttggggccaggtggacagggtttgaatgccagctctggaatctgttagtggggtgacctcagccaagtcacttaacacttctgaactttgttttctcttttgtgaaataaagaaaaaatgaatctacaaaaaaaaaaaaaaaaaa

SEQ ID NO: 34 - DNA, 1965 bp, Homo sapien
aggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccaggctgtgctgtcggccattgtgattgtcaacctgaagggaatgtttatgcagttctcagatctcccctttttctggagaaccagcaaaatagagctgaccatctggcttaccacttttgtgtcctccttgttcctgggattggactatggtttgatcactgctgtgatcattgctctgctgactgtgatttacagaacacagagctttcatacagagatgaccagaagatggagaccgtaggggaaatgccgtgtaatccaagcagttcaggctttggggccaggtggacagggtttgaatgccagctctggaatctgttagtggggtgacctcagccaagtcacttaacacttctgaactttgttttctcttttgtgaaataaagaaaaaatgaatctacaaaaaaaaaaaaaaaaaa

SEQ ID NO: 35 - DNA, 1422 bp, Homo sapien
aggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctaggctttcatacagagatgaccagaagatggagaccgtaggggaaatgccgtgtaatccaagcagttcaggctttggggccaggtggacagggtttgaatgccagctctggaatctgttagtggggtgacctcagccaagtcacttaacacttctgaactttgttttctcttttgtgaaataaagaaaaaatgaatctacaaaaaaaaaaaaaaaaaa

SEQ ID NO: 36 - DNA, 2671 bp, Homo sapien
aggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccaggctgtgctgtcggccattgtgattgtcaacctgaagggaatgtttatgcagttctcagatctcccctttttctggagaaccagcaaaatagagctgaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcagatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacgcctagcaagatgtattgtacttgtgtttttttaattgaatacttcaaagataaaaaaaaaaa

SEQ ID NO: 37 - DNA, 2374 bp, Homo sapien
tctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccagaccatctggcttaccacttttgtgtcctccttgttcctgggattggactatggtttgatcactgctgtgatcattgctctgctgactgtgatttacagaacacagaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcagatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacg

SEQ ID NO: 38 - DNA, 2444 bp, Homo sapien
tctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccagaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcagatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacg

SEQ ID NO: 39 - DNA, 2188 bp, Homo sapien
tctcccttctcctgctctcagcctcagtgatcaaggcttcagtgaactgcactggagctcccagcgggggatcttgtcccctgtcccgacttttgtgctgcacattggatctggtgacactcaggaaatgcttgtctccggctgttaaggaataatttcagagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagaccatctggcttaccacttttgtgtcctccttgttcctgggattggactatggtttgatcactgctgtgatcattgctctgctgactgtgatttacagaacacagaggtgaaagaaattcctggaataaaaatatttcaaataaatgcaccaatttactatgcaaatagcgacttgtatagcaatgcattaaaacgaaaggatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggatggggttataagcctctcatgaagttcataatttacacgttttaaatactagacgctagatttttttttctaagggtgaatactagtagtccaggcttgatttggagggtgaatgacg

SEQ ID NO: 40 - DNA, 2275 bp, Homo sapien
agagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccaggctgtgctgtcggccattgtgattgtcaacctgaagggaatgtttatgcagttctcagatctcccctttttctggagaaccagcaaaatagagctgaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcaacacaggatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggataagcc

SEQ ID NO: 41 - DNA, 2179 bp, Homo sapien
agagtactatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccagaccatctggcttaccacttttgtgtcctccttgttcctgggattggactatggtttgatcactgctgtgatcattgctctgctgactgtgatttacagaacacagagtccaagctacaaagtccttggaaagcttcctgaaactgatgtgtatattgatatagacgcatatgaggaggtgaaagaaattcctggaataaaaatatttcaaataaatgcaccaatttactatgcaaatagcgacttgtatagcaatgcattaaaacgaaagactggagtgaacccagcagtcatcatgggagcaaggagaaaggccatgcggaagtacgctaaggaagtcggaaatgcaaatatggccaacgcaactgttgtcaaagcaacacaggatgcagaagtagatggagaggatgctaccaagcctgaagaagaggatggtgaagtaaaatatcccccaatagtgatcaaaagcacatttcctgaggaaatgcaaagatttatgcccccaggggataacgtccacactgtcattttggatttcactcaagtcaattttattgattctgttggagtgaaaactctggcagggattgtaaaagaatatggagacgtcggtatatatgtatacttagcaggatgcagtgcacaagttgtgaatgacctcactcggaatagattttttgaaaatcctgccctatgggagctgctgttccacagcattcatgatgcagttttaggcagccaacttagagaggcacttgctgaacaggaagcctcggctcccccttcccaggaggacttggagcccaatgccactcctgccactcctgaggcatagatgaggacctcaccctaggataagcc

SEQ ID NO: 42 - DNA, 1554 bp, Homo sapien
gttcgttgcaacaaattgatgagcaatgcttttttataatgccaactttgtacaaaaaagttggcaccatggatcatgctgaagaaaatgaaatccttgcagcaacccagaggtactatgtggaaaggcctatctttagtcatccggtcctccaggaaagactacacacaaaggacaaggttcctgattccattgcggataagccgaaacaggcattcacatgtactcctaaaaaaataagaaatatcatttatatgttcctacccataactaaatggctgccagcatacaaattcaaggaatatgtgttgggtgacttggtctcaggcataagcacaggggtgcttcagcttcctcaaggcttagcctttgcaatgctggcagctgtgcctccaatatttggcctgtactcttcattttaccctgttatcatgtattgttttcttggaacctccagacacatatccataggtccttttgctgttattagcctgatgattggtggtgtagctgttcgattagtaccagatgatatagtcattccaggaggagtaaatgcaaccaatggcacagaggccagagatgccttgagagtgaaagtcgccatgtctgtgaccttactttcaggaatcattcagttttgcctaggtgtctgtaggtttggatttgtggccatatatctcacagagcctctggtccgtgggtttaccaccgcagcagctgtgcatgtcttcacctccatgttaaaatatctgtttggagttaaaacaaagcggtacagtggaatcttttccgtggtgtatagtacagttgctgtgttgcagaatgttaaaaacctcaacgtgtgttccctaggcgtcgggctgatggtttttggtttgctgttgggtggcaaggagtttaatgagagatttaaagagaaattgccggcgcctattcctttagagttctttgcggtcgtaatgggaactggcatttcagctgggtttaacttgaaagaatcatacaatgtggatgtcgttggaacacttcctctagggctgctacctccagccaatccggacaccagcctcttccaccttgtgtacgtagatgccattgccatagccatcgttggattttcagtgaccatctccatggccaagaccttagcaaataaacatggctaccaggttgacggcaatcaggagctcattgccctgggactgtgcaattccattggctcactcttccagaccttttcaatttcatgctccttgtctcgaagccttgttcaggagggaaccggtgggaagacacagcttgcaggttgtttggcctcattaatgattctgctggtcatattagcaactggattcctctttgaatcattgccccagaccatctggcttaccacttttgtgtcctccttgttcctgggattggactatggtttgatcactgctgtgatcattgctctgctgactgtgatttacagaacacagaggttgccaactttcttgtacaaagttggcattataagaaagcattgcttatcaatttgttgcaacgaac

SEQ ID NO: 43 - protein, 744 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 44 - protein, 685 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAFIQR

SEQ ID NO: 45 - protein, 516 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSFHTEMTRRWRP

SEQ ID NO: 46 - protein, 335 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGFHTEMTRRWRP

SEQ ID NO: 47 - protein, 712 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 48 - protein, 714 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKATQDAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 49 - protein, 473 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQR

SEQ ID NO: 50 - protein, 447 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQTIWLTTFVSSLFLGLDYGLITAVIIALLTVIYRTQR

SEQ ID NO: 51 - protein, 746 aa, Homo sapien
MDHAEENEILAATQRYYVERPIFSHPVLQERLHTKDKVPDSIADKLKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKATQDAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 52 - protein, 744 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 53 - protein, 685 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAFIQR

SEQ ID NO: 54 - protein, 516 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSFHTEMTRRWRP

SEQ ID NO: 55 - protein, 335 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGFHTEMTRRWRP

SEQ ID NO: 56 - protein, 712 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKADAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 57 - protein, 714 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKATQDAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA

SEQ ID NO: 58 - protein, 473 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQTIWLTTFVSSLFLGLDYGLITAVIIALLTVIYRTQR

SEQ ID NO: 59 - protein, 447 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQTIWLTTFVSSLFLGLDYGLITAVIIALLTVIYRTQR

SEQ ID NO: 60 - protein, 746 aa, Homo sapien
MDHAEENEILAATQRYYVERPIFSHPVLQERLHTKDKVPDSIADKPKQAFTCTPKKIRNIIYMFLPITKWLPAYKFKEYVLGDLVSGISTGVLQLPQGLAFAMLAAVPPIFGLYSSFYPVIMYCFLGTSRHISIGPFAVISLMIGGVAVRLVPDDIVIPGGVNATNGTEARDALRVKVAMSVTLLSGIIQFCLGVCRFGFVAIYLTEPLVRGFTTAAAVHVFTSMLKYLFGVKTKRYSGIFSVVYSTVAVLQNVKNLNVCSLGVGLMVFGLLLGGKEFNERFKEKLPAPIPLEFFAVVMGTGISAGFNLKESYNVDVVGTLPLGLLPPANPDTSLFHLVYVDAIAIAIVGFSVTISMAKTLANKHGYQVDGNQELIALGLCNSIGSLFQTFSISCSLSRSLVQEGTGGKTQLAGCLASLMILLVILATGFLFESLPQAVLSAIVIVNLKGMFMQFSDLPFFWRTSKIELTIWLTTFVSSLFLGLDYGLITAVIIALLTVIYRTQSPSYKVLGKLPETDVYIDIDAYEEVKEIPGIKIFQINAPIYYANSDLYSNALKRKTGVNPAVIMGARRKAMRKYAKEVGNANMANATVVKATQDAEVDGEDATKPEEEDGEVKYPPIVIKSTFPEEMQRFMPPGDNVHTVILDFTQVNFIDSVGVKTLAGIVKEYGDVGIYVYLAGCSAQVVNDLTRNRFFENPALWELLFHSIHDAVLGSQLREALAEQEASAPPSQEDLEPNATPATPEA